(19) United States
(12) Patent Application Publication
(10) Pub. No.: US 2009/0191193 A1
(43) Pub. Date: Jul. 30, 2009

(54) Aryl Vinyl Sulfides, Sulfones, Sulfoxides and Sulfonamides, Derivatives Thereof and Therapeutic Uses Thereof

(75) Inventors: E. Premkumar Reddy, Villanova, PA (US); M.V. Ramana Reddy, Upper Darby, PA (US)

Correspondence Address: DRINKER BIDDLE & REATH;ATTN: INTELLECTUAL PROPERTY GROUP, ONE LOGAN SQUARE, 18TH AND CHERRY STREETS, PHILADELPHIA, PA, 19103-6996, US

(73) Assignee: TEMPLE UNIVERSITY-OF THE COMMONWEALTH SYSTEM OF HIGHER EDUCATION, PHILADELPHIA, PA (US)

(21) Appl. No.: 12/309,620

(22) PCT Filed: Aug. 1, 2007
(86) PCT No.: PCT/US2007/017266

§ 371 Date: Jan. 22, 2009

Related U.S. Application Data
(60) Provisional application No. 60/835,146, filed on Aug. 2, 2006.

(51) Int. Cl. A61K 39/395; C07C 381/00; C07K 16/00; A61P 35/00

(52) U.S. Cl. 424/133.1; 564/440; 530/387.3

(57) ABSTRACT

Compounds useful as antiproliferative agents, including, for example, anticancer agents, according to formula I:

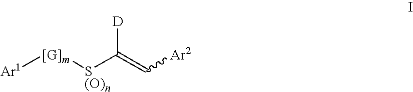

wherein Ar¹, Ar², D, G, [symbol] m and n are as defined herein, salts, antibody conjugates, pharmaceutical compositions, methods of treatment, synthetic processes, and intermediates useful in such processes are provided.

### CLAIM OF PRIORITY

This application claims the benefit of priority of U.S. Provisional Application Ser. No. 60/835,146, filed Aug. 2, 2006, which is incorporated herein by reference in its entirety.

### FIELD OF THE INVENTION

The invention relates to compounds, methods for their preparation, compositions including them and methods for the treatment of cellular proliferative disorders, including, but not limited to, cancer.

### BACKGROUND OF THE INVENTION

Cellular proliferative orders such as cancer are among the most common causes of death in developed countries. For diseases for which treatments exist, despite continuing advances, the existing treatments often have undesirable side effects and limited efficacy. Identifying new effective drugs for cellular proliferative disorders, including cancer, is a continuing focus of medical research.

### SUMMARY OF THE INVENTION

It has been found that certain compounds and compositions are useful for the treatment of cancer and other cellular proliferative disorders. The biologically active compounds of the invention are in the form of α,β-unsaturated sulfones, sulfoxides, thioethers, and sulfonamides.

In one aspect, the invention is a compound of formula I, or a salt thereof:

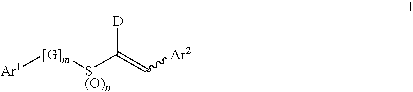

wherein:

Ar¹ is:

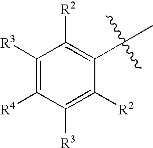

Ar² is selected from the group consisting of unsubstituted aryl, substituted aryl, unsubstituted heteroaryl and substituted heteroaryl; wherein, when Ar² is substituted aryl or substituted heteroaryl, said substituted aryl or substituted heteroaryl is aryl or heteroaryl substituted with one or more substituents independently selected from the group consisting of halogen, (C₁-C₆)alkyl, (C₂-C₆)alkenyl, (C₂-C₆)alkynyl, —(C₁-C₃)alkylene-Ar³, —OR⁷, —OAr³, —O(C₁-C₃)alkylene-Ar³, (C₁-C₃)fluoroalkoxy, —NO₂, —C≡N, —C(═O)(C₁-C₃)alkyl, —C(═O)OR⁷, —C(═O)NR₂, C(═NR⁷)NR⁸₂, —(C₁-C₃)alkylene-C(═O)OR⁷, —O(C₁-C₃)alkylene-C(═O)OR⁷, —(C₁-C₆)alkylene-OR⁷, —NR⁸₂, —P(═O)(OR⁷)₂, —OP(═O)(OR⁷)₂, —S(C₁-C₆)alkyl, —S(O)(C₁-C₆)alkyl, —SO₂(C₁-C₆)alkyl, —SO₂NR⁸₂, —OSO₂(C₁-C₆)alkyl, —OSO₂Ar³, —NHC(═O)(C₁-C₆)alkyl, —OC(═O)(C₁-C₃)alkyl, —O(C₂-C₆)alkylene-NR⁸₂ and (C₁-C₃)perfluoroalkyl;

D is —C≡N, —C(═O)NR⁸₂, or NO₂;

G is CR¹₂ or NR¹;

R¹ is independently selected from the group consisting of hydrogen and (C₁-C₆)alkyl;

each R² is independently selected from the group consisting of hydrogen, (C₁-C₆)alkyl, halogen, —OR⁷, —OAr³, —O(C₁-C₃)alkylene-Ar³, —C≡N, —NO₂, —C(═O)OR⁷, —C(═O)NR⁸₂, —C(═NR⁷)NR⁸₂, —(C₁-C₃)alkylene-C(═O)OR⁷, —O(C₁-C₃)alkylene-C(═O)OR⁷, —(C₁-C₆)alkylene-OR⁷, —P(═O)(OR⁷)₂, —OP(═O)(OR⁷)₂, —NHC(═O)(C₁-C₆)alkyl, —SO₂NR⁸₂, —OSO₂(C₁-C₆)alkyl, —OSO₂Ar³, —OC(═O)(C₁-C₃)alkyl, —O(C₂-C₆)alkylene-NR⁸₂, (C₁-C₃)perfluoroalkyl and —N(R⁶)-(M)_{y}-R⁵;

each R³ is independently selected from the group consisting of hydrogen, (C₁-C₆)alkyl, halogen, —OR⁷, —OAr³, —O(C₁-C₃)alkylene-Ar³, —C≡N, —NO₂, —C(═O)OR⁷, —C(═O)NR⁸₂, —C(═NR⁷)NR⁸₂, —(C₁-C₃)alkylene-C(═O)OR⁷, —O(C₁-C₃)alkylene-C(═O)OR⁷, —(C₁-C₆)alkylene-R⁷, —P(═O)(OR⁷)₂, —OP(═O)(OR⁷)₂, —NHC(═O)(C₁-C₆)alkyl, —SO₂NR⁸₂, —OSO₂(C₁-C₆)alkyl, —OSO₂Ar³, —OC(═O)(C₁-C₃)alkyl, —O(C₂-C₆)alkylene-NR⁸₂, (C₁-C₃)perfluoroalkyl and —N(R⁶)-(M)_{y}-R⁵;

R⁴ is selected from the group consisting of hydrogen, (C₁-C₆)alkyl, halogen, —OR⁷, —OAr³, —O(C₁-C₃)alkylene-Ar³, —C≡N, —NO₂, —C(═O)OR⁷, —C(═O)NR⁸₂, —C(═NR⁷)NR⁸₂, —(C₁-C₃)alkylene-C(═O)OR⁷, —O(C₁-C₃)alkylene-C(═O)OR⁷, —(C₁-C₆)alkylene-OR⁷, —P(═O)(OR⁷)₂, —OP(═O)(OR⁷)₂, —NHC(═O)(C₁-C₆)alkyl, —SO₂NR⁸₂, —OSO₂(C₁-C₆)alkyl, —OSO₂Ar³, —OC(═O)(C₁-C₃)alkyl, —O(C₂-C₆)alkylene-NR⁸₂, (C₁-C₃)perfluoroalkyl, and —N(R⁶)M_{y}(R⁵);

each Ar³ is phenyl optionally substituted with 1, 2, 3, 4, or 5 substituents independently selected from the group consisting of (C₁-C₃)alkyl, (C₁-C₃)alkoxy and halogen;

each M is a connecting group independently selected from the group consisting of —(C₁-C₆)alkylene-, —(CH₂)_{d}—V—(CH₂)_{d}—, —(CH₂)_{f}—W—(CH₂)_{g}— and -Z-;

each y is independently selected from the group consisting of 0 and 1;

each V is independently selected from the group consisting of arylene, heteroarylene, —C(═O)—, —C(═O)(C₁-C₆)perfluoroalkylene, —C(═O)—, —C(═S)—, —S(═O)—, —SO₂—, —C(═O)NR⁷—, —C(═S)NR⁷— and —SO₂NR⁷—;

each W is independently selected from the group consisting of —NR⁷—, —O— and each d is independently selected from the group consisting of 0, 1 and 2;

each e is independently selected from the group consisting of 0, 1 and 2;

each f is independently selected from the group consisting of 1, 2 and 3;

each g is independently selected from the group consisting of 0, 1 and 2;

-Z- is

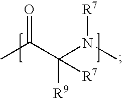

wherein the absolute configuration of -Z- is (S) or (R), or a mixture of (s) and (R);

each R⁵ is independently selected from the group consisting of hydrogen, unsubstituted aryl, substituted aryl, unsubstituted heterocyclic, substituted heterocyclic, —C(═O)OR⁷, —C(═O)NR⁸₂, —C(═NH)—NR⁸₂, —(C₁-C₆)perfluoroalkyl, —CF₂Cl, —P(═O)(OR⁷)₂, —CR⁷R¹⁰R¹¹ and a monovalent peptidyl group with a molecular weight of less than 1000, preferably with a molecular weight of less than 800, more preferably with a molecular weight of less than 600, most preferably with a molecular weight of less than 400, provided that when y is 0, R⁵ is not C(═O)OH; and

each R⁶ is independently selected from the group consisting of —H, —(C₁-C₆)alkyl and aryl(C₁-C₃)alkyl; or

optionally, within any occurrence of —N(R⁵)M_{y}(R⁶) or —N(R⁶)M_{y}(R⁵) where y is 1, independently of any other occurrence of —N(R⁵)M_{y}(R⁶) or —N(R⁶)M_{y}(R⁵), R¹ and R⁶ in combination represent a single bond and M is selected such that the resulting —N(R⁵)M_{y}(R⁶) or —N(R⁶)M_{y}(R⁵) moiety represents a 5, 6, or 7-membered ring heterocycle;

each R⁷ is independently selected from the group consisting of hydrogen and (C₁-C₆)alkyl;

each R⁸ is independently hydrogen or (C₁-C₆)alkyl; or, optionally, within any occurrence of NR⁸₂, independently of any other occurrence of NR⁸₂, two R⁸ groups in combination are —(CH₂)ₕ— or —(CH₂)ᵢX(CH₂)₂—;

wherein:
h is 4, 5, or 6;
i is 2 or 3;
X is O, S, NR⁷, or a single bond;

each R⁹ is independently selected from the group consisting of —H, —(C₁-C₆)alkyl, —(CH₂)₃—NH—C(NH₂)(═NH), —CH₂C(═O)NH₂, —CH₂C(═O)OH, —CH₂SH, —(CH₂)₂C(═O)—NH₂, —(CH₂)₂C(═O)OH, —CH₂-(2-imidazolyl), —(CH₂)₄—NH₂, —(CH₂)₂—S—CH₃, phenyl, CH₂-phenyl, —CH₂—OH, —CH(OH)—CH₃, —CH₂-(3-indolyl) and —CH₂-(4-hydroxyphenyl);

each R¹⁰ is independently selected from the group consisting of —H, —(C₁-C₆)alkyl, —C(═O)OR⁷, —C(═O)NR⁸₂, —OR⁷, —SR⁷, —OC(═O)(CH₂)₂C(═O)OR⁷, guanidino, —NR⁷₂, —NR⁷₃⁺, —N⁺ (CH₂CH₂OR⁷)₃, phenyl, substituted phenyl, heterocyclyl and substituted heterocyclyl;

each R¹¹ is independently selected from the group consisting of R⁹, halogen, —NR⁸₂ and heterocycles containing two nitrogen atoms;

wherein the substituents for the substituted aryl and substituted heterocyclic groups comprising or included within R⁵ and R¹⁰ are independently selected from the group consisting of halogen, (C₁-C₆)alkyl, —OR⁷, —NO₂, —C≡N, —C(═O)OR⁷, —C(═O)NR⁸₂, —C(═NR⁷)NR⁸₂, —(C₁-C₃)alkylene-C(═O)OR⁷, —O(C₁-C₃)alkylene-C(═O)OR⁷, —(C₁-C₆)alkylene-OR⁷, —NR⁸₂, —P(═O)(OR⁷)₂, —OP(═O)(OR⁷)₂, —SO₂NR⁸₂, —NHC(═O)(C₁-C₆)alkyl, —OC(═O)(C₁-C₃)alkyl, —O(C₂-C₆)alkylene-NR⁸₂ and (C₁-C₃)perfluoroalkyl;

m is 0 or 1, provided that if D is —C≡N, then m is 1;

n is 0, 1, or 2, provided that if G is NR¹ then n is 2;

[symbol] indicates a single bond, whereby the configuration of the S—C═C—Ar² double bond may be either E or Z;

with the provisos that:

(i) if Ar¹ is unsubstituted phenyl, D is CN, G is CH₂, m is 1, and n is 2, then Ar² is other than unsubstituted phenyl;

(ii) if Ar¹ is 4-chlorophenyl, D is CN, G is CH₂, m is 1, and n is 2, then Ar² is other than 3,5-di-(t-butyl)-4-hydroxyphenyl;

(iii) if Ar¹ is unsubstituted phenyl, D is CN, a is NH, m is 1, and n is 2, then Ar² is other than 4-chlorophenyl;

(iv) if Ar¹ is unsubstituted phenyl, D is CN, G is CH₂, m is 1, n is 2, and Ar² is substituted phenyl, then Ar² is substituted at the 4-position by other than hydroxyl;

(v) if Ar¹ is 3-trifluoromethoxyphenyl, D is CN, G is CH₂, m is 1, n is 2, and Ar² is substituted phenyl, then Ar² is substituted at the 4-position by other than hydroxyl;

(vi) if D is C(═O)NH₂, m is 0, and n is 2, then Ar¹ is other than unsubstituted phenyl; and

(vii) if Ar² is unsubstituted phenyl, D is C(═O)NH₂, m is 0, and n is 2, then R⁴ is other than NH₂, NHCHO or NHC(═O)alkyl.

In another aspect of the invention, there are provided processes for preparing compounds according to formula I, comprising condensing a compound of formula II with an aromatic aldehyde of formula III:

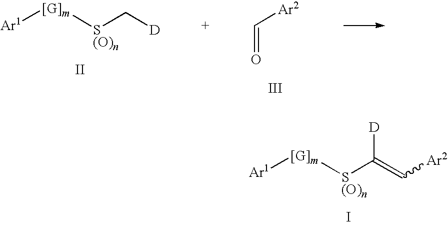

wherein Ar¹, Ar², G, R¹, m and n are as defined above for formula I.

In another aspect of the invention, novel compounds are provided which are useful in the synthesis of compounds of formula I. The novel intermediates are compounds of the formula IIA, or a salt thereof:

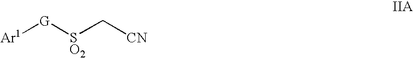

wherein G is CH₂ or NH, and Ar¹ is as defined above for the compounds of formula I.

Another aspect of the invention relates to antibody conjugates of compounds of formula I of the formula I-L-Ab, or a salt thereof, wherein I is a compound of formula I; Ab is an antibody; and -L- is a single bond or a linking group covalently linking said compound of formula I to said antibody.

In another aspect of the invention there are provided pharmaceutical compositions comprising a pharmaceutically acceptable carrier, and a compound according to formula I, or a pharmaceutically acceptable salt thereof. A pharmaceutical composition is additionally provided comprising a pharmaceutically acceptable carrier and at least one conjugate according to formula I-L-Ab, or a pharmaceutically acceptable salt thereof.

According to another embodiment of the invention, a method of treating an individual suffering from a cellular proliferative disorder, particularly cancer, is provided, comprising administering to said individual an effective amount of at least one compound according to formula I, or a pharmaceutically acceptable salt thereof, either alone, or in combination with a pharmaceutically acceptable carrier.

Also provided is a method of inducing apoptosis of cancer cells, preferably tumor cells, in an individual afflicted with cancer is provided, comprising administering to said individual an effective amount of at least one compound according to formula I, or a pharmaceutically acceptable salt thereof, either alone, or in combination with a pharmaceutically acceptable carrier.

According to another embodiment of the invention, a method of treating an individual suffering from a cellular proliferative disorder, particularly cancer, is provided, comprising administering to said individual an effective amount of at least one conjugate of the formula I-L-Ab, either alone, or in combination with a pharmaceutically acceptable carrier.

The invention is also directed to the use in medicine of a compound according to formula I, or a pharmaceutically acceptable salt thereof, or a conjugate according to formula I-L-Ab, or a pharmaceutically acceptable salt thereof.

The invention is also directed to the use of a compound according to formula I, or a pharmaceutically acceptable salt thereof, or a conjugate according to formula I-L-Ab, or a pharmaceutically acceptable salt thereof, in the preparation of a medicament for treatment of a cellular proliferative disorder, particularly cancer, or for inducing apoptosis of tumor cells in an individual affected with cancer.

### DETAILED DESCRIPTION OF THE INVENTION

According to the present invention, the sulfides, sulfones, sulfoxides, and sulfonamides and salts thereof which are described are believed to selectively inhibit proliferation of cancer cells, and kill various tumor cell types without killing (or with reduced killing of) normal cells. Cancer cells are killed at concentrations where normal cells may be temporarily growth-arrested but not killed.

The compounds of the invention are believed to inhibit the proliferation of tumor cells, and for some compounds, induce cell death. Cell death results from the induction of apoptosis. The compounds are believed effective against a broad range of tumor types, including but not limited to the following: ovarian cancer, breast cancer, prostate cancer, lung cancer, renal cancer, colorectal cancer, brain cancer and leukemia.

The compounds are also believed useful in the treatment of non-cancer cellular proliferative disorders, including but not limited to the following: hemangiomatosis in newborn, secondary progressive multiple sclerosis, chronic progressive myelodegenerative disease, neurofibromatosis, ganglioneuromatosis, keloid formation, Paget's disease of the bone, fibrocystic disease of the breast, uterine fibroids, Peyronie's disease, Dupuytren's disease, restenosis and cirrhosis.

### I. Definitions

### 1. General

As used in the specification and the appended claims, the singular forms “a,” “an” and “the” include plural referents unless the context clearly dictates otherwise.

As used herein, the terms “treat” and “treatment” are used interchangeably and are meant to indicate a postponement of development of a disorder and/or a reduction in the severity of symptoms that will or are expected to develop. The terms further include ameliorating existing symptoms, preventing additional symptoms, and ameliorating or preventing the underlying metabolic causes of symptoms.

As used herein, “individual” (as in the subject of the treatment) means both mammals and non-mammals. Mammals include, for example, humans; non-human primates, e.g. apes and monkeys; cattle; horses; sheep; and goats. Non-mammals include, for example, fish and birds.

The expression “effective amount”, when used to describe therapy to an individual suffering from a cancer or other cellular proliferative disorder, refers to the amount of a compound according to Formula I that inhibits the abnormal growth or proliferation, or alternatively induces apoptosis of cancer cells, preferably tumor cells, resulting in a therapeutically useful and selective cytotoxic effect on proliferative cells.

The term “cellular proliferative disorder” means a disorder wherein unwanted cell proliferation of one or more subsets of cells in a multicellular organism occurs. In some such disorders, cells are made by the organism at an atypically accelerated rate.

### 2. Chemical

In the following paragraphs some of the definitions include examples. The examples are intended to be illustrative, and not limiting.

The term “alkyl”, by itself or as part of another substituent means, unless otherwise stated, a straight, branched or cyclic chain hydrocarbon having the number of carbon atoms designated (i.e. C₁-C₆ means one to six carbons) and includes straight, branched chain or cyclic groups. Examples include: methyl, ethyl, propyl, isopropyl, butyl, isobutyl, tert-butyl, pentyl, neopentyl, hexyl, cyclohexyl and cyclopropylmethyl. Most preferred is (C₁-C₃)alkyl, particularly ethyl, methyl and isopropyl.

The term “alkenyl” employed alone or in combination with other terms, means, unless otherwise stated, a stable mono-unsaturated or di-unsaturated straight chain, branched chain or cyclic hydrocarbon group having the stated number of carbon atoms. Examples include vinyl, propenyl (allyl), crotyl, isopentenyl, butadienyl, 1,3-pentadienyl, 1,4-pentadienyl, cyclopentenyl, cyclopentadienyl and the higher homologs and isomers. A functional group representing an alkene is exemplified by —CH═CH—CH₂—.

“Substituted alkyl” or “substituted alkenyl” means alkyl or alkenyl, as defined above, substituted by one, two or three substituents selected from the group consisting of halogen, —OH, —NH₂, —N(CH₃)₂, —C(═O)OH, —C(═O)O(C₁-C₄)alkyl, trifluoromethyl, —C(═O)NH₂, —SO₂NH₂, —C(═NH)NH₂, —C≡N and —NO₂, preferably containing one or two substituents selected from halogen, —OH, NH₂, —N(CH₃)₂, trifluoromethyl, and —C(═O)OH, more preferably selected from halogen and —OH. Examples of substituted alkyls include, but are not limited to, 2,2-difluoropropyl, 2-carboxycyclopentyl and 3-chloropropyl.

The term “alkylene”, by itself or as part of another substituent means, unless otherwise stated, a divalent straight, branched or cyclic chain hydrocarbon.

The term “alkoxy” employed alone or in combination with other terms means, unless otherwise stated, an alkyl group having the designated number of carbon atoms, as defined above, connected to the rest of the molecule via an oxygen atom, such as, for example, methoxy, ethoxy, 1-propoxy, 2-propoxy (isopropoxy) and the higher homologs and isomers. Preferred are (C₁-C₃)alkoxy, particularly ethoxy and methoxy.

The term “carbamyl” means the group —C(═O)NRR′, wherein R and R′ are independently selected from hydrogen or a hydrocarbyl functional group, or wherein R and R′ combined form a heterocycle. Examples of carbamyl groups include: —C(═O)NH₂ and —C(═O)N(CH₃)₂.

The term “heteroalkyl” by itself or in combination with another term means, unless otherwise stated, a stable straight or branched chain alkyl group consisting of the stated number of carbon atoms and one or two heteroatoms selected from the group consisting of O, N, and S, and wherein the nitrogen and sulfur atoms may be optionally oxidized and the nitrogen heteroatom may be optionally quaternized. The heteroatom(s) may be placed at any position of the heteroalkyl group, including between the rest of the heteroalkyl group and the fragment to which it is attached, as well as attached to the most distal carbon atom in the heteroalkyl group. Examples include: —O—CH₂—CH₂-CH₃, —CH₂—CH₂CH₂—OH, —CH₂—CH₂—NH—CH₃, —CH₂—S—CH₂—CH₃, and —CH₂CH₂—S(═O)—CH₃. Up to two heteroatoms may be consecutive, such as, for example, —CH₂—NH—OCH₃, or —CH₂—CH₂—S—S—CH₃.

The term “heteroalkenyl” by itself or in combination with another term means, unless otherwise stated, a stable straight or branched chain monounsaturated or di-unsaturated hydrocarbon group consisting of the stated number of carbon atoms and one or two heteroatoms selected from the group consisting of O, N, and S, and wherein the nitrogen and sulfur atoms may optionally be oxidized and the nitrogen heteroatom may optionally be quaternized. Up to two heteroatoms may be placed consecutively. Examples include —CH═CH—O—CH₃, —CH═CH—CH₂—OH, —CH₂—CH═N—OCH₃, —CH═CH—N(CH₃)—CH₃, and —CH₂—CH═CH—CH₂—SH.

The terms “halo” or “halogen” by themselves or as part of another substituent mean, unless otherwise stated, a fluorine, chlorine, bromine, or iodine atom, preferably, fluorine, chlorine, or bromine, more preferably, fluorine or chlorine.

The term “(Cₓ-C_{y})perfluoroalkyl,” wherein x<y, means an alkyl group with a minimum of x carbon atoms and a maximum of y carbon atoms, wherein all hydrogen atoms are replaced by fluorine atoms. Preferred is —(C₁-C₆)perfluoroalkyl, more preferred is —(C₁-C₃)perfluoroalkyl, most preferred is —CF₃.

The term “(Cₓ-C_{y})perfluoroalkylene,” wherein x<y, means an alkyl group with a minimum of x carbon atoms and a maximum of y carbon atoms, wherein all hydrogen atoms are replaced by fluorine atoms. Preferred is —(C₁-C₆)perfluoroalkylene, more preferred is —(C₁-C₃)perfluoroalkylene, most preferred is —CF₂—.

The term “(Cₓ-C_{y})fluoroalkyl,” wherein x<y, means an alkyl group with a minimum of x carbon atoms and a maximum of y carbon atoms, wherein one or more atoms are replaced by fluorine atoms. Preferred is —(C₁-C₆) fluoroalkyl, more preferred is —(C₁-C₃)fluoroalkyl, most preferred is mono-, di-, or trifluoromethyl. Examples include —CH₂F, —CHF₂, —CF₃, —CH₂CF₃, and —CF₂CH₃.

The term “(Cₓ-C_{y})fluoroalkoxy” wherein x<y, means an alkoxy group with a minimum of x carbon atoms and a maximum of y carbon atoms, wherein one or more atoms are replaced by fluorine atoms. Preferred is —(C₁-C₆) fluoroalkoxy, more preferred is —(C₁-C₃)fluoroalkoxy, most preferred is mono-, di-, or tri-fluoromethoxy. Examples include —OCH₂F, —OCHF₂, —OCF₃, —OCH₂CF₃, and —OCF₂CH₃.

The term “phosphonato” means the group —PO(OH)₂.

The term “sulfamyl” means the group —SO₂NRR′, wherein R and R′ are independently selected from hydrogen or a hydrocarbyl group, or wherein R and R′ combined form a heterocycle. Examples of sulfamyl groups include: —SO₂NH₂, —SO₂N(CH₃)₂ and —SO₂NH(C₆H₅). Preferred are —SO₂NH₂, SO₂N(CH₃)₂ and —SO₂NHCH₃.

The term “aromatic” refers to a carbocycle or heterocycle having one or more polyunsaturated rings having aromatic character (i.e. having (4n+2) delocalized π (pi) electrons where n is an integer).

The term “aryl”, employed alone or in combination with other terms, means, unless otherwise stated, a carbocyclic aromatic system containing one or more rings (typically one, two or three rings) wherein such rings may be attached together in a pendent manner, such as a biphenyl, or may be fused, such as naphthalene. Examples include phenyl; anthracyl; and naphthyl. Preferred are phenyl and naphthyl, most preferred is phenyl.

The term “aryl-(C₁-C₃)alkyl” means a functional group wherein a one to three carbon alkylene chain is attached to an aryl group, e.g., —CH₂CH₂-phenyl. Preferred is aryl(CH₂)— and aryl(CH(CH₃))—. The term “substituted aryl-(C₁-C₃)alkyl” means an aryl-(C₁-C₃)alkyl functional group in which the aryl group is substituted. Preferred is substituted aryl(CH₂)—. Similarly, the term “heteroaryl(C₁-C₃)alkyl” means a functional group wherein a one to three carbon alkylene chain is attached to a heteroaryl group, e.g., —CH₂CH₂-pyridyl. Preferred is heteroaryl(CH₂)—. The term “substituted heteroaryl-(C₁-C₃)alkyl” means a heteroaryl-(C₁-C₃)alkyl functional group in which the heteroaryl group is substituted. Preferred is substituted heteroaryl(CH₂)—.

The term “arylene,” by itself or as part of another substituent means, unless otherwise stated, a structure formed by the removal of a hydrogen atom from two carbons in an arene. Preferred are phenyl arylenes, particularly 1,4-phenyl arylenes.

The term “heterocycle” or “heterocyclyl” or “heterocyclic” by itself or as part of another substituent means, unless otherwise stated, an unsubstituted or substituted, stable, mono- or multi-cyclic heterocyclic ring system which consists of carbon atoms and at least one heteroatom selected from the group consisting of N, O, and S, and wherein the nitrogen and sulfur heteroatoms may be optionally oxidized, and the nitrogen atom may be optionally quaternized. The heterocyclic system may be attached, unless otherwise stated, at any heteroatom or carbon atom which affords a stable structure.

The term “heteroaryl” or “heteroaromatic” refers to a heterocycle having aromatic character. A polycyclic heteroaryl may include one or more rings which are partially saturated. Examples include tetrahydroquinoline and 2,3-dihydrobenzofuryl. For compounds of formula I, the attachment point on ring Ar¹ or ring Ar² is understood to be on an atom which is part of an aromatic monocyclic ring or a ring component of a polycyclic aromatic which is itself an aromatic ring.

Examples of non-aromatic heterocycles include monocyclic groups such as: aziridine, oxirane, thiirane, azetidine, oxetane, thietane, pyrrolidine, pyrroline, imidazoline, pyrazolidine, dioxolane, sulfolane, 2,3-dihydrofuran, 2,5-dihydrofuran, tetrahydrofuran, thiophane, piperidine, 1,2,3,6-tetrahydropyridine, 1,4-dihydropyridine, piperazine, morpholine, thiomorpholine, pyran, 2,3-dihydropyran, tetrahydropyran, 1,4-dioxane, 1,3-dioxane, homopiperazine, homopiperidine, 1,3-dioxepane, 4,7-dihydro-1,3-dioxepin and hexamethyleneoxide.

Examples of heteroaryl groups include: pyridyl, pyrazinyl, pyrimidinyl, particularly 2- and 4-pyrimidinyl, pyridazinyl, thienyl, furyl, pyrrolyl, particularly 2-pyrrolyl, imidazolyl, thiazolyl, oxazolyl, pyrazolyl, particularly 3- and 5-pyrazolyl, isothiazolyl, 1,2,3-triazolyl, 1,2,4-triazolyl, 1,3,4-triazolyl, tetrazolyl, 1,2,3-thiadiazolyl, 1,2,3-oxadiazolyl, 1,3,4-thiadiazolyl and 1,3,4-oxadiazolyl.

Examples of polycyclic heterocycles include: indolyl, particularly 3-, 4-, 5-, 6- and 7-indolyl, indolinyl, quinolyl, tetrahydroquinolyl, isoquinolyl, particularly 1- and 5-isoquinolyl, 1,2,3,4-tetrahydroisoquinolyl, cinnolinyl, quinoxalinyl, particularly 2- and 5-quinoxalinyl, quinazolinyl, phthalazinyl, 1,8-naphthyridinyl, 1,4-benzodioxanyl, coumarin, dihydrocoumarin, benzofuryl, particularly 3-, 4-, 1,5-naphthyridinyl, 5-, 6- and 7-benzofuryl, 2,3-dihydrobenzofuryl, 1,2-benzisoxazolyl, benzothienyl, particularly 3-, 4-, 5-, 6-, and 7-benzothienyl, benzoxazolyl, benzthiazolyl, particularly 2-benzothiazolyl and 5-benzothiazolyl, purinyl, benzimidazolyl, particularly 2-benzimidazolyl, benztriazolyl, thioxanthinyl, carbazolyl, carbolinyl, acridinyl, pyrrolizidinyl, and quinolizidinyl.

The aforementioned listing of heterocyclyl and heteroaryl moieties is intended to be representative and not limiting.

The term “heteroarylene” by itself or as part of another substituent means, unless otherwise stated, an arylene containing at least one hetero atom. Preferred are five- or six-membered monocyclic heteroarylene. More preferred are heteroarylene moieties comprising heteroaryl rings selected from pyridine, piperazine, pyrimidine, pyrazine, furan, thiophene, pyrrole, thiazole, imidazole and oxazole.

For compounds of the present invention, when an aromatic or heteroaromatic ring is attached to a position and the ring comprises a polycyclic ring which is partially saturated, the attachment point on the aromatic or heteroaromatic ring is on a ring atom of an aromatic ring component of the polycyclic ring. For example on the partially saturated heteroaromatic ring, 1,2,3,4-tetrahydroisoquinoline, attachment points would be ring atoms at the 5-, 6-, 7- and 8-positions.

The aforementioned listing of heterocyclyl and heteroaryl moieties is intended to be representative and not limiting.

The term “hydrocarbyl” refers to any moiety comprising only hydrogen and carbon atoms. Preferred hydrocarbyl groups are (C₁-C₁₂)hydrocarbyl, more preferred are (C₁-C₇)hydrocarbyl, and most preferred are benzyl and (C₁-C₆) alkyl.

The term “substituted” means that an atom or group of atoms has replaced hydrogen as the substituent attached to another group. For aryl and heteroaryl groups, the term “substituted” refers to any level of substitution, namely mono-, di-, tri-, tetra-, or penta-substitution, where such substitution is permitted. The substituents are independently selected, and substitution may be at any chemically accessible position.

Where a substituent is an alkyl or alkoxy group, the carbon chain may be branched, straight or cyclic, with straight being preferred.

The term “antibody” is intended to encompass not only intact antigen-binding immunoglobulin molecules, but also to include antigen-binding fragments thereof such as Fab, Fab′ and F(ab′)₂ fragments, or any other fragment retaining the antigen-binding ability of an intact antibody.

The term “monospecific polyclonal antibody” means an antibody preparation comprising multiple antibody species having specificity for a single antigen.

The term “monovalent peptidyl group” refers to a peptide functional group as a substituent on a molecule of formula I. Such a functional group has a chemical structure that varies from the structure of the corresponding peptide in that the structural component of the peptide, i.e., an alpha amino group, a side chain amino group, an alpha carboxyl group or a side chain carboxyl group, will form a different functionality when bonded to the molecule of which it is to be a substituent. For example, for a peptide as shown below:

H₂N-Val-Pro-Ala-C(═O)OH

which is a substituent on a compound of formula I, the peptide is coupled to the compound of formula I such that a carboxyl moiety of said peptide is coupled to a free amine moiety on the formula I compound. Elimination of water results in the formation of an amide bond. As a practical result, the corresponding monovalent peptidyl substituent is shown to the left of the dotted line in the depiction below of the aforementioned peptide bonded to a compound of formula I:

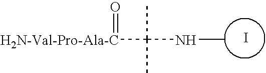

The monovalent peptide group may be attached via either an alpha- or a side chain amino group, or an alpha or side chain carboxyl group. The attachment point on the peptide group will depend on the functionality at the terminus of the connecting group M in a manner that is known to one of skill in the art (see the definition).

Specifically, the peptidyl group may be coupled to the M connecting group via an alpha amino or a side chain amino group when the M connecting group terminates in: —C(═O)—, —C(═S)—, —S(═O)—, or SO₂, i.e., when the variable e is zero.

Likewise, the peptidyl group may be coupled to the M connecting group via an alpha carboxy or a side chain carboxy group when the M connecting group terminates in: —C(═O)NR⁵—, —SO₂NR⁵—, —NR⁵—, —S— or —O—, i.e., when the variable e (or g) is zero.

### II. Compounds According to the Invention

In one aspect, the invention is a compound of formula I, or a salt thereof:

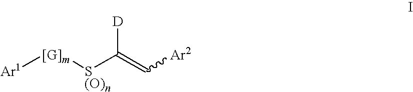

wherein:

Ar¹ is:

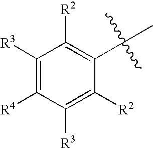

Ar² is selected from the group consisting of unsubstituted aryl, substituted aryl, unsubstituted heteroaryl and substituted heteroaryl; wherein, when Ar² is substituted aryl or substituted heteroaryl, said substituted aryl or substituted heteroaryl is aryl or heteroaryl substituted with one or more substituents independently selected from the group consisting of halogen, (C₁-C₆)alkyl, (C₂-C₆)alkenyl, (C₂-C₆)alkynyl, —(C₁-C₃)alkylene-Ar³, —OR⁷, —OAr³, —O(C₁-C₃)alkylene-Ar³, (C₁-C₃)fluoroalkoxy, —NO₂, —C—N, —C(═O)(C₁-C₃)alkyl, —C(═O)OR⁷, —C(═O)NR⁸₂—C(═NR⁷)NR⁸₂, —(C₁-C₃)alkylene-C(═O)OR⁷, —O(C₁-C₃)alkylene-C(═O)OR⁷, —(C₁-C₆)alkylene-OR⁷, —NR₂, —P(═O)(OR⁷)₂, —OP(═O)(OR⁷)₂, —S(C₁-C₆)alkyl, —S(O)(C₁-C₆)alkyl, —SO₂(C₁-C₆)alkyl, —SO₂NR⁸₂, —OSO₂(C₁-C₆)alkyl, —OSO₂Ar³, —NHC(═O)(C₁-C₆)alkyl, —OC(═O)(C₁-C₃)alkyl, —O(C₂-C₆)alkylene-NR⁸₂ and (C₁-C₃)perfluoroalkyl;

D is —C≡N, —C(═O)NR⁸₂, or NO₂;

G is CR¹₂ or NR¹;

R¹ is independently selected from the group consisting of hydrogen and (C₁-C₆)alkyl;

each R² is independently selected from the group consisting of hydrogen, (C₁-C₆)alkyl, halogen, —OR⁷, —OAr³, —O(C₁-C₃)alkylene-Ar³, —C≡N, —NO₂, —C(═O)OR⁷, —C(═O)NR⁸₂, —C(═NR⁷)NR⁸₂, —(C₁-C₃)alkylene-C(═O)OR⁷, —O(C₁-C₃)alkylene-C(═O)OR⁷, —(C₁-C₆)alkylene-OR⁷, —P(═O)(OR⁷)₂, —OP(═O)(OR⁷)₂, —NHC(═O)(C₁-C₆)alkyl, —SO₂NR⁸₂, —OSO₂(C₁-C₆)alkyl, —OSO₂Ar³, —OC(═O)(C₁-C₃)alkyl, —O(C₂-C₆)alkylene-NR⁸₂, (C₁-C₃)perfluoroalkyl and —N(R⁶)-(M)_{y}-R⁵;

each R³ is independently selected from the group consisting of hydrogen, (C₁-C₆)alkyl, halogen, —OR⁷, —OAr³, —O(C₁-C₃)alkylene-Ar³, —C≡N, —NO₂, —C(═O)OR⁷, —C(═O)NR⁸₂, —C(═NR⁷)NR⁸₂, —(C₁-C₃)alkylene-C(═O)OR⁷, —O(C₁-C₃)alkylene-C(═O)OR⁷, —(C₁-C₆)alkylene-OR⁷, —P(═O)(OR⁷)₂, —OP(═O)(OR⁷)₂, —NHC(═O)(C₁-C₆)alkyl, —SO₂NR⁸₂, —OSO₂(C₁-C₆)alkyl, —OSO₂Ar³, —OC(═O)(C₁-C₃)alkyl, —O(C₂-C₆)alkylene-NR⁸₂, (C₁-C₃)perfluoroalkyl and —N(R⁶)-(M)_{y}-R⁵;

R⁴ is selected from the group consisting of hydrogen, (C₁-C₆)alkyl, halogen, —OR⁷, —OAr³, —O(C₁-C₃)alkylene-Ar³, —C≡N, —NO₂, —C(═O)OR⁷, —C(═O)NR⁸₂, —C(═NR⁷)NR⁸₂, —(C₁-C₃)alkylene-C(═O)OR⁷, —O(C₁-C₃)alkylene-C(═O)OR⁷, —(C₁-C₆)alkylene-OR⁷, —P(═O)(OR⁷)₂, —OP(═O)(OR⁷)₂, —NHC(═O)(C₁-C₆)alkyl, —SO₂NR⁸₂, —OSO₂(C₁-C₆)alkyl, —OSO₂Ar³, —OC(═O)(C₁-C₃)alkyl, —O(C₂-C₆)alkylene-NR⁸₂, (C₁-C₃)perfluoroalkyl, —N(R⁵)M_{y}(R⁶) and —N(R⁶)M_{y}(R⁵);

each Ar³ is phenyl optionally substituted with 1, 2, 3, 4, or 5 substituents independently selected from the group consisting of (C₁-C₃)alkyl, (C₁-C₃)alkoxy and halogen;

each M is a connecting group independently selected from the group consisting of —(C₁-C₆)alkylene-, —(CH₂)_{d}—V—(CH₂)ₑ—, —(CH₂)_{f}—W—(CH₂)_{g}— and -Z-;

each y is independently selected from the group consisting of 0 and 1;

each V is independently selected from the group consisting of arylene, heteroarylene, —C(═O)—, —C(═O)(C₁-C₆)perfluoroalkylene, —C(═O)—, —C(═S)—, —S(═O)—, —SO₂—, —C(═O)NR⁷—, —C(═S)NR⁷— and —SO₂NR⁷—;

each W is independently selected from the group consisting of —NR⁷—, —O— and —S—;

each d is independently selected from the group consisting of 0, 1 and 2;

each e is independently selected from the group consisting of 0, 1 and 2;

each f is independently selected from the group consisting of 1, 2 and 3;

each g is independently selected from the group consisting of 0, 1 and 2;

-Z- is

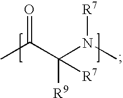

wherein the absolute configuration of -Z- is (S) or (R), or a mixture of (S) and (R);

each R⁵ is independently selected from the group consisting of hydrogen, unsubstituted aryl, substituted aryl, unsubstituted heterocyclic, substituted heterocyclic, —C(═O)OR⁷, —C(═O)NR⁸₂, —C(═NH)—NR⁸₂, —(C₁-C₆)perfluoroalkyl, —CF₂Cl, —P(═O)(OR⁷)₂, —CR⁷R¹⁰R¹¹ and a monovalent peptidyl group with a molecular weight of less than 1000, preferably with a molecular weight of less than 800, more preferably with a molecular weight of less than 600, most preferably with a molecular weight of less than 400, provided that when y is 0, R⁵ is not —C(═O)OH; and

each R⁶ is independently selected from the group consisting of —H, —(C₁-C₆)alkyl and aryl(C₁-C₃)alkyl; or

optionally, within any occurrence of —N(R⁵)M_{y}(R⁶) or —N(R⁶)M_{y}(R⁵) where y is 1, independently of any other occurrence of —N(R⁵)M_{y}(R⁶) or —N(R⁶)M_{y}(R⁵), R⁵ and R⁶ in combination represent a single bond and M is selected such that the resulting —N(R⁵)M_{y}(R⁶) or —N(R⁶)M_{y}(R⁵) moiety represents a 5, 6, or 7-membered ring heterocycle;

each R⁷ is independently selected from the group consisting of hydrogen and (C₁-C₆)alkyl;

each R⁸ is independently hydrogen or (C₁-C₆)alkyl; or, optionally, within any occurrence of NR⁸₂, independently of any other occurrence of NR⁸₂, two R⁸ groups in combination are —(CH₂)ₕ— or —(CH₂)ᵢX(CH₂)₂—;

wherein:
h is 4, 5, or 6;
i is 2 or 3;
X is O, S, NR⁷, or a single bond;

each R⁹ is independently selected from the group consisting of —H, —(C₁-C₆)alkyl, —(CH₂)₃—NH—C(NH₂)(═NH), —CH₂C(═O)NH₂, —CH₂C(═O)OH, —CH₂SH, —(CH₂)₂C(═O)—NH₂, —(CH₂)₂C(═O)OH, —CH₂-(2-imidazolyl), —(CH₂)₄—NH₂, —(CH₂)₂—S—CH₃, phenyl, CH₂-phenyl, —CH₂—OH, —CH(OH)—CH₃, —CH₂-(3-indolyl) and —CH₂-(4-hydroxyphenyl);

each R¹⁰ is independently selected from the group consisting of —H, —(C₁-C₆)alkyl, —C(═O)OR⁷, —C(═O)NR⁸₂, —OR⁷, —SR⁷, —OC(═O)(CH₂)₂C(═O)OR⁷, guanidino, —NR⁷₂, —NR⁷₃⁺, —N⁺(CH₂CH₂OR⁷)₃, phenyl, substituted phenyl, heterocyclyl and substituted heterocyclyl;

each R¹¹ is independently selected from the group consisting of R⁹, halogen, —NR⁸₂ and heterocycles containing two nitrogen atoms;

wherein the substituents for the substituted aryl and substituted heterocyclic groups comprising or included within R⁵ and R¹⁰ are independently selected from the group consisting of halogen, (C₁-C₆)alkyl, —OR⁷, —NO₂, —C≡N, —C(═O)OR⁷, —C(═O)NR⁸₂, —C(═NR⁷)NR⁸₂, —(C₁-C₃)alkylene-C(═O)OR⁷, —O(C₁-C₃)alkylene-C(═O)OR⁷, —(C₁-C₆)alkylene-OR⁷, —NR⁸₂, —P(═O)(OR⁷)₂, —OP(═O)(OR⁷)₂, —SO₂NR⁸₂, —NHC(═O)(C₁-C₆)alkyl, —OC(═O)(C₁-C₃)alkyl, —O(C₂-C₆)alkylene-NR⁸₂ and (C₁-C₃)perfluoroalkyl;

m is 0 or 1, provided that if D is —C≡N, then m is 1;

n is 0, 1, or 2, provided that if G is NR¹ then n is 2;

[symbol] indicates a single bond, whereby the configuration of the S—C═C—Ar² double bond may be either E or Z;

with the provisos that:

(i) if Ar¹ is unsubstituted phenyl, D is CN, G is CH₂, m is 1 and n is 2, then Ar² is other than unsubstituted phenyl;

(ii) if Ar¹ is 4-chlorophenyl, D is CN, G is CH₂, m is 1 and n is 2, then Ar² is other than 3,5-di-(t-butyl)-4-hydroxyphenyl;

(iii) if Ar¹ is unsubstituted phenyl, D is CN, G is NH, m is 1 and n is 2, then Ar² is other than 4-chlorophenyl;

(iv) if Ar¹ is unsubstituted phenyl, D is CN, G is CH₂, m is 1, n is 2 and Ar² is substituted phenyl, then Ar² is substituted at the 4-position by other than hydroxyl;

(v) if Ar¹ is 3-trifluoromethoxyphenyl, D is CN, 1 is CH₂, m is 1, n is 2 and Ar² is substituted phenyl, then Ar² is substituted at the 4-position by other than hydroxyl;

(vi) if D is C(═O)NH₂, m is 0 and n is 2, then Ar¹ is other than unsubstituted phenyl; and

(vii) if Ar² is unsubstituted phenyl, D is C(═O)NH₂, M is 0 and n is 2, then R⁴ is other than NH₂, NHCHO or NHC(═O)alkyl.

When R⁵ is a peptidyl group, the attachment point on the peptidyl group may be via a carboxyl group or through an amino group. Further, the carboxyl or amino groups may be either terminal carboxyl/amino groups or may be side chain groups such as, for example, the side chain amino group of lysine or the side chain carboxyl group of aspartic acid. The attachment point on the peptidyl group will correlate with the particular selection of the M connecting group. Thus, for R⁵ as a peptidyl group of molecular weight less than 1000, it is provided that:
(1) when V is —C(═O)—, —C(═S)—, —S(═O)— or —SO₂— and e is 0, then the peptidyl group is coupled to M through the peptide's amino terminus or through a side chain amino group to form an amide, thioamide, sulfinamide or sulfonamide, respectively;
(2) when V is —C(═O)NR⁷—, —SO₂NR⁷—, or —NR⁷— and e is 0, then the peptidyl group is coupled to M through the peptide's carboxy terminus or through a side chain carboxyl group to form an imide, sulfonimide, or carboxamide, respectively, and
(3) when W is —S— or —O— and g is 0, then the peptidyl group is coupled to M through the peptide's carboxy terminus or through a side chain carboxyl group to form a carbothioic acid ester or a carboxylic ester, respectively.

One particular embodiment of the invention comprises a compound of formula I, or a salt thereof, wherein M is -Z′-:
-Z′— is

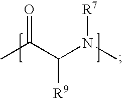

wherein:
the absolute stereochemistry of -Z′- is either S or R; and
each R⁹ is independently —H, —(C₁-C₆)alkyl, —(CH₂)₃—NH—C(NH₂)(═NH), —CH₂C(═O)NH₂, —CH₂C(═O)OH, —CH₂SH, —(CH₂)₂C(═O)—NH₂, —(CH₂)₂C(═O)OH, —CH₂-(2-imidazolyl), —(CH₂)₄—NH₂, —(CH₂)₂—S—CH₃, phenyl, CH₂-phenyl, —CH₂—OH, —CH(OH)—CH₃, —CH₂-(3-indolyl), —CH₂-(4-hydroxyphenyl).

Another particular embodiment of the invention comprises a compound of formula I, or a salt thereof, wherein each V is independently selected from the group consisting of: —C(═O)—, —C(═S)—, —S(═O)—, —SO₂—, —C(═O)NR⁷—, —C(═S)NR⁷— and —SO₂NR⁷—.

Another particular embodiment of the invention comprises a compound of formula I, or a salt thereof, wherein the stereochemistry of the double bond in the S—C═C—Ar² moiety is E.

Another particular embodiment of the invention comprises a compound of formula I, or a salt thereof, wherein m is 1.

Another particular embodiment of the invention comprises a compound of formula I, or a salt thereof, wherein n is 2.

Another particular embodiment of the invention comprises a compound of formula I, or a salt thereof, wherein Ar² is substituted or unsubstituted phenyl. In particular embodiments thereof, Ar² is phenyl substituted at the 4-position other than by hydroxy. In other embodiments of the invention, Ar² is substituted phenyl wherein all the substituents are other than hydroxy.

Other embodiments of the invention are those wherein Ar² is substituted phenyl wherein the substituents (other than hydrogen) of Ar² are selected from the group consisting of halogen, (C₁-C₆)alkoxy, —OAr³, preferably phenoxy and —O(C₁-C₃)alkylene-Ar³, preferably benzyloxy.

Other embodiments of the invention are those wherein Ar is substituted phenyl substituted with one, two, or three alkoxy groups, preferably at the 2-, 4- and/or 6-positions, for example 2-, 4-, or 6-monosubstituted, 2,4- or 2,6-disubstituted, or 2,4,6-trisubstituted. Also favoured is 3,4-disubstituted. The preferred alkoxy groups are methoxy and ethoxy. In preferred embodiments thereof, the alkoxy substituents are the only substituents of Ar² (i.e. the ring has hydrogen at other positions). Examples are those embodiments wherein Ar² is 2,6-dimethoxyphenyl, 2,4,6-trimethoxyphenyl or 4-ethoxy-3-methoxyphenyl.

Other embodiments of the invention are wherein Ar² is substituted phenyl substituted with one, two, three, four or five halogen atoms. In preferred embodiments thereof, the alkoxy substituents are the only substituents of Ar² (i.e. the ring has hydrogen at other positions). Examples are those embodiments wherein Ar² is 2,3,5-trichlorophenyl or 2,3,4,5,6-pentafluorophenyl.

Other embodiments of the invention are those wherein Ar¹ is mono- or di-substituted phenyl wherein the substituents of Ar¹ are independently selected from the group consisting of (C₁-C₆)alkyl, halogen, —OR⁷, preferably (C₁-C₆)alkoxy, preferably methoxy, —OAr³, preferably phenoxy, —O(C₁-C₃)alkylene-Ar¹, preferably benzyloxy, —OSO₂(C₁-C₆)alkyl, and —OSO₂Ar³, wherein Ar³ is preferably phenyl or p-tolyl, and —N(R⁶)-(M)_{y}-R⁵.

Other particular embodiments of the invention are those wherein each occurrence R³ is other than (C₁-C₃) perfluoroalkyl. Preferred are those wherein each occurrence of both R² and R³ is other than (C₁-C₃) perfluoroalkyl and R⁴ is other than (C₁-C₃) perfluoroalkyl.

Other embodiments of the invention those wherein Ar¹ is mono- or di-substituted phenyl. In preferred embodiments of the invention Ar¹ is substituted in at least the 2- or 4-positions, and is preferably substituted in at least the 4-position. The preferred substituents of the 2- and/or 4-positions (R² and R⁴ respectively) are (C₁-C₆)alkyl, halogen, —OR⁷, preferably (C₁-C₆)alkoxy, preferably methoxy, —OAr³, preferably phenoxy, —O(C₁-C₃)alkylene-Ar³, preferably benzyloxy, —OSO₂(C₁-C₆)alkyl, and —OSO₂Ar³, wherein Ar³ is preferably phenyl or p-tolyl. Also preferred are those embodiments wherein R⁴ is other than hydrogen, preferably halogen or —OR⁷, preferably alkoxy.

Other embodiments of the invention are those wherein Ar¹ is:

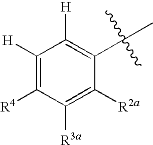

wherein:

R⁴ is selected from the group consisting of hydrogen, (C₁-C₆)alkyl, halogen, and —OR⁷, preferably alkoxy;

R^{3a} is selected from the group consisting of hydrogen, —OR⁷, —OAr³, preferably phenoxy, —O(C₁-C₃)alkylene-Ar³ preferably benzyloxy, —OSO₂(C₁-C₆)alkyl, —OSO₂Ar³ and —N(R⁶)-(M)_{y}-R⁵;

R^{2a} is selected from the group consisting of hydrogen, (C₁-C₆)alkyl, halogen, —OR⁷, preferably alkoxy, —OAr³, preferably phenoxy, —O(C₁-C₃)alkylene-Ar³, preferably benzyloxy, —OSO₂(C₁-C₆)alkyl, and —OSO₂Ar³; provided:

at least one of R^{2a} and R^{3a} and R⁴ is other than hydrogen; and

at least one of R^{2a} and R^{3a} is hydrogen.

In preferred embodiments thereof:

R⁴ is halogen or —OR⁷;

R^{3a} is selected from the group consisting of hydrogen, —OR⁷, and —N(R⁶)-(M)_{y}-R⁵; and

R^{2a} is hydrogen or halogen.

In other preferred embodiments thereof R^{2a} and R^{3a} are both hydrogen and R⁴ is halogen or —O(C₁-C₆)alkyl.

In other preferred embodiments thereof, R^{2a} is hydrogen, R^{3a} is —N(R⁶)-(M)_{y}-R⁵ and R⁴ is —O(C₁-C₆)alkyl.

Other embodiments of the invention are those compounds according to formula I wherein at least one R³ is —OR⁷, preferably alkoxy or —N(R⁶)-(M)_{y}-R⁵, and preferred embodiments are those wherein one R³ is —N(R⁶)-(M)_{y}-R⁵, and the other R³ is hydrogen. Preferred subembodiments thereof are those wherein one R⁴ is other than hydrogen, preferably halogen or —OR⁷, preferably alkoxy, and those wherein R² is hydrogen. In preferred subembodiments thereof, Ar¹ is 3,4-disubstituted phenyl wherein each R² is hydrogen, one R³ is hydrogen, one R³ is —OR⁷, preferably alkoxy or —N(R⁶)-(M)_{y}-R⁵ and R⁴ is other than hydrogen, preferably halogen or —OR⁷, preferably alkoxy.

Particular embodiments of the invention are those wherein Ar¹ is one of the aryl groups named the column labeled Ar¹ in Tables 1, 2, 3, 4, 5, or 6 herein, and preferred embodiments of the invention are those having Ar¹ groups found in at least one compound having an IC₅₀ of 25 μM or lower in at least one of the assays for which data are given in Table 9, with Ar¹ groups found in at least one compound having an IC₅₀ of 10 μM or lower being particularly preferred. Similarly, particular embodiments of the invention are those wherein Ar² is one of the aryl groups named the column labeled Ar² in Tables 1, 2, 3, 4, 5, or 6 herein, and preferred embodiments of the invention are those having Ar² groups found in at least one compound having an IC₅₀ of 25 μM or lower in at least one of the assays for which data are given in Table 9, with Ar² groups found in at least one compound having an IC₅₀ of 10 μM or lower being particularly preferred. Preferred combinations of Ar¹ and Ar² groups are those combinations found in at least one compound having an IC₅₀ of 25 μM or lower in at least one of the assays for which data are given in Table 9, and combinations found in at least one compound having an IC₅₀ of 10 μM or lower are particularly preferred.

Another particular embodiment of the invention comprises a compound of formula I, or a salt thereof, wherein R¹ is H.

Another particular embodiment of the invention comprises a compound of formula I, or a salt thereof, wherein D is —C≡N, m is 1, and n is 2.

Another particular embodiment of the invention comprises a compound of formula I, or a salt thereof, wherein D is —C≡N, G is CR¹₂, preferably CH₂, m is 1, and n is 2. Particular and preferred embodiments thereof include those wherein R¹, Ar¹, and Ar² are as described above for particular and preferred embodiments of the compounds according to Formula I. Particular embodiments also include those wherein Ar¹ is one of the aryl groups named the column labeled Ar¹ in Table 1 herein, and preferred embodiments include those having Ar¹ groups found in at least one Table 1 compound having an IC₅₀ of 25 μM or lower in at least one of the assays for which data are given in Table 9, with Ar¹ groups found in at least one Table 1 compound having an IC₅₀ of 10 μM or lower being particularly preferred. Similarly, particular embodiments also include those wherein Ar² is one of the aryl groups named the column labeled Ar² in Table 1 herein, with preferred Ar² groups found in at least one Table 1 compound having an IC₅₀ of 25 μM or lower in at least one of the assays for which data are given in Table 9, and Ar² groups found in at least one Table 1 compound having an IC₅₀ of 10 μM or lower being particularly preferred. Preferred Ar¹ groups include 4-methoxyphenyl, 4-fluorophenyl, 4-chlorophenyl, 4-bromophenyl, 3,4-dimethoxyphenyl, 3-amino-4-methoxyphenyl, and 3-fluoro-4-methoxyphenyl. Preferred Ar² groups include 2,4,6-trimethoxyphenyl and 2,3,4,5,6-pentafluorophenyl.

Another particular embodiment of the invention comprises a compound of formula I, or a salt thereof, wherein Ar² is substituted or unsubstituted phenyl, D is —C≡N, G is CR¹₂, preferably CH₂, m is 1, and n is 2.

Another particular embodiment of the invention comprises a compound of formula I, or a salt thereof, wherein D is —C≡N, G is CR¹₂, preferably CH₂, m is 1, n is 2, and R⁴ is other than hydrogen, preferably halogen or —OR⁷, preferably alkoxy.

Another particular embodiment of the invention comprises a compound of formula I, or a salt thereof, wherein D is —C≡N, G is CR¹₂, preferably CH₂, m is 1, n is 2, each occurrence of both R² and R³ is hydrogen, and R⁴ is other than hydrogen, preferably halogen or —OR⁷, preferably alkoxy.

Another particular embodiment of the invention comprises a compound of formula I, or a salt thereof, wherein D is —C≡N, G is CR¹₂, preferably CH₂, m is 0.1, n is 2, each occurrence R³ is other than (C₁-C₃) perfluoroalkyl.

Another particular embodiment of the invention comprises a compound of formula I, or a salt thereof, wherein D is —C≡N, G is CR¹₂, preferably CH₂, m is 1, n is 2, each occurrence of both R² and R³ is other than (C₁-C₃) perfluoroalkyl, and R⁴ is other than (C₁-C₃) perfluoroalkyl.

Another particular embodiment of the invention comprises a compound of formula I, or a salt thereof, wherein D is —C≡N, G is CR¹₂, preferably CH₂, m is 1, n is 2, each occurrence of both R² and R³ is hydrogen, and R⁴ is selected from the group consisting of halogen and —OR⁷, wherein R⁷ is preferably alkoxy.

Another particular embodiment of the invention comprises a compound of formula I, or a salt thereof, wherein D is —C≡N, G is CR¹₂, preferably CH₂, m is 1, n is 2, and Ar¹ is 4-methoxyphenyl.

Another particular embodiment of the invention comprises a compound of formula I, or a salt thereof, wherein D is —C≡N, G is CR¹₂, preferably CH₂, m is 1, n is 2, and Ar¹ is selected from the group consisting of 4-methoxyphenyl, 4-fluorophenyl, 4-chlorophenyl, 4-bromophenyl, 3,4-dimethoxyphenyl, 3-nitro-4-methoxyphenyl, 3-amino-4-methoxyphenyl, 3-fluoro-4-methoxyphenyl, and 2,4-dichlorophenyl.

Another particular embodiment of the invention comprises a compound of formula I, or a salt thereof, wherein D is —C≡N, G is CR¹₂, preferably CH₂, m is 1, and n is 2, Ar² is substituted phenyl substituted at the 4-position by other than hydroxy.

Another particular embodiment of the invention comprises a compound of formula I, or a salt thereof, wherein D is —C≡N, G is CR¹₂, preferably CH₂, m is 1, and n is 2, Ar² is substituted phenyl, and each substituent of Ar² is other than hydroxy.

Another particular embodiment of the invention comprises a compound of formula I, or a salt thereof, wherein D is —C≡N, G is CR¹₂, preferably CH₂, m is 1, n is 2, and Ar² is selected from the group consisting of 2-benzoxazolon-5-yl, 2-benzoxazolon-6-yl, 1,3-benzodioxole-5-yl, furyl, and thiophenyl.

Another particular embodiment of the invention comprises a compound of formula I, or a salt thereof, wherein D is —C≡N, G is CR¹₂, preferably CH₂, m is 1, n is 2, Ar² is substituted phenyl substituted at the 4-position by halogen, preferably fluorine or —OR⁷, preferably alkoxy.

Another particular embodiment of the invention comprises a compound of formula I, or a salt thereof, wherein D is —C≡N, G is CR¹₂, preferably CH₂, m is 1, and n is 2, and Ar² is selected from the group consisting of 2,3,4,5,6-pentafluorophenyl; 2,3,4-trichlorophenyl; 2,3,4-trimethoxyphenyl; 2,4,5-trimethoxyphenyl; 2,4,6-trimethoxyphenyl; 2,4,6-trimethoxyphenyl; 2,4-dichlorophenyl; 2,5-dimethylphenyl; 2,6-dimethoxyphenyl; 2-benzoxazolon-5-yl; 2-benzoxazolon-6-yl; 2-benzyloxyphenyl; 2-chloro-4-fluorophenyl; 2-chloro-4-fluorophenyl; 2-chlorophenyl; 2-fluoro-4-cyanophenyl; 2-fluoro-4-methoxyphenyl; 2-fluoro-4-methoxyphenyl; 2-fluoro-4-methoxyphenyl; 2-fluoro-4-nitrophenyl; 3,4,5-trimethoxyphenyl; 3,4-dichlorophenyl; 3,4-dihydroxyphenyl; 3,4-dimethoxyphenyl; 3,4-dimethylphenyl; 3,5-difluorophenyl; 3,5-dimethoxyphenyl; 3,5-dimethylphenyl; 3-furyl; 3-hydroxy-4-methoxyphenyl; 3-indolyl; 3-indolyl; 3-methyl-2-furyl; 3-methyl-2-furyl; 3-methyl-2-thiophen-2-yl; 3-nitro-4-fluorophenyl; 4-(N,N-dimethylamino)phenyl 4-acetoxyphenyl; 4-aminophenyl; 4-bromophenyl; 4-chlorophenyl; 4-ethoxy-3-methoxyphenyl; 4-ethoxycarbonyl-3,5-dimethylpyrrol-2-yl; 4-fluoro-3-methylphenyl; 4-fluorophenyl; 4-methanesulfenyl; 4-methoxyphenyl; 5-bromo-3-indolyl; 5-chloro-3-indolyl and phenyl.

Another particular embodiment of the invention comprises a compound of formula I, or a salt thereof, wherein D is —C≡N, G is CR¹₂, preferably CH₂, m is 1, and n is 2, and Ar² is selected from the group consisting of 2,4,6-trimethoxyphenyl and 2,3,4,5,6-pentafluorophenyl.

Another particular embodiment of the invention comprises a compound of formula I, or a salt thereof, wherein D is C(═O)NH₂.

Another particular embodiment of the invention comprises a compound of formula I, or a salt thereof, wherein D is —C(═O)NR⁸₂, G is CR¹₂, m is 0 or 1, and n is 2.

Another particular embodiment of the invention comprises a compound of formula I, or a salt thereof, wherein D is —C(═O)NH₂, G is CR¹₂, m is 0 or 1, and n is 2.

Another particular embodiment of the invention comprises a compound of formula I, or a salt thereof, wherein D is —C(═O)NR⁸₂, G is CH₂, m is 0 or 1, and n is 2.

Another particular embodiment of the invention comprises a compound of formula I, or a salt thereof, wherein D is C(═O)NH₂, G is CH₂, m is 0 or 1, and n is 2.

Another particular embodiment of the invention comprises a compound of formula I, or a salt thereof, wherein D is —NO₂, G is CH₂ m is 0 or 1, and n is 2.

Another particular embodiment of the invention comprises a compound of formula I, or a salt thereof, wherein D is —C≡N, G is CR¹₂, m is 1, and n is 1.

Another particular embodiment of the invention comprises a compound of formula I, or a salt thereof, wherein D is —C(═O)NR⁸₂, G is CR¹₂, m is 0 or 1, and n is 1.

Another particular embodiment of the invention comprises a compound of formula I, or a salt thereof, wherein D is —NO₂, G is CH₂ m is 0 or I, and n is 1.

Another particular embodiment of the invention comprises a compound of formula I, or a salt thereof, wherein D is —C≡N, G is CR¹₂, m is 1, and n is 0.

Another particular embodiment of the invention comprises a compound of formula I, or a salt thereof, wherein D is —CONR⁸₂, G is CR¹₂, m is 0 or 1, and n is 0.

Another particular embodiment of the invention comprises a compound of formula I, or a salt thereof, wherein D is —NO₂, G is CH₂ m is 0 or 1, and n is 0.

Another particular embodiment of the invention comprises a compound of formula I, or a salt thereof, wherein D is —C≡N, G is NR¹, preferably NH, m is 1, n is 2. Particular and preferred embodiments thereof include those wherein R¹, Ar¹, and Ar² are as described above for particular and preferred embodiments of the compounds according to Formula I. Particular embodiments also include those wherein Ar¹ is one of the aryl groups named the column labeled Ar¹ in Table 6 herein, and preferred embodiments include those having Ar¹ groups found in at least one Table 6 compound having an IC₅₀ of 25 μM or lower in at least one of the assays for which data are given in Table 9, with Ar¹ groups found in at least one Table 6 compound having an IC₅₀ of 10 μM or lower being particularly preferred. Similarly, particular embodiments also include those wherein Ar² is one of the aryl groups named the column labeled Ar² in Table 6 herein, with preferred Ar² groups found in at least one Table 6 compound having an IC₅₀ of 25 μM or lower in at least one of the assays for which data are given in Table 9, and Ar² groups found in at least one Table 6 compound having an IC₅₀ of 10 μM or lower being particularly preferred. Preferred Ar¹ groups include 4-methoxyphenyl, 4-fluorophenyl, 4-chlorophenyl, 4-bromophenyl, 2,4-dimethylphenyl and 2-phenoxyphenyl. Preferred Ar² groups include 2-benzyloxyphenyl, 2,4,6-trimethoxyphenyl, 4-ethoxy-3-methoxyphenyl, 2,3,5-trichlorophenyl and 2,3,4,5,6-pentafluorophenyl.

Another particular embodiment of the invention comprises a compound of formula I, or a salt thereof, wherein D is —C≡N, G is NR¹, preferably NH, m is 1, and n is 2.

Another particular embodiment of the invention comprises a compound of formula I, or a salt thereof, wherein Ar² is substituted or unsubstituted phenyl, D is —C≡N, G is NR¹, preferably NH, m is 1, and n is 2.

Another particular embodiment of the invention comprises a compound of formula I, or a salt thereof, wherein D is —C≡N, G is NR¹, preferably NH, m is 1, n is 2, and R⁴ is other than hydrogen, preferably halogen or —OR⁷, preferably alkoxy.

Another particular embodiment of the invention comprises a compound of formula I, or a salt thereof, wherein D is —C≡N, G is NR¹, preferably NH, m is 1, n is 2, each occurrence of both R² and R³ is hydrogen, and R⁴ is other than hydrogen, preferably halogen or —OR⁷, preferably alkoxy.

Another particular embodiment of the invention comprises a compound of formula I, or a salt thereof, wherein D is —C≡N, G is NR¹, preferably NH, m is 1, n is 2, each occurrence R³ is other than (C₁-C₃) perfluoroalkyl.

Another particular embodiment of the invention comprises a compound of formula I, or a salt thereof, wherein D is —C≡N, G is NR¹, preferably NH, m is 1, n is 2, each occurrence of both R² and R³ is other than (C₁-C₃) perfluoroalkyl, and R⁴ is other than (C₁-C₃) perfluoroalkyl.

Another particular embodiment of the invention comprises a compound of formula I, or a salt thereof, wherein D is —C≡N, G is NR¹, preferably NH, m is 1, n is 2, each occurrence of both R² and R³ is hydrogen, and R⁴ is selected from the group consisting of halogen and —OR⁷, wherein R⁷ is preferably alkoxy.

Another particular embodiment of the invention comprises a compound of formula I, or a salt thereof, wherein D is —C≡N, G is NR¹, preferably NH, and Ar¹ is 4-methoxyphenyl.

Another particular embodiment of the invention comprises a compound of formula I, or a salt thereof, wherein D is —C≡N, G is NR¹, preferably NH, m is 1, n is 2, and Ar¹ is selected from the group consisting of 4-methoxyphenyl, 4-fluorophenyl, 4-chlorophenyl, 4-bromophenyl, 2-phenoxyphenyl, 2,4-dimethylphenyl, 4-methoxy-3-(4-methylsulfonyloxy)phenyl, 3,4-dimethoxyphenyl, 3-nitro-4-methoxyphenyl, 3-amino-4-methoxyphenyl, 3-fluoro-4-methoxyphenyl, and 2,4-dichlorophenyl.

Another particular embodiment of the invention comprises a compound of formula I, or a salt thereof, wherein D is —C≡N, G is NR¹, preferably NH, m is 1, and n is 2, Ar² is substituted phenyl substituted at the 4-position by other than hydroxy.

Another particular embodiment of the invention comprises a compound of formula I, or a salt thereof, wherein D is —C≡N, G is NR¹, preferably NH, m is 1, and n is 2, Ar² is substituted phenyl, and each substituent of Ar² is other than hydroxy.

Another particular embodiment of the invention comprises a compound of formula I, or a salt thereof, wherein D is —C≡N, G is NR¹, preferably NH, m is 1, n is 2, Ar² is substituted phenyl substituted at the 4-position by halogen, preferably fluorine or —OR⁷, preferably alkoxy.

Another particular embodiment of the invention comprises a compound of formula I, or a salt thereof, wherein D is —C≡N, G is NR¹, preferably NH, m is 1, and n is 2, and Ar² is selected from the group consisting of 2,3,4,5,6-pentafluorophenyl; 2,3,4-trimethoxyphenyl; 2,3,5-trichlorophenyl; 2,4,5-trimethoxyphenyl; 2,4,6-trimethoxyphenyl; 2,4-difluorophenyl; 2,5-dimethoxyphenyl; 2,5-dimethylphenyl; 2,6-dimethoxyphenyl; 2-benzyloxyphenyl; 2-chlorophenyl; 2-fluoro-4-methoxyphenyl; 2-methoxyphenyl; 2-methoxyphenyl; 2-phenoxyphenyl; 3,4,5-trimethoxyphenyl; 3,4-dichlorophenyl; 3-chloro-4-fluorophenyl; 3-indolyl; 3-methylthiophen-2-yl; 3-methylthiophen-2-yl; 3-methylthiophen-2-yl; 3-nitro-4-hydroxyphenyl; 4-(N,N-dimethylamino)phenyl; 4-biphenyl-1-yl; 4-bromophenyl; 4-chlorophenyl; 4-ethoxy-3-methoxyphenyl; 4-fluorophenyl; 4-methoxyphenyl; 5-bromo-3-indolyl; and 5-methylthiophen-2-yl.

Another particular embodiment of the invention comprises a compound of formula I, or a salt thereof, wherein D is —C≡N, G is NR¹, preferably NH, m is 1, and n is 2, and Ar² is selected from the group consisting of 2,4,6-trimethoxyphenyl and 2,3,4,5,6-pentafluorophenyl.

Another particular embodiment of the invention comprises a compound of formula I, or a salt thereof, wherein D is —C(═O)NR⁸₂, G is NR¹, preferably NH, m is 1, and n is 2.

Another particular embodiment of the invention comprises a compound of formula I, or a salt thereof, wherein D is —NO₂, G is NR¹, preferably NH, m is 1, and n is 2.

Particular compounds that are embodiments of the invention include the following: (E)-2-[(4-methoxyphenyl)methanesulfonyl]-3-(2,4,6-tnmethoxyphenyl)acrylonitrile; (E)-2-[(4-methoxyphenyl)methanesulfonyl]-3-(2,4-difluorophenyl)acrylonitrile; (E)-2-[(4-methoxyphenyl)methanesulfonyl]-3-(3-nitro-4-fluorophenyl)acrylonitrile; (E)-2-[(4-bromophenyl)methanesulfonyl]-3-(2,4-difluorophenyl)acrylonitrile; (E)-2-[(4-methoxyphenyl)methanesulfonyl]-3-(4-aminophenyl)acrylonitrile; (E)-2-[(4-methoxyphenyl)methanesulfonyl]3-(3-amino-4-fluorophenyl)acrylonitrile; (E)-2-[(4-methoxyphenyl)methanesulfonyl]-3-(4-fluorophenyl)acrylonitrile; (E)-2-[(4-methoxyphenyl)methanesulfonyl]-3-(2,6-dimethoxyphenyl)acrylonitrile; (E)-2-[(4-methoxyphenyl)methanesulfonyl]-3-(2-methoxyphenyl)acrylonitrile; (E)-2-[(4-methoxyphenyl)methanesulfonyl]-3-(4-nitrophenyl)acrylonitrile; (E)-2-[(4-methoxyphenyl)methanesulfonyl]-3-(2,3,4,5,6-pentafluorophenyl)acrylonitrile; (E)-2-[(4-methoxyphenyl)methanesulfonyl]-3-(3-furyl)acrylonitrile; (E)-2-[(4-methoxyphenyl)methanesulfonyl]-3-(thiophen-3-yl)acrylonitrile; (E)-2-[(4-bromophenyl)methanesulfonyl]-3-(2-difluoromethoxyphenyl)acrylonitrile; (E)-2-[(4-methoxyphenyl)methanesulfonyl]-3-(2-chloro-4-fluorophenyl)acrylonitrile; (E)-2-[(4-bromophenyl)methanesulfonyl]-3-(2-fluoro-4-cyanophenyl)acrylonitrile; (E)-2-[(4-methoxyphenyl)methanesulfonyl]-3-(2-fluoro-4-cyanophenyl)acrylonitrile; (E)-2-[(4-bromophenyl)methanesulfonyl]-3-(2,6-dimethoxyphenyl)acrylonitrile; (E)-2-[(4-bromophenyl)methanesulfonyl]-3-(2,3,4,5,6-pentafluorophenyl)acrylonitrile; (E)-2-[(4-bromophenyl)methanesulfonyl]-3-(2,4,6-trimethoxyphenyl)acrylonitrile; (E)-2-[(4-chlorophenyl)methanesulfonyl]-3-(2,3,4,5,6-pentafluorophenyl)acrylonitrile; (E)-2-[(4-methoxyphenyl)methanesulfonyl]-3-(2-benzoxazolon-5-yl)acrylonitrile; (E)-2-[(4-methoxyphenyl)methanesulfonyl]3-(2-benzoxazolon-6-yl)acrylonitrile; (E)-2-[(4-chlorophenyl)methanesulfonyl]-3-(3,4-dihydroxyphenyl)acrylonitrile; (E)-2-[(4-methoxyphenyl)methanesulfonyl]-3-(3,4-dihydroxyphenyl)acrylonitrile; (E)-2-[(4-methoxyphenyl)methanesulfonyl]-3-(3,4-dimethoxyphenyl)acrylonitrile; (E)-2-[(4-methoxyphenyl)methanesulfonyl]-3-(1,3-benzodioxole-5-yl)acrylonitrile; (E)-2-[(3,4-dimethoxyphenyl)methanesulfonyl]-3-(3,4-dihydroxyphenyl)acrylonitrile; (E)-2-[(3,4-dimethoxyphenyl)methanesulfonyl]-3-(2-benzoxazolon-6-yl)acrylonitrile; (E)-2-[(3,4-dimethoxyphenyl)methanesulfonyl]-3-(2-benzoxazolon-5-yl)acrylonitrile; (E)-2-[(4-bromophenyl)methanesulfonyl]-3-(4-nitrophenyl)acrylonitrile; (E)-2-[(3,4 dimethoxyphenyl)methanesulfonyl]-3-(2,3,4,5,6-pentafluorophenyl)acrylonitrile; (E)-2-[(4-fluorophenyl)methanesulfonyl]-3-(2,3,4,5,6-pentafluorophenyl)acrylonitrile; (E)-2-[(3-nitro-4-methoxyphenyl)methanesulfonyl]-3-(2,4,6-trimethoxyphenyl)acrylonitrile; (E)-2-[(3-nitro-4-methoxyphenyl)methanesulfonyl]-3-(2,3,4,5,6-pentafluorophenyl)-acrylonitrile; and (E)-2-[(3-amino-4-methoxyphenyl)methanesulfonyl]-3-(2,4,6-trimethoxyphenyl)acrylonitrile; and salts thereof.

Compounds that are embodiments of the invention also include: (E)-2-[(4-methoxyphenyl)methanesulfonyl]-3-(2,4,6-trimethoxyphenyl)acrylonitrile; (E)-2-[(4 methoxyphenyl)methanesulfonyl]-3-(3-nitro-4-fluorophenyl)acrylonitrile; (E)-2-[(4-methoxyphenyl)methanesulfonyl]-3-(4-aminophenyl)acrylonitrile; (E)-2-[(4-methoxyphenyl)methanesulfonyl]-3-(2,6-dimethoxyphenyl)acrylonitrile; (E)-2-[(4-methoxyphenyl)methanesulfonyl]-3-(2,3,4,5,6-pentafluorophenyl)acrylonitrile; (E)-2-[(4-methoxyphenyl)methanesulfonyl]-3-(2-chloro-4-fluorophenyl)acrylonitrile; (E)-2-[(4-bromophenyl)methanesulfonyl]-3-(2-fluoro-4-cyanophenyl)acrylonitrile; (E)-2-[(4-bromophenyl)methanesulfonyl]-3-(2,6-dimethoxyphenyl)acrylonitrile; (E)-2-[(4-bromophenyl)methanesulfonyl]-3-(2,3,4,5,6-pentafluorophenyl)acrylonitrile; (E)-2-[(4-bromophenyl)methanesulfonyl]-3-(2,4,6-trimethoxyphenyl)acrylonitrile; (E)-2-[(4-chlorophenyl)methanesulfonyl]-3-(2,3,4,5,6-pentafluorophenyl)acrylonitrile; (E)-2-[(4-methoxyphenyl)methanesulfonyl]-3-(2-benzoxazolon-5-yl)acrylonitrile; (E)-2-[(4-methoxyphenyl)methanesulfonyl]-3-(2-benzoxazolon-6-yl)acrylonitrile; (E)-2-[(4-chlorophenyl)methanesulfonyl]-3-(3,4-dihydroxyphenyl)acrylonitrile; (E)-2-[(4-methoxyphenyl)methanesulfonyl]-3-(3,4-dihydroxyphenyl)acrylonitrile; (E)-2-[(3,4-dimethoxyphenyl)methanesulfonyl]-3-(2,3,4,5,6-pentafluorophenyl)acrylonitrile; (E)-2-[(4-fluorophenyl)methanesulfonyl]-3-(2,3,4,5,6-pentafluorophenyl)acrylonitrile; (E)-2-[(3-nitro-4-methoxyphenyl)methanesulfonyl]-3-(2,3,4,5,6-pentafluorophenyl)acrylonitrile; (E)-2-[(3-amino-4-methoxyphenyl)methanesulfonyl]-3-(2,4,6-trimethoxyphenyl)acrylonitrile; (E)-2-[(4-fluorophenyl)methanesulfonyl]-3-(4-ethoxycarbonyl-3,5-dimethylpyrrol-2-yl)acrylonitrile; (E)-2-[(4-methoxy-3-(4-methylphenylsulfonyloxy)phenyl)methanesulfonyl]-3-(2,3,4,5,6-pentafluorophenyl)acrylonitrile; (E)-2-[(3-fluoro-4-methoxyphenyl)methanesulfonyl]-3-(2,4,6-trimethoxyphenyl)acrylonitrile; (E)-2-[(4-chlorophenyl)methanesulfonyl]-3-(2,5-dimethylphenyl)acrylonitrile; (E)-2-[(4-chlorophenyl)methanesulfonyl]-3-(2-chloro-4-fluorophenyl)acrylonitrile; (E)-2-[(4-chlorophenyl)methanesulfonyl]-3-(4-fluoro-3-methylphenyl)acrylonitrile; (E)-2-[(4-chlorophenyl)methanesulfonyl]-3-(4-methanesulfenyl)acrylonitrile; (E)-2-[(4-chlorophenyl)methanesulfonyl]-3-(3-hydroxy-4-methoxyphenyl)acrylonitrile; (E)-2-[(4-chlorophenyl)methanesulfonyl]-3-(4-bromophenyl)acrylonitrile; (E)-2-[(4-chlorophenyl)methanesulfonyl]-3-(4-fluorophenyl)acrylonitrile; (E)-2-[(4-chlorophenyl)methanesulfonyl]-3-(2-fluoro-4-nitrophenyl)acrylonitrile; (E)-2-[(4-chlorophenyl)methanesulfonyl]-3-(2-fluoro-4-methoxyphenyl)acrylonitrile; (E)-2-[(4-chlorophenyl)methanesulfonyl]-3-(2,4-dichlorophenyl)acrylonitrile; (E)-2-[(4-chlorophenyl)methanesulfonyl]-3-(3,4-dichlorophenyl)acrylonitrile; (E)-2-[(4-chlorophenyl)methanesulfonyl]-3-(2,4,6-trimethoxyphenyl)acrylonitrile; (E)-2-[(4-chlorophenyl)methanesulfonyl]-3-(3-ethoxy-4-hydroxyphenyl)acrylonitrile; (E)-2-[(4-chlorophenyl)methanesulfonyl]-3-(4-ethoxy-3-methoxyphenyl)acrylonitrile; (E)-2-[(4-chlorophenyl)methanesulfonyl]-3-(3,5-dimethoxyphenyl)acrylonitrile; (E)-2-[(4-chlorophenyl)methanesulfonyl]-3-(4-acetoxyphenyl)acrylonitrile; (E)-2-[(4-chlorophenyl)methanesulfonyl]-3-(4-(N,N-dimethylamino)phenyl)acrylonitrile; (E)-2-[(4-chlorophenyl)methanesulfonyl]-3-(4-hydroxy-3-nitrophenyl)acrylonitrile; (E)-2-[(4-chlorophenyl)methanesulfonyl]-3-(4-chlorophenyl)acrylonitrile; (E)-2-[(4-fluorophenyl)methanesulfonyl]-3-(4-(N,N-dimethylamino)phenyl)acrylonitrile; (E)-2-[(2,4-dichlorophenyl)methanesulfonyl]-3-(2-fluoro-4-methoxyphenyl)acrylonitrile; (E)-2-[(4-chlorophenyl)methanesulfonyl]-3-(3,4-dimethylphenyl)acrylonitrile; (E-2-[(4-chlorophenyl)methanesulfonyl]-3-(3-indolyl)acrylonitrile; (E)-2-[(2,4-dichlorophenyl)methanesulfonyl]-3-(5-bromo-3-indolyl)acrylonitrile; (E)-2-[(4-chlorophenyl)methanesulfonyl]-3-(5-chloro-3-indolyl)acrylonitrile; (E)-2-[(4-chlorophenyl)methanesulfonyl]-3-(3-ethoxy-4-hydroxyphenyl)acrylonitrile; (E)-2-[(2,4-dichlorophenyl)methanesulfonyl]-3-(5-chloro-3-indolyl)acrylonitrile; (E)-2-[(2,4-dichlorophenyl)methanesulfonyl]-3-(4-methanesulfenyl)acrylonitrile; (E)-2-[(4-fluorophenyl)methanesulfonyl]-3-(5-chloro-3-indolyl)acrylonitrile; (E)-2-[(4-fluorophenyl)methanesulfonyl]-3-(5-bromo-3-indolyl)acrylonitrile; (E)-2-[(4-fluorophenyl)methanesulfonyl]-3-(2,4,6-trimethoxyphenyl)acrylonitrile; (E)-2-[(4-fluorophenyl)methanesulfonyl]-3-(3-hydroxy-4-methoxyphenyl)acrylonitrile; (E)-2-[(4-fluorophenyl)methanesulfonyl]-3-(3-indolyl)acrylonitrile; (E)-2-[(4-fluorophenyl)methanesulfonyl]-3-(2,4-dihydroxyphenyl)acrylonitrile; (E)-2-[(4-fluorophenyl)methanesulfonyl]-3-(4-acetoxyphenyl)acrylonitrile; (E)-2-[(4-fluorophenyl)methanesulfonyl]-3-(2,4-dichlorophenyl)acrylonitrile; (E)-2-[(4-fluorophenyl)methanesulfonyl]-3-(4-methanesulfenyl)acrylonitrile; (E)-2-[(4-fluorophenyl)methanesulfonyl]-3-(3,4-dimethylphenyl)acrylonitrile; (E)-2-[(4-fluorophenyl)methanesulfonyl]-3-(4-chlorophenyl)acrylonitrile; (E)-2-[(4-fluorophenyl)methanesulfonyl]-3-(2,5-dimethylphenyl)acrylonitrile; (E)-2-[(2,4-dichlorophenyl)methanesulfonyl]-3-(5-chloro-3-indolyl)acrylonitrile; (E)-2-[(2,4-dichlorophenyl)methanesulfonyl]-3-(2,4-dihydroxyphenyl)acrylonitrile; (E)-2-[(2,4-dichlorophenyl)methanesulfonyl]-3-(2,3,4-trimethoxyphenyl)acrylonitrile; (E)-2-[(2,4-dichlorophenyl)methanesulfonyl]-3-(4-ethoxy-3-methoxyphenyl)acrylonitrile; (E)-2-[(2,4-dichlorophenyl)methanesulfonyl]-3-(2-chloro-4-fluorophenyl)acrylonitrile; (E)-2-[(2,4-dichlorophenyl)methanesulfonyl]-3-(2,5-dimethylphenyl)acrylonitrile; (E)-2-[(2,4-dichlorophenyl)methanesulfonyl]-3-(4-bromophenyl)acrylonitrile; (E)-2-[(2,4-dichlorophenyl)methanesulfonyl]-3-(4-methoxyphenyl)acrylonitrile; (E)-2-[(4-bromophenyl)methanesulfonyl]-3-(3,5-dimethylphenyl)acrylonitrile; (E)-2-[(2,4-dichlorophenyl)methanesulfonyl]-3-(3-methyl-2-thiophen-2-yl)acrylonitrile; (E)-2-[(2,4-dichlorophenyl)methanesulfonyl]-3-(3-methyl-2-furyl)acrylonitrile; (E)-2-[(2,4-dichlorophenyl)methanesulfonyl]-3-(3-furyl)acrylonitrile; (E)-2-[(2,4-dichlorophenyl)methanesulfonyl]-3-(3,4,5-trimethoxyphenyl)acrylonitrile; (E)-2-[(2,4-dichlorophenyl)methanesulfonyl]-3-(2,5-dimethoxyphenyl)acrylonitrile; (E)-2-[(2,4-dichlorophenyl)methanesulfonyl]-3-(2,4,5-trimethoxyphenyl)acrylonitrile; (E)-2-[(2,4-dichlorophenyl)methanesulfonyl]-3-(3,4-dimethoxyphenyl)acrylonitrile; (E)-2-[(2,4-dichlorophenyl)methanesulfonyl]-3-(phenyl)acrylonitrile; (E)-2-[(4-chlorophenyl)methanesulfonyl]-3-(3-methyl-2-thiophen-2-yl)acrylonitrile; (E)-2-[(phenyl)methanesulfonyl]-3-(3-hydroxyphenyl)acrylonitrile; (E)-2-[(phenyl)methanesulfonyl]-3-(2-hydroxyphenyl)acrylonitrile; (E)-2-[(phenyl)methanesulfonyl]-3-(3-methyl-2-furyl)acrylonitrile; (E)-2-[(phenyl)methanesulfonyl]-3-(2-fluoro-4-methoxyphenyl)acrylonitrile; (E)-2-[(phenyl)methanesulfonyl]-3-(3,4,5-trimethoxyphenyl)acrylonitrile; (E)-2-[(phenyl)methanesulfonyl]-3-(3,4-dimethoxyphenyl)acrylonitrile; (E)-2-[(phenyl)methanesulfonyl]-3-(3,5-dimethoxyphenyl)acrylonitrile; (E)-2-[(phenyl)methanesulfonyl]-3-(2,5-dimethylphenyl)acrylonitrile; (E)-2-[(phenyl)methanesulfonyl]-3-(2-benzyloxyphenyl)acrylonitrile; (E)-2-[(phenyl)methanesulfonyl]-3-(4-hydroxy-3-nitrophenyl)acrylonitrile; (E-2-[(phenyl)methanesulfonyl]-3-(3,5-difluorophenyl)acrylonitrile; (E)-2-[(4-bromophenyl)methanesulfonyl]-3-(2-chlorophenyl)acrylonitrile; (E)-2-[(4-methoxyphenyl)methanesulfonyl]-3-(2,3,4-trichlorophenyl)acrylonitrile; (E)-2-[(4-methoxyphenyl)methanesulfonyl]-3-(3,4-dimethoxyphenyl)acrylonitrile; (E)-2-[(4-methoxyphenyl)methanesulfonyl]-3-(3,5-dimethoxyphenyl)acrylonitrile; and salts thereof.

Preferred embodiments of the invention include the following: (E)-2-[(4-methoxyphenyl)methanesulfonyl]-3-(2,4,6-trimethoxyphenyl)acrylonitrile; (E)-2-[(4-methoxyphenyl)methanesulfonyl]-3-(2,3,4,5,6-pentafluorophenyl)acrylonitrile; (E)-2-[(4-methoxyphenyl)methanesulfonyl]-3-(2-chloro-4-fluorophenyl)acrylonitrile; (E)-2-[(4-bromophenyl)methanesulfonyl]-3-(2-fluoro-4-cyanophenyl)acrylonitrile; (E)-2-[(4-methoxyphenyl)methanesulfonyl]-3-(2-fluoro-4-cyanophenyl)acrylonitrile; (E)-2-[(4-bromophenyl)methanesulfonyl]-3-(2,6-dimethoxyphenyl)acrylonitrile; (E)-2-[(4-bromophenyl)methanesulfonyl]-3-(2,3,4,5,6-pentafluorophenyl)acrylonitrile; (E)-2-[(4-bromophenyl)methanesulfonyl]-3-(2,4,6-trimethoxyphenyl)acrylonitrile; (E)-2-[(4-chlorophenyl)methanesulfonyl]-3-(2,3,4,5,6-pentafluorophenyl)acrylonitrile; (E)-2-[(4-methoxyphenyl)methanesulfonyl]-3-(2-benzoxazolon-5-yl)acrylonitrile; (E)-2-[(4-chlorophenyl)methanesulfonyl]-3-(3,4-dihydroxyphenyl)acrylonitrile; (E)-2-[(4-methoxyphenyl)methanesulfonyl]-3-(3,4-dihydroxyphenyl)acrylonitrile; (E)-2-[(3,4-dimethoxyphenyl)methanesulfonyl]-3-(2,3,4,5,6-pentafluorophenyl)acrylonitrile; (E)-2-[(4-fluorophenyl)methanesulfonyl]-3-(2,3,4,5,6-pentafluorophenyl)acrylonitrile; (E)-2-[(3-nitro-4-methoxyphenyl)methanesulfonyl]-3-(2,3,4,5,6-pentafluorophenyl)-acrylonitrile; (E)-2-[(3-amino-4-methoxyphenyl)methanesulfonyl]-3-(2,4,6-trimethoxyphenyl)acrylonitrile; (E)-2-[(3-fluoro-4-methoxyphenyl)methanesulfonyl]-3-(2,4,6-trimethoxyphenyl)acrylonitrile; (E)-2-[(4-chlorophenyl)methanesulfonyl]-3-(2,5-dimethylphenyl)acrylonitrile; (E)-2-[(4-chlorophenyl)methanesulfonyl]-3-(2-fluoro-4-nitrophenyl)acrylonitrile; (E)-2-[(4-fluorophenyl)methanesulfonyl]-3-(4-acetoxyphenyl)acrylonitrile; (E)-2-[(phenyl)methanesulfonyl]-3-(2,5-dimethylphenyl)acrylonitrile; (E)-2-[(4-chlorophenyl)methanesulfonyl]-3-(3,4-dichlorophenyl)acrylonitrile; (E)-2-[(4-chlorophenyl)methanesulfonyl]-3-(4-ethoxy-3-methoxyphenyl)acrylonitrile; (E)-2-[(4-chlorophenyl)methanesulfonyl]-3-(3-indolyl)acrylonitrile; (E)-2-[(4-chlorophenyl)methanesulfonyl]-3-(5-chloro-3-indolyl)acrylonitrile; (E)-2-[(4-fluorophenyl)methanesulfonyl]-3-(3,4-dimethylphenyl)acrylonitrile; (E)-2-[(2,4-dichlorophenyl)methanesulfonyl]-3-(5-chloro-3-indolyl)acrylonitrile; (E)-2-[(2,4-dichlorophenyl)methanesulfonyl]-3-(3,4,5-trimethoxyphenyl)acrylonitrile; (E)-2-[(phenyl)methanesulfonyl]-3-(3,5-dimethoxyphenyl)acrylonitrile and salts thereof.

Other particular embodiments of the invention include the following compounds: (E)-2-[(4-methoxyphenyl)methanesulfonyl]-3-(2,3,4,5,6-pentafluorophenyl)acrylamide; (E)-2-[(4-methoxyphenyl)methanesulfonyl]-3-(2-benzoxazolon-5-yl)acrylamide; (E)-2-[(4-chlorophenyl)methanesulfonyl]-3-(3,4-dihydroxyphenyl)acrylamide; (E)-2-[(4-methoxyphenyl)methanesulfonyl]-3-(3,4-dihydroxyphenyl)acrylamide; (E)-2-[(3,4-dimethoxyphenyl)methanesulfonyl]-3-(2,3,4,5,6-pentafluorophenyl)acrylamide; (E)-2-[(4-fluorophenyl)methanesulfonyl]-3-(2,3,4,5,6-pentafluorophenyl)acrylamide; (E)-2-[(3-nitro-4-methoxyphenyl)methanesulfonyl]-3-(2,3,4,5,6-pentafluorophenyl)acrylamide; and salts thereof.

Other particular embodiments of the invention include the following compounds: (E)-2-[(4-methoxyphenyl)methanesulfonyl]-3-(2,3,4,5,6-pentafluorophenyl)acrylamide; (E)-2-[(4-methoxyphenyl)methanesulfonyl]-3-(2-benzoxazolon-5-yl)acrylamide; (E)-2-[(4-chlorophenyl)methanesulfonyl]-3-(3,4-dihydroxyphenyl)acrylamide; (E)-2-[(4-methoxyphenyl)methanesulfonyl]-3-(3,4-dihydroxyphenyl)acrylamide; (E)-2-[(3,4-dimethoxyphenyl)methanesulfonyl]-3-(2,3,4,5,6-pentafluorophenyl)acrylamide; (E)-2-[(4-fluorophenyl)methanesulfonyl]-3-(2,3,4,5,6-pentafluorophenyl)acrylamide; (E)-2-[(3-nitro-4-methoxyphenyl)methanesulfonyl]-3-(2,3,4,5,6-pentafluorophenyl)acrylamide; and salts thereof.

Other particular embodiments of the invention include the following compounds: (E)-1,2,3,4,5-pentafluoro-6-{2-[(4-methoxyphenyl)methanesulfonyl]-2-nitrovinyl}-benzene; (E)-5-{2-[(4-methoxyphenyl)methanesulfonyl]-2-nitrovinyl}benzo[d]oxazol-2(3H)-one; (E)-4-{2-[(4-chlorophenyl)methanesulfonyl]-2-nitrovinyl}benzene-1,2-diol; (E)-4-{2-[(4-methoxyphenyl)methanesulfonyl]-2-nitrovinyl}benzene-1,2-diol; (E)-1,2,3,4,5-pentafluoro-6-{2-[(3,4-dimethoxyphenyl)methanesulfonyl]-2-nitrovinyl}-benzene; (E)-1,2,3,4,5-pentafluoro-6-{2-[(4-fluorophenyl)methanesulfonyl]-2-nitrovinyl}benzene; (E)-1,2,3,4,5-pentafluoro-6-{2-[(3-nitro-4-methoxyphenyl)methanesulfonyl]-2-nitrovinyl}-benzene; and salts thereof.

Other particular embodiments of the invention include the following compounds: (E)-1,2,3,4,5-pentafluoro-6-{2-[(4-methoxyphenyl)sulfonyl]-2-nitrovinyl}benzene; (E)-5{2-[(4-methoxyphenyl)sulfonyl]-2-nitrovinyl}benzo[d]oxazol-2(3H)-one; (E)-4-{2-[(4-chlorophenyl)sulfonyl]-2-nitrovinyl}benzene-1,2-diol; (E)-4-{2-[(4-methoxyphenyl)sulfonyl]-3-nitrovinyl}benzene-1,2-diol; (E)-1,2,3,4,5-pentafluoro-6-{2-[(3,4-dimethoxyphenyl)sulfonyl]-2-nitrovinyl}benzene; (E)-1,2,3,4,5-pentafluoro-6-{2-[(4-fluorophenyl)sulfonyl]-2-nitrovinyl}benzene; (E)-1,2,3,4,5-pentafluoro-6-{2-[(3-nitro-4-methoxyphenyl)sulfonyl]-2-nitrovinyl}benzene; and salts thereof.

Other particular embodiments of the invention include the following compounds: (E)-2-[(4-methoxyphenyl)methanesulfonyl]-3-(2,3,4,5,6-pentafluorophenyl)acrylonitrile; (E)-2-[(4-methoxyphenyl)methanesulfonyl]-3-(2-benzoxazolon-5-yl)acrylonitrile; (E)-2-[(4-chlorophenyl)methanesulfonyl]-3-(3,4-dihydroxyphenyl)acrylonitrile; (E)-2-[(4-methoxyphenyl)methanesulfonyl]-3-(3,4-dihydroxyphenyl)acrylonitrile; (E)-2-[(3,4-dimethoxyphenyl)methanesulfonyl]-3-(2,3,4,5,6-pentafluorophenyl)acrylonitrile; (E)-2-[(4-fluorophenyl)methanesulfonyl]-3-(2,3,4,5,6-pentafluorophenyl)acrylonitrile; (E)-2-[(3-nitro-4-methoxyphenyl)methanesulfonyl]-3-(2,3,4,5,6-pentafluorophenyl)acrylonitrile; and salts thereof.

Other particular embodiments of the invention include the following compounds: (E)-2-[(4-methoxyphenyl)methanesulfinyl]-3-(2,3,4,5,6-pentafluorophenyl)acrylamide; (E)-2-[(4-methoxyphenyl)methanesulfinyl]-3-(2-benzoxazolon-5-yl)acrylamide; (E)-2-[(4-chlorophenyl)methanesulfinyl]-3-(3,4-dihydroxyphenyl)acrylamide; (E)-2-[(4-methoxyphenyl)methanesulfinyl]-3-(3,4-dihydroxyphenyl)acrylamide; (E)-2-[(3,4-dimethoxyphenyl)methanesulfinyl]-3-(2,3,4,5,6-pentafluorophenyl)acrylamide; (E)-2-[(4-fluorophenyl)methanesulfinyl]-3-(2,3,4,5,6-pentafluorophenyl)acrylamide; (E)-2-[(3-nitro-4-methoxyphenyl)methanesulfinyl]-3-(2,3,4,5,6-pentafluorophenyl)acrylamide; and salts thereof.

Other particular embodiments of the invention include the following compounds: (E)-1,2,3,4,5-pentafluoro-6-{2-[(4-methoxyphenyl)methanesulfinyl]-2-nitrovinyl}benzene; (E)-5-{2-[(4-methoxyphenyl)methanesulfinyl]-2-nitrovinyl}benzo[d]oxazol-2(3H)-one; (E)-4-{2-[(4-chlorophenyl)methanesulfinyl]-2-nitrovinyl}benzene-1,2-diol; (E)-4-{2-[(4-methoxyphenyl)methanesulfinyl]-2-nitrovinyl}benzene-1,2-diol; (E)-1,2,3,4,5-pentafluoro-6-{2-[(3,4-dimethoxyphenyl)methanesulfinyl]-2-nitrovinyl}benzene; (E)-1,2,3,4,5-pentafluoro-6-{2[(4-fluorophenyl)methanesulfinyl]-2-nitrovinyl}benzene; (E)-1,2,3,4,5-pentafluoro-6-{2-[(3-nitro-4-methoxyphenyl)methanesulfinyl]-2-nitrovinyl}benzene; and salts thereof.

Other particular embodiments of the invention include the following compounds: (E)-1,2,3,4,5-pentafluoro-6-{2-[(4-methoxyphenyl)sulfinyl]-2-nitrovinyl}benzene; (E)-5-{2-[(4-methoxyphenyl)sulfinyl]-2-nitrovinyl}benzo[d]oxazol-2(3H)-one; (E)-4-{2-[(4-chlorophenyl)sulfinyl]-2-nitrovinyl}benzene-1,2-diol; (E)-4-{2-[(4-methoxyphenyl)sulfinyl]-2-nitrovinyl}benzene-1,2-diol; (E)-1,2,3,4,5-pentafluoro-6-{2-[(3,4-dimethoxyphenyl)sulfinyl]-2-nitrovinyl}benzene; (E)-1,2,3,4,5-pentafluoro-6-{2-[(4-fluorophenyl)sulfinyl]-2-nitrovinyl}benzene; (E)-1,2,3,4,5-pentafluoro-6-{2-[(3-nitro-4-methoxyphenyl)sulfinyl]-2-nitrovinyl}benzene and salts thereof.

Other particular embodiments of the invention include the following compounds: (E)-2-[(4-methoxyphenyl)methanesulfenyl]-3-(2,3,4,5,6-pentafluorophenyl)acrylonitrile; (E)-2-[(4-methoxyphenyl)methanesulfenyl]-3-(2-benzoxazolon-5-yl)acrylonitrile; (E)-2-[(4-chlorophenyl)methanesulfenyl]-3-(3,4-dihydroxyphenyl)acrylonitrile; (E)-2-[(4-methoxyphenyl)methanesulfenyl]-3-(3,4-dihydroxyphenyl)acrylonitrile; (E)-2-[(3,4-dimethoxyphenyl)methanesulfenyl]-3-(2,3,4,5,6-pentafluorophenyl)acrylonitrile; (E)-2-[(4-fluorophenyl)methanesulfenyl]-3-(2,3,4,5,6-pentafluorophenyl)acrylonitrile; (E)-2-[(3-nitro-4-methoxyphenyl)methanesulfenyl]-3-(2,3,4,5,6-pentafluorophenyl)acrylonitrile; and salts thereof.

Other particular embodiments of the invention include the following compounds: (E)-2-[(4-methoxyphenyl)methanesulfenyl]-3-(2,3,4,5,6-pentafluorophenyl)acrylamide; (E)-2-[(4-methoxyphenyl)methanesulfenyl]-3-(2-benzoxazolon-5-yl)acrylamide; (E)-2-[(4-chlorophenyl)methanesulfenyl]-3-(3,4-dihydroxyphenyl)acryl amide; (E)-2-[(4-methoxyphenyl)methanesulfenyl]-3-(3,4-dihydroxyphenyl)acrylamide; (E)-2-[(3,4-dimethoxyphenyl)methanesulfenyl]-3-(2,3,4,5,6-pentafluorophenyl)acrylamide; (E)-2-[(4-fluorophenyl)methanesulfenyl]-3-(2,3,4,5,6-pentafluorophenyl)acrylamide; (E)-2-[(3-nitro-4-methoxyphenyl)methanesulfenyl]-3-(2,3,4,5,6-pentafluorophenyl)acrylamide; and salts thereof.

Other particular embodiments of the invention include the following compounds: (E)-1,2,3,4,5-pentafluoro-6-{2-[(4-methoxyphenyl)methanesulfenyl]-2-nitrovinyl}benzene; (E)-5-{2-[(4-methoxyphenyl)methanesulfenyl]-2-nitrovinyl}benzo[d]oxazol-2(3H)-one; (E)-4-{2-[(4-chlorophenyl)methanesulfenyl]-2-nitrovinyl}benzene-1,2-diol; (E)-4-{2-[(4-methoxyphenyl)methanesulfenyl]-2-nitrovinyl}benzene-1,2-diol; (E)-1,2,3,4,5-pentafluoro-6-{2-[(3,4-dimethoxyphenyl)methanesulfenyl]-2-nitrovinyl}benzene; (E)-1,2,3,4,5-pentafluoro-6-{2-[(4-fluorophenyl)methanesulfenyl]-2-nitrovinyl}benzene; (E)-1,2,3,4,5-pentafluoro-6-{2-[(3-nitro-4-methoxyphenyl)methanesulfenyl]-2-nitrovinyl}benzene; and salts thereof.

Other particular embodiments of the invention include the following compounds: (E)-1,2,3,4,5-pentafluoro-6-{2-[(4-methoxyphenyl)sulfenyl]-2-nitrovinyl}benzene; (E)-5-{2-[(4-methoxyphenyl)sulfenyl]-2-nitrovinyl}benzo[d]oxazol-2(3H)-one; (E)-4-{2-[(4-chlorophenyl)sulfenyl]-2-nitrovinyl}benzene-1,2-diol; (E)-4-{2-[(4-methoxyphenyl)sulfenyl]-2-nitrovinyl}benzene-1,2-diol; (E)-1,2,3,4,5-pentafluoro-6-{2-[(3,4-dimethoxyphenyl)sulfenyl]-2-nitrovinyl}benzene; (E)-1,2,3,4,5-pentafluoro-6-{2-[(4-fluorophenyl)sulfenyl]-2-nitrovinyl}benzene; (E)-1,2,3,4,5-pentafluoro-6-{2-[(3-nitro-4-methoxyphenyl)sulfenyl]-2-nitrovinyl}benzene; and salts thereof.

Other particular embodiments of the invention include the following compounds: (E)-2-[(4-methoxyphenyl)sulfamoyl]-3-(2,3,4,5,6-pentafluorophenyl)acrylonitrile; (E)-2-[(4-methoxyphenyl)sulfamoyl]-3-(2-benzoxazolon-5-yl)acrylonitrile; (E)-2-[(4-chlorophenyl)sulfamoyl]-3-(3,4-dihydroxyphenyl)acrylonitrile; (E)-2-[(4 methoxyphenyl)sulfamoyl]-3-(3,4-dihydroxyphenyl)acrylonitrile; (E)-2-[(3,4-dimethoxyphenyl)sulfamoyl]-3-(2,3,4,5,6-pentafluorophenyl)acrylonitrile; (E)-2-[(4-fluorophenyl)sulfamoyl]-3-(2,3,4,5,6-pentafluorophenyl)acrylonitrile; (E)-2-[(3-nitro-4-methoxyphenyl)sulfamoyl]-3-(2,3,4,5,6-pentafluorophenyl)acrylonitrile; and salts thereof.

Other embodiments of the invention include the following compounds: (E)-2-[(4-methoxyphenyl)sulfamoyl]-3-(4-bromophenyl)acrylonitrile; (E)-2-[(4-methoxyphenyl)sulfamoyl]-3-(2,4,6-trimethoxyphenyl)acrylonitrile; (E)-2-[(4-methoxyphenyl)sulfamoyl]-3-(2,3,4,5,6-pentafluorophenyl)acrylonitrile; (E)-2-[(4-methoxyphenyl)sulfamoyl]-3-(2,6-dimethoxyphenyl)acrylonitrile; (E)-2-[(4-methoxy-3-(4-methylphenylsulfonyloxy)phenyl)sulfamoyl]-3-(2,3,4,5,6-pentafluorophenyl)acrylonitrile; (E)-2-[(4-chlorophenyl)sulfamoyl]-3-(4-methoxyphenyl)acrylonitrile; (E)-2-[(2-phenoxyphenyl)sulfamoyl]-3-(4-bromophenyl)acrylonitrile; (E)-2-[(2-phenoxyphenyl)sulfamoyl]-3-(3-indolyl)acrylonitrile; (E)-2-[(2-phenoxyphenyl)sulfamoyl]-3-(5-methylthiophen-2-yl)acrylonitrile; (E)-2-[(2-phenoxyphenyl)sulfamoyl]-3-(2-methoxyphenyl)acrylonitrile; (E)-2-[(4-methoxyphenyl)sulfamoyl]-3-(4-fluorophenyl)acrylonitrile; (E)-2-[(4-methoxyphenyl)sulfamoyl]-3-(2,5-dimethoxyphenyl)acrylonitrile; (E)-2-[(4-methoxyphenyl)sulfamoyl]-3-(2-methoxyphenyl)acrylonitrile; (E)-2-[(4-chlorophenyl)sulfamoyl]-3-(3-indolyl)acrylonitrile; (E)-2-[(2-phenoxyphenyl)sulfamoyl]-3-(4-chlorophenyl)acrylonitrile; (E)-2-[(2-phenoxyphenyl)sulfamoyl]-3-(2,4,6-trimethoxyphenyl)acrylonitrile; (E)-2-[(2-phenoxyphenyl)sulfamoyl]-3-(3-nitro-4-hydroxyphenyl)acrylonitrile; (E)-2-[(2-phenoxyphenyl)sulfamoyl]-3-(2-phenoxyphenyl)acrylonitrile; (E)-2-[(2-phenoxyphenyl)sulfamoyl]-3-(4-ethoxy-3-methoxyphenyl)acrylonitrile; (E)-2-[(4-bromophenyl)sulfamoyl]-3-(4-chlorophenyl)acrylonitrile; (E)-2-[(4-bromophenyl)sulfamoyl]-3-(2-benzyloxyphenyl)acrylonitrile; (E)-2-[(4-bromophenyl)sulfamoyl]-3-(3-indolyl)acrylonitrile; (E)-2-[(4-bromophenyl)sulfamoyl]-3-(2,4,6-trimethoxyphenyl)acrylonitrile; (E)-2-[(4-bromophenyl)sulfamoyl]-3-(4-(N,N-dimethylamino)phenyl)acrylonitrile; (E)-2-[(4-bromophenyl)sulfamoyl]-3-(4-bromophenyl)acrylonitrile; (E)-2-[(4-bromophenyl)sulfamoyl]-3-(2,3,4-trimethoxyphenyl)acrylonitrile; (E)-2-[(4-bromophenyl)sulfamoyl]-3-(4-ethoxy-3-methoxyphenyl)acrylonitrile; (E)-2-[(4-bromophenyl)sulfamoyl]-3-(4-biphenyl-1-yl)acrylonitrile; (E)-2-[(4-bromophenyl)sulfamoyl]-3-(2,3,4,5,6-pentafluorophenyl)acrylonitrile; (E)-2-[(4-bromophenyl)sulfamoyl]-3-(5-bromo-3-indolyl)acrylonitrile; (E)-2-[(4-bromophenyl)sulfamoyl]-3-(3-methylthiophen-2-yl)acrylonitrile; (E)-2-[(4-bromophenyl)sulfamoyl]-3-(2-methoxyphenyl)acrylonitrile; (E)-2-[(4-bromophenyl)sulfamoyl]-3-(2,4-difluorophenyl)acrylonitrile; (E)-2-[(4-bromophenyl)sulfamoyl]-3-(2-chlorophenyl)acrylonitrile; (E)-2-[(4-bromophenyl)sulfamoyl]-3-(2-hydroxyphenyl)acrylonitrile; (E)-2-[(4-bromophenyl)sulfamoyl]-3-(2,5-dimethylphenyl)acrylonitrile; (E)-2-[(4-fluorophenyl)sulfamoyl]-3-(2-benzyloxyphenyl)acrylonitrile; (E)-2-[(4-fluorophenyl)sulfamoyl]-3-(3-indolyl)acrylonitrile; (E)-2-[(4-fluorophenyl)sulfamoyl]-3-(2,4,6-trimethoxyphenyl)acrylonitrile; (E)-2-[(4-fluorophenyl)sulfamoyl]-3-(4-(N,N-dimethylamino)phenyl)acrylonitrile; (E)-2-[(4-fluorophenyl)sulfamoyl]-3-(2,3,4,5,6-pentafluorophenyl)acrylonitrile; (E)-2-[(4-fluorophenyl)sulfamoyl]-3-(2-chlorophenyl)acrylonitrile; (E)-2-[(4-fluorophenyl)sulfamoyl]-3-(3-methylthiophen-2-yl)acrylonitrile; (E)-2-[(4-fluorophenyl)sulfamoyl]-3-(2,5-dimethylphenyl)acrylonitrile; (E)-2-[(4-fluorophenyl)sulfamoyl]-3-(4-biphenyl-1-yl)acrylonitrile; (E)-2-[(4-fluorophenyl)sulfamoyl]-3-(2,4-difluorophenyl)acrylonitrile; (E)-2-[(4-fluorophenyl)sulfamoyl]-3-(2-fluoro-4-methoxyphenyl)acrylonitrile; (E)-2-[(4-fluorophenyl)sulfamoyl]-3-(4-bromophenyl)acrylonitrile; (E)-2-[(4-fluorophenyl)sulfamoyl]-3-(2,3,5-trichlorophenyl)acrylonitrile; (E)-2-[(4-fluorophenyl)sulfamoyl]-3-(3-chloro-4-fluorophenyl)acrylonitrile; (E)-2-[(4-fluorophenyl)sulfamoyl]-3-(4-chlorophenyl)acrylonitrile; (E)-2-[(4-fluorophenyl)sulfamoyl]-3-(5-bromo-3-indolyl)acrylonitrile; (E)-2-[(2,4-dimethylphenyl)sulfamoyl]-3-(2-benzyloxyphenyl)acrylonitrile; (E)-2-[(2,4-dimethylphenyl)sulfamoyl]-3-(3-indolyl)acrylonitrile; (E)-2-[(2,4-dimethylphenyl)sulfamoyl]-3-(2,4,6-trimethoxyphenyl)acrylonitrile; (E)-2-[(2,4-dimethylphenyl)sulfamoyl]-3-(4-(N,N-dimethylamino)phenyl)acrylonitrile; (E)-2-[(2,4-dimethylphenyl)sulfamoyl]-3-(2-chlorophenyl)acrylonitrile; (E)-2-[(2,4-dimethylphenyl)sulfamoyl]-3-(3-methylthiophen-2-yl)acrylonitrile; (E)-2-[(2,4-dimethylphenyl)sulfamoyl]-3-(2,4,5-trimethoxyphenyl)acrylonitrile; (E)-2-[(2,4-dimethylphenyl)sulfamoyl]-3-(2,5-dimethylphenyl)acrylonitrile; (E)-2-[(2,4-dimethylphenyl)sulfamoyl]-3-(4-biphenyl-1-yl)acrylonitrile; (E)-2-[(2,4-dimethylphenyl)sulfamoyl]-3-(2-fluoro-4-methoxyphenyl)acrylonitrile; (E)-2-[(2,4-dimethylphenyl)sulfamoyl]-3-(4-bromophenyl)acrylonitrile; (E)-2-[(2,4-dimethylphenyl)sulfamoyl]-3-(2,3,5-trichlorophenyl)acrylonitrile; (E)-2-[(2,4-dimethylphenyl)sulfamoyl]-3-(5-methylthiophen-2-yl)acrylonitrile; (E)-2-[(2,4-dimethylphenyl)sulfamoyl]-3-(2-methoxyphenyl)acrylonitrile; (E)-2-[(2,4-dimethylphenyl)sulfamoyl]-3-(4-chlorophenyl)acrylonitrile; (E)-2-[(2,4-dimethylphenyl)sulfamoyl]-3-(3,4,5-trimethoxyphenyl)acrylonitrile; (E)-2-[(2,4-dimethylphenyl)sulfamoyl]-3-(5-bromo-3-indolyl)acrylonitrile; (E)-2-[(2,4-dimethylphenyl)sulfamoyl]-3-(3,4-dichlorophenyl)acrylonitrile; (E)-2-[(2,4-dimethylphenyl)sulfamoyl]-3-(3-ethoxy-4-hydroxyphenyl)acrylonitrile; (E)-2-[(4-methoxyphenyl)sulfamoyl]-3-(2-benzyloxyphenyl)acrylonitrile; (E)-2-[(4-methoxyphenyl)sulfamoyl]-3-(3-indolyl)acrylonitrile; (E)-2-[(4-methoxyphenyl)sulfamoyl]-3-(2,4,6-trimethoxyphenyl)acrylonitrile; (E)-2-[(4-methoxyphenyl)sulfamoyl]-3-(4-(N,N-dimethylamino)phenyl)acrylonitrile; (E)-2-[(4-methoxyphenyl)sulfamoyl]-3-(2,5-dimethylphenyl)acrylonitrile; (E)-2-[(4-methoxyphenyl)sulfamoyl]-3-(2,4,5-trimethoxyphenyl)acrylonitrile; (E)-2-[(4-methoxyphenyl)sulfamoyl]-3-(2,3,4-trimethoxyphenyl)acrylonitrile; (E)-2-[(4-methoxyphenyl)sulfamoyl]-3-(4-biphenyl-1-yl)acrylonitrile; (E)-2-[(4-methoxyphenyl)sulfamoyl]-3-(3,4-dichlorophenyl)acrylonitrile; (E)-2-[(4-methoxyphenyl)sulfamoyl]-3-(4-hydroxyphenyl)acrylonitrile; (E)-2-[(4-methoxyphenyl)sulfamoyl]-3-(4-chlorophenyl)acrylonitrile; (E)-2-[(4-methoxyphenyl)sulfamoyl]-3-(4-bromophenyl)acrylonitrile; (E)-2-[(4-methoxyphenyl)sulfamoyl)-3-(2-hydroxyphenyl)acrylonitrile; (E)-2-[(4-methoxyphenyl)sulfamoyl]-3-(2,3,5-trichlorophenyl)acrylonitrile and salts thereof; Preferred embodiments of the invention include the following compounds: (E)-2-[(4-methoxyphenyl)sulfamoyl]-3-(4-bromophenyl)acrylonitrile; (E)-2-[(4-methoxyphenyl)sulfamoyl]-3-(2,4,6-trimethoxyphenyl)acrylonitrile; (E)-2-[(4-methoxyphenyl)sulfamoyl]-3-(2,3,4,5,6-pentafluorophenyl)acrylonitrile; (E)-2-[(4-methoxy-3-(4-methylphenylsulfonyloxy)phenyl)sulfamoyl]-3-(2,3,4,5,6-pentafluorophenyl)acrylonitrile; (E)-2-[(2-phenoxyphenyl)sulfamoyl]-3-(2-phenoxyphenyl)acrylonitrile; (E)-2-[(2-phenoxyphenyl)sulfamoyl]-3-(4-ethoxy-3-methoxyphenyl)acrylonitrile; (E)-2-[(4-bromophenyl)sulfamoyl]-3-(2,4,6-trimethoxyphenyl)acrylonitrile; (E)-2-[(4-bromophenyl)sulfamoyl]-3-(4-biphenyl-1-yl)acrylonitrile; (E)-2-[(4-bromophenyl)sulfamoyl]-3-(2,3,4,5,6-pentafluorophenyl)acrylonitrile; (E)-2-[(4-fluorophenyl)sulfamoyl]-3-(2-benzyloxyphenyl)acrylonitrile; (E)-2-[(4-fluorophenyl)sulfamoyl]-3-(2,3,4,5,6-pentafluorophenyl)acrylonitrile; (E)-2-[(4-fluorophenyl)sulfamoyl]-3-(2-chlorophenyl)acrylonitrile; (E)-2-[(4-fluorophenyl)sulfamoyl]-3-(4-biphenyl-1-yl)acrylonitrile; (E)-2-[(4-fluorophenyl)sulfamoyl]-3-(2,3,5-trichlorophenyl)acrylonitrile; (E)-2-[(2,4-dimethylphenyl)sulfamoyl]-3-(2-benzyloxyphenyl)acrylonitrile; (E)-2-[(2,4-dimethylphenyl)sulfamoyl]-3-(2-chlorophenyl)acrylonitrile; (E)-2-[(2,4-dimethylphenyl)sulfamoyl]-3-(3-methylthiophen-2-yl)acrylonitrile; (E)-2-[(2,4-dimethylphenyl)sulfamoyl]-3-(4-bromophenyl)acrylonitrile; (E)-2-[(2,4-dimethylphenyl)sulfamoyl]-3-(2,3,5-trichlorophenyl)acrylonitrile; (E)-2-[(2,4-dimethylphenyl)sulfamoyl]-3-(5-bromo-3-indolyl)acrylonitrile; (E)-2-[(4-methoxyphenyl)sulfamoyl]-3-(2-benzyloxyphenyl)acrylonitrile; (E)-2-[(4-methoxyphenyl)sulfamoyl]-3-(2,4,6-trimethoxyphenyl)acrylonitrile; (E)-2-[(4-methoxyphenyl)sulfamoyl]-3-(2,3,5-trichlorophenyl)acrylonitrile; (E)-2-[(4 methoxyphenyl)sulfamoyl]-3-(2,5-dimethoxyphenyl)acrylonitrile; and salts thereof.

Preferred embodiments of the invention include the following compounds: (E)-2-[(4-methoxyphenyl)sulfamoyl]-3-(4-bromophenyl)acrylonitrile; (E)-2-[(4-methoxyphenyl)sulfamoyl]-3-(2,4,6-trimethoxyphenyl)acrylonitrile; (E)-2-[(4-methoxyphenyl)sulfamoyl]-3-(2,3,4,5,6-pentafluorophenyl)acrylonitrile; (E)-2-[(4-methoxy-3-(4-methylphenylsulfonyloxy)phenyl)sulfamoyl]-3-(2,3,4,5,6-pentafluorophenyl)acrylonitrile; (E)-2-[(2-phenoxyphenyl)sulfamoyl]-3-(2-phenoxyphenyl)acrylonitrile; (E)-2-[(2-phenoxyphenyl)sulfamoyl]-3-(4-ethoxy-3-methoxyphenyl)acrylonitrile; (E)-2-[(4-bromophenyl)sulfamoyl]-3-(2,4,6-trimethoxyphenyl)acrylonitrile; (E)-2-[(4-bromophenyl)sulfamoyl]-3-(4-biphenyl-1-yl)acrylonitrile; (E)-2-[(4-bromophenyl)sulfamoyl]-3-(2,3,4,5,6-pentafluorophenyl)acrylonitrile; (E)-2-[(4-fluorophenyl)sulfamoyl]-3-(2-benzyloxyphenyl)acrylonitrile; (E)-2-[(4-fluorophenyl)sulfamoyl]-3-(2,3,4,5,6-pentafluorophenyl)acrylonitrile; (E)-2-[(4-fluorophenyl)sulfamoyl]-3-(2-chlorophenyl)acrylonitrile; (E)-2-[(4-fluorophenyl)sulfamoyl]-3-(4-biphenyl-1-yl)acrylonitrile; (E)-2-[(4-fluorophenyl)sulfamoyl]-3-(2,3,5-trichlorophenyl)acrylonitrile; (E)-2-[(2,4-dimethylphenyl)sulfamoyl]-3-(2-benzyloxyphenyl)acrylonitrile; (E)-2-[(2,4-dimethylphenyl)sulfamoyl]-3-(2-chlorophenyl)acrylonitrile; (E)-2-[(2,4-dimethylphenyl)sulfamoyl]-3-(3-methylthiophen-2-yl)acrylonitrile; (E)-2-[(2,4-dimethylphenyl)sulfamoyl]-3-(4-bromophenyl)acrylonitrile; (E)-2-[(2,4-dimethylphenyl)sulfamoyl]-3-(2,3,5-trichlorophenyl)acrylonitrile; (E)-2-[(2,4-dimethylphenyl)sulfamoyl]-3-(5-bromo-3-indolyl)acrylonitrile; (E)-2-[(4-methoxyphenyl)sulfamoyl]-3-(2-benzyloxyphenyl)acrylonitrile; (E)-2-[(4-methoxyphenyl)sulfamoyl]-3-(2,4,6-trimethoxyphenyl)acrylonitrile; (E)-2-[(4-methoxyphenyl)sulfamoyl]-3-(2,3,5-trichlorophenyl)acrylonitrile; (E)-2-[(4-methoxyphenyl)sulfamoyl]-3-(2,5-dimethoxyphenyl)acrylonitrile; and salts thereof.

Other particular embodiments of the invention include the following compounds: (E)-2-[(4-methoxyphenyl)sulfamoyl]-3-(2,3,4,5,6-pentafluorophenyl)acrylamide; (E)-2-[(4-methoxyphenyl)sulfamoyl]-3-(2-benzoxazolon-5-yl)acrylamide; (E)-2-[(4-chlorophenyl)sulfamoyl]-3-(3,4-dihydroxyphenyl)acrylamide; (E)-2-[(4-methoxyphenyl)sulfamoyl]-3-(3,4-dihydroxyphenyl)acrylamide; (E)-2-[(3,4-dimethoxyphenyl)sulfamoyl]-3-(2,3,4,5,6-pentafluorophenyl)acrylamide; (E)-2-[(4-fluorophenyl)sulfamoyl]-3-(2,3,4,5,6-pentafluorophenyl)acrylamide; (E)-2-[(3-nitro-4-methoxyphenyl)sulfamoyl]-3-(2,3,4,5,6-pentafluorophenyl)acrylamide; and salts thereof.

Other particular embodiments of the invention include the following compounds: (E)-N-(4-methoxyphenyl)-1-nitro-2-(2,3,4,5,6-pentafluorophenyl)ethenesulfonamide; (E) —N-(4-methoxyphenyl)-1-nitro-2-(2-oxo-2,3-dihydrobenzo[d]oxazol-5-yl)ethenesulfonamide; (E)-N-(4-chlorophenyl)-2-(3,4-dihydroxyphenyl)-1-nitroethenesulfonamide; (E)-N-(4-methoxyphenyl)-2-(3,4-dihydroxyphenyl)-1-nitroethenesulfonamide; (E)-N-(3,4-dimethoxyphenyl)-1-nitro-2-(2,3,4,5,6-pentafluorophenyl)ethenesulfonamide; (E)-N-(4-fluorophenyl)-1-nitro-2-(2,3,4,5,6-pentafluorophenyl)ethenesulfonamide; (E)-N-(3-nitro-4-fluorophenyl)-1-nitro-2-(2,3,4,5,6-pentafluorophenyl)ethenesulfonamide; and salts thereof.

It is to be understood that other particular and preferred embodiments of the invention will combine the features of the particular and preferred embodiments of the invention explicitly described above. Embodiments defined by such combinations are contemplated as particular embodiments of the invention.

### III. Intermediates and Processes for Preparing Compounds of the Invention

In another aspect of the invention, there are provided processes for preparing compounds according to formula I, intermediates that are useful in the preparation of such compounds, and processes for preparing such intermediates.

In the text, formulae and schemes that follow, unless otherwise indicated Ar¹, Ar², G, R¹, m and n are as defined above for formula I.

A process is provided for the synthesis of compounds according to formula I comprising condensing a compound of formula II with an aromatic aldehyde of formula III.

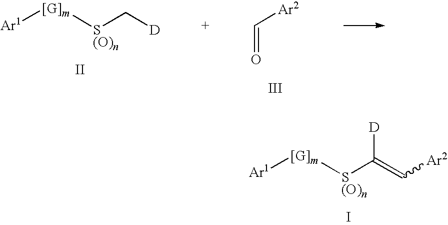

Particular embodiments of this process include those wherein:
G is CR¹₂, m is 1, and D is —C≡N; or
G is CR¹₂, m is 0 or 1, and D is —C(═O)NR⁸₂; or
G is CH₂, m is 0 or 1, and D is —NO₂; or
G is NR¹, m is 1, and D is —C≡N.

Other particular embodiments of this process are those wherein n is 2.

The condensation may be achieved by treatment with acid or base catalysts or reagents. The reaction is preferably carried out in an appropriate solvent. The reactions are typically carried out at a temperature between 0° C. and the reflux temperature of the solvent, which is typically about 100° C. Depending on the substrates, heating the reaction mixture, and/or removal of water may be beneficial. For example, when n is 2, a preferred method of carrying out the reaction is by heating in toluene in the presence of catalytic amounts of piperidine and a carboxylic acid with removal of water using a Dean Stark trap.

Certain intermediates of formula II are novel, and are useful in preparing compounds of formula I. Such intermediates are provided as an aspect of the invention.

In particular, there are provided novel compounds of formula II wherein m=1, n=2, and D is —C≡N. An embodiment of this aspect of the invention is compounds of formula II wherein:

m is 1, n is 2, D is —C≡N, G is CH₂, and Ar¹ is phenyl; or

m is 1, n is 2, D is —C≡N, G is NH, and Ar¹ is phenyl.

Processes are provided for the synthesis of certain compounds according to formula I comprising oxidation of other compounds of formula I, and for the synthesis of intermediate compounds of formula II comprising oxidation of other compounds of formula II.

A process is provided for the synthesis of compounds according to formula I wherein n is 2 and G is CR¹₂, comprising oxidizing a corresponding compound of formula I wherein n is 0 or 1.

A process is provided for the synthesis of compounds according to formula I wherein n is 1 and G is CR¹₂, comprising oxidizing a corresponding compound of formula I wherein n is 0.

A process provided for the synthesis of compounds according to formula II, wherein n is 2 and G is CR¹₂, comprising oxidizing a corresponding compound of formula II wherein n is 0 or 1.

A process is also provided for the synthesis of compounds according to formula II wherein n is 1 and G is CR¹₂, comprising oxidizing a corresponding compound of formula II wherein n is 0.

The aforementioned oxidation processes are carried out by reacting the starting material with an appropriate oxidizing agent in a suitable solvent at an appropriate temperature. Suitable solvents for such oxidation processes typically include alcohols and acetic acid. Suitable oxidizing agents typically include hydrogen peroxide, carboxylic peracids, such as m-chloroperoxybenzoic acid, or persulfate salts, such as potassium peroxymonosulfate. The reactions are typically carried out at a temperature between 0° C. and the reflux temperature of the solvent, which is typically about 100° C. The person skilled in the art will know how to select suitable oxidizing agents and reaction conditions. For example, under mild conditions such as low temperature and using a limiting amount of oxidizing agent, selective oxidation of thioethers to sulfoxides can often be achieved, whereas under more forcing conditions such as using excess oxidizing agent, higher temperature, or prolonged reaction times oxidation of thioethers or sulfoxides to sulfones can be achieved. Certain reagents (e.g. sodium periodate) are known to oxidize thioethers selectively to sulfoxides.

A process is also provided for the synthesis of compounds according to formula II wherein n is 0 and G is CHR¹, comprising coupling a mercaptan of formula IV, wherein G is CHR¹, with a compound of formula V, wherein X is leaving group.

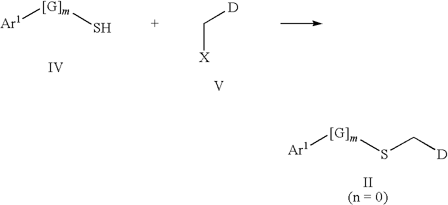

A process is also provided for the synthesis of compounds according to formula II wherein m is 1, n is 0, and G is CR¹₂, comprising coupling a compound of formula VI, wherein X is leaving group, with a mercaptan of formula VII, wherein X is leaving group.

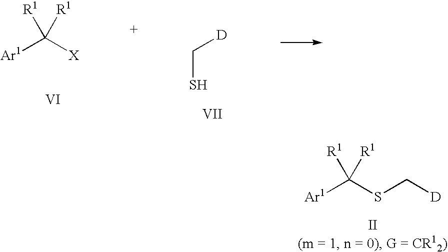

Suitable leaving groups X in the compounds of formula V and VII include halogen, particularly chlorine, bromine, and iodine, and sulfonate groups, particularly methanesulfonate, p-toluenesulfonate, and trifluoromethanesulfonate. The coupling reactions are typically performed using a basic catalyst or reagent in a suitable solvent at a suitable temperature. Suitable bases include alkali metal hydroxide or alkoxide salts such as sodium hydroxide or methoxide, and tertiary amines such as triethylamine or N,N-diisopropylethylamine. Suitable solvents include alcohols, such as methanol, or dichloromethane. The reactions are typically carried out at a temperature between 0° C. and the reflux temperature of the solvent, which is typically about 100° C. For example, in a typical procedure, the reactions would be conducted by treatment of the mercaptan with a solution of sodium hydroxide in methanol followed by addition of the compound V or VII.

A process is also provided for the synthesis of compounds according to formula II wherein m is 1, n is 2, and G is NR¹, comprising coupling an aminoaromatic compound of formula VIII, with a compound of formula IX, wherein X is leaving group.

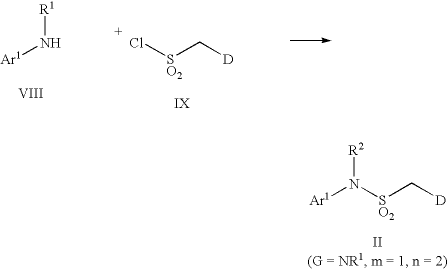

The coupling reactions are typically performed using a basic catalyst or reagent in a suitable solvent at a suitable temperature. Suitable bases include tertiary amines such as triethylamine or N,N-diisopropylethylamine, or pyridine. Typically, at least one equivalent of base would be used because hydrogen chloride is used in the reaction. Suitable solvents include pyridine or dichloromethane. The reactions are typically carried out at a temperature between 0° C. and the reflux temperature of the solvent, which is typically about 100° C. The reaction is preferably carried out at about between 0° C. and about 10° C. For example, in a typical procedure, the reactions would be conducted by adding the sulfenyl chloride to a solution containing the aromatic amine and triethylamine in dichloromethane at about 10° C.

A process is also provided for the synthesis of compounds according to formula II wherein D is —C(═O)NR⁸₂, comprising forming an amide from a carboxylic acid derivative of formula II wherein D is C(═O)OH.

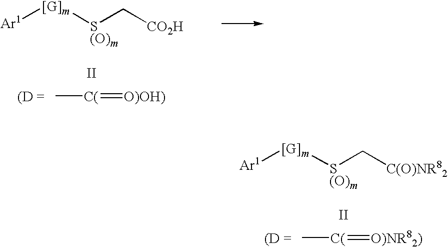

The amide-forming reactions are performed, for example, by directly heating the carboxylic acid with imidazole under microwave irradiation (for D=—C(═O)NH₂). Alternative ways of carrying out the same transformation include reacting a suitable derivative of the carboxylic acid an amine of formula R⁸₂NH. Suitable derivatives of the carboxylic acid include the acid chloride, anhydrides, including mixed anhydrides such as the pivaloyl derivative, and esters, including activated esters such as the N-hydroxybenzotriazole ester. Such derivatives may be pre-formed or formed in situ. For example, the coupling reaction may be preformed under mild conditions by treating the carboxylic acid and the amine in a suitable solvent with a suitable coupling agent. Suitable coupling agents include carbodiimides, for example 1,3-dicyclohexylcarbodiimide, 1-(3-dimethylaminopropyl-3-ethylcarbodiimide hydrochloride; phosphonium reagents, for example benzotriazol-1-yloxytris(dimethylamino)phosphonium hexafluorophosphate or benzotriazol-1-yloxytripyrrolidinophosphonium hexafluorophosphate; and uronium reagents, for example O-benzotriazol-1-yl-N,N,N′,N′-tetramethyluronium tetrafluoroborate.

Compounds of formula II wherein D is —C(═O)OH may be prepared via modification of the procedures described above for the synthesis of intermediates of formula II, substituting a suitable acetic acid derivative as the compound formula V, VII, or IX. Suitable acetic acid derivatives include the free acid (V, VII, or IX wherein D is C(═O)OH), or esters (V, VII, or IX wherein D is —C(O)O-alkyl). If an ester is used, the ester is subsequently hydrolyzed to the free acid, for example by hydrolysis using an alkali metal hydroxide solution.

A process is also provided for the synthesis of compounds according to formula II wherein D is —C(O)NH₂, comprising hydrolyzing a nitrile of formula II wherein D is —C≡N.

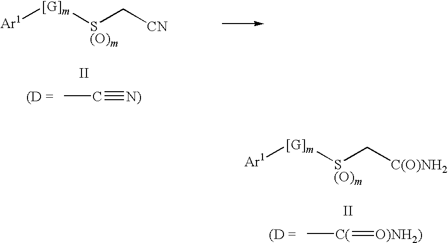

A process is also provided for the synthesis of compounds according to formula II wherein D is C(═O)OH, comprising hydrolyzing a nitrile of formula II wherein D is C≡N.

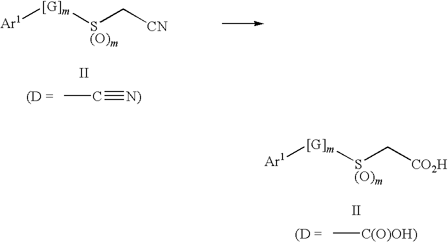

The hydrolysis reactions can be carried out under a variety of acid- or base-catalyzed conditions. As between the acid and the amide as the desired product, the reaction conditions can be chosen so as to give the desired product with mild conditions enabling the amide to be obtained, while with harsher conditions the intermediate amide is hydrolyzed to the acid. For example, when the amide is desired reagents of choice include concentrated sulfuric acid, or alternatively aqueous sodium hydroxide containing hydrogen peroxide. Hydrolysis to the acid can be achieved, for example, by heating with aqueous hydrochloric acid, aqueous sulfuric acid, or aqueous sodium hydroxide.

Certain intermediates of formula II are novel, and are useful in preparing compounds of formula I. Such intermediates are provided as an aspect of the invention. In particular, there are provided novel compounds of formula II wherein m=1, n=2, and D is —C≡N.

Compounds of formula III are either commercially available, known in the literature, or may be prepared by methods known to one skilled in the art. Methods used for the formation of aromatic aldehydes include, for example, formylation of aromatic compounds, including electrophilic formylation, organometallically-catalyzed formylation using carbon monoxide, or lithiation followed by reaction with an N,N-dialkylformamide and hydrolysis. See, e.g., the reactions referenced for the formation of aldehydes in Advanced Organic Chemistry, by Jerry March (3d Edition, John Wiley & Sons, 1985), p. 1147-1148.

Mercaptans of formula IV wherein G is CR¹₂ are either commercially available, known in the literature, or may be prepared by methods known to one skilled in the art. For example, mercaptans can be produced by the reduction of sulfonic acid or sulfonyl chlorides, which, in the case of aromatic sulfonyl halides (m=0), can be produced by electrophilic sulfonation or chlorosulfonation of aromatic rings. Other methods include nucleophilic substitution of compounds with a suitable leaving group such as halides (i.e. compounds of formula VI) with a suitable divalent sulfur compound. The reaction is typically performed with compounds such as thiolacetic acid or thiourea, which perform the substitution to give initially a protected intermediate (such as a thiolacetate, or thiouronium salt) which can be subsequently converted to the mercaptan, for example by hydrolysis. The nucleophilic substitution is in general particularly facile with benzylic-type compounds where the substitution occurs at a position alpha to an aromatic ring (i.e. with compounds VI where m is 1). See, e.g., the reactions referenced for the formation of mercaptans in Advanced Organic Chemistry, by Jerry March (3^{rd} Edition, John Wiley & Sons, 1985), p. 1168; The Chemistry of the Thiol Group, by S. Patai, Ed. (Wiley-Interscience, New York, 1974).

Mercaptans of formula VII are likewise commercially available, known in the literature, or may be prepared by methods known to one skilled in the art. For example, the mercaptans of formula VII can be prepared from compounds of formula V by nucleophilic substitution of the leaving group X with thiolacetic acid or thiourea followed by hydrolysis of the resulting protected intermediate.

Compounds of formula V are commercially available, known in the literature, or may be prepared by methods known to one skilled in the art. For example, chloroacetonitrile, bromonitromethane, and 2-chloroacetamide are all available for purchase from Sigma-Aldrich.

Compounds of formula VI are likewise commercially available, known in the literature, or may be prepared by methods known to one skilled in the art. For example, —CH₂— groups alpha to an aromatic ring can be readily halogenated under free radical conditions. Alternatively, appropriate X groups could be introduced by conversion of the corresponding alcohol (by conversion of OH to halogen, or treatment with a sulfonyl chloride such as p-toluenesulfonyl chloride), which can be prepared by a variety of methods, for example Friedel Crafts acylation, as illustrated in the scheme below:

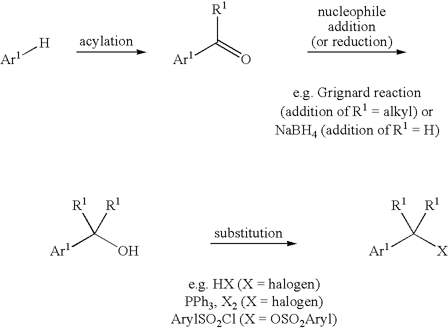

Aromatic amines of formula VIII are also commercially available, known in the literature, or may be prepared by methods known to one skilled in the art. Classically such compounds are available via nitration of the aromatic ring and reduction of the resulting nitro compound to an amino group, which can then be further functionalized. Alternatively, the functionalized amino groups can be introduced by substitution of an appropriate leaving group such as halogen on the aromatic ring Ar¹. Such substitution reactions may be performed under basic conditions with fluoride as a preferred leaving group. Alternatively such substitution reactions can be performed under mild conditions using organometallic catalysis. See, e.g., S. Buchwald et al., J. Organomet. Chem., 1999, 576, 125; “Practical Palladium Catalysts for C—N and C—O Bond Formation” in Topics in Current Chemistry; edited by N. Miyaura; Springer-Verlag: Berlin, Germany, 2001, Vol. 219, 131-209.

Compounds of formula IX may be prepared from compounds of formula V by a two-step procedure involving, first, substitution of the X group with sulfite to form the corresponding sulfonic acid X (as a salt), followed by halogenation of the resulting sulfonic acid, for example using phosphorus oxychloride and phosphorus pentachloride. See, e.g., M. P. Sammes, GB Patent No. 1252903 (describing the preparation of the compound of formula XI wherein D is —C≡N).

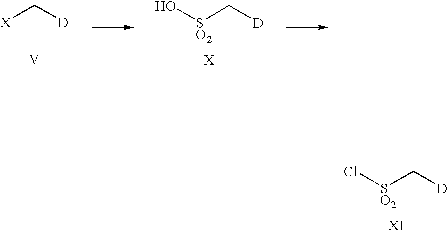

In the compounds described above, some functional groups on the aromatic rings, in particular aromatic amine nitrogens, are further derivatizable. Derivatives of aromatic amino groups which are useful in the present invention include, for example: acylation to form carboxamide, carbamate, and urea derivatives; sulfonylation to form sulfonamides, sulfonyl ureas, and sulfamoyl esters; imine formation for formation of imines and for alkylation or arylation (or heteroarylation) via reductive amination; alkylation to form mono- or di-alkylamino derivatives, palladium catalyzed cross coupling to form N-aryl (or N-heteroaryl) derivatives by coupling with aromatic halides or aromatic pseudo halides such as aromatic triflates. Derivatives may also include conjugates to biological molecules such as antibodies to yield macro molecules capable of being directed to a desired site of action thereby reducing or precluding side effects associated with interaction of a drug prepared from a compound of the present invention with tissues and cells which are not proliferating abnormally.

The above-described reactions, unless otherwise noted, are usually conducted at a pressure of about one to about three atmospheres, preferably at ambient pressure (about one atmosphere).

The present invention further embraces isolated compounds according to formula I. The expression “isolated compound” refers to a preparation of a compound of formula I, or a mixture of compounds according to formula I, wherein the isolated compound has been separated from the reagents used, and/or byproducts formed, in the synthesis of the compound or compounds. “Isolated” does not mean that the preparation is technically pure (homogeneous), but it is sufficiently pure to compound in a form in which it can be used therapeutically. Preferably an “isolated compound” refers to a preparation of a compound of formula I or a mixture of compounds according to formula I, which contains the named compound or mixture of compounds according to formula I in an amount of at least 10 percent by weight of the total weight. Preferably the preparation contains the named compound or mixture of compounds in an amount of at least 50 percent by weight of the total weight; more preferably at least 80 percent by weight of the total weight; and most preferably at least 90 percent, at least 95 percent or at least 98 percent by weight of the total weight of the preparation.

In other preferred embodiments, the compound of formula I, and compositions containing the compound, including pharmaceutical compositions, are substantially free of pharmaceutically unacceptable contaminants. A pharmaceutically unacceptable contaminant is a substance which, if present in more than an insubstantial amount, would render the compound or composition unsuitable for use as a pharmaceutical for therapeutic administration. Examples include toxic materials such as halogenated solvents and heavy metals, and potentially infectious materials such as bacteria, fungi, viruses, and bacterial and fungal spores.

The compounds of the invention and intermediates may be isolated from their reaction mixtures and purified by standard techniques such as filtration, liquid-liquid extraction, solid phase extraction, distillation, recrystallization or chromatography, including flash column chromatography, or HPLC. The preferred method for purification of the compounds according to formula I or salts thereof comprises crystallizing the compound or salt from a solvent to form, preferably, a crystalline form of the compounds or salts thereof. Following crystallization, the crystallization solvent is removed by a process other than evaporation, for example filtration or decanting, and the crystals are then preferably washed using pure solvent (or a mixture of pure solvents). Preferred solvents for crystallization include water, alcohols, particularly alcohols containing up to four carbon atoms such as methanol, ethanol, isopropanol, and butan-1-ol, butan-2-ol, and 2-methyl-2-propanol, ethers, for example diethyl ether, diisopropyl ether, t-butyl methyl ether, 1,2-dimethoxyethane, tetrahydrofuran and 1,4-dioxane, carboxylic acids, for example formic acid and acetic acid, and hydrocarbon solvents, for example pentane, hexane, toluene, and mixtures thereof, particularly aqueous mixtures such as aqueous ethanol. Pure solvents, preferably at least analytical grade, and more preferably pharmaceutical grade are preferably used. In a preferred embodiment of the processes of the invention, the products are so isolated. In the compounds of the invention according to formula I or salt thereof, and pharmaceutical compositions thereof, the compound according to formula I or salt thereof is preferably in or prepared from a crystalline form, preferably prepared according to such a process.

The synthetic methods described above reflect a convergent synthesis strategy. Thus the Ar¹ and Ar² components may be synthesized and elaborated separately prior to coupling the two components to form the target compounds. These convergent synthetic schemes allow for arrangement of the assembly steps of the backbone of the target compounds and derivatization of derivatizable functionalities to accommodate functional group sensitivity and/or to allow for functional groups or elements to be introduced either before or after the assembly of the backbone of the target compounds via the coupling reactions described.

It will be appreciated by one skilled in the art that certain aromatic substituents in the compounds of the invention, intermediates used in the processes described above, or precursors thereto, may be introduced by employing aromatic substitution reactions to introduce or replace a substituent, or by using functional group transformations to modify an existing substituent, or a combination thereof. Such reactions may be effected either prior to or immediately following the processes mentioned above, and are included as part of the process aspect of the invention. The reagents and reaction conditions for such procedures are known in the art. Specific examples of procedures which may be employed include, but are not limited to, electrophilic functionalization of an aromatic ring, for example via nitration, halogenation, or acylation; transformation of a nitro group to an amino group, for example via reduction, such as by catalytic hydrogenation; acylation, alkylation, or sulfonylation of an amino or hydroxyl group; replacement of an amino group by another functional group via conversion to an intermediate diazonium salt followed by nucleophilic or free radical substitution of the diazonium salt; or replacement of a halogen by another group, for example via nucleophilic or organometallically-catalyzed substitution reactions.

Additionally, in the aforesaid processes, certain functional groups which would be sensitive to the reaction conditions may be protected by protecting groups. A protecting group is a derivative of a chemical functional group which would otherwise be incompatible with the conditions required to perform a particular reaction which, after the reaction has been carried out, can be removed to re-generate the original functional group, which is thereby considered to have been “protected”. Any chemical functionality that is a structural component of any of the reagents used to synthesize compounds of this invention may be optionally protected with a chemical protecting group if such a protecting group is useful in the synthesis of compounds of this invention. The person skilled in the art knows when protecting groups are indicated, how to select such groups, and processes that can be used for selectively introducing and selectively removing them, because methods of selecting and using protecting groups have been extensively documented in the chemical literature. Techniques for selecting, incorporating and removing chemical protecting groups may be found, for example, in Protective Groups in Organic Synthesis by Theodora W. Greene, Peter G. M. Wuts, John Wiley & Sons Ltd., the entire disclosure of which is incorporated herein by reference.

In addition to use of a protecting group, sensitive functional groups may be introduced as synthetic precursors to the functional group desired in the intermediate or final product. An example of this is an aromatic nitro (—NO₂) group. The aromatic nitro group goes not undergo any of the nucleophilic reactions of an aromatic amino group. However, the nitro group can serve as the equivalent of a protected amino group because it is readily reduced to the amino group under mild conditions that are selective for the nitro group over most other functional groups.

It will be appreciated by one skilled in the art that the processes described are not the exclusive means by which compounds of the invention may be synthesized and that an extremely broad repertoire of synthetic organic reactions is available to be potentially employed in synthesizing compounds of the invention. The person skilled in the art knows how to select and implement appropriate synthetic routes. Suitable synthetic methods may be identified by reference to the literature, including reference sources such as Comprehensive Organic Synthesis, Ed. B. M. Trost and I. Fleming (Pergamon Press, 1991), Comprehensive Organic Functional Group Transformations, Ed. A. R. Katritzky, O. Meth-Cohn, and C. W. Rees (Pergamon Press, 1996), Comprehensive Organic Functional Group Transformations II, Ed. A. R. Katritzky and R. J. K. Taylor (Editor) (Elsevier, 2^{nd} Edition, 2004), Comprehensive Heterocyclic Chemistry, Ed. A. R. Katritzky and C. W. Rees (Pergamon Press, 1984), and Comprehensive Heterocyclic Chemistry II, Ed. A. R. Katritzky, C. W. Rees, and E. F. V. Scriven (Pergamon Press, 1996).

### IV. Antibody Conjugates

Another aspect of the invention relates to antibody conjugates of compounds of formula I.

In another embodiment of the invention there is provided a conjugate of the formula I-L-Ab, or a salt thereof, wherein I is a compound of formula I, or an embodiment thereof; Ab is an antibody; and -L- is a single bond or a linking group covalently linking said compound of formula I to said antibody.

In a preferred sub-embodiment of the aforesaid conjugates of the formula I-L-Ab, said antibody (Ab) is a monoclonal antibody or a monospecific polyclonal antibody.

In a more preferred sub-embodiment of the aforesaid conjugates of the formulae I-L-Ab, the aforesaid antibody (Ab) is a tumor-specific antibody.

Antibodies, preferably monoclonal antibodies and monospecific polyclonal antibodies, and most preferably tumor-specific antibodies, may be covalently linked to compounds of the present invention. A “tumor-specific antibody” is an antibody which specifically binds to a tumor antigen, e.g., an antigen on a tumor cell.

The covalent linker between a compound of formula I and an antibody may, in its simplest form, comprise a single covalent bond connecting the compound of formula I to the antibody. More commonly the compound of formula I is attached to the antibody using a suitable bifunctional linking reagent. The term “bifunctional linking reagent” refers generally to a molecule that comprises two reactive moieties which are connected by a spacer element. The term “reactive moieties”, in this context, refers to chemical functional groups capable of coupling with an antibody or a compound of formula I by reacting with functional groups on the antibody and the compound of formula I.

An example of a covalent bond formed as a linker between a compound of formula I and an antibody is a disulfide bond formed by the oxidation of an antibody and a compound of formula I, wherein R¹ is a peptidyl group containing one or more cysteine amino acids. The cysteine residues can be oxidized to form disulfide links by dissolving 1 mg of the a suitable compound of formula I and 0.5 equivalents of the desired antibody in 1.5 ml of 0.1% (v/v) 17.5 mM acetic acid, pH 8.4, followed by flushing with nitrogen and then 0.01 M K₂Fe(CN)₆. After incubation for one hour at room temperature, the adduct peptide is purified by HPLC.

Another example of a suitable covalent bond formed as a linker between a compound of formula I and an antibody is an amide bond formed by reacting an amino group on a compound of the invention with a carboxylic acid group which forms part of the primary structure of the antibody (Ab) (such as, for example a glutamic or aspartic amino acid residue). Alternately, an amide bond could be formed if the reacting moieties were reversed, i.e., the compound of formula I could contain a carboxylic acid functionality and react with an amino functionality within the Ab structure.

Alternatively, a compound of formula I and an antibody Ab may be covalently linked using a bifunctional linking reagent. In one such embodiment of the present invention, a compound of formula I wherein R⁵ is a peptidyl group is coupled to an antibody using a bifunctional linking reagent.

For example, adducts can be prepared by first preparing S-(—N-hexylsuccinimido)-modified derivatives of an antibody and of a compound of formula I, according to the method of Cheronis et al., J. Med. Chem. 37: 348 (1994) (the entire disclosure of which is incorporated herein by reference). N-hexylmaleimide, a precursor for the modified antibody and compound of formula I, is prepared from N-(methoxycarbonyl)maleimide and N-hexylamine by mixing the two compounds in saturated NaHCO₃ at 0° C. according to the procedure of Bodanszky and Bodanszky, The Practice of Peptide Synthesis; Springer-Verlag, New York, pp. 29-31 (1984) (the entire disclosure of which is incorporated herein by reference). The product of the resulting reaction mixture is isolated by extraction into ethyl acetate, followed by washing with water, dried over Na₂SO₄, and is then concentrated in vacuo to produce N-hexylmaleimide as a light yellow oil. S—(N-Hexylsuccinimido)-modified antibody and formula I compound are then prepared from a cysteine-containing peptide and N-hexylmaleimide by mixing one part peptide with 1.5 parts N-hexylmaleimide in N,N-dimethylformamide (3.3 mL/mM peptide) followed by addition to 30 volumes of 0.1 M ammonium bicarbonate, pH 7.5. The S-alkylation reaction carried out in this manner is complete in 30 minutes. The resulting S-(N-hexylsuccinimido)-modified peptide monomer is purified by preparative reverse-phase HPLC, followed by lyophilization as a fluffy, white powder.

Bis-succinimidohexane peptide heterodimers (wherein one peptide is the antibody and the other peptide is a formula I compound wherein. R⁵ is a peptidyl group), may be prepared according to the method of Cheronis et al., supra from cysteine-substituted peptides. A mixture of one part bismaleimidohexane is made with two parts peptide monomer in N,N-dimethylformamide (3.3 mL/mM peptide) followed by addition to 0.1 ammonium bicarbonate, pH 7.5. The reaction mixture is stirred at room temperature and is usually completed within 30 minutes. The resulting bis-succinimidohexane peptide dimer is purified by preparative reverse-phase HPLC. After lyophilization the material is a fluffy, white powder.

Covalently linked adducts of the general formula I-L-Ab of the present invention may be prepared by utilizing homo-bifunctional linking reagents (wherein the two reactive moieties are the same), such as, for example, disuccinimidyl tartrate, disuccinimidyl suberate, ethylene glycolbis-(succinimidyl succinate), 1,5-difluoro-2,4-dinitrobenzene (“DFNB”), 4,4′-diisothiocyano-2,2′-disulfonic acid stilbene (“DIDS”), and bis-maleimidohexane (“BMH”). The linking reaction occurs randomly between the Ab and a compound of formula I having a peptidyl group as R⁵.

Alternatively, hetero-bifunctional linking reagents may be employed. Such agents include, for example, N-succinimidyl-3-(2-pyridyldithio)propionate (“SPDP”), sulfosuccinimidyl-2-(p-azidosalicylamido)ethyl-1-3′-dithiopropionate (“SASD”, Pierce Chemical Company, Rockford, Ill.), N-maleimidobenzoyl-N-hydroxy-succinimidyl ester (“MBS”), m-maleimidobenzoylsulfosuccinimide ester (“sulfo-MBS”), N-succinimidyl(4-iodoacetyl)aminobenzoate (“SIAB”), succinimidyl 4-(N-maleimidomethyl)-cyclohexane-1-carboxylate (“SMCC”), succinimidyl-4-(p-maleimidophenyl)butyrate (“SMPB”), sulfosuccinimidyl(4-iodoacetyl)amino-benzoate (“sulfo-SIAB”), sulfosuccinimidyl 4-(N-maleimidomethyl)cyclohexane-1-carboxylate (“sulfo-SMCC”), sulfosuccinimidyl 4-(p-maleimidophenyl)-butyrate (“sulfo-SMPB”), bromoacetyl-p-aminobenzoyl-N-hydroxy-succinimidyl ester, iodoacetyl-N-hydroxysuccinimidyl ester, and the like.

For hetero-bifunctional linking, a compound of formula I is derivatized with, for example, the N-hydroxysuccinimidyl portion of the bifunctional reagent, and the resulting derivatized compound is purified by chromatography. Next, a suitable tumor-specific Mab is reacted with the second functional group of the bifunctional linking reagent, assuring a directed sequence of binding between components of the desired adduct

Typical hetero-bifunctional linking agents for forming protein-protein conjugates have an amino-reactive N-hydroxysuccinimide ester (NHS-ester) as one functional group and a sulfhydryl reactive group as the other functional group. First, epsilon-amino groups of surface lysine residues of either the Mab or the formula I compound are acylated with the NHS-ester group of the cross-linking agent. The remaining component, possessing free sulfhydryl groups, is reacted with the sulfhydryl reactive group of the cross-linking agent to form a covalently cross-linked dimer. Common thiol reactive groups include, for example, maleimides, pyridyl disulfides, and active halogens. For example, MBS contains a NHS-ester as the amino reactive group, and a maleimide moiety as the sulfhydryl reactive group.

Photoactive hetero-bifunctional linking reagents, e.g., photoreactive phenyl azides, may also be employed. One such reagent, SASD, may be linked to either a Mab or to a formula I compound wherein R⁵ is a peptidyl group, via its NHS-ester group. The conjugation reaction is carried out at pH 7 at room temperature for about 10 minutes. Molar ratios between about 1 and about 20 of the cross-linking agent to the compounds to be linked may be used.

Numerous bifunctional linkers, useful as linkers (-L-), exist which have been used specifically for coupling small molecules to monoclonal antibodies, and many of these are commercially available. Examples include N-succinimidyl-3-(2-pyridyldithio)-propionate (SPDP), 2-iminothiolane (2-IT), 3-(4-carboxamidophenyldithio)propionthioimidate (CDPT), N-succinimidyl-acetylthioacetate (SATA), ethyl-5-acetyl-propionthioimidate (AMPT) and N-succinimidyl-3-(4-carboxamidophenyldithio)propionate (SCDP). Procedures for preparation of immunoconjugates using these linkers is detailed in Cattel, et al, “Toxin-Targeted Design for Anticancer Therapy II: Preparation and Biological Comparison of Different Chemically Linked Gelonin-Antibody Conjugates”, J. Pharm. Sci., 1993, 82, 699-704, the entire disclosure of which is incorporated herein by reference.

### V. Pharmaceutical Compositions

In another aspect of the invention there are provided pharmaceutical compositions comprising a pharmaceutically acceptable carrier, and a compound according to formula I, or a pharmaceutically acceptable salt thereof:

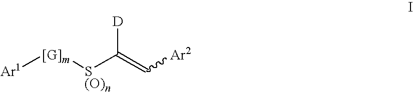

wherein:

Ar¹ is:

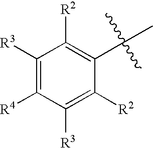

Ar² is selected from the group consisting of unsubstituted aryl, substituted aryl, unsubstituted heteroaryl and substituted heteroaryl; wherein, when Ar² is substituted aryl or substituted heteroaryl, said substituted aryl or substituted heteroaryl is aryl or heteroaryl substituted with one or more substituents independently selected from the group consisting of halogen, (C₁-C₆)alkyl, (C₂-C₆)alkenyl, (C₂-C₆)alkynyl, —(C₁-C₃)alkylene-Ar³, —OR⁷, —OAr³, —O(C₁-C₃)alkylene-Ar³, (C₁-C₃)fluoroalkoxy, —NO₂, —C≡N, —C(═O)(C₁-C₃)alkyl, —C(═O)OR⁷, —C(═O)NR⁸₂, —C(═NR⁷)NR⁸₂, —(C₁-C₃)alkylene-C(═O)OR⁷, —O(C₁-C₃)alkylene-C(═O)OR⁷, —(C₁-C₆)alkylene-OR⁷, —NR⁸₂, —P(═O)(OR⁷)₂, —OP(═O)(OR⁷)₂, —S(C₁-C₆)alkyl, —S(O)(C₁-C₆)alkyl, —SO₂(C₁-C₆)alkyl, —SO₂NR⁸₂, —OSO₂(C₁-C₆)alkyl, —OSO₂Ar³, —NHC(═O)(C₁-C₆)alkyl, —OC(═O)(C₁-C₃)alkyl, —O(C₂-C₆)alkylene-NR⁸₂ and (C₁-C₃)perfluoroalkyl;

D is —C≡N, —C(═O)NR⁸₂, or NO₂;

G is CR¹₂ or NR¹;

R¹ is independently selected from the group consisting of hydrogen and (C₁-C₆)alkyl;

each R² is independently selected from the group consisting of hydrogen, (C₁-C₆)alkyl, halogen, —OR⁷, —OAr³, —O(C₁-C₃)alkylene-Ar³, —C≡N, —NO₂, —C(═O)OR⁷, —C(═O)NR⁸₂, —C(═NR⁷)NR⁸₂, —(C₁-C₃)alkylene-C(═O)OR⁷, —O(C₁-C₃)alkylene-C(═O)OR⁷, —(C₁-C₆)alkylene-OR⁷, —P(═O)(OR⁷)₂, —OP(═O)(OR⁷)₂, —NHC(═O)(C₁-C₆)alkyl, —SO₂NR⁸₂, —OSO₂(C₁-C₆)alkyl, —OSO₂Ar³, —OC(═O)(C₁-C₃)alkyl, —O(C₂-C₆)alkylene-NR (C₁-C₃)perfluoroalkyl and —N(R⁶)-(M)_{y}-R⁵;

each R³ is independently selected from the group consisting of hydrogen, (C₁-C₆)alkyl, halogen, —OR⁷, —OAr³, —O(C₁-C₃)alkylene-Ar³, —C≡N, —NO₂, —C(═O)OR⁷, —C(═O)NR⁸₂, —C(═NR⁷)NR⁸₂, —(C₁-C₃)alkylene-C(═O)OR⁷, —O(C₁-C₃)alkylene-C(═O)OR⁷, —(C₁-C₆)alkylene-OR⁷, —P(═O)(OR⁷)₂, —OP(═O)(OR⁷)₂, —NHC(═O)(C₁-C₆)alkyl, —SO₂NR³₂, —OSO₂(C₁-C₆)alkyl, —OSO₂Ar³, —OC(═O)(C₁-C₃)alkyl, —O(C₂-C₆)alkylene-NR⁸₂, (C₁-C₃)perfluoroalkyl and —N(R⁶)-(M)_{y}-R⁵;

R⁴ is selected from the group consisting of hydrogen, (C₁-C₆)alkyl, halogen, —OR⁷, —OAr³, —O(C₁-C₃)alkylene-Ar³, —C≡N, —NO₂, —C(═O)OR⁷, —C(═O)NR⁸₂, —C(═NR⁷)NR⁸₂, —(C₁-C₃)alkylene-C(═O)OR⁷, —O(C₁-C₃)alkylene-C(═O)OR⁷, —(C₁-C₆)alkylene-OR⁷, —P(═O)(OR⁷)₂, —OP(═O)(OR⁷)₂, —NHC(═O)(C₁-C₆)alkyl, —SO₂NR⁸₂, —OSO₂(C₁-C₆)alkyl, —OSO₂Ar³, —OC(═O)(C₁-C₃)alkyl, —O(C₂-C₆)alkylene-NR⁸₂, (C₁-C₃)perfluoroalkyl, —N(R⁵)M_{y}(R⁶) and —N(R⁶)M_{y}(R⁵);

each Ar³ is phenyl optionally substituted with 1, 2, 3, 4, or 5 substituents independently selected from the group consisting of (C₁-C₃)alkyl, (C₁-C₃)alkoxy and halogen;

each M is a connecting group independently selected from the group consisting of —(C₁-C₆)alkylene-, —(CH₂)_{d}—V—(CH₂)—, —(CH₂)_{f}—W—(CH₂)_{g} and -Z-;

each y is independently selected from the group consisting of 0 and 1;

each V is independently selected from the group consisting of arylene, heteroarylene, —C(═O)—, —C(═O)(C₁-C₆)perfluoroalkylene, —C(═O)—, —C(═S)—, —S(O)—, —SO₂—, —C(═O)NR⁷—, —C(═S)NR⁷— and —SO₂NR⁷—;

each W is independently selected from the group consisting of —NR⁷—, —O— and —S—;

each d is independently selected from the group consisting of 0, 1 and 2;

each e is independently selected from the group consisting of 0, 1 and 2;

each f is independently selected from the group consisting of 1, 2 and 3;

each g is independently selected from the group consisting of 0, 1 and 2;

-Z- is

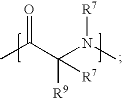

wherein the absolute configuration of -Z- is (S) or (R), or a mixture of (S) and (R);

each R⁵ is independently selected from the group consisting of hydrogen, unsubstituted aryl, substituted aryl, unsubstituted heterocyclic, substituted heterocyclic, —C(═O)OR⁷, —C(═O)NR⁸₂, —C(═NH)—NR⁸₂, —(C₁-C₆)perfluoroalkyl, —CF₂Cl, —P(═O)(OR⁷)₂, —CR⁷R¹⁰R¹¹ and a monovalent peptidyl group with a molecular weight of less than 1000, preferably with a molecular weight of less than 800, more preferably with a molecular weight of less than 600, most preferably with a molecular weight of less than 400, provided that when y is 0, R⁵ is not —C(═O)OH; and

each R⁶ is independently selected from the group consisting of —H, —(C₁-C₆)alkyl and aryl(C₁-C₃)alkyl; or

optionally, within any occurrence of —N(R⁵)M_{y}(R⁶) or —N(R⁶)M_{y}(R⁵) where y is 1, independently of any other occurrence of —N(R⁵)M_{y}(R⁶) or —N(R⁶)M_{y}(R⁵), R⁵ and R⁶ in combination represent a single bond and M is selected such that the resulting —N(R⁵)M_{y}(R⁶) or —N(R⁶)M_{y}(R⁵) moiety represents a 5, 6, or 7-membered ring heterocycle;

each R⁷ is independently selected from the group consisting of hydrogen and (C₁-C₆)alkyl;

each R⁸ is independently hydrogen or (C₁-C₆)alkyl; or, optionally, within any occurrence of NR⁸₂, independently of any other occurrence of NR⁸₂, two R⁸ groups in combination are —(CH₂)ₕ— or —(CH₂)ᵢX(CH₂)₂—;

wherein:
h is 4, 5, or 6;
i is 2 or 3;
X is O, S, NR⁷, or a single bond;

each R⁹ is independently selected from the group consisting of —H, —(C₁-C₆)alkyl, —(CH₂)₃—NH—C(NH₂)(═NH), —CH₂C(═O)NH₂, —CH₂C(═O)OH, —CH₂SH, —(CH₂)₂C(═O)—NH₂, —(CH₂)₂C(═O)OH, —CH₂-(2-imidazolyl), —(CH₂)₄—NH₂, —(CH₂)₂—S—CH₃, phenyl, CH₂-phenyl, —CH₂—OH, —CH(OH)—CH₃, —CH₂-(3-indolyl) and —CH₂-(4-hydroxyphenyl);

each R¹⁰ is independently selected from the group consisting of —H, —(C₁-C₆)alkyl, —C(═O)OR⁷, —C(═O)NR⁸₂, —OR⁷, —SR⁷, —OC(═O)(CH₂)₂C(═O)OR⁷, guanidino, —NR⁷₂, —NR⁷₃⁺, —N⁺(CH₂CH₂OR⁷)₃, phenyl, substituted phenyl, heterocyclyl and substituted heterocyclyl;

each R¹¹ is independently selected from the group consisting of R⁹, halogen, —NR⁸₂ and heterocycles containing two nitrogen atoms;

wherein the substituents for the substituted aryl and substituted heterocyclic groups comprising or included within R⁵ and R¹⁰ are independently selected from the group consisting of halogen, (C₁-C₆)alkyl, —OR⁷, —NO₂, —C≡N, —C(═O)OR⁷, —C(═O)NR⁸₂, —C(═NR⁷)NR⁸, —(C₁-C₃)alkylene-C(═O)OR⁷, —O(C₁-C₃)alkylene-C(═O)OR⁷, —(C₁-C₆)alkylene-OR⁷, —NR⁸₂, —P(═O)(OR⁷)₂, —OP(═O)(OR⁷)₂, —SO₂NR⁸₂, —NHC(═O)(C₁-C₆)alkyl, —OC(═O)(C₁-C₃)alkyl, —O(C₂-C₆)alkylene-NR⁸₂ and (C₁-C₃)perfluoroalkyl;

m is 0 or 1, provided that if D is —C≡N, then m is 1;

n is 0, 1, or 2, provided that if G is NR¹ then n is 2;

[symbol] indicates a single bond, whereby the configuration of the S—C═C—Ar² double bond may be either E or Z;

with the provisos that:
(i) if Ar¹ is unsubstituted phenyl, D is CN, G is CH₂, m is 1, and n is 2, then Ar² is other than unsubstituted phenyl;
(ii) if Ar¹ is 4-chlorophenyl, D is CN, G is CH₂, m is 1, and n is 2, then Ar² is other than 3,5-di-(t-butyl)-4-hydroxyphenyl;
(iii) if Ar¹ is unsubstituted phenyl, D is CN, G is CH₂, m is 1, n is 2, and Ar² is substituted phenyl, then Ar² is substituted at the 4-position by other than hydroxyl;
(iv) if Ar¹ is 3-trifluoromethoxyphenyl, D is CN, G is CH₂, m is 1, n is 2, and Ar² is substituted phenyl, then Ar² is substituted at the 4-position by other than hydroxyl; and
(v) if Ar² is unsubstituted phenyl, D is C(═O)NH₂, m is 0, and n is 2, then R⁴ is other than NH₂, NHCHO or NHC(═O)alkyl.

When R⁵ is a peptidyl group, the attachment point on the peptidyl group may be via a carboxyl group or through an amino group, as described above for the compounds of formula I.

Particular embodiments of the composition of the invention are those wherein D is C(═O)NH₂, m is 0, and n is 2, and Ar¹ is unsubstituted phenyl. Other particular embodiments are those wherein D is C(═O)NH₂, m is 0, and n is 2, and Ar¹ is other than unsubstituted phenyl.

Other particular embodiments of this aspect of the invention include those comprising a pharmaceutically acceptable carrier and the embodiments of a compound of formula I, or a pharmaceutically acceptable salt thereof, as described above.

Preferred embodiments of this aspect of the invention include those comprising a pharmaceutically acceptable carrier and the preferred embodiments of a compound of formula I, or a pharmaceutically acceptable salt thereof, described above.

A pharmaceutical composition is additionally provided comprising a pharmaceutically acceptable carrier and at least one conjugate according to formula I-L-Ab, or a pharmaceutically acceptable salt thereof.

The compounds of the invention may be administered in the form of a pharmaceutical composition, in combination with a pharmaceutically acceptable carrier. The active ingredient in such formulations may comprise from 0.1 to 99.99 weight percent. “Pharmaceutically acceptable carrier” means any carrier, diluent or excipient which is compatible with the other ingredients of the formulation and not deleterious to the recipient.

The active agent is preferably administered with a pharmaceutically acceptable carrier selected on the basis of the selected route of administration and standard pharmaceutical practice. The active agent may be formulated into dosage forms according to standard practices in the field of pharmaceutical preparations. See Alphonso Gennaro, ed., Remington's Pharmaceutical Sciences, 18th Edition (1990), Mack Publishing Co., Easton, Pa. Suitable dosage forms may comprise, for example, tablets, capsules, solutions, parenteral solutions, troches, suppositories, or suspensions.

For parenteral administration, the active agent may be mixed with a suitable carrier or diluent such as water, an oil (particularly a vegetable oil), ethanol, saline solution, aqueous dextrose (glucose) and related sugar solutions, glycerol, or a glycol such as propylene glycol or polyethylene glycol. Solutions for parenteral administration preferably contain a water soluble salt of the active agent. Stabilizing agents, antioxidant agents and preservatives may also be added. Suitable antioxidant agents include sulfite, ascorbic acid, citric acid and its salts, and sodium EDTA. Suitable preservatives include benzalkonium chloride, methyl- or propyl-paraben, and chlorbutanol. The composition for parenteral administration may take the form of an aqueous or non-aqueous solution, dispersion, suspension or emulsion.

For oral administration, the active agent may be combined with one or more solid inactive ingredients for the preparation of tablets, capsules, pills, powders, granules or other suitable oral dosage forms. For example, the active agent may be combined with at least one excipient such as fillers, binders, humectants, disintegrating agents, solution retarders, absorption accelerators, wetting agents absorbents or lubricating agents. According to one tablet embodiment, the active agent may be combined with carboxymethylcellulose calcium, magnesium stearate, mannitol and starch, and then formed into tablets by conventional tableting methods.

The specific dose of a compound according to the invention to obtain therapeutic benefit for treatment of a cellular proliferative disorder will, of course, be determined by the particular circumstances of the individual patient including the size, weight, age and sex of the patient, the nature and stage of the cellular proliferative disorder, the aggressiveness of the cellular proliferative disorder, and the route of administration of the compound.

For example, a daily dosage from about 0.05 to about 50 mg/kg/day may be utilized, more preferably from about 0.1 to about 10 mg/kg/day. Higher or lower doses are also contemplated as it may be necessary to use dosages outside these ranges in some cases. The daily dosage may be divided, such as being divided equally into two to four times per day daily dosing. The compositions are preferably formulated in a unit dosage form, each dosage containing from about 1 to about 500 mg, more typically, about 10 to about 100 mg of active agent per unit dosage. The term “unit dosage form” refers to physically discrete units suitable as a unitary dosage for human subjects and other mammals, each unit containing a predetermined quantity of active material calculated to produce the desired therapeutic effect, in association with a suitable pharmaceutical excipient.

The pharmaceutical compositions of the present invention may also be formulated so as to provide slow or controlled release of the active ingredient therein using, for example, hydropropylmethyl cellulose in varying proportions to provide the desired release profile, other polymer matrices, gels, permeable membranes, osmotic systems, multilayer coatings, microparticles, liposomes and/or microspheres.

In general, a controlled-release preparation is a pharmaceutical composition capable of releasing the active ingredient at the required rate to maintain constant pharmacological activity for a desirable period of time. Such dosage forms provide a supply of a drug to the body during a predetermined period of time and thus maintain drug levels in the therapeutic range for longer periods of time than conventional non-controlled formulations.

U.S. Pat. No. 5,674,533 discloses controlled-release pharmaceutical compositions in liquid dosage forms for the administration of moguisteine, a potent peripheral antitussive. U.S. Pat. No. 5,059,595 describes the controlled-release of active agents by the use of a gastro-resistant tablet for the therapy of organic mental disturbances. U.S. Pat. No. 5,591,767 describes a liquid reservoir transdermal patch for the controlled administration of ketorolac, a non-steroidal anti-inflammatory agent with potent analgesic properties. U.S. Pat. No. 5,120,548 discloses a controlled-release drug delivery device comprised of swellable polymers. U.S. Pat. No. 5,073,543 describes controlled-release formulations containing a trophic factor entrapped by a ganglioside-liposome vehicle. U.S. Pat. No. 5,639,476 discloses a stable solid controlled-release formulation having a coating derived from an aqueous dispersion of a hydrophobic acrylic polymer. Biodegradable microparticles are known for use in controlled-release formulations. U.S. Pat. No. 5,354,566 discloses a controlled-release powder that contains the active ingredient. U.S. Pat. No. 5,733,566, describes the use of polymeric microparticles that release antiparasitic compositions.

The controlled-release of the active ingredient may be stimulated by various inducers, for example pH, temperature, enzymes, water, or other physiological conditions or compounds. Various mechanisms of drug release exist. For example, in one embodiment, the controlled-release component may swell and form porous openings large enough to release the active ingredient after administration to a patient. The term “controlled-release component” in the context of the present invention is defined herein as a compound or compounds, such as polymers, polymer matrices, gels, permeable membranes, liposomes and/or microspheres, that facilitate the controlled-release of the active ingredient in the pharmaceutical composition. In another embodiment, the controlled-release component is biodegradable, induced by exposure to the aqueous environment, pH, temperature, or enzymes in the body. In another embodiment, sol-gels may be used, wherein the active ingredient is incorporated into a sol-gel matrix that is a solid at room temperature. This matrix is implanted into a patient, preferably a mammal, having a body temperature high enough to induce gel formation of the sol-gel matrix, thereby releasing the active ingredient into the patient.

The components used to formulate the pharmaceutical compositions are of high purity and are substantially free of potentially harmful contaminants (e.g., at least National Food grade, generally at least analytical grade, and more typically at least pharmaceutical grade). Particularly for human consumption, the composition is preferably manufactured or formulated under Good Manufacturing Practice standards as defined in the applicable regulations of the U.S. Food and Drug Administration. For example, suitable formulations may be sterile and/or substantially isotonic and/or in full compliance with all Good Manufacturing Practice regulations of the U.S. Food and Drug Administration.

### VI. Methods of Treatment of Cellular Proliferative Disorders Using Compounds of the Invention

According to another aspect of the invention, methods of treating an individual suffering from a cellular proliferative disorder, particularly cancer, or of inducing apoptosis of cancer cells, particularly tumor cells, in an individual suffering cancer are provided, said methods comprising administering to said individual an effective amount of at least one compound according to formula I:

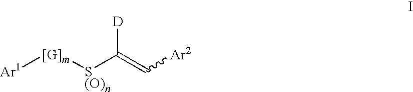

wherein:

Ar¹ is:

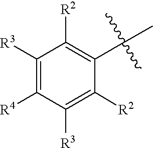

Ar² is selected from the group consisting of unsubstituted aryl, substituted aryl, unsubstituted heteroaryl and substituted heteroaryl; wherein, when Ar² is substituted aryl or substituted heteroaryl, said substituted aryl or substituted heteroaryl is aryl or heteroaryl substituted with one or more substituents independently selected from the group consisting of halogen, (C₁-C₆)alkyl, (C₂-C₆)alkenyl, (C₂-C₆)alkynyl, —(C₁-C₃)alkylene-Ar³, —OR⁷, —OAr³, —O(C₁-C₃)alkylene-Ar³, (C₁-C₃)fluoroalkoxy, —NO₂, —C≡N, —C(═O)(C₁-C₃)alkyl, —C(═O)OR⁷, —C(═O)NR⁸₂, —C(═NR⁷)NR⁸₂, —(C₁-C₃)alkylene-C(═O)OR⁷, —O(C₁-C₃)alkylene-C(═O)OR⁷, —(C₁-C₆)alkylene-OR⁷, NR⁸₂—P(═O)(OR⁷)₂, —OP(═O)(OR⁷)₂, —S(C₁-C₆)alkyl, —S(O)(C₁-C₆)alkyl, —SO₂(C₁-C₆)alkyl, —SO₂NR⁸₂, —OSO₂(C₁-C₆)alkyl, —OSO₂Ar³, —NHC(═O)(C₁-C₆)alkyl, —OC(═O)(C₁-C₃)alkyl, —O(C₂-C₆)alkylene-NR⁸₂ and (C₁-C₃)perfluoroalkyl;

D is —C≡N, —C(═O)NR⁸₂, or NO₂;

G is CR¹₂ or NR¹;

R¹ is independently selected from the group consisting of hydrogen and (C₁-C₆)alkyl;

each R² is independently selected from the group consisting of hydrogen, (C₁-C₆)alkyl, halogen, —OR⁷, —OAr³, —O(C₁-C₃)alkylene-Ar³, —C≡N, —NO₂, —C(═O)OR⁷, —C(═O)NR⁸₂, —C(═NR⁷)NR⁸₂, —(C₁-C₃)alkylene-C(═O)OR⁷, —O(C₁-C₃)alkylene-C(═O)OR⁷, —(C₁-C₆)alkylene-OR⁷, —P(═O)(OR⁷)₂, —OP(═O)(OR⁷)₂, —NHC(═O)(C₁-C₆)alkyl, —SO₂NR⁸₂, —OSO₂(C₁-C₆)alkyl, —OSO₂Ar³, —OC(═O)(C₁-C₃)alkyl, —O(C₂-C₆)alkylene-NR⁸₂, (C₁-C₃)perfluoroalkyl and —N(R⁶)-(M)_{y}-R⁵;

each R³ is independently selected from the group consisting of hydrogen, (C₁-C₆)alkyl, halogen, —OR⁷, —OAr³, —O(C₁-C₃)alkylene-Ar³, —C≡N, —NO₂, —C(═O)OR⁷, —C(═O)NR⁸₂, —C(═NR⁷)NR⁸₂, —(C₁-C₃)alkylene-C(═O)OR⁷, —O(C₁-C₃)alkylene-C(═O)OR⁷, —(C₁-C₆)alkylene-OR⁷, —P(═O)(OR⁷)₂, —OP(═O)(OR⁷)₂, —NHC(═O)(C₁-C₆)alkyl, —SO₂NR⁸₂, —OSO₂(C₁-C₆)alkyl, —OSO₂Ar³, —OC(═O)(C₁-C₃)alkyl, —O(C₂-C₆)alkylene-NR⁸₂, (C₁-C₃)perfluoroalkyl and —N(R⁶)-(M)_{y}-R⁵;

R⁴ is selected from the group consisting of hydrogen, (C₁-C₆)alkyl, halogen, —OR⁷, —OAr³, —O(C₁-C₃)alkylene-Ar³, —C≡N, —NO₂, —C(═O)OR⁷, —C(═O)NR⁸₂, —C(═NR⁷)NR⁸₂, —(C₁-C₃)alkylene-C(═O)OR⁷, —O(C₁-C₃)alkylene-C(═O)OR⁷, —(C₁-C₆)alkylene-OR⁷, —P(═O)(OR⁷)₂, —OP(═O)(OR⁷)₂, —NHC(═O)(C₁-C₆)alkyl, —SO₂NR⁸₂, —OSO₂(C₁-C₆)alkyl, —OSO₂Ar³, —OC(═O)(C₁-C₃)alkyl, —O(C₂-C₆)alkylene-NR⁸₂, (C₁-C₃)perfluoroalkyl, —N(R⁶)M_{y}(R⁶) and —N(R⁶)M_{y}(R⁵);

each Ar³ is phenyl optionally substituted with 1, 2, 3, 4, or 5 substituents independently selected from the group consisting of (C₁-C₃)alkyl, (C₁-C₃)alkoxy and halogen;

each M is a connecting group independently selected from the group consisting of —(C₁-C₆)alkylene-, —(CH₂)_{d}—V—(CH₂)ₑ—, —(CH₂)_{f}—W—(CH₂)_{g}— and -Z-;

each y is independently selected from the group consisting of 0 and 1;

each V is independently selected from the group consisting of arylene, heteroarylene, —C(═O)—, —C(═O)(C₁-C₆)perfluoroalkylene, —C(═O)—, —C(═S)—, —S(═O)—, —SO₂—, —C(═O)NR⁷—, —C(═S)NR⁷— and —SO₂NR⁷—;

each W is independently selected from the group consisting of —NR⁷—, —O— and —S—;

each d is independently selected from the group consisting of 0, 1 and 2;

each e is independently selected from the group consisting of 0, 1 and 2;

each f is independently selected from the group consisting of 1, 2 and 3;

each g is independently selected from the group consisting of 0, 1 and 2;

-Z- is

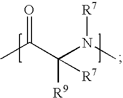

wherein the absolute configuration of -Z- is (S) or (R), or a mixture of (S) and (R);

each R⁵ is independently selected from the group consisting of hydrogen, unsubstituted aryl, substituted aryl, unsubstituted heterocyclic, substituted heterocyclic, —C(═O)OR⁷, —C(═O)NR⁸₂, —C(—NH)—NR⁸₂, —(C₁-C₆)perfluoroalkyl, —CF₂Cl, —P(═O)(OR⁷)₂, —CR⁷R¹⁰R¹¹ and a monovalent peptidyl group with a molecular weight of less than 1000, preferably with a molecular weight of less than 800, more preferably with a molecular weight of less than 600, most preferably with a molecular weight of less than 400,

provided that when y is 0, R⁵ is not —C(═O)OH; and

each R⁶ is independently selected from the group consisting of —H, —(C₁-C₆)alkyl and aryl(C₁-C₃)alkyl; or

optionally, within any occurrence of —N(R⁵)M_{y}(R⁶) or —N(R⁶)M_{y}(R⁵) where y is 1, independently of any other occurrence of —N(R⁵)M_{y}(R⁶) or —N(R⁶)M_{y}(R⁵), R⁵ and R⁶ in combination represent a single bond and M is selected such that the resulting —N(R⁵)M_{y}(R⁶) or —N(R⁶)M_{y}(R⁵) moiety represents a 5, 6, or 7-membered ring heterocycle;

each R⁷ is independently selected from the group consisting of hydrogen and (C₁-C₆)alkyl;

each R⁸ is independently hydrogen or (C₁-C₆)alkyl; or, optionally, within any occurrence of NR⁸₂, independently of any other occurrence of NR⁸₂, two R⁸ groups in combination are —(CH₂)ₕ— or —(CH₂)ᵢX(CH₂)₂—;

wherein:
h is 4, 5, or 6;
i is 2 or 3;
X is O, S, NR⁷, or a single bond;

each R⁹ is independently selected from the group consisting of —H, —(C₁-C₆)alkyl, —(CH₂)₃—NH—C(NH₂)(═NH), —CH₂C(═O)NH₂, —CH₂C(═O)OH, —CH₂SH, —(CH₂)₂C(═O)—NH₂, —(CH₂)₂C(═O)OH, —CH₂-(2-imidazolyl), —(CH₂)₄—NH₂, —(CH₂)₂—S—CH₃, phenyl, CH₂-phenyl, —CH₂—OH, —CH(OH)—CH₃, —CH₂-(3-indolyl) and —CH₂-(4-hydroxyphenyl);

each R¹⁰ is independently selected from the group consisting of —H, —(C₁-C₆)alkyl, —C(═O)OR⁷, —C(═O)NR⁸₂, —OR⁷, —SR⁷, —OC(═O)(CH₂)₂C(═O)OR⁷, guanidino, —NR⁷₂, —NR⁷₃⁺, —N⁺(CH₂CH₂OR⁷)₃, phenyl, substituted phenyl, heterocyclyl and substituted heterocyclyl;

each R¹¹ is independently selected from the group consisting of R⁹, halogen, —NR⁸₂ and heterocycles containing two nitrogen atoms;

wherein the substituents for the substituted aryl and substituted heterocyclic groups comprising or included within R⁵ and R¹⁰ are independently selected from the group consisting of halogen, (C₁-C₆)alkyl, —OR⁷, —NO₂, —C≡N, —C(═O)OR⁷, —C(═O)NR⁸₂, —C(—NR⁷)NR⁸₂, —(C₁-C₃)alkylene-C(═O)OR⁷, —O(C₁-C₃)alkylene-C(═O)OR⁷, —(C₁-C₆)alkylene-OR⁷, —NR⁸₂, —P(═O)(OR⁷)₂, —OP(═O)(OR⁷)₂, —SO₂NR⁸₂, —NHC(═O)(C₁-C₆)alkyl, —OC(═O)(C₁-C₃)alkyl, —O(C₂-C₆)alkylene-NR⁸₂ and (C₁-C₃)perfluoroalkyl;

m is 0 or 1, provided that if D is —C≡N, then m is 1;

n is 0, 1, or 2, provided that if G is NR¹ then n is 2;

[symbol] indicates a single bond, whereby the configuration of the S—C≡C—Ar² double bond may be either E or Z;

with the provisos that:

(i) if Ar¹ is unsubstituted phenyl, D is CN, G is CH₂, m is 1, and n is 2, then Ar² is other than unsubstituted phenyl;
(ii) if Ar¹ is 4-chlorophenyl, D is CN, G is CH₂, m is 1, and n is 2, then Ar² is other than 3,5-di-(t-butyl)-4-hydroxyphenyl;
(iii) if Ar¹ is unsubstituted phenyl, D is CN, G is CH₂, m is 1, n is 2, and Ar² is substituted phenyl, then Ar² is substituted at the 4-position by other than hydroxyl; and
(iv) if Ar¹ is 3-trifluoromethoxyphenyl, D is CN, G is CH₂, m is 1, n is 2, and Ar² is substituted phenyl, then Ar² is substituted at the 4-position by other than hydroxyl;

or a pharmaceutically acceptable salt thereof, either alone, or in combination with a pharmaceutically acceptable carrier.

According an embodiment of this aspect of the invention, a method of treating an individual suffering from a cellular proliferative disorder, particularly cancer, is provided, comprising administering to said individual an effective amount of at least one compound according to formula I, or a pharmaceutically acceptable salt thereof, either alone, or in combination with a pharmaceutically acceptable carrier.

According to an embodiment of this aspect of the invention, a method of inducing apoptosis of cancer cells, preferably tumor cells, in an individual afflicted with cancer is provided, comprising administering to said individual an effective amount of at least one compound according to formula I or a pharmaceutically acceptable salt thereof, either alone, or in combination with a pharmaceutically acceptable carrier.

According to another embodiment of this aspect of the invention, a method of treating an individual suffering from a cellular proliferative disorder, particularly cancer, is provided, comprising administering to said individual an effective amount of at least one conjugate of the formula I-L-Ab, either alone, or in combination with a pharmaceutically acceptable carrier.

The invention is also directed to the use in medicine of a compound according to formula I, or a pharmaceutically acceptable salt thereof, or a conjugate according to formula I-L-Ab, or a pharmaceutically acceptable salt thereof.

The invention is also directed to the use of a compound according to formula I, or a pharmaceutically acceptable salt thereof, or a conjugate according to formula I-L-Ab, or a pharmaceutically acceptable salt thereof, or a composition comprising such a compound, conjugate, or salt, in the preparation of a medicament for treatment of a cellular proliferative disorder, particularly cancer, or for inducing apoptosis of tumor cells in an individual affected with cancer.

Particular and preferred embodiments of these aspects of the invention are those wherein the compound of formula I used in the method of treatment, either alone or as part of a composition, or as a component of the antibody conjugate, is any embodiment of the compounds of formula I described herein, including particular and preferred embodiments of the compound of formula I in the description of the compounds and compositions of the invention as provided herein.

The compounds according to the invention may be administered to individuals (mammals, including animals and humans) afflicted with a cellular proliferative disorder such as cancer, malignant and benign tumors, blood vessel proliferative disorders, autoimmune disorders, and fibrotic disorders.

The compounds are believed effective against a broad range of tumor types, including but not limited to the following: ovarian cancer; cervical cancer; breast cancer; prostate cancer; testicular cancer, lung cancer, renal cancer; colorectal cancer; skin cancer; brain cancer; leukemia, including acute myeloid leukemia, chronic myeloid leukemia, acute lymphoid leukemia, and chronic lymphoid leukemia.

More particularly, cancers that may be treated by the compounds, compositions and methods of the invention include, but are not limited to, the following:
cardiac cancers, including, for example sarcoma, e.g., angiosarcoma, fibrosarcoma, rhabdomyosarcoma, and liposarcoma; myxoma; rhabdomyoma; fibroma; lipoma and teratoma;
lung cancers, including, for example, bronchogenic carcinoma, e.g., squamous cell, undifferentiated small cell, undifferentiated large cell, and adenocarcinoma; alveolar and bronchiolar carcinoma; bronchial adenoma; sarcoma; lymphoma; chondromatous hamartoma; and mesothelioma;
gastrointestinal cancer, including, for example, cancers of the esophagus, e.g., squamous cell carcinoma, adenocarcinoma, leiomyosarcoma, and lymphoma; cancers of the stomach, e.g., carcinoma, lymphoma, and leiomyosarcoma; cancers of the pancreas, e.g., ductal adenocarcinoma, insulinoma, glucagonoma, gastrinoma, carcinoid tumors, and vipoma; cancers of the small bowel, e.g., adenocarcinoma, lymphoma, carcinoid tumors, Kaposi's sarcoma, leiomyoma, hemangioma, lipoma, neurofibroma, and fibroma; cancers of the large bowel, e.g., adenocarcinoma, tubular adenoma, villous adenoma, hamartoma, and leiomyoma;
genitourinary tract cancers, including, for example, cancers of the kidney, e.g., adenocarcinoma, Wilm's tumor (nephroblastoma), lymphoma, and leukemia; cancers of the bladder and urethra, e.g., squamous cell carcinoma, transitional cell carcinoma, and adenocarcinoma; cancers of the prostate, e.g., adenocarcinoma, and sarcoma; cancer of the testis, e.g., seminoma, teratoma, embryonal carcinoma, teratocarcinoma, choriocarcinoma, sarcoma, interstitial cell carcinoma, fibroma, fibroadenoma, adenomatoid tumors, and lipoma;
liver cancers, including, for example, hepatoma, e.g., hepatocellular carcinoma; cholangiocarcinoma; hepatoblastoma; angiosarcoma; hepatocellular adenoma; and hemangioma;
bone cancers, including, for example, osteogenic sarcoma (osteosarcoma), fibrosarcoma, malignant fibrous histiocytoma, chondrosarcoma, Ewing's sarcoma, malignant lymphoma (reticulum cell sarcoma), multiple myeloma, malignant giant cell tumor chordoma, osteochrondroma (osteocartilaginous exostoses), benign chondroma, chondroblastoma, chondromyxofibroma, osteoid osteoma and giant cell tumors;
nervous system cancers, including, for example, cancers of the skull, e.g., osteoma, hemangioma, granuloma, xanthoma, and osteitis deformans; cancers of the meninges, e.g., meningioma, meningiosarcoma, and gliomatosis; cancers of the brain, e.g., astrocytoma, medulloblastoma, glioma, ependymoma, germinoma (pinealoma), glioblastoma multiform, oligodendroglioma, schwannoma, retinoblastoma, and congenital tumors; and cancers of the spinal cord, e.g., neurofibroma, meningioma, glioma, and sarcoma;
gynecological cancers, including, for example, cancers of the uterus, e.g., endometrial carcinoma; cancers of the cervix, e.g., cervical carcinoma, and pre-tumor cervical dysplasia; cancers of the ovaries, e.g., ovarian carcinoma, including serous cystadenocarcinoma, mucinous cystadenocarcinoma, unclassified carcinoma, granulosa-thecal cell tumors, Sertoli-Leydig cell tumors, dysgerminoma, and malignant teratoma; cancers of the vulva, e.g., squamous cell carcinoma, intraepithelial carcinoma, adenocarcinoma, fibrosarcoma, and melanoma; cancers of the vagina, e.g., clear cell carcinoma, squamous cell carcinoma, botryoid sarcoma, and embryonal rhabdomyosarcoma; and cancers of the fallopian tubes, e.g., carcinoma;
hematologic cancers, including, for example, cancers of the blood, e.g., acute myeloid leukemia, chronic myeloid leukemia, acute lymphoblastic leukemia, chronic lymphocytic leukemia, myeloproliferative diseases, multiple myeloma, and myelodysplastic syndrome, Hodgkin's lymphoma, non-Hodgkin's lymphoma (malignant lymphoma) and Waldenström's macroglobulinemia;
skin cancers, including, for example, malignant melanoma, basal cell carcinoma, squamous cell carcinoma, Kaposi's sarcoma, moles dysplastic nevi, lipoma, angioma, dermatofibroma, keloids, psoriasis; and
adrenal gland cancers, including, for example, neuroblastoma.

Cancers may be solid tumors that may or may not be metastatic. Cancers may also occur, as in leukemia, as a diffuse tissue. Thus, the term “tumor cell”, as provided herein, includes a cell afflicted by any one of the above identified disorders.

The compounds are also believed useful in the treatment of non-cancer cellular proliferative disorders, that is, cellular proliferative disorders which are characterized by benign indications. Such disorders may also be known as “cytoproliferative” or “hyperproliferative” in that cells are made by the body at an atypically elevated rate. Non-cancer cellular proliferative disorders believed treatable by compounds according to the invention include, for example: hemangiomatosis in newborn, secondary progressive multiple sclerosis, atherosclerosis, chronic progressive myelodegenerative disease, neurofibromatosis, ganglioneuromatosis, keloid formation, Paget's disease of the bone, fibrocystic disease of the breast, uterine fibroids, Peyronie's disease, Dupuytren's disease, restenosis, benign proliferative breast disease, benign prostatic hyperplasia, X-linked lymphocellular proliferative disorder (Duncan disease), post-transplantation lymphocellular proliferative disorder (PTLD), macular degeneration, and retinopathies, such as diabetic retinopathies and proliferative vitreoretinopathy (PVR)

Other non-cancer cellular proliferative disorders believed treatable by compounds according to the invention include the presence of pre-cancerous lymphoproliferative cells associated with an elevated risk of progression to a cancerous disorder. Many non-cancerous lymphocellular proliferative disorders are associated with latent viral infections such as Epstein-Barr virus (EBV) and Hepatitis C. These disorders often begin as a benign pathology and progress into lymphoid neoplasia as a function of time.

### VII. Salts of Compounds According to the Invention

The compounds of the present invention may take the form of salts. The term “salts” embraces addition salts of free acids or free bases which are compounds of the invention. The term “pharmaceutically-acceptable salt” refers to salts which possess toxicity profiles within a range that affords utility in pharmaceutical applications. Pharmaceutically unacceptable salts may nonetheless possess properties such as high crystallinity, which have utility in the practice of the present invention, such as for example utility in process of synthesis, purification or formulation of compounds of the invention.

Suitable pharmaceutically-acceptable acid addition salts may be prepared from an inorganic acid or from an organic acid. Examples of inorganic acids include hydrochloric, hydrobromic, hydriodic, nitric, carbonic, sulfuric, and phosphoric acids. Appropriate organic acids may be selected from aliphatic, cycloaliphatic, aromatic, araliphatic, heterocyclic, carboxylic and sulfonic classes of organic acids, examples of which include formic, acetic, propionic, succinic, glycolic, gluconic, lactic, malic, tartaric, citric, ascorbic, glucuronic, maleic, fumaric, pyruvic, aspartic, glutamic, benzoic, anthranilic, 4-hydroxybenzoic, phenylacetic, mandelic, embonic (pamoic), methanesulfonic, ethanesulfonic, benzenesulfonic, pantothenic, trifluoromethanesulfonic, 2-hydroxyethanesulfonic, p-toluenesulfonic, sulfanilic, cyclohexylaminosulfonic, stearic, alginic, β-hydroxybutyric, salicylic, galactaric and galacturonic acid. Examples of pharmaceutically unacceptable acid addition salts include, for example, perchlorates and tetrafluoroborates.

Suitable pharmaceutically acceptable base addition salts of compounds of the invention include, for example, metallic salts including alkali metal, alkaline earth metal and transition metal salts such as, for example, calcium, magnesium, potassium, sodium and zinc salts. Pharmaceutically acceptable base addition salts also include organic salts made from basic amines such as, for example, N,N′-dibenzylethylenediamine, chloroprocaine, choline, diethanolamine, ethylenediamine, meglumine (N-methylglucamine) and procaine. Examples of pharmaceutically unacceptable base addition salts include lithium salts and cyanate salts. All of these salts may be prepared by conventional means from the corresponding compound according to Formula I by reacting, for example, the appropriate acid or base with the compound according to Formula I.

### VIII. Routes of Administration of Compounds of the Invention

The compounds may be administered by any route, including oral, rectal, sublingual, and parenteral administration. Parenteral administration includes, for example, intravenous, intramuscular, intraarterial, intraperitoneal, intranasal, intravaginal, intravesical (e.g., to the bladder), intradermal, transdermal, topical or subcutaneous administration. Also contemplated within the scope of the invention is the instillation of a drug in the body of the patient in a controlled formulation, with systemic or local release of the drug to occur at a later time. For example, the drug may be localized in a depot for controlled release to the circulation, or for release to a local site of tumor growth.

One or more compounds useful in the practice of the present inventions may be administered simultaneously, by the same or different routes, or at different times during treatment. The compounds may be administered before, along with, or after other medications, including other antiproliferative compounds.

The treatment may be carried out for as long a period as necessary, either in a single, uninterrupted session, or in discrete sessions. The treating physician will know how to increase, decrease, or interrupt treatment based on patient response. According to one embodiment, treatment is carried out for from about four to about sixteen weeks. The treatment schedule may be repeated as required.

### IX. Isomerism in Compounds of the Invention

### 1. Geometrical Isomerism

The compounds of the invention are characterized by isomerism resulting from the presence of an olefinic double bond. This isomerism is commonly referred to as cis-trans isomerism, but the more comprehensive naming convention employs E and Z designations. The compounds are named according to the Cahn-Ingold-Prelog system, described in the IUPAC 1974 Recommendations, Section E: Stereochemistry, in Nomenclature of Organic Chemistry, John Wiley & Sons, Inc., New York, N.Y., 4^{th} ed., 1992, pp. 127-38, the entire contents of which is incorporated herein by reference. Using this system of nomenclature, the four groups about a double bond are prioritized according to a series of rules wherein various functional groups are ranked. The isomer with the two higher ranking groups on the same side of the double bond is designated Z and the other isomer, in which the two higher ranking groups are on opposite sides of the double bond, is designated E. Both E and Z configurations are included in the scope of the compounds of the present invention. The E configuration is preferred.

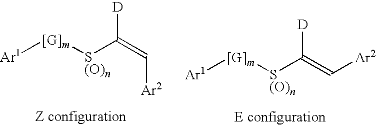

### 2. Optical Isomerism

It will be understood that when compounds of the present invention contain one or more chiral centers, the compounds may exist in, and may be isolated as pure enantiomeric or diastereomeric forms or as racemic mixtures. The present invention therefore includes any possible enantiomers, diastereomers, racemates or mixtures thereof of the compounds of the invention which are biologically active in the treatment of cancer or other proliferative disease states.

The isomers resulting from the presence of a chiral center comprise a pair of non-superimposable isomers that are called “enantiomers.” Single enantiomers of a pure compound are optically active, i.e., they are capable of rotating the plane of plane polarized light. Single enantiomers are designated according to the Cahn-Ingold-Prelog system. Once the priority ranking of the four groups is determined, the molecule is oriented so that the lowest ranking group is pointed away from the viewer. Then, if the descending rank order of the other groups proceeds clockwise, the molecule is designated (R) and if the descending rank of the other groups proceeds counterclockwise, the molecule is designated (S). In the example in Scheme 7, the Cahn-Ingold-Prelog ranking is A>B>C>D. The lowest ranking atom, D is oriented away from the viewer.

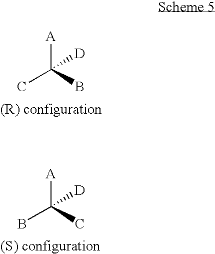

The present invention is meant to encompass diastereomers as well as their racemic and resolved, diastereomerically and enantiomerically pure forms and salts thereof. Diastereomeric pairs may be resolved by known separation techniques including normal and reverse phase chromatography, and crystallization.

“Isolated optical isomer” means a compound which has been substantially purified from the corresponding optical isomer(s) of the same formula. Preferably, the isolated isomer is at least about 80%, more preferably at least 90% pure, even more preferably at least 98% pure, most preferably at least about 99% pure, by weight.

Isolated optical isomers may be purified from racemic mixtures by well-known chiral separation techniques. According to one such method, a racemic mixture of a compound having the structure of Formula I, or a chiral intermediate thereof, is separated into 99% wt. % pure optical isomers by HPLC using a suitable chiral column, such as a member of the series of DAICEL® CHIRALPAK® family of columns (Daicel Chemical Industries, Ltd., Tokyo, Japan). The column is operated according to the manufacturer's instructions.

### 3. Rotational Isomerism

It is understood that due to chemical properties (i.e., resonance lending some double bond character to the C—N bond) of restricted rotation about the amide bond linkage (as illustrated below) it is possible to observe separate rotamer species and even, under some circumstances, to isolate such species. It is further understood that certain structural elements, including steric bulk or substituents on the amide nitrogen, may enhance the stability of a rotamer to the extent that a compound may be isolated as, and exist indefinitely, as a single stable rotamer. The present invention therefore includes any possible stable rotamers of formula I which are biologically active in the treatment of cancer or other proliferative disease states.

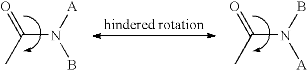

### 4. Regioisomerism

The preferred compounds of the present invention have a particular spatial arrangement of substituents on the aromatic rings, which is related to the structure activity relationship demonstrated by the compound class. Often such substitution arrangement is denoted by a numbering system; however, numbering systems are often not consistent between different ring systems. In six-membered aromatic systems, the spatial arrangements are specified by the common nomenclature “para” for 1,4-substitution, “meta” for 1,3-substitution and “ortho” for 1,2-substitution as shown below.

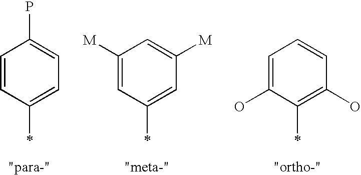

### EXAMPLES

The following non-limiting examples are provided to illustrate the invention. In the synthetic pathways and methods that follow, reference to Ar or Ar′ and to the term “aryl” is intended to include substituted and unsubstituted aryl, and also substituted and unsubstituted heteroaryl. The illustrated synthetic pathways are applicable to other embodiments of the invention. The synthetic procedures described as “general methods” describe what it is believed will be typically effective to perform the synthesis indicated. However, the person skilled in the art will appreciate that it may be necessary to vary the procedures for any given embodiment of the invention. For example, reaction monitoring, such as by using thin layer chromatography, or HPLC may be used to determine the optimum reaction time. Products may be purified by conventional techniques that will vary, for example, according to the amount of side products produced and the physical properties of the compounds. On a laboratory scale, recrystallisation from a suitable solvent, column chromatography, normal or reverse phase HPLC, or distillation are all techniques which may be useful. The person skilled in the art will appreciate how to vary the reaction conditions to synthesize any given compound within the scope of the invention without undue experimentation. See, e.g., Vogel's Textbook of Practical Organic Chemistry, by A. I. Vogel, et al, Experimental Organic Chemistry: Standard and Microscale, by L. M. Harwood et al. (2^{nd} Ed., Blackwell Scientific Publications, 1998), and Advanced Practical Organic Chemistry, by J. Leonard, et al. (2^{nd} Edition, CRC Press 1994).

### SYNTHESIS EXAMPLES

### Synthesis Example 1

### Synthesis of 3-Aryl-2-(arylmethanesulfonyl)acrylonitriles

### Step 1. Preparation of 2-(Arylmethanethio)acetonitrile

2-(Arylmethanethio)acetonitriles may be prepared by Method A or Method B.

### Method A:

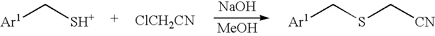

An arylmethyl mercaptan (5 mmol) is added slowly through the dropping funnel to a stirred solution of sodium hydroxide (5 mmol) in methanol (50 mL) in a 100 mL two-necked round-bottomed flask equipped with a reflux condenser. A vigorous reaction occurs immediately. On completion of the addition, and when the reaction is no longer exothermic, chloroacetonitrile (5 mmol) is added in portions. The cooled reaction mixture is stirred at room temperature for 3 hours then poured onto crushed ice. If a solid product forms, it is typically collected by filtration, washed with ice-cold water and dried. If a solid product is not formed, the mixture is typically extracted with ethyl acetate, dried over anhydrous sodium sulfate and concentrated to obtain the 2-(arylmethanethio)acetonitrile.

### Method B:

This alternative procedure for forming 2-(arylmethanethio)acetonitriles involves a multi-step synthesis.

### (a) Preparation of 2-(Acetylthio)acetonitrile

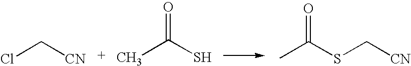

Triethylamine was added dropwise over a period of approximately two hours to a cold (−30° C.) stirred solution of chloroacetonitrile (1.0 mol) and thiolacetic acid (1.0 mol) in dichloromethane (200 mL). After the addition was completed, the solution was stirred at −30° C. for 5 min, then allowed to warm slowly to room temperature. Water (20 mL) was added to the reaction mixture and the organic layer was washed with 10% dilute acetic acid (2×30 mL) and water (2×30 mL). The combined extracts were dried over sodium sulfate and solvent was removed under a vacuum. The orange oil which formed was used without any further purification.

### (b) Preparation of 2-mercaptoacetonitrile

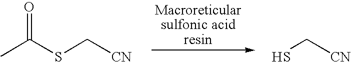

A mixture containing 2-acetylthioacetonitrile (0.25 mol), methanol (300 mL) and dried macroreticular sulfonic acid resin (Amberlyst® 15) (8.7 g) was heated with stirring under reflux under a nitrogen atmosphere for 20 hours. The resulting mixture was allowed to cool and then filtered and a catalytic amount of fresh macroreticular sulfonic acid resin (Amberlyst® 15) (0.5 g) was added to stabilize the product 2-mercaptoacetonitrile. The solvent was removed under vacuum without heating and the resulting 2-mercaptoacetonitrile was used without further purification.

### (c) Preparation of 2-(arylmethanethio)acetonitrile

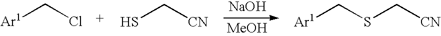

2-Mercaptoacetonitrile (0.5 mol) is added slowly through the dropping funnel into a stirred solution of sodium hydroxide (0.5 mol) in methanol (125 mL) in a 250 mL two-necked round-bottomed flask equipped with a reflux condenser. A vigorous reaction immediately occurs. On completion of the addition and when the reaction is no longer exothermic, an arylmethyl chloride (0.5 mol) is added in portions, and the resulting solution is stirred at room temperature for 3 hours. The reaction mixture is cooled and then poured into crushed ice. If a solid product is formed, it is typically collected by filtration, washed with ice-cold water, and dried. Otherwise, the solution is typically extracted with ethyl acetate, dried over anhydrous sodium sulfate and concentrated to obtain the desired product.

### Step 2. Preparation of 2-(arylmethanesulfonyl)acetonitrile

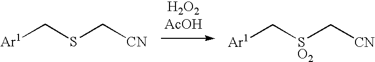

30% Hydrogen peroxide (10 mL) is added slowly to an ice-cold solution of the 2-(arylmethanethio)acetonitrile (5.0 g) in glacial acetic acid (35 mL). The reaction mixture is kept at room temperature for 18 hours, then poured onto crushed ice. If a solid product is formed, it is typically collected by filtration, washed with ice-cold water, and dried. Otherwise, the solution is typically extracted with ethyl acetate, dried over anhydrous sodium sulfate and concentrated to obtain the desired product.

### Step 3. Synthesis of 3-aryl-2-(arylmethanesulfonyl)acrylonitriles

Two methods for the synthesis of 3-aryl-2-(arylmethanesulfonyl)acrylonitriles by the condensation of a 2-(arylmethanesulfonyl)acetonitrile with an arenecarboxaldehyde are described below.

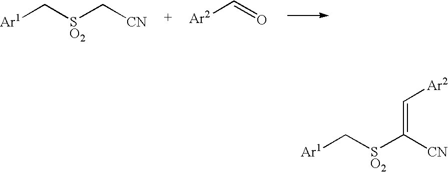

### Method A:

A mixture of a 2-(arylmethanesulfonyl)acetonitrile (10 mmol), an arenecarboxaldehyde (10 mmol), glacial acetic acid (15 mL) and a catalytic amount of benzylamine (200 μL) is heated under reflux for about 2 to 8 hours. The reaction mixture is cooled. If the product precipitates, it is typically collected by filtration and filtered and dried. If the product does not precipitate, the mixture is typically diluted with ether, and successively washed with a saturated solution of sodium bisulfite (2×15 mL), dilute hydrochloric acid (2×20 mL) and water, dried and evaporated under vacuum. The crude product is typically purified by recrystallization from 2-propanol.

### Method B:

A mixture of a 2-(arylmethanesulfonyl)acetonitrile (1.0 eq.), an arenecarboxaldehyde (1.1 eq.), benzoic acid (0.15 eq.) and piperidine (0.13 eq.) in toluene (50 mL) is heated under refluxed for 2 to 3 hours with continuous removal of water using a Dean-Stark water separator. The reaction mixture is allowed to cool to room temperature. In a typical work-up, water is added, the mixture is extracted with ethyl acetate, and the organic layer is washed with saturated aqueous sodium bicarbonate, dilute hydrochloric acid, and water, and is then dried, filtered, and evaporated under vacuum. The crude product is typically purified by recrystallization from 2-propanol.

### Synthesis Example 2

### Synthesis of 1-Aryl-2-(arylmethanesulfonyl)-2-nitroethenes and 1-Aryl-2-(arylsulfonyl)-2-nitroethenes

### Step 1. Preparation of (Arylmethanethio)nitromethanes and (Arylthio)nitromethanes

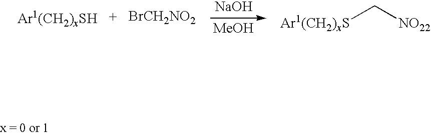

An arylmethyl or aryl mercaptan (5 mmol) is added slowly through the dropping funnel to a stirred solution of sodium hydroxide (5 mmol) in methanol (50 mL) in a 100 mL two-necked round-bottomed flask equipped with a reflux condenser. On completion of the addition, and when the reaction is no longer exothermic, bromonitromethane (5 mmol) is added in portions then the reaction mixture is stirred at room temperature for 3 hours. The reaction mixture is then poured onto crushed ice. If a solid product is formed, it is typically collected by filtration, washed with ice-cold water and dried. If a solid product is not formed, the mixture is typically extracted with ethyl acetate, dried over anhydrous sodium sulfate and concentrated to obtain the desired product.

### Step 2. Preparation of (Arylmethanesulfonyl)nitromethanes and (Arylsulfonyl)nitromethanes

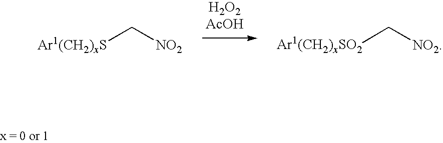

30% Hydrogen peroxide (10 mL) is slowly added to an ice-cold solution of a (arylmethanethio)nitromethane or (arylthio)nitromethane (5.0 g) in glacial acetic acid (35 mL) in a 100 mL flask. The reaction mixture is kept at room temperature for 18 hours, then the reaction mixture is poured onto crushed ice. If a solid product is formed, it is typically collected by filtration, washed with ice-cold water and dried. If a solid product is not formed, the mixture is typically extracted with ethyl acetate, dried over anhydrous sodium sulfate and concentrated to obtain the desired product.

### Step 3. Synthesis of 1-Aryl-2-(arylmethanesulfonyl)-2-nitroethenes and 1-Aryl-2-(arylsulfonyl)-2-nitroethenes

Two methods for the synthesis of 1-aryl-2-(arylmethanesulfonyl)-2-nitroethenes and 1-aryl-2-(arylsulfonyl)-2-nitroethenes by the condensation of (arylmethanesulfonyl)nitromethane or (arylsulfonyl)nitromethane with arenecarboxaldehydes are described below.

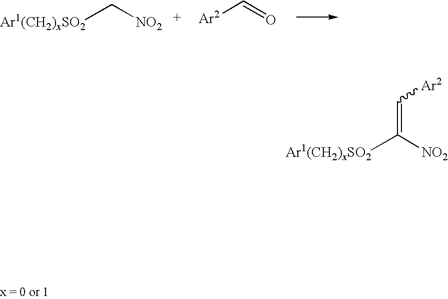

### Method A:

A mixture of (arylmethanesulfonyl)nitromethane or (arylsulfonyl)nitromethane (10 mmol), an arenecarboxaldehyde (10 mmol), glacial acetic acid (15 mL) and a catalytic amount of benzylamine (200 μL) is heated under reflux for about 2-8 hours. When the reaction is complete, the reaction mixture is allowed to cool, any solid product that forms is separated by filtration and dried. If a solid product does not precipitate, the mixture is typically diluted with ether and successively washed with a saturated solution of sodium bisulfite (2×15 mL), dilute hydrochloric acid (2×20 mL) and finally with water, dried and the solvent is removed under vacuum to yield a crude product. The final product is typically purified by recrystallization of the crude product from 2-propanol.

### Method B:

General procedure: A mixture of (arylmethanesulfonyl)nitromethane or (arylsulfonyl)nitromethane (1.0 eq.), arenecarboxaldehyde (1.1 eq.), benzoic acid (0.15 eq.) and piperidine (0.13 eq.) in toluene is heated under reflux for about 2-3 hours with continuous removal of water using a Dean-Stark water separator. When the reaction is complete, the reaction mixture is allowed to cool to room temperature, water is added and the product is extracted, typically with ethyl acetate. The organic layer is washed with saturated sodium bicarbonate solution, dilute hydrochloric acid, and water and dried. The solvent is removed under a vacuum to yield a crude product which is typically purified by recrystallization from 2-propanol to obtain the desired product.

### Synthesis Example 3

### Synthesis of 1-Cyano-2-aryl-N-arylethenesulfonamides

### Step 1. Preparation of 2-Chlorosulfonylacetonitrile

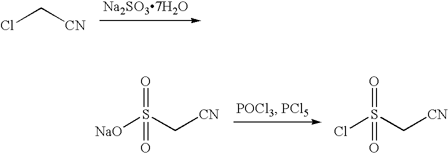

2-Chlorosulfonylacetonitrile is prepared following the procedure of Sammes (as described in patent GB1252903). Chloroacetonitrile (7.65 g) is added to a suspension of sodium sulfite heptahydrate (25 g) in water (200 ml) and the mixture is stirred vigorously at room temperature until a clear homogeneous solution is obtained. The solvent is removed under vacuum and the salt is dried in an oven at 80° C.

The crude sodium salt (19 g) is suspended in phosphorus oxychloride (35 ml) and finely powdered phosphorus pentachloride (21 g) is added. The mixture is heated on a water bath at 70° C. with vigorous stirring for 3 hours with exclusion of moisture. The mixture is allowed to cool to room temperature, then filtered to remove precipitated sodium chloride. Excess phosphorus oxychloride is removed under vacuum. The residual oil is distilled under high vacuum collecting the fraction boiling at 78-82° C. at 0.15 mm Hg to yield pure 2-chlorosulfonylacetonitrile.

### Step 2. Preparation of 1-cyano-N-arylmethanesulfonamide

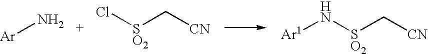

A solution of an aromatic amine (20 mmol) and triethylamine (20 mmol) in dichloromethane (100 ml) is kept at 10° C. for 15 minutes. A solution of 2-chlorosulfonylacetonitrile (20 mmol) in dichloromethane (30 ml) is added dropwise to the cooled solution of the amines. After the addition is complete, the reaction mixture is stirred at room temperature for 3 hours. After the reaction is complete (as indicated by TLC), water is added to the reaction mixture which is stirred for 15 minutes. The reaction mixture is typically partitioned between aqueous sodium bicarbonate, and dichloromethane, the organic layer is separated, dried, concentrated and purified by chromatography on silica gel to obtain a 1-cyano-N-arylmethanesulfonamide.

### Step 3. Synthesis of 1-Cyano-2-aryl-N-arylethenesulfoniamides

Two methods for the synthesis of 1-cyano-2-aryl-N-arylethenesulfoniamides by the condensation of 1-cyano-N-arylmethanesulfonamides with arenecarboxaldehydes are described below.

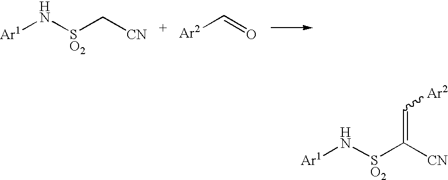

### Method A:

A mixture of a 1-cyano-N-arylmethanesulfonamide (10 mmol), an arenecarboxaldehyde (10 mmol), glacial acetic acid (15 mL) and a catalytic amount of benzylamine (200 μL) is heated under reflux for about 2-8 hours. When the reaction is complete, the reaction mixture is allowed to cool, any solid product that forms is separated by filtration and dried. If a solid product does not precipitate, the mixture is typically diluted with ether and successively washed with a saturated solution of sodium bisulfite (2×15 mL), dilute hydrochloric acid (2×20 mL) and finally with water, dried and the solvent is removed under vacuum to yield a crude product. The final product is typically purified by recrystallization of the crude product from 2-propanol.

### Method B:

A mixture 1 of a 1-cyano-N-arylmethanesulfonamide (1.0 eq.), an arenecarboxaldehyde (1.1 eq.), benzoic acid (0.15 eq.) and piperidine (0.13 eq.) in toluene is heated under reflux for about 2-3 hours with continuous removal of water using a Dean-Stark water separator. When the reaction is complete, the reaction mixture is allowed to cool to room temperature, water is added and the product is extracted, typically with ethyl acetate. The organic layer is washed with saturated sodium bicarbonate solution, dilute hydrochloric acid, water and dried. The solvent is removed under a vacuum to yield a crude product which is typically purified by recrystallization from 2-propanol to obtain the desired product.

### Synthesis Example 4

### Synthesis of 3-Aryl-2-arylmethanesulfonyl-acrylamides and 3-Aryl-2-arylsulfonyl-acrylamides

### Step 1. Preparation of 2-(Arylmethanesulfonyl)acetamides and 2-(Arylsulfonyl)acetamides

### Method A

Method A is a two-step process where an arylmethyl mercaptan or aryl mercaptan is reacted with chloroacetamide to produce a 2-(arylmethanethio)acetamide which is then oxidized to form a 2-(arylmethanesulfonyl)acetamide.

### Step A1. Preparation of 2-(Arylmethanethio)acetamides and 2-(Arylthio)acetamides

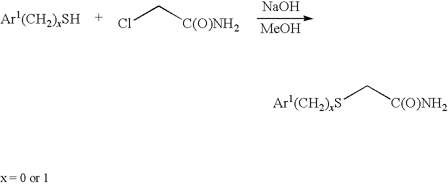

An arylmethyl mercaptan or aryl mercaptan (5 mmol) is added slowly through the dropping funnel to a stirred solution of sodium hydroxide (5 mmol) in methanol (50 mL) in a 100 mL two-necked round-bottomed flask equipped with a reflux condenser. A vigorous reaction occurs immediately. On completion of the addition, and when the reaction is no longer exothermic, 2-chloroacetamide (5 mmol) is added in portions. The cooled reaction mixture is stirred at room temperature for 3 hours then poured onto crushed ice. If a solid product is formed, it is typically collected by filtration, washed with ice-cold water and dried. If a solid product is not formed, the mixture is typically extracted with ethyl acetate, dried over anhydrous sodium sulfate and concentrated to obtain the 2-(arylmethanethio)acetamide or 2-(arylthio)acetamide.

### Step A2. Preparation of 2-(Arylmethanesulfonyl)acetamides and 2-(Arylsulfonyl)acetamides

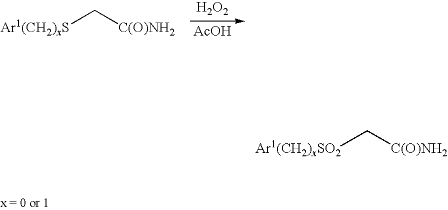

30% Hydrogen peroxide (10 mL) is added slowly to an ice-cold solution of a 2-(arylmethanethio)acetamide or 2-(arylthio)acetamide (5.0 g) in glacial acetic acid (35 mL). The reaction mixture is kept at room temperature for 18 hours, and then is poured onto crushed ice. The product typically precipitates as a solid and is collected by filtration, washed with water and dried to yield the desired product. If a solid product is not formed, the mixture is extracted, typically with ethyl acetate, dried over anhydrous sodium sulfate and concentrated to obtain the 2-(arylmethanethio)acetamide or 2-(arylthio)acetamide.

### Method B:

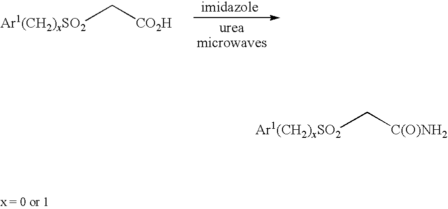

A mixture of a 2-(arylmethanesulfonyl)acetic acid or 2-(arylsulfonyl)acetic acid (10 mmol), urea (20 mmol) and imidazole (10 mmol) is ground with a mortar and pestle. The ground mixture is transferred into a glass Petri dish and exposed to microwave irradiation in a domestic microwave oven (300 W) for 3 to 5 minutes. The resulting crude product is extracted, typically with ethyl acetate, and purified by column chromatography to yield the desired product.

### Step 2. Synthesis of 3-aryl-2-(arylmethanesulfonyl)acrylamides and 3-aryl-2-(arylsulfonyl)acrylamides

Two methods for the synthesis of 3-aryl-2-(arylmethanesulfonyl)acrylamides and 3-aryl-2-(arylsulfonyl)acrylamides are described below.

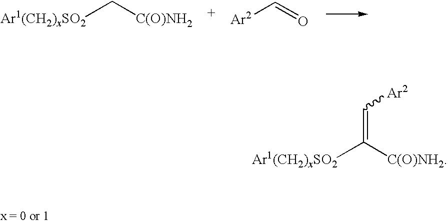

### Method A:

A mixture of a 2-(arylmethanesulfonyl)acetamide or 2-(arylsulfonyl)acetamide (10 mmol), an arenecarboxaldehyde (10 mmol), glacial acetic acid (15 mL) and a catalytic amount of benzylamine (200 μL) is heated under reflux for about 2-8 hours. When the reaction is complete, the reaction mixture is allowed to cool, any solid product that forms is separated by filtration and dried. If a solid product does not precipitate, the mixture is typically diluted with ether and successively washed with a saturated solution of sodium bisulfite (2×15 mL), dilute hydrochloric acid (2×20 mL) and finally with water, dried and the solvent is removed under vacuum to yield a crude product. The final product is typically purified by recrystallization of the crude product from 2-propanol.

### Method B:

A mixture of the 2-(arylmethanesulfonyl)acetamide or 2-(arylsulfonyl)acetamide (1.0 eq.), arenecarboxaldehyde (1.1 eq.), benzoic acid (0.15 eq.) and piperidine (0.13 eq.) in toluene is heated under reflux for about 2-3 hours with continuous removal of water using a Dean-Stark water separator. When the reaction is complete, the reaction mixture is allowed to cool to room temperature, water is added and the product is extracted, typically with ethyl acetate. The organic layer is washed with saturated sodium bicarbonate solution, dilute hydrochloric acid, water and dried. The solvent is removed under a vacuum to yield a crude product which is typically purified by recrystallization from 2-propanol to obtain the desired product.

### Synthetic Example 5

### Synthesis of 3-aryl-2-(N-arylsulfamoyl)acrylamides

### Step 1. Preparation of 2-(N-arylsulfamoyl)acetamide

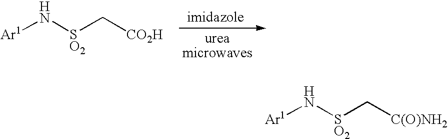

A mixture of the 2-(N-arylsulfamoyl)acetic acid (10 mmol), urea (20 mmol) and imidazole (10 mmol) is ground with a mortar and pestle. The ground mixture is transferred into a glass Petri dish and exposed to microwave irradiation in a domestic microwave oven (300 W) for 3 to 5 minutes. The resulting crude product is extracted, typically with ethyl acetate, and purified by column chromatography to yield the desired product.

### Step 2. Synthesis of 3-aryl-2-(N-arylsulfamoyl)acrylamides

Two methods for the synthesis of 3-aryl-2-(N-arylsulfamoyl)acrylamides are described below.

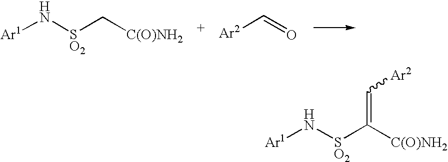

### Method A:

A mixture of a 2-(arylmethanesulfonyl)acetamide (10 mmol), an arenecarboxaldehyde (10 mmol), glacial acetic acid (15 mL) and a catalytic amount of benzylamine (200 μL) is heated under reflux for about 2-8 hours. When the reaction is complete, the reaction mixture is allowed to cool, any solid product that forms is separated by filtration and dried. If a solid product does not precipitate, the mixture is typically diluted with ether and successively washed with a saturated solution of sodium bisulfite (2×15 mL), dilute hydrochloric acid (2×20 mL) and finally with water, dried and the solvent is removed under vacuum to yield a crude product. The final product is typically purified by recrystallization of the crude product from 2-propanol.

### Method B:

General procedure: A mixture of a 2-(arylmethanesulfonyl)acetamide (1.0 eq.), an arenecarboxaldehyde (1.1 eq.), benzoic acid (0.15 eq.) and piperidine (0.13 eq.) in toluene is heated under reflux for about 2-3 hours with continuous removal of water using a Dean-Stark water separator. When the reaction is complete, the reaction mixture is allowed to cool to room temperature, water is added and the product is extracted, typically with ethyl acetate. The organic layer is washed with saturated sodium bicarbonate solution, dilute hydrochloric acid, and water and dried. The solvent is removed under a vacuum to yield a crude product which is typically purified by recrystallization from 2-propanol to obtain the desired product.

### Synthetic Example 6

### Synthesis of 3-aryl-2-(arylmethanethio)acrylonitriles

Two methods for the synthesis of 3-aryl-2-(arylmethanethio)acrylonitriles are described below.

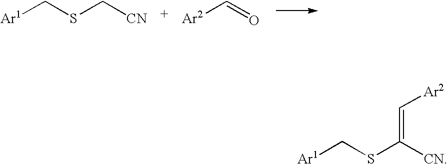

### Method A:

General Procedure: A mixture of the 2-(arylmethanethio)acetonitrile, arenecarboxaldehyde (10 mmol), glacial acetic acid (15 mL) and a catalytic amount of benzylamine (200 μL) is heated under reflux for about 2-8 hours. When the reaction is complete, the mixture is diluted with ether and successively washed with a saturated solution of sodium bisulfite, dilute hydrochloric acid and finally with water, dried and the solvent is removed under vacuum to yield a crude product. The final product is typically purified by column chromatography.

### Method B:

General procedure: A mixture of 2-(arylmethanethio)acetonitrile (1.0 eq.), arenecarboxaldehyde (1.1 eq.), benzoic acid (0.15 eq.) and piperidine (0.13 eq.) in toluene is heated under reflux for about 2-3 hours with continuous removal of water using a Dean-Stark water separator. When the reaction is complete, the mixture is diluted with ether and successively washed with a saturated solution of sodium bisulfite, dilute hydrochloric acid and finally with water, dried and the solvent is removed under vacuum to yield a crude product. The final product is typically purified by column chromatography.

### Synthetic Example 7

### Synthesis of 1-Aryl-2-(arylmethanethio)-2-nitroethenes and 1-Aryl-2-(arylthio)-2-nitroethenes

Two methods for the synthesis of 1-aryl-2-(arylmethanethio)-2-nitroethenes and 1-aryl-2-(arylthio)-2-nitroethenes are described below.

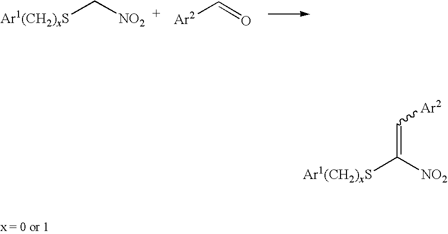

### Method A:

A mixture of an (arylmethanethio)nitromethane or a (arylthio)nitromethane (10 mmol), an arenecarboxaldehyde (10 mmol), glacial acetic acid (15 mL) and a catalytic amount of benzylamine (200 μL) is heated under reflux for about 2-8 hours. When the reaction is complete, the mixture is diluted, typically with ether, and successively washed with a saturated solution of sodium bisulfite, dilute hydrochloric acid and finally with water, dried and the solvent is removed under vacuum to yield a crude product. The final product is typically purified by column chromatography.

### Method B:

A mixture of an (arylmethanethio)nitromethane or an (arylthio)nitromethane (1.0 eq.), an arenecarboxaldehyde (1.1 eq.), benzoic acid (0.15 eq.) and piperidine (0.13 eq.) in toluene is heated under reflux for about 2-3 hours with continuous removal of water using a Dean-Stark water separator. When the reaction is complete, the mixture is diluted, typically with ether and successively washed with a saturated solution of sodium bisulfite, dilute hydrochloric acid and finally with water, dried and the solvent is removed under vacuum to yield a crude product. The final product is typically purified by column chromatography.

### Synthetic Example 8

### Synthesis of 3-aryl-2-(arylmethanethio)acrylamides and 3-aryl-2-(arylthio)acrylamides

Two methods for the synthesis of 3-aryl-2-(arylmethanethio)acrylamides and 3-aryl-2-(arylthio)acrylamides are described below.

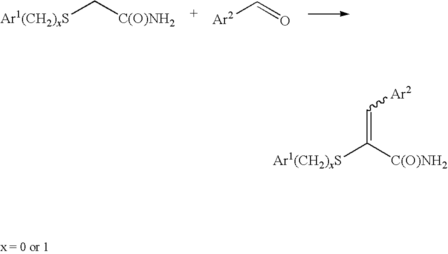

### Method A:

A mixture of a 2-(arylmethanethio)acetonitrile, an arenecarboxaldehyde (10 mmol), glacial acetic acid (15 mL) and a catalytic amount of benzylamine (200 μL) is heated under reflux for about 2-8 hours. When the reaction is complete, the mixture is diluted, typically with ether, and successively washed with a saturated solution of sodium bisulfite, dilute hydrochloric acid and finally with water, dried and the solvent is removed under vacuum to yield a crude product. The final product is typically purified by column chromatography.

### Method B:

A mixture of 2-(arylmethanethio)acetamide (1.0 eq.), an arenecarboxaldehyde (1.1 eq.), benzoic acid (0.15 eq.) and piperidine (0.13 eq.) in toluene is heated under reflux for about 2-3 hours with continuous removal of water using a Dean-Stark water separator. When the reaction is complete, the mixture is diluted, typically with ether, and successively washed with a saturated solution of sodium bisulfite, dilute hydrochloric acid and finally with water, dried and the solvent is removed under vacuum to yield a crude product. The final product is typically purified by column chromatography on silica.

### Synthetic Example 9

### Synthesis of 3-aryl-2-(arylmethanesulfinyl)acrylonitriles

### Step 1. Preparation of 2-(arylmethanethio)acetonitriles

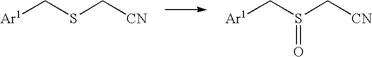

m-Chloroperbenzoic acid (1.0 eq.) is added slowly to a solution of the 2-(arylmethanethio)acetonitrile (1.0 eq.) in dichloromethane which is stirred at 0° C. Stirring is continued at 0° C. for 1 hour. The solution is diluted with dichloromethane and washed with aqueous sodium carbonate. The organic layer is dried (MgSO₄) and concentrated in vacuo to yield the crude sulfoxide, which is typically purified by column chromatography on silica.

### Step 2. Preparation of 3-aryl-2-(arylmethanesulfinyl)acrylonitriles

Two methods for the synthesis of 3-aryl-2-(arylmethanesulfinyl)acrylonitriles are described below.

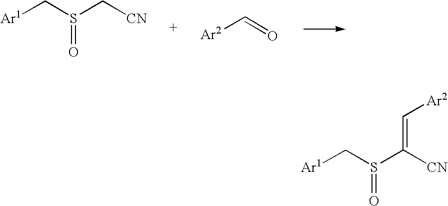

### Method A:

A mixture of a 2-(arylmethanesulfinyl)acetonitrile (10 mmol), an arenecarboxaldehyde (10 mmol), glacial acetic acid (15 mL) and a catalytic amount of benzylamine (200 μL) is heated under reflux for about 2-8 hours. When the reaction is complete, the mixture is diluted, typically with ether, and successively washed with a saturated solution of sodium bisulfite, dilute hydrochloric acid and finally with water, dried and the solvent is removed under vacuum to yield a crude product. The final product is typically purified by column chromatography on silica.

### Method B:

A mixture of a 2-(arylmethanesulfinyl)acetonitrile (1.0 eq.), an arenecarboxaldehyde (1.1 eq.), benzoic acid (0.15 eq.) and piperidine (0.13 eq.) in toluene is heated under reflux for about 2-3 hours with continuous removal of water using a Dean-Stark water separator. When the reaction is complete, the mixture is diluted, typically with ether, and successively washed with a saturated solution of sodium bisulfite, dilute hydrochloric acid and finally with water, dried and the solvent is removed under vacuum to yield a crude product. The final product is typically purified by column chromatography on silica.

### Synthetic Example 10

### Synthesis of 1-Aryl-2-(arylmethanesulfinyl)-2-nitroethenes and 1-Aryl-2-(arylsulfinyl)-2-nitroethenes

### Step 1. Preparation of (Arylmethanesulfinyl)nitromethenes and (Arylsulfinyl)nitromethanes

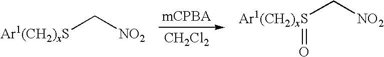

m-Chloroperbenzoic acid (1.0 eq.) is added slowly to a solution of an (arylmethanethio)nitromethane or an (arylthio)nitromethane (1.0 eq.) in dichloromethane which is stirred at 0° C. Stirring is continued at 0° C. for 1 hour. The solution is diluted with dichloromethane and washed with aqueous sodium carbonate. The organic layer is dried (MgSO₄) and concentrated in vacuo to yield the crude sulfoxide, which is typically purified by column chromatography on silica.

### Step 2. Preparation of 1-Aryl-2-(arylmethanesulfinyl)-2-nitroethenes and 1-Aryl-2-(arylsulfinyl)-2-nitroethenes

Two methods for the synthesis of 1-aryl-2-(arylmethanesulfinyl)-2-nitroethenes and 1-aryl-2-(arylsulfinyl)-2-nitroethenes are described below.

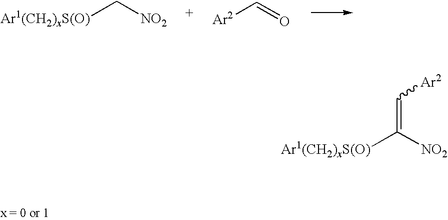

### Method A:

A mixture of an (arylmethanesulfinyl)nitromethane or an (arylsulfinyl)nitromethane (10 mmol), an arenecarboxaldehyde (10 mmol), glacial acetic acid (15 mL) and a catalytic amount of benzylamine (200 μL) is heated under reflux for about 2-8 hours. When the reaction is complete, the mixture is diluted, typically with ether, and successively washed with a saturated solution of sodium bisulfite, dilute hydrochloric acid and finally with water, dried and the solvent is removed under vacuum to yield a crude product. The final product is typically purified by column chromatography.

### Method B:

A mixture of an (arylmethanesulfinyl)nitromethane or an (arylsulfinyl)nitromethane (1.0 eq.), an arenecarboxaldehyde (1.1 eq.), benzoic acid (0.15 eq.) and piperidine (0.13 eq.) in toluene is heated under reflux for about 2-3 hours with continuous removal of water using a Dean-Stark water separator. When the reaction is complete, the mixture is diluted, typically with ether, and successively washed with a saturated solution of sodium bisulfite, dilute hydrochloric acid and finally with water, dried and the solvent is removed under vacuum to yield a crude product. The final product is typically purified by column chromatography on silica.

### Synthetic Example 11

### Synthesis of 3-aryl-2-(arylmethanesulfinyl)acrylamides and 3-aryl-2-(arylsulfinyl)acrylamides

### Step 1. Preparation of 2-(arylmethanethio)acrylamide or 2-(arylthio)acrylamide

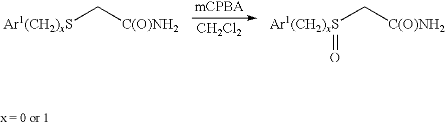

m-Chloroperbenzoic acid (1.0 eq.) is added slowly to a solution of a 2-(arylmethanethio)acetonitrile (1.0 eq.) in dichloromethane which is stirred at 0° C. Stirring is continued at 0° C. for 1 hour. The solution is diluted with dichloromethane and washed with aqueous sodium carbonate. The organic layer is dried (MgSO₄) and concentrated in vacuo to give the crude sulfoxide, which is typically purified by column chromatography on silica.

### Step 2. Preparation of 3-aryl-2-(arylmethanesulfinyl)acrylamide or 3-aryl-2-(arylsulfinyl)acrylamide

Two methods for the synthesis of 3-aryl-2-(arylmethanethio)acrylamides or 3-aryl-2-(arylthio)acrylamides are described below.

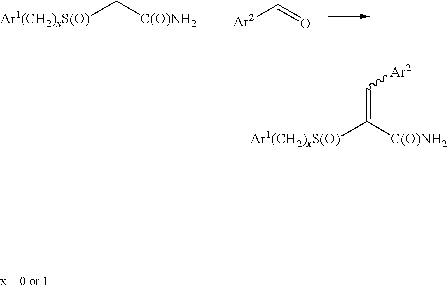

### Method A:

A mixture of a 2-(arylmethanethio)acetamide or 2-(arylthio)acetamide (10 mmol), an arenecarboxaldehyde (10 mmol), glacial acetic acid (15 mL) and a catalytic amount of benzylamine (200 μL) is heated under reflux for about 2-8 hours. When the reaction is complete, the mixture is diluted, typically with ether, and successively washed with a saturated solution of sodium bisulfite, dilute hydrochloric acid and finally with water, dried and the solvent is removed under vacuum to yield a crude product. The final product is typically purified by column chromatography.

### Method B:

General procedure: A mixture of a 2-(arylmethanethio)acetamide or 2-(arylthio)acetamide (1.0 eq.), an arenecarboxaldehyde (1.1 eq.), benzoic acid (0.15 eq.) and piperidine (0.13 eq.) in toluene is heated under reflux for about 2-3 hours with continuous removal of water using a Dean-Stark water separator. When the reaction is complete, the mixture is diluted, typically with ether, and successively washed with a saturated solution of sodium bisulfite, dilute hydrochloric acid and finally with water, dried and the solvent is removed under vacuum to yield a crude product. The final product is typically purified by column chromatography.

### COMPOUND EXAMPLES

The representative compounds listed in Tables 1-6 are shown by way of illustration, and are not intended to limit the scope of the invention. The compounds are prepared by the methods described above.

TABLE 1
Compound Examples

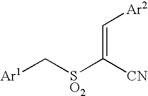

| | | | m.p. | Yield |
|---|---|---|---|---|
| Example | Ar¹ | Ar² | (° C.) | (%) |
| 1 | 4-methoxyphenyl | 2,4,6-trimethoxyphenyl | 178-180 | 66 |
| 2 | 4-methoxyphenyl | 2,4-difluorophenyl | 38-140 | 52 |
| 3 | 4-methoxyphenyl | 3-nitro-4-fluorophenyl | 98-200 | 62 |
| 4 | 4-bromophenyl | 2,4-difluorophenyl | 146-148 | 58 |
| 5 | 4-methoxyphenyl | 4-aminophenyl | 140-142 | 51 |
| 6 | 4-methoxyphenyl | 3-amino-4-fluorophenyl | 146-148 | 54 |
| 7 | 4-methoxyphenyl | 4-fluorophenyl | 171-173 | 59 |
| 8 | 4-methoxyphenyl | 2,6-dimethoxyphenyl | 108-110 | 62 |
| 9 | 4-methoxyphenyl | 2-methoxyphenyl | 143-145 | 59 |
| 10 | 4-methoxyphenyl | 4-nitrophenyl | 174-176 | 63 |
| 11 | 4-methoxyphenyl | 2,3,4,5,6-pentafluorophenyl | 110-112 | 50 |
| 12 | 4-methoxyphenyl | 3-furyl | 150-153 | 56 |
| 13 | 4-methoxyphenyl | thiophen-3-yl | 142-144 | 58 |
| 14 | 4-bromophenyl | 2-(difluoromethoxy)phenyl | 204-206 | 60 |
| 15 | 4-methoxyphenyl | 2-chloro-4-fluorophenyl | 138-140 | 62 |
| 16 | 4-bromophenyl | 2-fluoro-4-cyanophenyl | 202-204 | 58 |
| 17 | 4-methoxyphenyl | 2-fluoro-4-cyanophenyl | 112-114 | 59 |
| 18 | 4-bromophenyl | 2,6-dimethoxyphenyl | 156-158 | 58 |
| 19 | 4-bromophenyl | 2,3,4,5,6-pentafluorophenyl | 146-148 | 52 |
| 20 | 4-bromophenyl | 2,4,6-trimethoxyphenyl | 158-160 | 58 |
| 21 | 4-chlorophenyl | 2,3,4,5,6-pentafluorophenyl | 154-156 | 51 |
| 22 | 4-methoxyphenyl | 2-benzoxazolon-5-yl | 210-212 | 53 |
| 23 | 4-methoxyphenyl | 2-benzoxazolon-6-y1 | 208-210 | 56 |
| 24 | 4-chlorophenyl | 3,4-dihydroxyphenyl | 178-180 | 52 |
| 25 | 4-methoxyphenyl | 3,4-dihydroxyphenyl | 144-146 | 58 |
| 26 | 4-methoxyphenyl | 3,4-dimethoxyphenyl | 156-158 | 57 |
| 27 | 4-metboxyphenyl | 1,3-benzodioxole-5-yl | 186-188 | 61 |
| 28 | 3,4-dimethoxyphenyl | 3,4-dihydroxyphenyl | 196-198 | 53 |
| 29 | 3,4-dimethoxyphenyl | 2-benzoxazolon-6-yl | ND | 56 |
| 30 | 3,4-dimethoxyphenyl | 2-benzoxazolon-5-yl | 174-176 | 61 |
| 31 | 4-bromophenyl | 4-nitrophenyl | 138-142 | 59 |
| 32 | 3,4-dimethoxyphenyl | 2,3,4,5,6-pentafluorophenyl | LIQUID | 63 |
| 33 | 4-fluorophenyl | 2,3,4,5,6-pentafluorophenyl | 156-158 | 52 |
| 34 | 3-nitro-4-methoxyphenyl | 2,4,6-trimethoxyphenyl | 184-186 | 56 |
| 35 | 3-nitro-4-methoxyphenyl | 2,3,4,5,6-pentafluorophenyl | 144-146 | 58 |
| 36 | 3-amino-4-methoxyphenyl | 2,4,6-trimethoxyphenyl | 198-200 | 52 |
| 37 | 4-fluorophenyl | 4-ethoxycarbonyl-3,5- | ND | — |
| | | dimethylpyrrol-2-yl | | |
| 38 | 4-methoxy-3-(4- | 2,3,4,5,6-pentafluorophenyl | 170-172 | — |
| | methylphenylsulfonyloxy)phenyl | | | |
| 39 | 3-fluoro-4-methoxyphenyl | 2,4,6-trimethoxyphenyl | 162-164 | — |
| 40 | 4-chlorophenyl | 2,5-dimethylphenyl | 123-126 | — |
| 41 | 4-chlorophenyl | 2-chloro-4-fluorophenyl | 124-126 | — |
| 42 | 4-chlorophenyl | 4-fluoro-3-methylphenyl | 140-145 | — |
| 43 | 4-chlorophenyl | 4-methanesulfenyl | 178-190 | — |
| 44 | 4-chlorophenyl | 3-hydroxy-4-methoxyphenyl | 155-165 | — |
| 45 | 4-chlorophenyl | 4-bromophenyl | 168-180 | — |
| 46 | 4-chlorophenyl | 2,3,4-trimethoxyphenyl | 120-128 | — |
| 47 | 4-chlorophenyl | 4-fluorophenyl | 145-150 | — |
| 48 | 4-chlorophenyl | 2-fluoro-4-nitrophenyl | 243-253 | — |
| 49 | 4-chlorophenyl | 2-fluoro-4-methoxyphenyl | 145-155 | — |
| 50 | 4-chlorophenyl | 4-methoxyphenyl | 143-152 | — |
| 51 | 4-chlorophenyl | 2,4-dichlorophenyl | 121-126 | — |
| 52 | 4-chlorophenyl | 3,4-dichlorophenyl | 145-155 | — |
| 53 | 4-chlorophenyl | 2,4,6-trimethoxyphenyl | 177-180 | — |
| 54 | 4-chlorophenyl | 3-ethoxy-4-hydroxyphenyl | 155-169 | — |
| 55 | 4-chlorophenyl | 4-ethoxy-3-methoxyphenyl | 143-148 | — |
| 56 | 4-chlorophenyl | 3,5-dimethoxyphenyl | 155-165 | — |
| 57 | 4-chlorophenyl | 4-acetoxyphenyl | 192-202 | — |
| 58 | 4-chlorophenyl | 4-(N,N-dimethylamino)phenyl | 208-206 | — |
| 59 | 4-chlorophenyl | 4-hydroxy-3-nitrophenyl | 195-205 | — |
| 60 | 4-chlorophenyl | 2,4-dihydroxyphenyl | 250-255 | — |
| 61 | 4-chlorophenyl | 4-chlorophenyl | 145-168 | — |
| 62 | 4-chlorophenyl | 2,3,4-trichlorophenyl | 145-155 | — |
| 63 | 2,4-dichlorophenyl | 5-methylthiophen-2-yl | 118-124 | — |
| 64 | 4-fluorophenyl | 4-(N,N-dimethylamino)phenyl | 135-145 | — |
| 65 | 2,4-dichlorophenyl | 2-fluoro-4-methoxyphenyl | 140-155 | — |
| 66 | 4-chlorophenyl | 3,4-dimethylphenyl | 191-202 | — |
| 67 | 4-chlorophenyl | 4-biphenyl-1-yl | 208-220 | — |
| 68 | 4-chlorophenyl | 3-indolyl | 200-205 | — |
| 69 | 4-chlorophenyl | phenyl | 142-157 | — |
| 70 | 4-chlorophenyl | 2-benzyloxyphenyl | 129-137 | — |
| 71 | 2,4-dichlorophenyl | 5-bromo-3-indolyl | 264-275 | — |
| 72 | 4-chlorophenyl | 5-bromo-3-indolyl | 240-250 | — |
| 73 | 4-chlorophenyl | 5-chloro-3-indolyl | 172-182 | — |
| 74 | 4-chlorophenyl | 3-ethoxy-4-hydroxyphenyl | 150-165 | — |
| 75 | 2,4-dichlorophenyl | 5-chloro-3-indolyl | 282-292 | — |
| 76 | 2,4-dichlorophenyl | 4-methanesulfenyl | 133-145 | — |
| 77 | 4-fluorophenyl | 5-chloro-3-indolyl | 250-265 | — |
| 78 | 4-fluorophenyl | 5-bromo-3-indolyl | 260-270 | — |
| 79 | 4-fluorophenyl | 2,4,6-trimethoxyphenyl | 182-192 | — |
| 80 | 4-fluorophenyl | 3-hydroxy-4-methoxyphenyl | 193-205 | — |
| 81 | 4-fluorophenyl | 3-indolyl | 140-152 | — |
| 82 | 4-fluorophenyl | 4-bromophenyl | 155-168 | — |
| 83 | 4-fluorophenyl | 2,4-dihydroxyphenyl | 225-234 | — |
| 84 | 4-fluorophenyl | 4-acetoxyphenyl | 116-125 | — |
| 85 | 4-fluorophenyl | 2,4-dichlorophenyl | 153-163 | — |
| 86 | 4-fluorophenyl | 3,5-dimethoxyphenyl | 120-123 | — |
| 87 | 4-fluorophenyl | 4-methanesulfenyl | 160-164 | — |
| 88 | 4-fluorophenyl | 3,4-dimethylphenyl | 122-132 | — |
| 89 | 4-fluorophenyl | 4-chlorophenyl | 150-155 | — |
| 90 | 4-fluorophenyl | 2,5-dimethylphenyl | 122-130 | — |
| 91 | 2,4-dichlorophenyl | 5-chloro-3-indolyl | 150-161 | — |
| 92 | 2,4-dichlorophenyl | 2,4-dihydroxyphenyl | 220-230 | — |
| 93 | 2,4-dichlorophenyl | 2,3,4-trimethoxyphenyl | 150-165 | — |
| 94 | 2,4-dichlorophenyl | 4-ethoxy-3-methoxyphenyl | 126-131 | — |
| 95 | 2,4-dichlorophenyl | 2-chloro-4-fluorophenyl | 142-148 | — |
| 96 | 2,4-dichlorophenyl | 2,5-dimethylphenyl | 142-149 | — |
| 97 | 2,4-dichlorophenyl | 3,4-dimethylphenyl | 126-142 | — |
| 98 | 2,4-dichlorophenyl | 4-bromophenyl | 126-138 | — |
| 99 | 2,4-dichlorophenyl | 4-methoxyphenyl | 114-127 | — |
| 100 | 2,4-dichlorophenyl | 4-chlorophenyl | 119-125 | — |
| 101 | 4-bromophenyl | 3,5-dimethylphenyl | 169-172 | — |
| 102 | 2,4-dichlorophenyl | 3-methyl-2-thiophen-2-yl | 134-138 | — |
| 103 | 2,4-dichlorophenyl | 3-methyl-2-furyl | 184-189 | — |
| 104 | 2,4-dichlorophenyl | 3-furyl | 116-122 | — |
| 105 | 2,4-dichlorophenyl | 3,4,5-trimethoxyphenyl | 147-152 | — |
| 106 | 2,4-dichlorophenyl | 2,5-dimethoxyphenyl | 132-137 | — |
| 107 | 2,4-dichlorophenyl | 2,4,5-trimethoxyphenyl | 164-170 | — |
| 108 | 2,4-dichlorophenyl | 2-hydroxyphenyl | 219-223 | — |
| 109 | 2,4-dichlorophenyl | 4-hydroxy-3-nitrophenyl | 169-171 | — |
| 110 | 2,4-dichlorophenyl | 3,4-dimethoxyphenyl | 149-153 | — |
| 111 | 2,4-dichlorophenyl | 2,4-dimethoxyphenyl | 165-170 | — |
| 112 | 2,4-dichlorophenyl | phenyl | 219-223 | — |
| 113 | 2,4-dichlorophenyl | 2-hydroxy-4-methoxyphenyl | 242-247 | — |
| 114 | 3-bromophenyl | 3-methyl-2-furyl | 122-126 | — |
| 115 | 3-bromophenyl | 3-furyl | 128-132 | — |
| 116 | 3-bromophenyl | 3,4-dihydroxyphenyl | 119-121 | — |
| 117 | 3-bromophenyl | 3-hydroxyphenyl | 160-165 | — |
| 118 | 4-bromophenyl | 5-methylthiophen-2-yl | 142-148 | — |
| 119 | 4-bromophenyl | 2,5-dimethylphenyl | 124-128 | — |
| 120 | 4-chlorophenyl | 3-methyl-2-thiophen-2-yl | 171-175 | — |
| 121 | 4-chlorophenyl | 2-hydroxyphenyl | 171-176 | — |
| 122 | 4-chlorophenyl | 3-furyl | 150-154 | — |
| 123 | 4-chlorophenyl | 5-methylthiophen-2-yl | 148-152 | — |
| 124 | phenyl | 3-hydroxyphenyl | 132-135 | — |
| 125 | phenyl | 2-hydroxyphenyl | 135-138 | — |
| 126 | phenyl | 2-methoxyphenyl | 169-172 | — |
| 127 | phenyl | 3-methyl-2-thiophen-2-yl | 152-156 | — |
| 128 | phenyl | 3-methyl-2-furyl | 118-121 | — |
| 129 | phenyl | 2-fluoro-4-methoxyphenyl | 161-166 | — |
| 130 | phenyl | 2-chlorophenyl | 155-160 | — |
| 131 | phenyl | 3,4,5-trimethoxyphenyl | 142-146 | — |
| 132 | phenyl | 2,4-dimethoxyphenyl | 125-130 | — |
| 133 | phenyl | 3,4-dimethoxyphenyl | 170-174 | — |
| 134 | phenyl | 3,5-dimethoxyphenyl | 152-156 | — |
| 135 | phenyl | 2,5-dimethoxyphenyl | 140-143 | — |
| 136 | phenyl | 2,5-dimethylphenyl | 126-129 | — |
| 137 | phenyl | 2-benzyloxyphenyl | 93-97 | — |
| 138 | phenyl | 4-biphenyl-1-yl | 139-142 | — |
| 139 | phenyl | 4-hydroxy-3-nitrophenyl | 159-162 | — |
| 140 | 4-methoxyphenyl | 4-biphenyl-1-yl | 188-192 | — |
| 141 | 4-methoxyphenyl | 4-methoxyphenyl | 163-167 | — |
| 142 | phenyl | 2,4-difluorophenyl | 133-135 | — |
| 143 | phenyl | 3,5-difluorophenyl | 148-150 | — |
| 144 | 4-bromophenyl | 2-chlorophenyl | 143-147 | — |
| 145 | 4-fluorophenyl | 2-chlorophenyl | 159-163 | — |
| 146 | 4-methoxyphenyl | 2,3,4-trichlorophenyl | 136-138 | — |
| 147 | 4-methoxyphenyl | 2,4-dimethoxyphenyl | 156-160 | — |
| 148 | 4-methoxyphenyl | 3,4-dimethoxyphenyl | 155-160 | — |
| 149 | 4-methoxyphenyl | 3,5-dimethoxyphenyl | 135-140 | — |

TABLE 2
Compound Examples

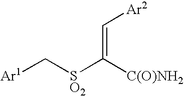

| Ex- | | | m.p. | Yield |
|---|---|---|---|---|
| ample | Ar¹ | Ar² | (° C.) | (%) |
| 150 | 2,4-dichlorophenyl | 4-chlorophenyl | 201-205 | 34 |
| 151 | 2,4-dichlorophenyl | 3,4-dimethylphenyl | 193-196 | 34 |
| 152 | 2,4-dichlorophenyl | 2,4,6-trimethoxyphenyl | 201-204 | 60 |
| 153 | 2,4-dichlorophenyl | 4-bromophenyl | 195-196 | 40 |
| 154 | 2,4-dichlorophenyl | 3-hydroxy-4- | 185-189 | 52 |
| | | methoxyphenyl | | |
| 155 | 4-fluorophenyl | 2,3-dichlorophenyl | 188-193 | 42 |
| 156 | 2-chlorophenyl | 2,4-dichlorophenyl | 158-164 | 48 |
| 157 | 4-chlorophenyl | 2,5-dimethylphenyl | 79-82 | 33 |
| 158 | 4-chlorophenyl | 4-ethoxy-3- | 195-197 | 55 |
| | | methoxyphenyl | | |
| 159 | 4-nitrophenyl | 4-ethoxy-3- | 235-240 | 80 |
| | | methoxyphenyl | | |
| 160 | 4-fluorophenyl | 2,5-dimethylphenyl | 172-176 | 52 |
| 161 | 4-fluorophenyl | 4-bromophenyl | 199-200 | 38 |
| 162 | 4-fluorophenyl | 2,4,5-trifluorophenyl | 168-170 | 71 |
| 163 | 4-chlorophenyl | 3,5-dimethylphenyl | 172-176 | 50 |
| 164 | 4-fluorophenyl | 4-ethoxy-3- | 197-202 | 64 |
| | | methoxyphenyl | | |
| 165 | 4-chlorophenyl | 2,4-dichlorophenyl | 206-210 | 28 |
| 166 | 4-chlorophenyl | 2-chloro-4-fluorophenyl | 194-199 | 20 |
| 167 | 2,4-dichlorophenyl | 4-acetylphenyl | 207-210 | 34 |
| 168 | 2,4-dichlorophenyl | 4-ethoxy-3- | 245-247 | 58 |
| | | methoxyphenyl | | |
| 169 | 4-nitrophenyl | 2,3-dichlorophenyl | 200-202 | 37 |
| 170 | 4-chlorophenyl | 4-flurophenyl | 176-180 | 62 |
| 171 | 4-methoxyphenyl | 2,4,6-trimethoxyphenyl | 168-171 | 71 |
| 172 | 4-methoxyphenyl | 4-fluorophenyl | 158-161 | 54 |
| 173 | 4-methoxyphenyl | 2,6-dimethoxyphenyl | 166-168 | 65 |
| 174 | 4-chlorophenyl | 2,4,6-trimethoxyphenyl | 176-178 | 59 |
| 175 | 4-chlorophenyl | 2,6-dimethoxyphenyl | 150-154 | 64 |
| 176 | 4-chlorophenyl | 4-nitrophenyl | 190-192 | 62 |

TABLE 3
Compound Examples

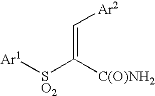

| | | | m.p. | Yield |
|---|---|---|---|---|
| Example | Ar¹ | Ar² | (° C.) | (%) |
| 177 | phenyl | 4-fluorophenyl | 151-154 | 58 |
| 178 | phenyl | 2,4,6-trimethoxyphenyl | 164-167 | 68 |
| 179 | phenyl | 2,6-dimethoxyphenyl | 168-172 | 64 |
| 180 | phenyl | 4-nitrophenyl | — | — |
| 181 | 4-methoxyphenyl | 4-fluorophenyl | 154-156 | 58 |
| 182 | 4-methoxyphenyl | 2,4,6-trimethoxyphenyl | 172-175 | 57 |
| 183 | 4-methoxyphenyl | 2,6-dimethoxyphenyl | 160-162 | 61 |
| 184 | 4-methoxyphenyl | 4-nitrophenyl | 172-174 | 62 |

TABLE 4
Compound Examples

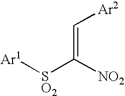

| | | | m.p. | Yield |
|---|---|---|---|---|
| Example | Ar¹ | Ar² | (° C.) | (%) |
| 185 | 4-methylphenyl | 2,4,6-trimethoxyphenyl | 192-194 | 62 |
| 186 | 4-bromophenyl | 2,4,6-trimethoxyphenyl | 184-186 | 68 |
| 187 | phenyl | 2,4,6-trimethoxyphenyl | 176-180 | 58 |

TABLE 5
Compound Examples

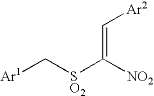

| | | | m.p. | Yield |
|---|---|---|---|---|
| Example | Ar¹ | Ar² | (° C.) | (%) |
| 188 | 4-methoxyphenyl | 2,4,6-trimethoxyphenyl | — | — |
| 189 | 4-fluorophenyl | 2,4,6-trimethoxyphenyl | — | — |

TABLE 6
Compound Examples

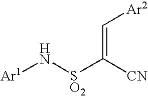

| | | | m.p. | Yield |
|---|---|---|---|---|
| Example | Ar¹ | Ar² | (° C.) | (%) |
| 190 | 4-methoxyphenyl | 4-bromophenyl | 106-107 | — |
| 191 | 4-methoxyphenyl | 2,4,6-trimethoxyphenyl | 134-136 | — |
| 192 | 4-methoxyphenyl | 2,3,4,5,6-pentafluorophenyl | 100-102 | — |
| 193 | 4-methoxyphenyl | 2,6-dimethoxyphenyl | 154-156 | — |
| 194 | 4-methoxy-3-(4- | 2,3,4,5,6-pentafluorophenyl | 112-114 | — |
| | methylphenylsulfonyloxy)phenyl | | | |
| 195 | 4-chlorophenyl | 4-methoxyphenyl | 94-96 | — |
| 196 | 2-phenoxyphenyl | 4-bromophenyl | 129-134 | — |
| 197 | 2-phenoxyphenyl | 3-indolyl | 210-212 | — |
| 198 | 2-phenoxyphenyl | 5-methylthiophen-2-yl | 141-143 | — |
| 199 | 2-phenoxyphenyl | 2-methoxyphenyl | 116-118 | — |
| 200 | 4-methoxyphenyl | 4-fluorophenyl | 107-109 | — |
| 201 | 4-methoxyphenyl | 3,5-dimethoxyphenyl | 167-168 | — |
| 202 | 4-methoxyphenyl | 2,5-dimethoxyphenyl | 156-158 | — |
| 203 | 4-methoxyphenyl | 3,4-dimethylphenyl | 152-154 | — |
| 204 | 4-methoxyphenyl | 2-methoxyphenyl | 133-135 | — |
| 205 | 4-chlorophenyl | 3-indolyl | 216-217 | — |
| 206 | 2-phenoxyphenyl | 4-(N,N-dimethylamino)phenyl | 158-160 | — |
| 207 | 2-phenoxyphenyl | 4-chlorophenyl | 136-138 | — |
| 208 | 2-phenoxyphenyl | 2,4,6-trimethoxyphenyl | 154-156 | — |
| 209 | 2-phenoxyphenyl | 3-nitro-4-hydroxyphenyl | 83-85 | — |
| 210 | 2-phenoxyphenyl | 2-phenoxyphenyl | 138-140 | — |
| 211 | 2-phenoxyphenyl | 4-ethoxy-3-methoxyphenyl | 142-144 | — |
| 212 | 4-bromophenyl | 4-chlorophenyl | 169-171 | — |
| 213 | 4-bromophenyl | 2-benzyloxyphenyl | 128-130 | — |
| 214 | 4-bromophenyl | 3-indolyl | 208-210 | — |
| 215 | 4-bromophenyl | 2,4,6-trimethoxyphenyl | 197-199 | — |
| 216 | 4-bromophenyl | 4-(N,N-dimethylamino)phenyl | 190-192 | — |
| 217 | 4-bromophenyl | 4-bromophenyl | 174-176 | — |
| 218 | 4-bromophenyl | 2,3,4-trimethoxyphenyl | 146-148 | — |
| 219 | 4-bromophenyl | 4-ethoxy-3-methoxyphenyl | 188-190 | — |
| 220 | 4-bromophenyl | 4-biphenyl-1-yl | 186-188 | — |
| 221 | 4-bromophenyl | 2,3,4,5,6-pentafluorophenyl | 174-176 | — |
| 222 | 4-bromophenyl | 5-bromo-3-indolyl | 242-253 | — |
| 223 | 4-bromophenyl | 3-nitro-4-hydroxyphenyl | >250 | — |
| 224 | 4-bromophenyl | 3-methylthiophen-2-yl | 176-179 | — |
| 225 | 4-bromophenyl | 2-methoxyphenyl | 187-189 | — |
| 226 | 4-bromophenyl | 2,4-difluorophenyl | 165-166 | — |
| 227 | 4-bromophenyl | 2-chlorophenyl | 192-194 | — |
| 228 | 4-bromophenyl | 2-hydroxyphenyl | 169-170 | — |
| 229 | 4-bromophenyl | 2,5-dimethylphenyl | 172-174 | — |
| 230 | 4-fluorophenyl | 2-benzyloxyphenyl | 127-129 | — |
| 231 | 4-fluorophenyl | 3-indolyl | 202-204 | — |
| 232 | 4-fluorophenyl | 2,4,6-trimethoxyphenyl | 169-171 | — |
| 233 | 4-fluorophenyl | 4-(N,N-dimethylamino)phenyl | 179-180 | — |
| 234 | 4-fluorophenyl | 2,3,4,5,6-pentafluorophenyl | 150-152 | — |
| 235 | 4-fluorophenyl | 2-chlorophenyl | 182-184 | — |
| 236 | 4-fluorophenyl | 3-methylthiophen-2-yl | 179-181 | — |
| 237 | 4-fluorophenyl | 2,4,5-trimethoxyphenyl | 184-186 | — |
| 238 | 4-fluorophenyl | 2,5-dimethylphenyl | 161-164 | — |
| 239 | 4-fluorophenyl | 4-biphenyl-1-yl | 159-162 | — |
| 240 | 4-fluorophenyl | 2,4-difluorophenyl | 135-139 | — |
| 241 | 4-fluorophenyl | 2-fluoro-4-methoxyphenyl | 142-144 | — |
| 242 | 4-fluorophenyl | 4-bromophenyl | 130-132 | — |
| 243 | 4-fluorophenyl | 2,3,5-trichlorophenyl | 184-186 | — |
| 244 | 4-fluorophenyl | 3,4-dimethylphenyl | 141-142 | — |
| 245 | 4-fluorophenyl | 3-chloro-4-fluorophenyl | 118-120 | — |
| 246 | 4-fluorophenyl | 5-methylthiophen-2-yl | 133-134 | — |
| 247 | 4-fluorophenyl | 2-methoxyphenyl | 152-154 | — |
| 248 | 4-fluorophenyl | 4-chlorophenyl | 108-110 | — |
| 249 | 4-fluorophenyl | 3,4,5-trimethoxyphenyl | 179-182 | — |
| 250 | 4-fluorophenyl | 5-bromo-3-indolyl | 167-169 | — |
| 251 | 4-fluorophenyl | 4-ethoxy-3-methoxyphenyl | 150-152 | — |
| 252 | 2,4-dimethylphenyl | 2-benzyloxyphenyl | 177-179 | — |
| 253 | 2,4-dimethylphenyl | 3-indolyl | 192-194 | — |
| 254 | 2,4-dimethylphenyl | 2,4,6-trimethoxyphenyl | 132-134 | — |
| 255 | 2,4-dimethylphenyl | 4-(N,N-dimethylamino)phenyl | 168-171 | — |
| 256 | 2,4-dimethylphenyl | 2-chlorophenyl | 116-119 | — |
| 257 | 2,4-dimethylphenyl | 3-methylthiophen-2-yl | 166-168 | — |
| 258 | 2,4-dimethylphenyl | 2,4,5-trimethoxyphenyl | 161-163 | — |
| 259 | 2,4-dimethylphenyl | 2,5-dimethylphenyl | 139-141 | — |
| 260 | 2,4-dimethylphenyl | 4-biphenyl-1-yl | 168-170 | — |
| 261 | 2,4-dimethylphenyl | 2-fluoro-4-methoxyphenyl | 142-144 | — |
| 262 | 2,4-dimethylphenyl | 4-bromophenyl | 164-166 | — |
| 263 | 2,4-dimethylphenyl | 2,3,5-trichlorophenyl | 171-173 | — |
| 264 | 2,4-dimethylphenyl | 3,4-dimethylphenyl | 166-169 | — |
| 265 | 2,4-dimethylphenyl | 5-methylthiophen-2-yl | 158-160 | — |
| 266 | 2,4-dimethyiphenyl | 2-methoxyphenyl | 136-139 | — |
| 267 | 2,4-dimethylphenyl | 4-chlorophenyl | 158-161 | — |
| 268 | 2,4-dimethylphenyl | 3,4,5-trimethoxyphenyl | 178-180 | — |
| 269 | 2,4-dimethylphenyl | 5-bromo-3-indolyl | 231-235 | — |
| 270 | 2,4-dimethylphenyl | 4-methoxyphenyl | 127-129 | — |
| 271 | 2,4-dimethylphenyl | 4-hydroxyphenyl | 152-154 | — |
| 272 | 2,4-dimethylphenyl | 2,3,4-trimethoxyphenyl | 124-125 | — |
| 273 | 2,4-dimethylphenyl | 4-ethoxy-3-methoxyphenyl | 146-150 | — |
| 274 | 2,4-dimethylphenyl | 3,4-dichlorophenyl | 141-143 | — |
| 275 | 2,4-dimethylphenyl | 3-ethoxy-4-hydroxyphenyl | 146-148 | — |
| 276 | 2,4-dimethylphenyl | 4-methylsulfenyl | 135-137 | — |
| 277 | 4-methoxyphenyl | 2-benzyloxyphenyl | 111-112 | — |
| 278 | 4-methoxyphenyl | 3-indolyl | 201-205 | — |
| 279 | 4-methoxyphenyl | 2,4,6-trimethoxyphenyl | 151-154 | — |
| 280 | 4-methoxyphenyl | 4-(N,N-dimethylamino)phenyl | 166-169 | — |
| 281 | 4-methoxyphenyl | 3-methylthiophen-2-yl | 154-156 | — |
| 282 | 4-methoxyphenyl | 2,5-dimethylphenyl | 125-127 | — |
| 283 | 4-methoxyphenyl | 5-methylthiophen-2-yl | 118-121 | — |
| 284 | 4-methoxyphenyl | 2,4,5-trimethoxyphenyl | 171-173 | — |
| 285 | 4-methoxyphenyl | 2,3,4-trimethoxyphenyl | 140-142 | — |
| 286 | 4-methoxyphenyl | 4-biphenyl-1-yl | 119-120 | — |
| 287 | 4-methoxyphenyl | 4-ethoxy-3-methoxyphenyl | 111-113 | — |
| 288 | 4-methoxyphenyl | 3,4-dichlorophenyl | 84-86 | — |
| 289 | 4-methoxyphenyl | 4-hydroxyphenyl | 181-182 | — |
| 290 | 4-methoxyphenyl | 4-chlorophenyl | 95-96 | — |
| 291 | 4-methoxyphenyl | 4-bromophenyl | 102-103 | — |
| 292 | 4-methoxyphenyl | 2-hydroxyphenyl | 197-198 | — |
| 293 | 4-methoxyphenyl | 4-methylsulfenyl | 119-120 | — |
| 294 | 4-methoxyphenyl | 2,3,5-trichlorophenyl | 180-181 | — |

TABLE 7
NMR Spectral Data for Selected Table 1 Compounds.

| | —CH | Aromatic | Other | Melting | Yield |
|---|---|---|---|---|---|
| Example | (δ, ppm) | (δ, ppm) | (δ, ppm) | point ° C. | (%) |
| 1 | 7.95 | 6.05-7.35 | 3.79-3.88 (OCH₃), 4.40 (CH₂) | 178-180 | 66 |
| 2 | 8.00 | 6.90-8.20 | 3.80 (OCH₃), 4.25 (CH₂) | 138-140 | 52 |
| 4 | 8.00 | 6.85-8.19 | 4.41 (CH₂) | 146-148 | 58 |
| 7 | 8.13 | 6.95-8.09 | 4.81 (CH₂) | 171-173 | 59 |
| 10 | 7.69 | 6.82-8.30 | 3.74 (OCH₃), 4.47 (CH₂) | 174-176 | 63 |
| 11 | 7.77 | 7.03-7.47 | 3.94 (OCH₃), 4.66 (CH₂) | 110-112 | 50 |
| 12 | 7.92 | 6.86-7.64 | 3.79 (OCH₃), 4.41 (CH₂) | 150-153 | 56 |
| 13 | 7.84 | 6.65-7.52 | 3.57 (OCH₃), 4.23 (CH₂) | 142-144 | 58 |
| 22 | 7.89 | 6.93-7.93 | 3.79 (OCH₃), 4.66 (CH₂) | 210-212 | 53 |
| 23 | 7.90 | 6.91-7.87 | 3.79 (OCH₃), 4.67 (CH₂) | 208-210 | 56 |
| 24 | 7.73 | 6.89-8.05 | 4.72 (CH₂) | 178-180 | 52 |
| 25 | 7.65 | 6.92-7.63 | 3.79 (OCH₃), 4.60 (CH₂) | 144-146 | 58 |
| 26 | 7.76 | 6.89-7.68 | 3.79-3.96 (OCH₃); 4.63 (CH₂) | 156-158 | 57 |
| 27 | 7.75 | 6.89-7.61 | 3.79 (OCH₃); 4.63 (CH₂); 6.23 | 186-188 | 61 |
| | | | (OCH₂O) | | |
| 28 | 7.66 | 6.92-7.64 | 3.74-3.79 (OCH₃); 4.59 (CH₂) | 196-198 | 53 |
| 31 | 7.77 | 7.19-8.30 | 4.46 (CH₂) | 138-142 | 59 |
| 32 | 7.59 | 6.76-6.88 | 3.79-3.81 (OCH₃), 4.45 (CH₂) | LIQUID | 63 |
| 33 | 7.78 | 7.15-7.54 | 4.61 (CH₂) | 156-158 | 52 |
| 34 | 8.00 | 6.10-7.65 | 3.90-4.97 (OCH₃), 4.54 (CH₂) | 184-186 | 56 |
| 35 | 8.00 | 7.20-7.82 | 4.01 (OCH₃), 4.62 (CH$$) | 144-146 | 58 |

TABLE 8
Systematic Chemical Names of Compounds Listed in Tables 1-6.

| Example | Compound Name |
|---|---|
| 1 | (E)-2-[(4-methoxyphenyl)methanesulfonyl]-3-(2,4,6-trimethoxyphenyl)acrylonitrile |
| 2 | (E)-2-[(4-methoxyphenyl)methanesulfonyl]-3-(2,4-difluorophenyl)acrylonitrile |
| 3 | (E)-2-[(4-methoxyphenyl)methanesulfonyl]-3-(3-nitro-4-fluorophenyl)acrylonitrile |
| 4 | (E)-2-[(4-bromophenyl)methanesulfonyl]-3-(2,4-difluorophenyl)acrylonitrile |
| 5 | (E)-2-[(4-methoxyphenyl)methanesulfonyl]-3-(4-aminophenyl)acrylonitrile |
| 6 | (E)-2-[(4-methoxyphenyl)methanesulfonyl]-3-(3-amino-4-fluorophenyl)acrylonitrile |
| 7 | (E)-2-[(4-methoxyphenyl)methanesulfonyl]-3-(4-fluorophenyl)acrylonitrile |
| 8 | (E)-2-[(4-methoxyphenyl)methanesulfonyl]-3-(2,6-dimethoxyphenyl)acrylonitrile |
| 9 | (E)-2-[(4-methoxyphenyl)methanesulfonyl]-3-(2-methoxyphenyl)acrylonitrile |
| 10 | (E)-2-[(4-methoxyphenyl)methanesulfonyl]-3-(4-nitrophenyl)acrylonitrile |
| 11 | (E)-2-[(4-methoxyphenyl)methanesulfonyl]-3-(2,3,4,5,6-pentafluorophenyl)acrylonitrile |
| 12 | (E)-2-[(4-methoxyphenyl)methanesulfonyl]-3-(3-furyl)acrylonitrile |
| 13 | (E)-2-[(4-methoxyphenyl)methanesulfonyl]-3-(thiophen-3-yl)acrylonitrile |
| 14 | (E)-2-[(4-bromophenyl)methanesulfonyl]-3-(2-(difluoromethoxy)phenyl)acrylonitrile |
| 15 | (E)-2-[(4-methoxyphenyl)methanesulfonyl]-3-(2-chloro-4-fluorophenyl)acrylonitrile |
| 16 | (E)-2-[(4-bromophenyl)methanesulfonyl]-3-(2-fluoro-4-cyanophenyl)acrylonitrile |
| 17 | (E)-2-[(4-methoxyphenyl)methanesulfonyl]-3-(2-fluoro-4-cyanophenyl)acrylonitrile |
| 18 | (E)-2-[(4-bromophenyl)methanesulfonyl]-3-(2,6-dimethoxyphenyl)acrylonitrile |
| 19 | (E)-2-[(4-bromophenyl)methanesulfonyl]-3-(2,3,4,5,6-pentafluorophenyl)acrylonitrile |
| 20 | (E)-2-[(4-bromophenyl)methanesulfonyl]-3-(2,4,6-trimethoxyphenyl)acrylonitrile |
| 21 | (E)-2-[(4-chlorophenyl)methanesulfonyl]-3-(2,3,4,5,6-pentafluorophenyl)acrylonitrile |
| 22 | (E)-2-[(4-methoxyphenyl)methanesulfonyl]-3-(2-benzoxazolon-5-yl)acrylonitrile |
| 23 | (E)-2-[(4-methoxyphenyl)methanesulfonyl]-3-(2-benzoxazolon-6-yl)acrylonitrile |
| 24 | (E)-2-[(4-chlorophenyl)methanesulfonyl]-3-(3,4-dihydroxyphenyl)acrylonitrile |
| 25 | (E)-2-[(4-methoxyphenyl)methanesulfonyl]-3-(3,4-dihydroxyphenyl)acrylonitrile |
| 26 | (E)-2-[(4-methoxyphenyl)methanesulfonyl]-3-(3,4-dimethoxyphenyl)acrylonitrile |
| 27 | (E)-2-[(4-methoxyphenyl)methanesulfonyl]-3-(1,3-benzodioxole-5-yl)acrylonitrile |
| 28 | (E)-2-[(3,4-dimethoxyphenyl)methanesulfonyl]-3-(3,4-dihydroxyphenyl)acrylonitrile |
| 29 | (E)-2-[(3,4-dimethoxyphenyl)methanesulfonyl]-3-(2-benzoxazolon-6-yl)acrylonitrile |
| 30 | (E)-2-[(3,4-dimethoxyphenyl)methanesulfonyl]-3-(2-benzoxazolon-5-yl)acrylonitrile |
| 31 | (E)-2-[(4-bromophenyl)methanesulfonyl]-3-(4-nitrophenyl)acrylonitrile |
| 32 | (E)-2-[(3,4-dimethoxyphenyl)methanesulfonyl]-3-(2,3,4,5,6- |
| | pentafluorophenyl)acrylonitrile |
| 33 | (E)-2-[(4-fluorophenyl)methanesulfonyl]-3-(2,3,4,5,6-pentafluorophenyl)acrylonitrile |
| 34 | (E)-2-[(3-nitro-4-methoxyphenyl)methanesulfonyl]-3-(2,4,6-trimethoxyphenyl)acrylonitrile |
| 35 | (E)-2-[(3-nitro-4-methoxyphenyl)methanesulfonyl]-3-(2,3,4,5,6- |
| | pentafluorophenyl)acrylonitrile |
| 36 | (E)-2-[(3-amino-4-methoxyphenyl)methanesulfonyl]-3-(2,4,6- |
| | trimethoxyphenyl)acrylonitrile |
| 37 | (E)-2-[(4-fluorophenyl)methanesulfonyl]-3-(4-ethoxycarbonyl-3,5-dimethylpyrrol-2- |
| | yl)acrylonitrile |
| 38 | (E)-2-[(4-methoxy-3-(4-methylphenylsulfonyloxy)phenyl)methanesulfonyl]-3-(2,3,4,5,6- |
| | pentafluorophenyl)acrylonitrile |
| 39 | (E)-2-[(3-fluoro-4-methoxyphenyl)methanesulfonyl]-3-(2,4,6- |
| | trimethoxyphenyl)acrylonitrile |
| 40 | (E)-2-[(4-chlorophenyl)methanesulfonyl]-3-(2,5-dimethylphenyl)acrylonitrile |
| 41 | (E)-2-[(4-chlorophenyl)methanesulfonyl]-3-(2-chloro-4-fluorophenyl)acrylonitrile |
| 42 | (E)-2-[(4-chlorophenyl)methanesulfonyl]-3-(4-fluoro-3-methylphenyl)acrylonitrile |
| 43 | (E)-2-[(4-chlorophenyl)methanesulfonyl]-3-(4-methanesulfenyl)acrylonitrile |
| 44 | (E)-2-[(4-chlorophenyl)methanesulfonyl]-3-(3-hydroxy-4-methoxyphenyl)acrylonitrile |
| 45 | (E)-2-[(4-chlorophenyl)methanesulfonyl]-3-(4-bromophenyl)acrylonitrile |
| 46 | (E)-2-[(4-chlorophenyl)methanesulfonyl]-3-(2,3,4-trimethoxyphenyl)acrylonitrile |
| 47 | (E)-2-[(4-chlorophenyl)methanesulfonyl]-3-(4-fluorophenyl)acrylonitrile |
| 48 | (E)-2-[(4-chlorophenyl)methanesulfonyl]-3-(2-fluoro-4-nitrophenyl)acrylonitrile |
| 49 | (E)-2-[(4-chlorophenyl)methanesulfonyl]-3-(2-fluoro-4-methoxyphenyl)acrylonitrile |
| 50 | (E)-2-[(4-chlorophenyl)methanesulfonyl]-3-(4-methoxyphenyl)acrylonitrile |
| 51 | (E)-2-[(4-chlorophenyl)methanesulfonyl]-3-(2,4-dichlorophenyl)acrylonitrile |
| 52 | (E)-2-[(4-chlorophenyl)methanesulfonyl]-3-(3,4-dichlorophenyl)acrylonitrile |
| 53 | (E)-2-[(4-chlorophenyl)methanesulfonyl]-3-(2,4,6-trimethoxyphenyl)acrylonitrile |
| 54 | (E)-2-[(4-chlorophenyl)methanesulfonyl]-3-(3-ethoxy-4-hydroxyphenyl)acrylonitrile |
| 55 | (E)-2-[(4-chlorophenyl)methanesulfonyl]-3-(4-ethoxy-3-methoxyphenyl)acrylonitrile |
| 56 | (E)-2-[(4-chlorophenyl)methanesulfonyl]-3-(3,5-dimethoxyphenyl)acrylonitrile |
| 57 | (E)-2-[(4-chlorophenyl)methanesulfonyl]-3-(4-acetoxyphenyl)acrylonitrile |
| 58 | (E)-2-[(4-chlorophenyl)methanesulfonyl]-3-(4-(N,N-dimethylamino)phenyl)acrylonitrile |
| 59 | (E)-2-[(4-chlorophenyl)methanesulfonyl]-3-(4-hydroxy-3-nitrophenyl)acrylonitrile |
| 60 | (E)-2-[(4-chlorophenyl)methanesulfonyl]-3-(2,4-dihydroxyphenyl)acrylonitrile |
| 61 | (E)-2-[(4-chlorophenyl)methanesulfonyl]-3-(4-chlorophenyl)acrylonitrile |
| 62 | (E)-2-[(4-chlorophenyl)methanesulfonyl]-3-(2,3,4-trichlorophenyl)acrylonitrile |
| 63 | (E)-2-[(2,4-dichlorophenyl)methanesulfonyl]-3-(5-methylthiophen-2-yl)acrylonitrile |
| 64 | (E)-2-[(4-fluorophenyl)methanesulfonyl]-3-(4-(N,N-dimethylamino)phenyl)acrylonitrile |
| 65 | (E)-2-[(2,4-dichlorophenyl)methanesulfonyl]-3-(2-fluoro-4-methoxyphenyl)acrylonitrile |
| 66 | (E)-2-[(4-chlorophenyl)methanesulfonyl]-3-(3,4-dimethylphenyl)acrylonitrile |
| 67 | (E)-2-[(4-chlorophenyl)methanesulfonyl]-3-(4-biphenyl-1-yl)acrylonitrile |
| 68 | (E)-2-[(4-chlorophenyl)methanesulfonyl]-3-(3-indolyl)acrylonitrile |
| 69 | (E)-2-[(4-chlorophenyl)methanesulfonyl]-3-(phenyl)acrylonitrile |
| 70 | (E)-2-[(4-chlorophenyl)methanesulfonyl]-3-(2-benzyloxyphenyl)acrylonitrile |
| 71 | (E)-2-[(2,4-dichlorophenyl)methanesulfonyl]-3-(5-bromo-3-indolyl)acrylonitrile |
| 72 | (E)-2-[(4-chlorophenyl)methanesulfonyl]-3-(5-bromo-3-indolyl)acrylonitrile |
| 73 | (E)-2-[(4-chlorophenyl)methanesulfonyl]-3-(5-chloro-3-indolyl)acrylonitrile |
| 74 | (E)-2-[(4-chlorophenyl)methanesulfonyl]-3-(3-ethoxy-4-hydroxyphenyl)acrylonitrile |
| 75 | (E)-2-[(2,4-dichlorophenyl)methanesulfonyl]-3-(5-chloro-3-indolyl)acrylonitrile |
| 76 | (E)-2-[(2,4-dichlorophenyl)methanesulfonyl]-3-(4-methanesulfenyl)acrylonitrile |
| 77 | (E)-2-[(4-fluorophenyl)methanesulfonyl]-3-(5-chloro-3-indolyl)acrylonitrile |
| 78 | (E)-2-[(4-fluorophenyl)methanesulfonyl]-3-(5-bromo-3-indolyl)acrylonitrile |
| 79 | (E)-2-[(4-fluorophenyl)methanesulfonyl]-3-(2,4,6-trimethoxyphenyl)acrylonitrile |
| 80 | (E)-2-[(4-fluorophenyl)methanesulfonyl]-3-(3-hydroxy-4-methoxyphenyl)acrylonitrile |
| 81 | (E)-2-[(4-fluorophenyl)methanesulfonyl]-3-(3-indolyl)acrylonitrile |
| 82 | (E)-2-[(4-fluorophenyl)methanesulfonyl]-3-(4-bromophenyl)acrylonitrile |
| 83 | (E)-2-[(4-fluorophenyl)methanesulfonyl]-3-(2,4-dihydroxyphenyl)acrylonitrile |
| 84 | (E)-2-[(4-fluorophenyl)methanesulfonyl]-3-(4-acetoxyphenyl)acrylonitrile |
| 85 | (E)-2-[(4-fluorophenyl)methanesulfonyl]-3-(2,4-dichlorophenyl)acrylonitrile |
| 86 | (E)-2-[(4-fluorophenyl)methanesulfonyl]-3-(3,5-dimethoxyphenyl)acrylonitrile |
| 87 | (E)-2-[(4-fluorophenyl)methanesulfonyl]-3-(4-methanesulfenyl)acrylonitrile |
| 88 | (E)-2-[(4-fluorophenyl)methanesulfonyl]-3-(3,4-dimethylphenyl)acrylonitrile |
| 89 | (E)-2-[(4-fluorophenyl)methanesulfonyl]-3-(4-chlorophenyl)acrylonitrile |
| 90 | (E)-2-[(4-fluorophenyl)methanesulfonyl]-3-(2,5-dimethylphenyl)acrylonitrile |
| 91 | (E)-2-[(2,4-dichlorophenyl)methanesulfonyl]-3-(5-chloro-3-indolyl)acrylonitrile |
| 92 | (E)-2-[(2,4-dichlorophenyl)methanesulfonyl]-3-(2,4-dihydroxyphenyl)acrylonitrile |
| 93 | (E)-2-[(2,4-dichlorophenyl)methanesulfonyl]-3-(2,3,4-trimethoxyphenyl)acrylonitrile |
| 94 | (E)-2-[(2,4-dichlorophenyl)methanesulfonyl]-3-(4-ethoxy-3-methoxyphenyl)acrylonitrile |
| 95 | (E)-2-[(2,4-dichlorophenyl)methanesulfonyl]-3-(2-chloro-4-fluorophenyl)acrylonitrile |
| 96 | (E)-2-[(2,4-dichlorophenyl)methanesulfonyl]-3-(2,5-dimethylphenyl)acrylonitrile |
| 97 | (E)-2-[(2,4-dichlorophenyl)methanesulfonyl]-3-(3,4-dimethylphenyl)acrylonitrile |
| 98 | (E)-2-[(2,4-dichlorophenyl)methanesulfonyl]-3-(4-bromophenyl)acrylonitrile |
| 99 | (E)-2-[(2,4-dichlorophenyl)methanesulfonyl]-3-(4-methoxyphenyl)acrylonitrile |
| 100 | (E)-2-[(2,4-dichlorophenyl)methanesulfonyl]-3-(4-chlorophenyl)acrylonitrile |
| 101 | (E)-2-[(4-bromophenyl)methanesulfonyl]-3-(3,5-dimethylphenyl)acrylonitrile |
| 102 | (E)-2-[(2,4-dichlorophenyl)methanesulfonyl]-3-(3-methyl-2-thiophen-2-yl)acrylonitrile |
| 103 | (E)-2-[(2,4-dichlorophenyl)methanesulfonyl]-3-(3-methyl-2-furyl)acrylonitrile |
| 104 | (E)-2-[(2,4-dichlorophenyl)methanesulfonyl]-3-(3-furyl)acrylonitrile |
| 105 | (E)-2-[(2,4-dichlorophenyl)methanesulfonyl]-3-(3,4,5-trimethoxyphenyl)acrylonitrile |
| 106 | (E)-2-[(2,4-dichlorophenyl)methanesulfonyl]-3-(2,5-dimethoxyphenyl)acrylonitrile |
| 107 | (E)-2-[(2,4-dichlorophenyl)methanesulfonyl]-3-(2,4,5-trimethoxyphenyl)acrylonitrile |
| 108 | (E)-2-[(2,4-dichlorophenyl)methanesulfonyl]-3-(2-hydroxyphenyl)acrylonitrile |
| 109 | (E)-2-[(2,4-dichlorophenyl)methanesulfonyl]-3-(4-hydroxy-3-nitrophenyl)acrylonitrile |
| 110 | (E)-2-[(2,4-dichlorophenyl)methanesulfonyl]-3-(3,4-dimethoxyphenyl)acrylonitrile |
| 111 | (E)-2-[(2,4-dichlorophenyl)methanesulfonyl]-3-(2,4-dimethoxyphenyl)acrylonitrile |
| 112 | (E)-2-[(2,4-dichlorophenyl)methanesulfonyl]-3-(phenyl)acrylonitrile |
| 113 | (E)-2-[(2,4-dichlorophenyl)methanesulfonyl]-3-(2-hydroxy-4-methoxyphenyl)acrylonitrile |
| 114 | (E)-2-[(3-bromophenyl)methanesulfonyl]-3-(3-methyl-2-furyl)acrylonitrile |
| 115 | (E)-2-[(3-bromophenyl)methanesulfonyl]-3-(3-furyl)acrylonitrile |
| 116 | (E)-2-[(3-bromophenyl)methanesulfonyl]-3-(3,4-dihydroxyphenyl)acrylonitrile |
| 117 | (E)-2-[(3-bromophenyl)methanesulfonyl]-3-(3-hydroxyphenyl)acrylonitrile |
| 118 | (E)-2-[(4-bromophenyl)methanesulfonyl]-3-(5-methylthiophen-2-yl)acrylonitrile |
| 119 | (E)-2-[(4-bromophenyl)methanesulfonyl]-3-(2,5-dimethylphenyl)acrylonitrile |
| 120 | (E)-2-[(4-chlorophenyl)methanesulfonyl]-3-(3-methyl-2-thiophen-2-yl)acrylonitrile |
| 121 | (E)-2-[(4-chlorophenyl)methanesulfonyl]-3-(2-hydroxyphenyl)acrylonitrile |
| 122 | (E)-2-[(4-chlorophenyl)methanesulfonyl]-3-(3-furyl)acrylonitrile |
| 123 | (E)-2-[(4-chlorophenyl)methanesulfonyl]-3-(5-methylthiophen-2-yl)acrylonitrile |
| 124 | (E)-2-[(phenyl)methanesulfonyl]-3-(3-hydroxyphenyl)acrylonitrile |
| 125 | (E)-2-[(phenyl)methanesulfonyl]-3-(2-hydroxyphenyl)acrylonitrile |
| 126 | (E)-2-[(phenyl)methanesulfonyl]-3-(2-methoxyphenyl)acrylonitrile |
| 127 | (E)-2-[(phenyl)methanesulfonyl]-3-(3-methyl-2-thiophen-2-yl)acrylonitrile |
| 128 | (E)-2-[(phenyl)methanesulfonyl]-3-(3-methyl-2-furyl)acrylonitrile |
| 129 | (E)-2-[(phenyl)methanesulfonyl]-3-(2-fluoro-4-methoxyphenyl)acrylonitrile |
| 130 | (E)-2-[(phenyl)methanesulfonyl]-3-(2-chlorophenyl)acrylonitrile |
| 131 | (E)-2-[(phenyl)methanesulfonyl]-3-(3,4,5-trimethoxyphenyl)acrylonitrile |
| 132 | (E)-2-[(phenyl)methanesulfonyl]-3-(2,4-dimethoxyphenyl)acrylonitrile |
| 133 | (E)-2-[(phenyl)methanesulfonyl]-3-(3,4-dimethoxyphenyl)acrylonitrile |
| 134 | (E)-2-[(phenyl)methanesulfonyl]-3-(3,5-dimethoxyphenyl)acrylonitrile |
| 135 | (E)-2-[(phenyl)methanesulfonyl]-3-(2,5-dimethoxyphenyl)acrylonitrile |
| 136 | (E)-2-[(phenyl)methanesulfonyl]-3-(2,5-dimethylphenyl)acrylonitrile |
| 137 | (E)-2-[(phenyl)methanesulfonyl]-3-(2-benzyloxyphenyl)acrylonitrile |
| 138 | (E)-2-[(phenyl)methanesulfonyl]-3-(4-biphenyl-1-yl)acrylonitrile |
| 139 | (E)-2-[(phenyl)methanesulfonyl]-3-(4-hydroxy-3-nitrophenyl)acrylonitrile |
| 140 | (E)-2-[(4-methoxyphenyl)methanesulfonyl]-3-(4-biphenyl-1-yl)acrylonitrile |
| 141 | (E)-2-[(4-methoxyphenyl)methanesulfonyl]-3-(4-methoxyphenyl)acrylonitrile |
| 142 | (E)-2-[(phenyl)methanesulfonyl]-3-(2,4-difluorophenyl)acrylonitrile |
| 143 | (E)-2-[(phenyl)methanesulfonyl]-3-(3,5-difluorophenyl)acrylonitrile |
| 144 | (E)-2-[(4-bromophenyl)methanesulfonyl]-3-(2-chlorophenyl)acrylonitrile |
| 145 | (E)-2-[(4-fluorophenyl)methanesulfonyl]-3-(2-chlorophenyl)acrylonitrile |
| 146 | (E)-2-[(4-methoxyphenyl)methanesulfonyl]-3-(2,3,4-trichlorophenyl)acrylonitrile |
| 147 | (E)-2-[(4-methoxyphenyl)methanesulfonyl]-3-(2,4-dimethoxyphenyl)acrylonitrile |
| 148 | (E)-2-[(4-methoxyphenyl)methanesulfonyl]-3-(3,4-dimethoxyphenyl)acrylonitrile |
| 149 | (E)-2-[(4-methoxyphenyl)methanesulfonyl]-3-(3,5-dimethoxyphenyl)acrylonitrile |
| 150 | (E)-2-[(2,4-dichlorophenyl)methanesulfonyl]-3-(4-chlorophenyl)acrylamide |
| 151 | (E)-2-[(2,4-dichlorophenyl)methanesulfonyl]-3-(3,4-dimethylphenyl)acrylamide |
| 152 | (E)-2-[(2,4-dichlorophenyl)methanesulfonyl]-3-(2,4,6-trimethoxyphenyl)acrylamide |
| 153 | (E)-2-[(2,4-dichlorophenyl)methanesulfonyl]-3-(4-bromophenyl)acrylamide |
| 154 | (E)-2-[(2,4-dichlorophenyl)methanesulfonyl]-3-(3-hydroxy-4-methoxyphenyl)acrylamide |
| 155 | (E)-2-[(4-fluorophenyl)methanesulfonyl]-3-(2,3-dichlorophenyl)acrylamide |
| 156 | (E)-2-[(2-chlorophenyl)methanesulfonyl]-3-(2,4-dichlorophenyl)acrylamide |
| 157 | (E)-2-[(4-chlorophenyl)methanesulfonyl]-3-(2,5-dimethylphenyl)acrylamide |
| 158 | (E)-2-[(4-chlorophenyl)methanesulfonyl]-3-(4-ethoxy-3-methoxyphenyl)acrylamide |
| 159 | (E)-2-[(4-nitrophenyl)methanesulfonyl]-3-(4-ethoxy-3-methoxyphenyl)acrylamide |
| 160 | (E)-2-[(4-fluorophenyl)methanesulfonyl]-3-(2,5-dimethylphenyl)acrylamide |
| 161 | (E)-2-[(4-fluorophenyl)methanesulfonyl]-3-(4-bromophenyl)acrylamide |
| 162 | (E)-2-[(4-fluorophenyl)methanesulfonyl]-3-(2,4,5-trifluorophenyl)acrylamide |
| 163 | (E)-2-[(4-chlorophenyl)methanesulfonyl]-3-(3,5-dimethylphenyl)acrylamide |
| 164 | (E)-2-[(4-fluorophenyl)methanesulfonyl]-3-(4-ethoxy-3-methoxyphenyl)acrylamide |
| 165 | (E)-2-[(4-chlorophenyl)methanesulfonyl]-3-(2,4-dichlorophenyl)acrylamide |
| 166 | (E)-2-[(4-chlorophenyl)methanesulfonyl]-3-(2-chloro-4-fluorophenyl)acrylamide |
| 167 | (E)-2-[(2,4-dichlorophenyl)methanesulfonyl]-3-(4-acetylphenyl)acrylamide |
| 168 | (E)-2-[(2,4-dichlorophenyl)methanesulfonyl]-3-(4-ethoxy-3-methoxyphenyl)acrylamide |
| 169 | (E)-2-[(4-nitrophenyl)methanesulfonyl]-3-(2,3-dichlorophenyl)acrylamide |
| 170 | (E)-2-[(4-chlorophenyl)methanesulfonyl]-3-(4-flurophenyl)acrylamide |
| 171 | (E)-2-[(4-methoxyphenyl)methanesulfonyl]-3-(2,4,6-trimethoxyphenyl)acrylamide |
| 172 | (E)-2-[(4-methoxyphenyl)methanesulfonyl]-3-(4-fluorophenyl)acrylamide |
| 173 | (E)-2-[(4-methoxyphenyl)methanesulfonyl]-3-(2,6-dimethoxyphenyl)acrylamide |
| 174 | (E)-2-[(4-chlorophenyl)methanesulfonyl]-3-(2,4,6-trimethoxyphenyl)acrylamide |
| 175 | (E)-2-[(4-chlorophenyl)methanesulfonyl]-3-(2,6-dimethoxyphenyl)acrylamide |
| 176 | (E)-2-[(4-chlorophenyl)methanesulfonyl]-3-(4-nitrophenyl)acrylamide |
| 177 | (E)-2-(phenyl)sulfonyl-3-(4-fluorophenyl)acrylamide |
| 178 | (E)-2-(phenyl)sulfonyl-3-(2,4,6-trimethoxyphenyl)acrylamide |
| 179 | (E)-2-(phenyl)sulfonyl-3-(2,6-dimethoxyphenyl)acrylamide |
| 180 | (E)-2-(phenyl)sulfonyl-3-(4-nitrophenyl)acrylamide |
| 181 | (E)-2-(4-methoxyphenyl)sulfonyl-3-(4-fluorophenyl)acrylamide |
| 182 | (E)-2-(4-methoxyphenyl)sulfonyl-3-(2,4,6-trimethoxyphenyl)acrylamide |
| 183 | (E)-2-(4-methoxyphenyl)sulfonyl-3-(2,6-dimethoxyphenyl)acrylamide |
| 184 | (E)-2-(4-methoxyphenyl)sulfonyl-3-(4-nitrophenyl)acrylamide |
| 185 | (E)-1-(4-methylphenyl)sulfonyl-2-(2,4,6-trimethoxyphenyl)-1-nitroethene |
| 186 | (E)-1-(4-bromophenyl)sulfonyl-2-(2,4,6-trimethoxyphenyl)-1-nitroethene |
| 187 | (E)-1-(phenyl)sulfonyl-2-(2,4,6-trimethoxyphenyl)-1-nitroethene |
| 188 | (E)-1-[(4-methoxyphenyl)methanesulfonyl]-2-(2,4,6-trimethoxyphenyl)-1-nitroethene |
| 189 | (E)-1-[(4-fluorophenyl)methanesulfonyl]-2-(2,4,6-trimethoxyphenyl)-1-nitroethene |
| 190 | (E)-2-[(4-methoxyphenyl)sulfamoyl]-3-(4-bromophenyl)acrylonitrile |
| 191 | (E)-2-[(4-methoxyphenyl)sulfamoyl]-3-(2,4,6-trimethoxyphenyl)acrylonitrile |
| 192 | (E)-2-[(4-methoxyphenyl)sulfamoyl]-3-(2,3,4,5,6-pentafluorophenyl)acrylonitrile |
| 193 | (E)-2-[(4-methoxyphenyl)sulfamoyl]-3-(2,6-dimethoxyphenyl)acrylonitrile |
| 194 | (E)-2-[(4-methoxy-3-(4-methylphenylsulfonyloxy)phenyl)sulfamoyl]-3-(2,3,4,5,6- |
| | pentafluorophenyl)acrylonitrile |
| 195 | (E)-2-[(4-chlorophenyl)sulfamoyl]-3-(4-methoxyphenyl)acrylonitrile |
| 196 | (E)-2-[(2-phenoxyphenyl)sulfamoyl]-3-(4-bromophenyl)acrylonitrile |
| 197 | (E)-2-[(2-phenoxyphenyl)sulfamoyl]-3-(3-indolyl)acrylonitrile |
| 198 | (E)-2-[(2-phenoxyphenyl)sulfamoyl]-3-(5-methylthiophen-2-yl)acrylonitrile |
| 199 | (E)-2-[(2-phenoxyphenyl)sulfamoyl]-3-(2-methoxyphenyl)acrylonitrile |
| 200 | (E)-2-[(4-methoxyphenyl)sulfamoyl]-3-(4-fluorophenyl)acrylonitrile |
| 201 | (E)-2-[(4-methoxyphenyl)sulfamoyl]-3-(3,5-dimethoxyphenyl)acrylonitrile |
| 202 | (E)-2-[(4-methoxyphenyl)sulfamoyl]-3-(2,5-dimethoxyphenyl)acrylonitrile |
| 203 | (E)-2-[(4-methoxyphenyl)sulfamoyl]-3-(3,4-dimethylphenyl)acrylonitrile |
| 204 | (E)-2-[(4-methoxyphenyl)sulfamoyl]-3-(2-methoxyphenyl)acrylonitrile |
| 205 | (E)-2-[(4-chlorophenyl)sulfamoyl]-3-(3-indolyl)acrylonitrile |
| 206 | (E)-2-[(2-phenoxyphenyl)sulfamoyl]-3-(4-(N,N-dimethylamino)phenyl)acrylonitrile |
| 207 | (E)-2-[(2-phenoxyphenyl)sulfamoyl]-3-(4-chlorophenyl)acrylonitrile |
| 208 | (E)-2-[(2-phenoxyphenyl)sulfamoyl]-3-(2,4,6-trimethoxyphenyl)acrylonitrile |
| 209 | (E)-2-[(2-phenoxyphenyl)sulfamoyl]-3-(3-nitro-4-hydroxyphenyl)acrylonitrile |
| 210 | (E)-2-[(2-phenoxyphenyl)sulfamoyl]-3-(2-phenoxyphenyl)acrylonitrile |
| 211 | (E)-2-[(2-phenoxyphenyl)sulfamoyl]-3-(4-ethoxy-3-methoxyphenyl)acrylonitrile |
| 212 | (E)-2-[(4-bromophenyl)sulfamoyl]-3-(4-chlorophenyl)acrylonitrile |
| 213 | (E)-2-[(4-bromophenyl)sulfamoyl]-3-(2-benzyloxyphenyl)acrylonitrile |
| 214 | (E)-2-[(4-bromophenyl)sulfamoyl]-3-(3-indolyl)acrylonitrile |
| 215 | (E)-2-[(4-bromophenyl)sulfamoyl]-3-(2,4,6-trimethoxyphenyl)acrylonitrile |
| 216 | (E)-2-[(4-bromophenyl)sulfamoyl]-3-(4-(N,N-dimethylamino)phenyl)acrylonitrile |
| 217 | (E)-2-[(4-bromophenyl)sulfamoyl]-3-(4-bromophenyl)acrylonitrile |
| 218 | (E)-2-[(4-bromophenyl)sulfamoyl]-3-(2,3,4-trimethoxyphenyl)acrylonitrile |
| 219 | (E)-2-[(4-bromophenyl)sulfamoyl]-3-(4-ethoxy-3-methoxyphenyl)acrylonitrile |
| 220 | (E)-2-[(4-bromophenyl)sulfamoyl]-3-(4-biphenyl-1-yl)acrylonitrile |
| 221 | (E)-2-[(4-bromophenyl)sulfamoyl]-3-(2,3,4,5,6-pentafluorophenyl)acrylonitrile |
| 222 | (E)-2-[(4-bromophenyl)sulfamoyl]-3-(5-bromo-3-indolyl)acrylonitrile |
| 223 | (E)-2-[(4-bromophenyl)sulfamoyl]-3-(3-nitro-4-hydroxyphenyl)acrylonitrile |
| 224 | (E)-2-[(4-bromophenyl)sulfamoyl]-3-(3-methylthiophen-2-yl)acrylonitrile |
| 225 | (E)-2-[(4-bromophenyl)sulfamoyl]-3-(2-methoxyphenyl)acrylonitrile |
| 226 | (E)-2-[(4-bromophenyl)sulfamoyl]-3-(2,4-difluorophenyl)acrylonitrile |
| 227 | (E)-2-[(4-bromophenyl)sulfamoyl]-3-(2-chlorophenyl)acrylonitrile |
| 228 | (E)-2-[(4-bromophenyl)sulfamoyl]-3-(2-hydroxyphenyl)acrylonitrile |
| 229 | (E)-2-[(4-bromophenyl)sulfamoyl]-3-(2,5-dimethylphenyl)acrylonitrile |
| 230 | (E)-2-[(4-fluorophenyl)sulfamoyl]-3-(2-benzyloxyphenyl)acrylonitrile |
| 231 | (E)-2-[(4-fluorophenyl)sulfamoyl]-3-(3-indolyl)acrylonitrile |
| 232 | (E)-2-[(4-fluorophenyl)sulfamoyl]-3-(2,4,6-trimethoxyphenyl)acrylonitrile |
| 233 | (E)-2-[(4-fluorophenyl)sulfamoyl]-3-(4-(N,N-dimethylamino)phenyl)acrylonitrile |
| 234 | (E)-2-[(4-fluorophenyl)sulfamoyl]-3-(2,3,4,5,6-pentafluorophenyl)acrylonitrile |
| 235 | (E)-2-[(4-fluorophenyl)sulfamoyl]-3-(2-chlorophenyl)acrylonitrile |
| 236 | (E)-2-[(4-fluorophenyl)sulfamoyl]-3-(3-methylthiophen-2-yl)acrylonitrile |
| 237 | (E)-2-[(4-fluorophenyl)sulfamoyl]-3-(2,4,5-trimethoxyphenyl)acrylonitrile |
| 238 | (E)-2-[(4-fluorophenyl)sulfamoyl]-3-(2,5-dimethylphenyl)acrylonitrile |
| 239 | (E)-2-[(4-fluorophenyl)sulfamoyl]-3-(4-biphenyl-1-yl)acrylonitrile |
| 240 | (E)-2-[(4-fluorophenyl)sulfamoyl]-3-(2,4-difluorophenyl)acrylonitrile |
| 241 | (E)-2-[(4-fluorophenyl)sulfamoyl]-3-(2-fluoro-4-methoxyphenyl)acrylonitrile |
| 242 | (E)-2-[(4-fluorophenyl)sulfamoyl]-3-(4-bromophenyl)acrylonitrile |
| 243 | (E)-2-[(4-fluorophenyl)sulfamoyl]-3-(2,3,5-trichlorophenyl)acrylonitrile |
| 244 | (E)-2-[(4-fluorophenyl)sulfamoyl]-3-(3,4-dimethylphenyl)acrylonitrile |
| 245 | (E)-2-[(4-fluorophenyl)sulfamoyl]-3-(3-chloro-4-fluorophenyl)acrylonitrile |
| 246 | (E)-2-[(4-fluorophenyl)sulfamoyl]-3-(5-methylthiophen-2-yl)acrylonitrile |
| 247 | (E)-2-[(4-fluorophenyl)sulfamoyl]-3-(2-methoxyphenyl)acrylonitrile |
| 248 | (E)-2-[(4-fluorophenyl)sulfamoyl]-3-(4-chlorophenyl)acrylonitrile |
| 249 | (E)-2-[(4-fluorophenyl)sulfamoyl]-3-(3,4,5-trimethoxyphenyl)acrylonitrile |
| 250 | (E)-2-[(4-fluorophenyl)sulfamoyl]-3-(5-bromo-3-indolyl)acrylonitrile |
| 251 | (E)-2-[(4-fluorophenyl)sulfamoyl]-3-(4-ethoxy-3-methoxyphenyl)acrylonitrile |
| 252 | (E)-2-[(2,4-dimethylphenyl)sulfamoyl]-3-(2-benzyloxyphenyl)acrylonitrile |
| 253 | (E)-2-[(2,4-dimethylphenyl)sulfamoyl]-3-(3-indolyl)acrylonitrile |
| 254 | (E)-2-[(2,4-dimethylphenyl)sulfamoyl]-3-(2,4,6-trimethoxyphenyl)acrylonitrile |
| 255 | (E)-2-[(2,4-dimethylphenyl)sulfamoyl]-3-(4-(N,N-dimethylamino)phenyl)acrylonitrile |
| 256 | (E)-2-[(2,4-dimethylphenyl)sulfamoyl]-3-(2-chlorophenyl)acrylonitrile |
| 257 | (E)-2-[(2,4-dimethylphenyl)sulfamoyl]-3-(3-methylthiophen-2-yl)acrylonitrile |
| 258 | (E)-2-[(2,4-dimethylphenyl)sulfamoyl]-3-(2,4,5-trimethoxyphenyl)acrylonitrile |
| 259 | (E)-2-[(2,4-dimethylphenyl)sulfamoyl]-3-(2,5-dimethylphenyl)acrylonitrile |
| 260 | (E)-2-[(2,4-dimethylphenyl)sulfamoyl]-3-(4-biphenyl-1-yl)acrylonitrile |
| 261 | (E)-2-[(2,4-dimethylphenyl)sulfamoyl]-3-(2-fluoro-4-methoxyphenyl)acrylonitrile |
| 262 | (E)-2-[(2,4-dimethylphenyl)sulfamoyl]-3-(4-bromophenyl)acrylonitrile |
| 263 | (E)-2-[(2,4-dimethylphenyl)sulfamoyl]-3-(2,3,5-trichlorophenyl)acrylonitrile |
| 264 | (E)-2-[(2,4-dimethylphenyl)sulfamoyl]-3-(3,4-dimethylphenyl)acrylonitrile |
| 265 | (E)-2-[(2,4-dimethylphenyl)sulfamoyl]-3-(5-methylthiophen-2-yl)acrylonitrile |
| 266 | (E)-2-[(2,4-dimethylphenyl)sulfamoyl]-3-(2-methoxyphenyl)acrylonitrile |
| 267 | (E)-2-[(2,4-dimethylphenyl)sulfamoyl]-3-(4-chlorophenyl)acrylonitrile |
| 268 | (E)-2-[(2,4-dimethylphenyl)sulfamoyl]-3-(3,4,5-trimethoxyphenyl)acrylonitrile |
| 269 | (E)-2-[(2,4-dimethylphenyl)sulfamoyl]-3-(5-bromo-3-indolyl)acrylonitrile |
| 270 | (E)-2-[(2,4-dimethylphenyl)sulfamoyl]-3-(4-methoxyphenyl)acrylonitrile |
| 271 | (E)-2-[(2,4-dimethylphenyl)sulfamoyl]-3-(4-hydroxyphenyl)acrylonitrile |
| 272 | (E)-2-[(2,4-dimethylphenyl)sulfamoyl]-3-(2,3,4-trimethoxyphenyl)acrylonitrile |
| 273 | (E)-2-[(2,4-dimethylphenyl)sulfamoyl]-3-(4-ethoxy-3-methoxyphenyl)acrylonitrile |
| 274 | (E)-2-[(2,4-dimethylphenyl)sulfamoyl]-3-(3,4-dichlorophenyl)acrylonitrile |
| 275 | (E)-2-[(2,4-dimethylphenyl)sulfamoyl]-3-(3-ethoxy-4-hydroxyphenyl)acrylonitrile |
| 276 | (E)-2-[(2,4-dimethylphenyl)sulfamoyl]-3-(4-methylsulfenyl)acrylonitrile |
| 277 | (E)-2-[(4-methoxyphenyl)sulfamoyl]-3-(2-benzyloxyphenyl)acrylonitrile |
| 278 | (E)-2-[(4-methoxyphenyl)sulfamoyl]-3-(3-indolyl)acrylonitrile |
| 279 | (E)-2-[(4-methoxyphenyl)sulfamoyl]-3-(2,4,6-trimethoxyphenyl)acrylonitrile |
| 280 | (E)-2-[(4-methoxyphenyl)sulfamoyl]-3-(4-(N,N-dimethylamino)phenyl)acrylonitrile |
| 281 | (E)-2-[(4-methoxyphenyl)sulfamoyl]-3-(3-methylthiophen-2-yl)acrylonitrile |
| 282 | (E)-2-[(4-methoxyphenyl)sulfamoyl]-3-(2,5-dimethylphenyl)acrylonitrile |
| 283 | (E)-2-[(4-methoxyphenyl)sulfamoyl]-3-(5-methylthiophen-2-yl)acrylonitrile |
| 284 | (E)-2-[(4-methoxyphenyl)sulfamoyl]-3-(2,4,5-trimethoxyphenyl)acrylonitrile |
| 285 | (E)-2-[(4-methoxyphenyl)sulfamoyl]-3-(2,3,4-trimethoxyphenyl)acrylonitrile |
| 286 | (E)-2-[(4-methoxyphenyl)sulfamoyl]-3-(4-biphenyl-1-yl)acrylonitrile |
| 287 | (E)-2-[(4-methoxyphenyl)sulfamoyl]-3-(4-ethoxy-3-methoxyphenyl)acrylonitrile |
| 288 | (E)-2-[(4-methoxyphenyl)sulfamoyl]-3-(3,4-dichlorophenyl)acrylonitrile |
| 289 | (E)-2-[(4-methoxyphenyl)sulfamoyl]-3-(4-hydroxyphenyl)acrylonitrile |
| 290 | (E)-2-[(4-methoxyphenyl)sulfamoyl]-3-(4-chlorophenyl)acrylonitrile |
| 291 | (E)-2-[(4-methoxyphenyl)sulfamoyl]-3-(4-bromophenyl)acrylonitrile |
| 292 | (E)-2-[(4-methoxyphenyl)sulfamoyl]-3-(2-hydroxyphenyl)acrylonitrile |
| 293 | (E)-2-[(4-methoxyphenyl)sulfamoyl]-3-(4-methylsulfenyl)acrylonitrile |
| 294 | (E)-2-[(4-methoxyphenyl)sulfamoyl]-3-(2,3,5-trichlorophenyl)acrylonitrile |

### Effect of the Compounds of the Invention on Tumor Cell Lines.

The effect of the compounds of the invention on tumor cells was determined by the assay described by Latham et al., Oncogene 12:827-837 (1996). Tumor cells DU145 (prostate cancer), K562 (chronic myelogenous leukaemia), BT20 (breast carcinoma), H157 (non small cell lung carcinoma), or DLD1 (colon carcinoma) were plated in 6-well dishes at a cell density of 1.0×10⁵ cells per 35-mm² well. The plated cells were treated 24 hours later with a compound of the invention dissolved in DMSO at multiple concentrations ranging from 100 nM to 10 μM. The total number of viable cells was determined 96 hours later by trypsinizing the wells and counting the number of viable cells, as determined by trypan blue exclusion, using a hemacytometer. Normal HFL cells were treated with the same compounds under the same conditions of concentration and time. The normal cells displayed growth inhibition but no appreciable cell death.

Representative examples of activities of compounds of the invention in the cell lines are listed in Table 9.

TABLE 9
Representative Examples of Activities of Compounds
of the Invention in Cell Lines:

| Compound | DU145 | K562 | BT20 | H157 | DLD1 |
|---|---|---|---|---|---|
| Example | (IC₅₀/μM) | (IC₅₀/μM) | (IC₅₀/μM) | (IC₅₀/μM | (IC₅₀/μM) |
| 1 | 25 | — | — | — | — |
| 2 | 100 | — | — | — | — |
| 3 | 50 | — | — | — | — |
| 5 | 75 | — | — | — | — |
| 7 | 100 | — | — | — | — |
| 8 | 75 | — | — | — | — |
| 11 | 25 | 1 | — | — | — |
| 15 | 25 | — | — | — | — |
| 16 | 75 | 10 | — | — | — |
| 18 | 25 | 10 | — | — | — |
| 19 | 50 | 10 | — | — | — |
| 20 | 75 | 10 | — | — | — |
| 21 | 50 | 25 | — | — | — |
| 22 | 10 | — | — | — | — |
| 23 | 50 | — | — | — | — |
| 24 | 10 | — | — | — | — |
| 25 | 10 | — | — | — | — |
| 26 | 100 | — | — | — | — |
| 27 | 100 | — | — | — | — |
| 29 | 100 | — | — | — | — |
| 30 | 100 | — | — | — | — |
| 32 | 10 | — | — | — | — |
| 33 | 2.5 | — | — | — | — |
| 35 | 25 | 1 | — | — | — |
| 36 | 25 | 5 | — | — | — |
| 37 | 50-100 | 50-100 | — | — | — |
| 38 | >100 | 50-100 | — | — | — |
| 39 | 1-10 | 1-10 | — | — | — |
| 40 | 25-50 | 1-10 | — | — | — |
| 41 | 50-100 | 50-100 | — | — | — |
| 42 | 50-100 | 50-100 | — | — | — |
| 43 | >100 | 50-100 | — | — | — |
| 44 | >100 | 50-100 | — | — | — |
| 45 | 50-100 | 50-100 | — | — | — |
| 46 | >100 | >100 | — | — | — |
| 47 | 50-100 | 50-100 | — | — | — |
| 48 | 1-10 | 1-10 | — | — | — |
| 49 | >100 | 50-100 | — | — | — |
| 50 | >100 | >100 | — | — | — |
| 51 | 25-50 | 25-50 | — | — | — |
| 52 | 25-50 | 10-25 | — | — | — |
| 53 | 50-100 | 50-100 | — | — | — |
| 54 | >100 | 50-100 | — | — | — |
| 55 | 10-25 | 10-25 | — | — | — |
| 56 | 50-100 | 25-50 | — | — | — |
| 57 | 25-50 | 25-50 | — | — | — |
| 58 | 50-100 | 25-50 | — | — | — |
| 59 | 50-100 | 50-100 | — | — | — |
| 60 | >100 | >100 | — | — | — |
| 61 | 50-100 | 25-50 | — | — | — |
| 62 | >100 | 50-100 | — | — | — |
| 63 | >100 | >100 | — | — | — |
| 64 | 50-100 | 50-100 | — | — | — |
| 65 | 50-100 | 50-100 | — | — | — |
| 66 | 50-100 | 50-100 | — | — | — |
| 67 | >100 | >100 | — | — | — |
| 68 | 25-50 | 10-25 | — | — | — |
| 69 | >100 | >100 | — | — | — |
| 70 | >100 | >100 | — | — | — |
| 71 | 50-100 | 50-100 | — | — | — |
| 72 | >100 | >100 | — | — | — |
| 73 | 25-50 | 10-25 | — | — | — |
| 74 | 50-100 | 50-100 | — | — | — |
| 75 | 50-100 | 50-100 | — | — | — |
| 76 | 50-100 | 50-100 | — | — | — |
| 77 | 50-100 | 50-100 | — | — | — |
| 78 | 50-100 | 50-100 | — | — | — |
| 79 | >100 | 50-100 | — | — | — |
| 80 | >100 | 50-100 | — | — | — |
| 81 | 50-100 | 50-100 | — | — | — |
| 82 | >100 | >100 | — | — | — |
| 83 | 50-100 | 50-100 | — | — | — |
| 84 | 1-10 | 1-10 | — | — | — |
| 85 | >100 | 50-100 | — | — | — |
| 86 | >100 | >100 | — | — | — |
| 87 | 25-50 | 25-50 | — | — | — |
| 88 | 25-50 | 10-25 | — | — | — |
| 89 | 50-100 | 50-100 | — | — | — |
| 90 | >100 | 50-100 | — | — | — |
| 91 | 10-25 | 10-25 | — | — | — |
| 92 | 50-100 | 25-50 | — | — | — |
| 93 | 25-50 | 25-50 | — | — | — |
| 94 | 50-100 | 25-50 | — | — | — |
| 95 | 50-100 | 50-100 | — | — | — |
| 96 | >100 | 50-100 | — | — | — |
| 97 | >100 | >100 | — | — | — |
| 98 | 50-100 | 25-50 | — | — | — |
| 99 | >100 | 50-100 | — | — | — |
| 100 | >100 | >100 | — | — | — |
| 101 | >100 | 50-100 | — | — | — |
| 102 | >100 | 50-100 | — | — | — |
| 103 | >100 | 50-100 | — | — | — |
| 104 | >100 | 50-100 | — | — | — |
| 105 | 25-50 | 10-25 | — | — | — |
| 106 | 50-100 | 50-100 | — | — | — |
| 107 | 50-100 | >100 | — | — | — |
| 108 | >100 | >100 | — | — | — |
| 109 | >100 | >100 | — | — | — |
| 110 | 50-100 | 25-50 | — | — | — |
| 111 | >100 | >100 | — | — | — |
| 112 | 50-100 | 50-100 | — | — | — |
| 113 | >100 | >100 | — | — | — |
| 114 | >100 | >100 | — | — | — |
| 115 | >100 | >100 | — | — | — |
| 116 | >100 | >100 | — | — | — |
| 117 | >100 | >100 | — | — | — |
| 118 | >100 | >100 | — | — | — |
| 119 | >100 | >100 | — | — | — |
| 120 | 50-100 | 25-50 | — | — | — |
| 121 | >100 | >100 | — | — | — |
| 122 | >100 | >100 | — | — | — |
| 123 | >100 | >100 | — | — | — |
| 124 | >100 | 50-100 | — | — | — |
| 125 | 50-100 | >100 | — | — | — |
| 126 | >100 | >100 | — | — | — |
| 127 | >100 | >100 | — | — | — |
| 128 | >100 | 50-100 | — | — | — |
| 129 | >100 | 50-100 | — | — | — |
| 130 | >100 | >100 | — | — | — |
| 131 | >100 | 50-100 | — | — | — |
| 132 | >100 | >100 | — | — | — |
| 133 | >100 | 50-100 | — | — | — |
| 134 | 50-100 | 10-25 | — | — | — |
| 135 | >100 | >100 | — | — | — |
| 136 | 25-50 | 1-10 | — | — | — |
| 137 | >100 | 50-100 | — | — | — |
| 138 | >100 | >100 | — | — | — |
| 139 | >100 | 50-100 | — | — | — |
| 140 | >100 | >100 | — | — | — |
| 141 | >100 | >100 | — | — | — |
| 142 | >100 | >100 | — | — | — |
| 143 | >100 | 25-50 | — | — | — |
| 144 | >100 | 25-50 | — | — | — |
| 145 | >100 | >100 | — | — | — |
| 146 | 50-100 | 25-50 | — | — | — |
| 147 | >100 | >100 | — | — | — |
| 148 | 50-100 | 25-50 | — | — | — |
| 149 | >100 | 50-100 | — | — | — |
| 150 | >20 | — | >20 | >20 | >20 |
| 172 | >20 | — | >20 | >20 | >20 |
| 173 | >20 | — | >20 | >20 | >20 |
| 174 | >20 | — | >20 | >20 | >20 |
| 175 | 10-20 | — | 10-20 | 10-20 | 10-20 |
| 176 | >20 | — | >20 | >20 | >20 |
| 177 | 1-10 | — | 1-10 | 1-10 | 1-10 |
| 179 | 10-20 | — | 10-20 | 10-20 | 10-20 |
| 183 | >20 | — | >20 | >20 | >20 |
| 184 | >20 | — | >20 | >20 | >20 |
| 185 | 50 | — | — | — | — |
| 186 | 25 | — | — | — | — |
| 188 | <1 | 2 | — | — | — |
| 189 | 2.5 | 5 | — | — | — |
| 190 | 50 | 50 | — | — | — |
| 191 | 1-10 | 1-10 | — | — | — |
| 192 | 10-25 | 1-10 | — | — | — |
| 194 | 1-10 | 1-10 | — | — | — |
| 195 | 50-100 | 50-100 | — | — | — |
| 196 | 50-100 | 50-100 | — | — | — |
| 197 | 50-100 | 50-100 | — | — | — |
| 198 | 50-100 | 50-100 | — | — | — |
| 199 | >100 | 50-100 | — | — | — |
| 200 | >100 | 50-100 | — | — | — |
| 201 | >100 | — | — | — | — |
| 202 | 50-100 | 10-25 | — | — | — |
| 203 | >100 | — | — | — | — |
| 204 | >100 | 50-100 | — | — | — |
| 205 | 50-100 | 25-50 | — | — | — |
| 206 | >100 | >100 | — | — | — |
| 207 | 50-100 | 50-100 | — | — | — |
| 208 | 50-100 | 25-50 | — | — | — |
| 209 | >100 | 50-100 | — | — | — |
| 210 | 25-50 | 1-10 | — | — | — |
| 211 | 1-10 | 1-10 | — | — | — |
| 212 | 25-50 | 25-50 | — | — | — |
| 213 | 25-50 | 25-50 | — | — | — |
| 214 | 50-100 | 25-50 | — | — | — |
| 215 | 25-50 | 10-25 | — | — | — |
| 216 | 50-100 | 25-50 | — | — | — |
| 217 | 50-100 | 25-50 | — | — | — |
| 218 | >100 | 50-100 | — | — | — |
| 219 | 50-100 | 50-100 | — | — | — |
| 220 | 50-100 | 10-25 | — | — | — |
| 221 | 1-10 | <1 | — | — | — |
| 222 | 25-50 | 25-50 | — | — | — |
| 223 | >100 | >100 | — | — | — |
| 224 | 50-100 | 50-100 | — | — | — |
| 225 | 50-100 | 50-100 | — | — | — |
| 226 | 25-50 | 25-50 | — | — | — |
| 227 | 50-100 | 25-50 | — | — | — |
| 228 | 25-50 | 25-50 | — | — | — |
| 229 | 50-100 | 25-50 | — | — | — |
| 230 | 25-50 | 10-25 | — | — | — |
| 231 | 50-100 | 25-50 | — | — | — |
| 232 | 50-100 | 25-50 | — | — | — |
| 233 | >100 | 50-100 | — | — | — |
| 234 | 1-10 | <1 | — | — | — |
| 235 | 25-50 | 10-25 | — | — | — |
| 236 | >100 | 50-100 | — | — | — |
| 237 | >100 | >100 | — | — | — |
| 238 | >100 | 50-100 | — | — | — |
| 239 | 50-100 | 10-25 | — | — | — |
| 240 | 50-100 | 50-100 | — | — | — |
| 241 | >100 | 50-100 | — | — | — |
| 242 | 50-100 | 50-100 | — | — | — |
| 243 | 10-25 | 10-25 | — | — | — |
| 244 | >100 | >100 | — | — | — |
| 245 | 50-100 | 50-100 | — | — | — |
| 246 | >100 | >100 | — | — | — |
| 247 | >100 | >100 | — | — | — |
| 248 | >100 | 50-100 | — | — | — |
| 249 | >100 | >100 | — | — | — |
| 250 | >100 | 50-100 | — | — | — |
| 251 | >100 | >100 | — | — | — |
| 252 | 25-50 | 10-25 | — | — | — |
| 253 | 50-100 | 25-50 | — | — | — |
| 254 | 50-100 | 50-100 | — | — | — |
| 255 | 25-50 | 25-50 | — | — | — |
| 256 | 25-50 | 1-10 | — | — | — |
| 257 | 50-100 | 10-25 | — | — | — |
| 258 | >100 | 50-100 | — | — | — |
| 259 | >100 | 50-100 | — | — | — |
| 260 | 50-100 | 25-50 | — | — | — |
| 261 | >100 | 50-100 | — | — | — |
| 262 | 25-50 | 10-25 | — | — | — |
| 263 | <1 | <1 | — | — | — |
| 264 | >100 | >100 | — | — | — |
| 265 | 50-100 | 25-50 | — | — | — |
| 266 | >100 | 50-100 | — | — | — |
| 267 | 50-100 | 50-100 | — | — | — |
| 268 | >100 | 50-100 | — | — | — |
| 269 | 1-10 | 1-10 | — | — | — |
| 270 | >100 | >100 | — | — | — |
| 271 | >100 | >100 | — | — | — |
| 272 | >100 | >100 | — | — | — |
| 273 | >100 | >100 | — | — | — |
| 274 | 50-100 | 50-100 | — | — | — |
| 275 | >100 | 50-100 | — | — | — |
| 276 | >100 | >100 | — | — | — |
| 277 | 10-25 | 10-25 | — | — | — |
| 278 | 50-100 | 50-100 | — | — | — |
| 279 | 25-50 | 10-25 | — | — | — |
| 280 | >100 | 50-100 | — | — | — |
| 281 | >100 | >100 | — | — | — |
| 282 | 50-100 | 50-100 | — | — | — |
| 283 | >100 | >100 | — | — | — |
| 284 | >100 | 25-50 | — | — | — |
| 285 | >100 | 50-100 | — | — | — |
| 286 | 50-100 | 50-100 | — | — | — |
| 287 | >100 | >100 | — | — | — |
| 288 | 50-100 | 25-50 | — | — | — |
| 289 | >100 | 50-100 | — | — | — |
| 290 | 50-100 | 25-50 | — | — | — |
| 291 | 50-100 | 50-100 | — | — | — |
| 292 | >100 | 50-100 | — | — | — |
| 293 | >100 | >100 | — | — | — |
| 294 | — | <1 | — | — | — |

All references cited herein are incorporated by reference. The present invention may be embodied in other specific forms without departing from the spirit or essential attributes thereof and, accordingly, reference should be made to the appended claims, rather than to the foregoing specification, as indication the scope of the invention.

1. A compound of formula I, or a salt thereof: 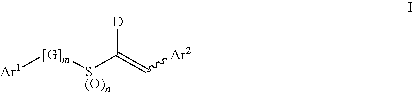
wherein:
Ar¹ is: 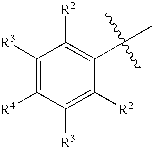
Ar² is selected from the group consisting of unsubstituted aryl, substituted aryl, unsubstituted heteroaryl and substituted heteroaryl; wherein, when Ar² is substituted aryl or substituted heteroaryl, said substituted aryl or substituted heteroaryl is aryl or heteroaryl substituted with one or more substituents independently selected from the group consisting of halogen, (C₁-C₆)alkyl, (C₂-C₆)alkenyl, (C₂-C₆)alkynyl, —(C₁-C₃)alkylene-Ar³, —OR⁷, —OAr³, —O(C₁-C₃)alkylene-Ar³, (C₁-C₃)fluoroalkoxy, —NO₂, —C≡N, —C(═O)(C₁-C₃)alkyl, —C(═O)OR⁷, —C(═O)NR⁸₂, —C(═NR⁷)NR⁸₂, —(C₁-C₃)alkylene-C(═O)OR⁷, —O(C₁-C₃)alkylene-C(═O)OR⁷, —(C₁-C₆)alkylene-OR⁷, —NR⁸₂, —P(═O)(OR⁷)₂, —OP(═O)(OR⁷)₂, —S(C₁-C₆)alkyl, —S(O)(C₁-C₆)alkyl —SO₂(C₁-C₆)alkyl, —SO₂NR⁸₂, —OSO₂(C₁-C₆)alkyl, —OSO₂Ar³, —NHC(═O)(C₁-C₆)alkyl, —OC(═O)(C₁-C₃)alkyl, —O(C₂-C₆)alkylene-NR⁸₂ and (C₁-C₃)perfluoroalkyl;
D is —C≡N, —C(═O)NR⁸₂, or NO₂;
G is CR¹₂ or NR¹;
R¹ is independently selected from the group consisting of hydrogen and (C₁-C₆)alkyl;
each R¹ is independently selected from the group consisting of hydrogen, (C₁-C₆)alkyl, halogen, —OR⁷, —OAr³, —O(C₁-C₃)alkylene-Ar³, —C≡N, —NO₂, —C(═O)OR⁷, —C(═O)NR⁸₂, —C(═NR⁷)NR⁸₂, —(C₁-C₃)alkylene-C(═O)OR⁷, —O(C₁-C₃)alkylene-C(═O)OR⁷, —(C₁-C₆)alkylene-OR⁷, —P(═O)(OR⁷)₂, —OP(═O)(OR⁷)₂, —NHC(═O)(C₁-C₆)alkyl, —SO₂NR⁸₂, —OSO₂(C₁-C₆)alkyl, —OSO₂Ar³, —OC(═O)(C₁-C₃)alkyl, —O(C₂-C₆)alkylene-NR⁸₂, (C₁-C₃)perfluoroalkyl and —N(R⁶)-(M)_{y}-R⁵;
each R³ is independently selected from the group consisting of hydrogen, (C₁-C₆)alkyl, halogen, —OR⁷, —OAr³, —O(C₁-C₃)alkylene-Ar³, —C≡N, —NO₂, —C(═O)OR⁷, —C(═O)NR⁸₂, —C(═NR⁷)NR⁸₂, —(C₁-C₃)alkylene-C(═O)OR⁷, —O(C₁-C₃)alkylene-C(═O)OR⁷, —(C₁-C₆)alkylene-OR⁷, —P(═O)(OR⁷)₂, —OP(═O)(OR⁷)₂—NHC(═O)(C₁-C₆)alkyl, —SO₂NR⁸₂, —OSO₂(C₁-C₆)alkyl, —OSO₂Ar³, —OC(═O)(C₁-C₃)alkyl, —O(C₂-C₆)alkylene-NR⁸₂, (C₁-C₃)perfluoroalkyl and —N(R⁶)-(M)_{y}-R⁵;
R⁴ is selected from the group consisting of hydrogen, (C₁-C₆)alkyl, halogen, —OR⁷, —OAr³, —O(C₁-C₃)alkylene-Ar³, —C≡N, —NO₂, —C(═O)OR⁷, —C(═O)NR⁸₂, C(═NR⁷)NR⁸—(C₁-C₃)alkylene-C(═O)OR⁷, —O(C₁-C₃)alkylene-C(═O)OR⁷, —(C₁-C₆)alkylene-OR⁷, —P(═O)(OR⁷)₂, —OP(═O)(OR⁷)₂, —NHC(═O)(C₁-C₆)alkyl, —SO₂NR⁸₂, —OSO₂(C₁-C₆)alkyl, —OSO₂Ar³, —OC(═O)(C₁-C₃)alkyl, —O(C₂-C₆)alkylene-NR⁸₂, (C₁-C₃)perfluoroalkyl and —N(R⁶)M (R⁵);
each Ar³ is phenyl optionally substituted with 1, 2, 3, 4, or 5 substituents independently selected from the group consisting of (C₁-C₃)alkyl, (C₁-C₃)alkoxy and halogen;
each M is a connecting group independently selected from the group consisting of —(C₁-C₆)alkylene-, —(CH₂)_{d}—V—(CH₂)ₑ—, —(CH₂)^{f}—W—(CH₂)_{g}— and -Z-;
each y is independently selected from the group consisting of 0 and 1;
each V is independently selected from the group consisting of arylene, heteroarylene, —C(═O)—, —C(═O)(C₁-C₆)perfluoroalkylene, —C(═O)—, —C(═S)—, —S(═O)—, —SO₂—, —C(═O)NR⁷—, —C(═S)NR⁷— and —SO₂NR⁷—;
each W is independently selected from the group consisting of —NR⁷—, —O— and —S—;
each d is independently selected from the group consisting of 0, 1 and 2;
each e is independently selected from the group consisting of 0, 1 and 2;
each f is independently selected from the group consisting of 1, 2 and 3;
each g is independently selected from the group consisting of 0, 1 and 2;
-Z- is 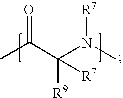
wherein the absolute configuration of -Z- is (S) or (R), or a mixture of (S) and (R);
each R⁵ is independently selected from the group consisting of hydrogen, unsubstituted aryl, substituted aryl, unsubstituted heterocyclic, substituted heterocyclic, —C(═O)OR⁷, —C(═O)NR⁸₂, —C(═NH)—NR⁸₂, —(C₁-C₆)perfluoroalkyl, —CF₂Cl, —P(═O)(OR⁷)₂, —CR⁷R¹⁰R¹¹ and a monovalent peptidyl group with a molecular weight of less than 1000, provided that when y is 0, R⁵ is not —C(═O)OH; and
each R⁶ is independently selected from the group consisting of —H, —(C₁-C₆)alkyl and aryl(C₁-C₃)alkyl; or
optionally, within any occurrence of —N(R⁶)M_{y}(R⁵) where y is 1, independently of any other occurrence of —N(R⁶)M_{y}(R⁵), R⁵ and R⁶ in combination represent a single bond and M is selected such that the resulting —N(R⁶)M_{y}(R⁵) moiety represents a 5, 6, or 7-membered ring heterocycle;
each R⁷ is independently selected from the group consisting of hydrogen and (C₁-C₆)alkyl;
each R⁸ is independently hydrogen or (C₁-C₆)alkyl; or, optionally, within any occurrence of NR⁸₂, independently of any other occurrence of NR⁸₂, two R⁸ groups in combination are —(CH₂)ₕ— or —(CH₂)ᵢX(CH₂)₂—;
wherein:
h is 4, 5, or 6;
i is 2 or 3;
X is O, S, NR⁷, or a single bond;
each R⁹ is independently selected from the group consisting of —H, —(C₁-C₆)alkyl, —(CH₂)₃—NH—C(NH₂)(═NH), —CH₂C(═O)NH₂, —CH₂C(═O)OH, —CH₂SH, —(CH₂)₂C(═O)—NH₂, —(CH₂)₂C(═O)OH, —CH₂-(2-imidazolyl), —(CH₂)₄—NH₂, —(CH₂)₂—S—CH₃, phenyl, CH₂-phenyl, —CH₂—OH, —CH(OH)—CH₃, —CH₂-(3-indolyl) and —CH₂-(4-hydroxyphenyl);
each R¹⁰ is independently selected from the group consisting of —H, —(C₁-C₆)alkyl, —C(═O)OR⁷, —C(═O)NR⁸₂, —OR⁷, —SR⁷, —OC(═O)(CH₂)₂C(═O)OR⁷ guanidino, —NR⁷₂, —NR⁷₃⁺, —N⁺(CH₂CH₂OR⁷)₃, phenyl, substituted phenyl, heterocyclyl and substituted heterocyclyl;
each R¹¹ is independently selected from the group consisting of R⁹, halogen, —NR⁸₂ and heterocycles containing two nitrogen atoms;
wherein the substituents for the substituted aryl and substituted heterocyclic groups comprising or included within R⁵ and R¹⁰ are independently selected from the group consisting of halogen, (C₁-C₆)alkyl, —OR⁷, —NO₂, —C≡N, —C(═O)OR⁷, —C(═O)NR⁸₂, —C(═NR⁷)NR⁸₂, —(C₁-C₃)alkylene-C(═O)OR⁷, —O(C₁-C₃)alkylene-C(═O)OR⁷, —(C₁-C₆)alkylene-OR⁷, —NR⁸₂, —P(═O)(OR⁷)₂, —OP(═O)(OR⁷)₂, —SO₂NR⁸₂, —NHC(═O)(C₁-C₆)alkyl, —OC(═O)(C₁-C₃)alkyl, —O(C₂-C₆)alkylene-NR⁸₂ and (C₁-C₃)perfluoroalkyl;
m is 0 or 1, provided that if D is —C≡N, then m is 1;
n is 0, 1, or 2, provided that if G is NR¹ then n is 2; [symbol]
indicates a single bond, whereby the configuration of the S—C═C—Ar² double bond may be either E or Z;
with the provisos that:
(i) if Ar¹ is unsubstituted phenyl, D is CN, G is CH₂, m is 1 and n is 2, then Ar² is other than unsubstituted phenyl;
(ii) if Ar¹ is 4-chlorophenyl, D is CN, G is CH₂, m is 1 and n is 2, then Ar² is other than 3,5-di-(t-butyl)-4-hydroxyphenyl;
(iii) if Ar¹ is unsubstituted phenyl, D is CN, G is NH, m is 1 and n is 2, then Ar² is other than 4-chlorophenyl;
(iv) if Ar¹ is unsubstituted phenyl, D is CN, G is CH₂, m is 1, n is 2 and Ar² is substituted phenyl, then Ar² is substituted at the 4-position by other than hydroxyl;
(v) if Ar¹ is 3-trifluoromethoxyphenyl, D is CN, G is CH₂, m is 1, n is 2 and Ar² is substituted phenyl, then Ar² is substituted at the 4-position by other than hydroxyl;
(vi) if D is C(═O)NH₂, m is 0 and n is 2, then Ar¹ is other than unsubstituted phenyl; and
(vii) if Ar² is unsubstituted phenyl, D is C(═O)NH₂, m is 0 and n is 2, then R⁴ is other than NH₂, NHCHO or NHC(═O)alkyl.

2. A compound according to claim 1, or a salt thereof, wherein the configuration of the double bond in the S—C═C—Ar² moiety is E.

3. A compound according to claim 2, or a salt thereof, wherein m is 1.

4. A compound according to claim 2, or a salt thereof, wherein n is 2.

5. A compound according to claim 2, or a salt thereof, wherein Ar² is substituted or unsubstituted phenyl.

6. A compound according to claim 5, or a salt thereof, provided that when Ar² is substituted phenyl, Ar² is substituted at the 4-position by other than hydroxy.

7. A compound according to claim 6, or a salt thereof, wherein Ar² is substituted phenyl wherein the substituents of Ar² are independently selected from the group consisting of halogen, (C₁-C₆)alkoxy, —OAr³ and —O(C₁-C₃)alkylene-Ar³.

8. A compound according to claim 7, or a salt thereof, wherein Ar² is selected from the group consisting of phenyl substituted with one, two or three (C₁-C₆)alkoxy groups and phenyl substituted with one, two, three, four, or five halogen atoms.

9. A compound according to claim 8, or a salt thereof, wherein Ar² is selected from the group 2,4,6-trimethoxyphenyl and 2,3,4,5,6-pentafluorophenyl.

10. A compound according to claim 2, or a salt thereof, wherein each occurrence of both R² and R³ is other than (C₁-C₃)perfluoroalkyl and R⁴ is other than (C₁-C₃)perfluoroalkyl.

11. A compound according to claim 10, or a salt thereof, wherein Ar¹ is mono- or di-substituted phenyl wherein the substituents of Ar¹ are independently selected from the group consisting of (C₁-C₆)alkyl, halogen, —OR⁷, —OAr¹, —O(C₁-C₃)alkylene-Ar³, —OSO₂(C₁-C₆)alkyl, —OSO₂Ar¹ and —N(R⁶)-(M)ₙ-R⁵.

12. A compound according to claim 11, or a salt thereof, wherein Ar¹ is: 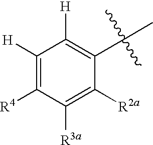
wherein:
R⁴ is selected from the group consisting of hydrogen, (C₁-C₆)alkyl, halogen, and —OR⁷;
R^{3a} is selected from the group consisting of hydrogen, —OR⁷, —OAr¹, —O(C₁-C₃)alkylene-Ar³, —OSO₂(C₁-C₆)alkyl, —OSO₂Ar³ and —N(R⁶)-(M)ₙ-R⁵;
R^{2a} is selected from the group consisting of hydrogen, (C₁-C₆)alkyl, halogen, —OR⁷, —OAr³, —O(C₁-C₃)alkylene-Ar³, —OSO₂(C₁-C₆)alkyl and —OSO₂Ar³; provided:
at least one of R^{2a}, R^{3a} and R⁴ is other than hydrogen; and
at least one of R^{2a} and R^{3a} is hydrogen.

13. A compound according to claim 12, or a salt thereof, wherein
R⁴ is halogen or —OR⁷;
R^{3a} is selected from the group consisting of hydrogen, —OR⁷ and —N(R⁶)-(M)_{y}-R⁵; and
R^{2a} is hydrogen or halogen.

14. A compound according to claim 12, or a salt thereof, wherein R^{2a} and R^{3a} are both hydrogen and R⁴ is halogen or —O(C₁-C₆)alkyl.

15. A compound according to claim 7, or a salt thereof, wherein Ar¹ is mono- or di-substituted phenyl wherein the substituents of Ar¹ are independently selected from the group consisting of (C₁-C₆)alkyl, halogen, —OR⁷, —OAr³, —O(C₁-C₃)alkylene-Ar³, —OSO₂(C₁-C₆)alkyl, —OSO₂Ar³ and —N(R⁶)-(M)_{y}-R⁵.

16. A compound according to claim 15, or a salt thereof, wherein Ar¹ is: 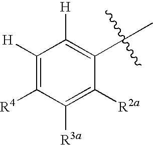
wherein:
R⁴ is selected from the group consisting of hydrogen, (C₁-C₆)alkyl, halogen and —OR⁷;
R^{3a} is selected from the group consisting of hydrogen, —OR⁷, —OAr³, —O(C₁-C₃)alkylene-Ar³, —OSO₂(C₁-C₆)alkyl, —OSO₂Ar³ and —N(R⁶)-(M)_{y}-R⁵;
R^{2a} is selected from the group consisting of hydrogen, (C₁-C₆)alkyl, halogen, —OR⁷, —OAr³, —O(C₁-C₃)alkylene-Ar³, —OSO₂(C₁-C₆)alkyl and —OSO₂Ar³; provided:
at least one of R^{2a}, R^{3a} and R⁴ is other than hydrogen; and at least one of R^{2a} and R^{3a} is hydrogen.

17. A compound according to claim 16, or a salt thereof, wherein
R⁴ is halogen or —OR⁷;
R^{3a} is selected from the group consisting of hydrogen, —OR⁷, and —N(R⁶)-(M)_{y}-R⁵; and
R^{2a} is hydrogen or halogen.

18. A compound according to claim 17, or a salt thereof, wherein Ar² is selected from the group consisting of phenyl substituted with one, two or three (C₁-C₆)alkoxy groups and phenyl substituted with one, two, three, four, or five halogen atoms.

19. A compound according to claim 18, or a salt thereof, wherein R^{2a} and R^{3a} are both hydrogen and R⁴ is halogen or —O(C₁-C₆)alkyl.

20. A compound according to claim 18, or a salt thereof, wherein R^{2a} is hydrogen, R^{3a} is —N(R⁶)-(M)_{y}-R⁵, and R⁴ is —O(C₁-C₆)alkyl.

21. A compound according to claim 2, or a salt thereof, wherein R¹ is H.

22. A compound according to claim 2, or a salt thereof, wherein D is —C≡N, G is CR¹₂ μm is 1, and n is 2.

23. A compound according to claim 22, or a salt thereof, wherein G is CH₂.

24. A compound according to claim 22, or a salt thereof, wherein Ar² is substituted or unsubstituted phenyl.

25. A compound according to claim 24, or a salt thereof, wherein G is CH₂.

26. A compound according to claim 24, or a salt thereof, provided that when Ar² is substituted phenyl, Ar² is substituted at the 4-position by other than hydroxy.

27. A compound according to claim 26, or a salt thereof, wherein G is CH₂.

28. A compound according to claim 26, or a salt thereof, wherein Ar² is substituted phenyl wherein the substituents of Ar² are independently selected from the group consisting of halogen, (C₁-C₆)alkoxy, —OAr³ and —O(C₁-C₃)alkylene-Ar³.

29. A compound according to claim 28, or a salt thereof, wherein G is CH₂.

30. A compound according to claim 28, or a salt thereof, wherein Ar² is selected from the group consisting of phenyl substituted with one, two or three (C₁-C₆)alkoxy groups and phenyl substituted with one, two, three, four, or five halogen atoms.

31. A compound according to claim 30, or a salt thereof, wherein G is CH₂.

32. A compound according to claim 22, or a salt thereof, wherein each occurrence of both R² and R³ is other than (C₁-C₃)perfluoroalkyl and R⁴ is other than (C₁-C₃)perfluoroalkyl.

33. A compound according to claim 32, or a salt thereof, wherein G is CH₂.

34. A compound according to claim 32, or a salt thereof, wherein Ar¹ is mono- or di-substituted phenyl wherein the substituents of Ar¹ are independently selected from the group consisting of (C₁-C₆)alkyl, halogen, —OR⁷, —OAr³, —O(C₁-C₃)alkylene-Ar³, —OSO₂(C₁-C₆)alkyl, —OSO₂Ar³ and —N(R⁶)-(M)_{y}-R⁵.

35. A compound according to claim 34, or a salt thereof, wherein G is CH₂.

36. A compound according to claim 34, or a salt thereof, wherein Ar¹ is: 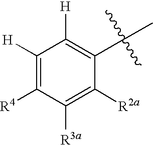
wherein:
R⁴ is selected from the group consisting of hydrogen, (C₁-C₆)alkyl;
halogen; and —OR⁷;
R^{3a} is selected from the group consisting of hydrogen, —OR⁷, —OAr⁷; —O(C₁-C₃)alkylene-Ar³, —OSO₂(C₁-C₆)alkyl, —OSO₂Ar³ and —N(R⁶)-(M)-R⁵;
R^{2a} is selected from the group consisting of hydrogen, (C₁-C₆)alkyl, halogen, —OR⁷, —OAr³, —O(C₁-C₃)alkylene-Ar³, —OSO₂(C₁-C₆)alkyl and —OSO₂Ar³; provided:
at least one of R^{2a}, R^{3a} and R⁴ is other than hydrogen; and
at least one of R^{2a} and R³ is hydrogen.

37. A compound according to claim 36, or a salt thereof, wherein G is CH₂.

38. A compound according to claim 36, or a salt thereof, wherein
R⁴ is halogen or —OR⁷;
R^{3a} is selected from the group consisting of hydrogen, —OR⁷ and —N(R⁶)-(M)_{y}-R⁵; and
R^{2a} is hydrogen or halogen.

39. A compound according to claim 38, or a salt thereof, wherein G is CH₂.

40. A compound according to claim 38, or a salt thereof, wherein R^{2a} and R^{3a} are both hydrogen and R⁴ is halogen or —O(C₁-C₆)alkyl.

41. A compound according to claim 40, or a salt thereof, wherein G is CH₂.

42. A compound according to claim 28, or a salt thereof, wherein Ar¹ is mono- or di-substituted phenyl wherein the substituents of Ar¹ are independently selected from the group consisting of (C₁-C₆)alkyl; halogen; —OR⁷; —OAr³, —O(C₁-C₃)alkylene-Ar³; —OSO₂(C₁-C₆)alkyl; —OSO₂Ar³; and —N(R⁶)-(M)_{y}-R⁵.

43. A compound according to claim 42, or a salt thereof, wherein G is CH₂.

44. A compound according to claim 42, or a salt thereof, wherein Ar¹ is: 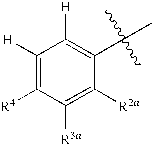
wherein:
R⁴ is selected from the group consisting of hydrogen, (C₁-C₆)alkyl, halogen and —OR⁷;
R^{3a} is selected from the group consisting of hydrogen, —OR⁷, —OAr³, —O(C₁-C₃)alkylene-Ar³, —OSO₂(C₁-C₆)alkyl, —OSO₂Ar³ and —N(R⁶)-(M)_{y}-R⁵;
R^{2a} is selected from the group consisting of hydrogen, (C₁-C₆)alkyl, halogen, —OR⁷, —OAr³, —O(C₁-C₃)alkylene-Ar³, —OSO₂(C₁-C₆)alkyl and —OSO₂Ar³; provided:
at least one of R^{2a}, R^{3a} and R⁴ is other than hydrogen; and
at least one of R^{2a} and R^{3a} is hydrogen.

45. A compound according to claim 44, or a salt thereof, wherein G is CH₂.

46. A compound according to claim 44, or a salt thereof, wherein
R⁴ is halogen or —OR⁷;
R^{3a} is selected from the group consisting of hydrogen, —OR⁷ and —N(R⁶)-(M)_{y}-R⁵; and
R^{2a} is hydrogen or halogen.

47. A compound according to claim 46, or a salt thereof, wherein G is CH₂.

48. A compound according to claim 47, or a salt thereof, wherein Ar² is selected from the group consisting of phenyl substituted with one, two or three (C₁-C₆)alkoxy groups and phenyl substituted with one, two, three, four, or five halogen atoms.

49. A compound according to claim 48, or a salt thereof, wherein G is CH₂.

50. A compound according to claim 48, or a salt thereof, wherein R^{2a} and R^{3a} are both hydrogen and R⁴ is halogen or —O(C₁-C₆)alkyl.

51. A compound according to claim 50, or a salt thereof, wherein G is CH₂.

52. A compound according to claim 48, or a salt thereof, wherein R^{2a} is hydrogen, R^{3a} is —N(R⁶)-(M)_{y}-R⁵, and R⁴ is —O(C₁-C₆)alkyl.

53. A compound according to claim 52, or a salt thereof, wherein G is CH₂.

54. A compound according to claim 22, or a salt thereof, wherein Ar¹ is selected from the group consisting of 4-methoxyphenyl, 4-fluorophenyl, 4-chlorophenyl, 4-bromophenyl, 3,4-dimethoxyphenyl, 3-amino-4-methoxyphenyl; and 3-fluoro-4-methoxyphenyl.

55. A compound according to claim 54, or a salt thereof, wherein G is CH₂.

56. A compound according to claim 22, or a salt thereof, wherein Ar² is selected from the group 2,4,6-trimethoxyphenyl and 2,3,4,5,6-pentafluorophenyl.

57. A compound according to claim 56, or a salt thereof, wherein G is CH₂.

58. A compound according to claim 22, or a salt thereof, wherein Ar² is selected from the group consisting of 2,3,4,5,6-pentafluorophenyl; 2,3,4-trichlorophenyl; 2,3,4-trimethoxyphenyl; 2,4,5-trimethoxyphenyl; 2,4,6-trimethoxyphenyl; 2,4,6-trimethoxyphenyl; 2,4-dichlorophenyl; 2,5-dimethylphenyl; 2,6-dimethoxyphenyl; 2-benzoxazolon-5-yl; 2-benzoxazolon-6-yl; 2-benzyloxyphenyl; 2-chloro-4-fluorophenyl; 2-chloro-4-fluorophenyl; 2-chlorophenyl; 2-fluoro-4-cyanophenyl; 2-fluoro-4-methoxyphenyl; 2-fluoro-4-methoxyphenyl; 2-fluoro-4-methoxyphenyl; 2-fluoro-4-nitrophenyl; 3,4,5-trimethoxyphenyl; 3,4-dichlorophenyl; 3,4-dihydroxyphenyl; 3,4-dimethoxyphenyl; 3,4-dimethylphenyl; 3,5-difluorophenyl; 3,5-dimethoxyphenyl; 3,5-dimethylphenyl; 3-furyl; 3-hydroxy-4-methoxyphenyl; 3-indolyl; 3-indolyl; 3-methyl-2-furyl; 3-methyl-2-furyl; 3-methyl-2-thiophen-2-yl; 3-nitro-4-fluorophenyl; 4-(N,N-dimethylamino)phenyl 4-acetoxyphenyl; 4-aminophenyl; 4-bromophenyl; 4-chlorophenyl; 4-ethoxy-3-methoxyphenyl; 4-ethoxycarbonyl-3,5-dimethylpyrrol-2-yl; 4-fluoro-3-methylphenyl; 4-fluorophenyl; 4-methanesulfenyl; 4-methoxyphenyl; 5-bromo-3-indolyl; 5-chloro-3-indolyl and phenyl.

59. A compound according to claim 58, or a salt thereof, wherein G is CH₂.

60. A compound according to claim 58, or a salt thereof, wherein Ar² is selected from the group 2,4,6-trimethoxyphenyl and 2,3,4,5,6-pentafluorophenyl.

61. A compound according to claim 60, or a salt thereof, wherein G is CH₂.

62. A compound according to claim 22, selected from the group consisting of (E)-2-[(4-methoxyphenyl)methanesulfonyl]-3-(2,4,6-trimethoxyphenyl)acrylonitrile; (E)-2-[(4-methoxyphenyl)methanesulfonyl]-3-(3-nitro-4-fluorophenyl)acrylonitrile; (E)-2-[(4-methoxyphenyl)methanesulfonyl]-3-(4-aminophenyl)acrylonitrile; (E)-2-[(4-methoxyphenyl)methanesulfonyl]-3-(2,6-dimethoxyphenyl)acrylonitrile; (E)-2-[(4-methoxyphenyl)methanesulfonyl]-3-(2,3,4,5,6-pentafluorophenyl)acrylonitrile; (E)-2-[(4-methoxyphenyl)methanesulfonyl]-3-(2-chloro-4-fluorophenyl)acrylonitrile; (E)-2-[(4-bromophenyl)methanesulfonyl]-3-(2-fluoro-4-cyanophenyl)acrylonitrile; (E)-2-[(4-bromophenyl)methanesulfonyl]-3-(2,6-dimethoxyphenyl)acrylonitrile; (E)-2-[(4-bromophenyl)methanesulfonyl]-3-(2,3,4,5,6-pentafluorophenyl)acrylonitrile; (E)-2-[(4-bromophenyl)methanesulfonyl]-3-(2,4,6-trimethoxyphenyl)acrylonitrile; (E)-2-[(4-chlorophenyl)methanesulfonyl]-3-(2,3,4,5,6-pentafluorophenyl)acrylonitrile; (E)-2-[(4-methoxyphenyl)methanesulfonyl]-3-(2-benzoxazolon-5-yl)acrylonitrile; (E)-2-[(4-methoxyphenyl)methanesulfonyl]-3-(2-benzoxazolon-6-yl)acrylonitrile; (E)-2-[(4-chlorophenyl)methanesulfonyl]-3-(3,4-dihydroxyphenyl)acrylonitrile; (E)-2-[(4-methoxyphenyl)methanesulfonyl]-3-(3,4-dihydroxyphenyl)acrylonitrile; (E)-2-[(3,4-dimethoxyphenyl)methanesulfonyl]-3-(2,3,4,5,6-pentafluorophenyl)acrylonitrile; (E)-2-[(4-fluorophenyl)methanesulfonyl]-3-(2,3,4,5,6-pentafluorophenyl)acrylonitrile; (E)-2-[(3-nitro-4-methoxyphenyl)methanesulfonyl]-3-(2,3,4,5,6-pentafluorophenyl)acrylonitrile; (E)-2-[(3-amino-4-methoxyphenyl)methanesulfonyl]-3-(2,4,6-trimethoxyphenyl)acrylonitrile; (E)-2-[(4-fluorophenyl)methanesulfonyl]-3-(4-ethoxycarbonyl-3,5-dimethylpyrrol-2-yl)acrylonitrile; (E)-2-[(4-methoxy-3-(4-methylphenylsulfonyloxy)phenyl)methanesulfonyl]-3-(2,3,4,5,6-pentafluorophenyl)acrylonitrile; (E)-2-[(3-fluoro-4-methoxyphenyl)methanesulfonyl]-3-(2,4,6-trimethoxyphenyl)acrylonitrile; (E)-2-[(4-chlorophenyl)methanesulfonyl]-3-(2,5-dimethylphenyl)acrylonitrile; (E)-2-[(4-chlorophenyl)methanesulfonyl]-3-(2-chloro-4-fluorophenyl)acrylonitrile; (E)-2-[(4-chlorophenyl)methanesulfonyl]-3-(4-fluoro-3-methylphenyl)acrylonitrile; (E)-2-[(4-chlorophenyl)methanesulfonyl]-3-(4-methanesulfenyl)acrylonitrile; (E)-2-[(4-chlorophenyl)methanesulfonyl]-3-(3-hydroxy-4-methoxyphenyl)acrylonitrile; (E)-2-[(4-chlorophenyl)methanesulfonyl]-3-(4-bromophenyl)acrylonitrile; (E)-2-[(4-chlorophenyl)methanesulfonyl]-3-(4-fluorophenyl)acrylonitrile; (E)-2-[(4-chlorophenyl)methanesulfonyl]-3-(2-fluoro-4-nitrophenyl)acrylonitrile; (E)-2-[(4-chlorophenyl)methanesulfonyl]-3-(2-fluoro-4-methoxyphenyl)acrylonitrile; (E)-2-[(4-chlorophenyl)methanesulfonyl]-3-(2,4-dichlorophenyl)acrylonitrile; (E)-2-[(4-chlorophenyl)methanesulfonyl]-3-(3,4-dichlorophenyl)acrylonitrile; (E)-2-[(4-chlorophenyl)methanesulfonyl]-3-(2,4,6-trimethoxyphenyl)acrylonitrile; (E)-2-[(4-chlorophenyl)methanesulfonyl]-3-(3-ethoxy-4-hydroxyphenyl)acrylonitrile; (E)-2-[(4-chlorophenyl)methanesulfonyl]-3-(4-ethoxy-3-methoxyphenyl)acrylonitrile; (E)-2-[(4-chlorophenyl)methanesulfonyl]-3-(3,5-dimethoxyphenyl)acrylonitrile; (E)-2-[(4-chlorophenyl)methanesulfonyl]-3-(4-acetoxyphenyl)acrylonitrile; (E)-2-[(4-chlorophenyl)methanesulfonyl]-3-(4-(N,N-dimethylamino)phenyl)acrylonitrile; (E)-2-[(4-chlorophenyl)methanesulfonyl]-3-(4-hydroxy-3-nitrophenyl)acrylonitrile; (E)-2-[(4-chlorophenyl)methanesulfonyl]-3-(4-chlorophenyl)acrylonitrile; (E)-2-[(4-fluorophenyl)methanesulfonyl]-3-(4-(N,N-dimethylamino)phenyl)acrylonitrile; (E)-2-[(2,4-dichlorophenyl)methanesulfonyl]-3-(2-fluoro-4-methoxyphenyl)acrylonitrile; (E)-2-[(4-chlorophenyl)methanesulfonyl]-3-(3,4-dimethylphenyl)acrylonitrile; (E)-2-[(4-chlorophenyl)methanesulfonyl]-3-(3-indolyl)acrylonitrile; (E)-2-[(2,4-dichlorophenyl)methanesulfonyl]-3-(5-bromo-3-indolyl)acrylonitrile; (E)-2-[(4-chlorophenyl)methanesulfonyl]-3-(5-chloro-3-indolyl)acrylonitrile; (E)-2-[(4-chlorophenyl)methanesulfonyl]-3-(3-ethoxy-4-hydroxyphenyl)acrylonitrile; (E)-2-[(2,4-dichlorophenyl)methanesulfonyl]-3-(5-chloro-3-indolyl)acrylonitrile; (E)-2-[(2,4-dichlorophenyl)methanesulfonyl]-3-(4-methanesulfenyl)acrylonitrile; (E)-2-[(4-fluorophenyl)methanesulfonyl]-3-(5-chloro-3-indolyl)acrylonitrile; (E)-2-[(4-fluorophenyl)methanesulfonyl]-3-(5-bromo-3-indolyl)acrylonitrile; (E)-2-[(4-fluorophenyl)methanesulfonyl]-3-(2,4,6-trimethoxyphenyl)acrylonitrile; (E)-2-[(4-fluorophenyl)methanesulfonyl]-3-(3-hydroxy-4-methoxyphenyl)acrylonitrile; (E)-2-[(4-fluorophenyl)methanesulfonyl]-3-(3-indolyl)acrylonitrile; (E)-2-[(4-fluorophenyl)methanesulfonyl]-3-(2,4-dihydroxyphenyl)acrylonitrile; (E)-2-[(4-fluorophenyl)methanesulfonyl]-3-(4-acetoxyphenyl)acrylonitrile; (E)-2-[(4-fluorophenyl)methanesulfonyl]-3-(2,4-dichlorophenyl)acrylonitrile; (E)-2-[(4-fluorophenyl)methanesulfonyl]-3-(4-methanesulfenyl)acrylonitrile; (E)-2-[(4-fluorophenyl)methanesulfonyl]-3-(3,4-dimethylphenyl)acrylonitrile; (E)-2-[(4-fluorophenyl)methanesulfonyl]-3-(4-chlorophenyl)acrylonitrile; (E)-2-[(4-fluorophenyl)methanesulfonyl]-3-(2,5-dimethylphenyl)acrylonitrile; (E)-2-[(2,4-dichlorophenyl)methanesulfonyl]-3-(5-chloro-3-indolyl)acrylonitrile; (E)-2-[(2,4-dichlorophenyl)methanesulfonyl]-3-(2,4-dihydroxyphenyl)acrylonitrile; (E)-2-[(2,4-dichlorophenyl)methanesulfonyl]-3-(2,3,4-trimethoxyphenyl)acrylonitrile; (E)-2-[(2,4-dichlorophenyl)methanesulfonyl]-3-(4-ethoxy-3-methoxyphenyl)acrylonitrile; (E)-2-[(2,4-dichlorophenyl)methanesulfonyl]-3-(2-chloro-4-fluorophenyl)acrylonitrile; (E)-2-[(2,4-dichlorophenyl)methanesulfonyl]-3-(2,5-dimethylphenyl)acrylonitrile; (E)-2-[(2,4-dichlorophenyl)methanesulfonyl]-3-(4-bromophenyl)acrylonitrile; (E)-2-[(2,4-dichlorophenyl)methanesulfonyl]-3-(4-methoxyphenyl)acrylonitrile; (E)-2-[(4-bromophenyl)methanesulfonyl]-3-(3,5-dimethylphenyl)acrylonitrile; (E)-2-[(2,4-dichlorophenyl)methanesulfonyl]-3-(3-methyl-2-thiophen-2-yl)acrylonitrile; (E)-2-[(2,4-dichlorophenyl)methanesulfonyl]-3-(3-methyl-2-furyl)acrylonitrile; (E)-2-[(2,4-dichlorophenyl)methanesulfonyl]-3-(3-furyl)acrylonitrile; (E)-2-[(2,4-dichlorophenyl)methanesulfonyl]-3-(3,4,5-trimethoxyphenyl)acrylonitrile; (E)-2-[(2,4-dichlorophenyl)methanesulfonyl]-3-(2,5-dimethoxyphenyl)acrylonitrile; (E)-2-[(2,4-dichlorophenyl)methanesulfonyl]-3-(2,4,5-trimethoxyphenyl)acrylonitrile; (E)-2-[(2,4-dichlorophenyl)methanesulfonyl]-3-(3,4-dimethoxyphenyl)acrylonitrile; (E)-2-[(2,4-dichlorophenyl)methanesulfonyl]-3-(phenyl)acrylonitrile; (E)-2-[(4-chlorophenyl)methanesulfonyl]-3-(3-methyl-2-thiophen-2-yl)acrylonitrile; (E)-2-[(phenyl)methanesulfonyl]-3-(3-hydroxyphenyl)acrylonitrile; (E)-2-[(phenyl)methanesulfonyl]-3-(2-hydroxyphenyl)acrylonitrile; (E)-2-[(phenyl)methanesulfonyl]-3-(3-methyl-2-furyl)acrylonitrile; (E)-2-[(phenyl)methanesulfonyl]-3-(2-fluoro-4-methoxyphenyl)acrylonitrile; (E)-2-[(phenyl)methanesulfonyl]-3-(3,4,5-trimethoxyphenyl)acrylonitrile; (E)-2-[(phenyl)methanesulfonyl]-3-(3,4-dimethoxyphenyl)acrylonitrile; (E)-2-[(phenyl)methanesulfonyl]-3-(3,5-dimethoxyphenyl)acrylonitrile; (E)-2-[(phenyl)methanesulfonyl]-3-(2,5-dimethylphenyl)acrylonitrile; (E)-2-[(phenyl)methanesulfonyl]-3-(2-benzyloxyphenyl)acrylonitrile; (E)-2-[(phenyl)methanesulfonyl]-3-(4-hydroxy-3-nitrophenyl)acrylonitrile; (E)-2-[(phenyl)methanesulfonyl]-3-(3,5-difluorophenyl)acrylonitrile; (E)-2-[(4-bromophenyl)methanesulfonyl]-3-(2-chlorophenyl)acrylonitrile; (E)-2-[(4-methoxyphenyl)methanesulfonyl]-3-(2,3,4-chlorophenyl)acrylonitrile; (E)-2-[(4-methoxyphenyl)methanesulfonyl]-3-(3,4-trichlorophenyl)acrylonitrile; (E)-2-[(4-methoxyphenyl)methanesulfonyl]-3-(3,4-dimethoxyphenyl)acrylonitrile; (E)-2-[(4-methoxyphenyl)methanesulfonyl]-3-(3,5-dimethoxyphenyl)acrylonitrile; and salts thereof.

63. A compound according to claim 22 selected from the group consisting of (E)-2-[(4-methoxyphenyl)methanesulfonyl]-3-(2,4,6-trimethoxyphenyl)acrylonitrile; (E)-2-[(4-methoxyphenyl)methanesulfonyl]-3-(2,3,4,5,6-pentafluorophenyl)acrylonitrile; (E)-2-[(4-methoxyphenyl)methanesulfonyl]-3-(2-chloro-4-fluorophenyl)acrylonitrile; (E)-2-[(4-bromophenyl)methanesulfonyl]-3-(2-fluoro-4-cyanophenyl)acrylonitrile; (E)-2-[(4-methoxyphenyl)methanesulfonyl]-3-(2-fluoro-4-cyanophenyl)acrylonitrile; E)-2-[(4-bromophenyl)methanesulfonyl]-3-(2,6-dimethoxyphenyl)acrylonitrile; (E)-2-[(4-bromophenyl)methanesulfonyl]-3-(2,3,4,5,6-pentafluorophenyl)acrylonitrile; (E)-2-[(4-bromophenyl)methanesulfonyl]-3-(2,4,6-trimethoxyphenyl)acrylonitrile; (E)-2-[(4-chlorophenyl)methanesulfonyl]-3-(2,3,4,5,6-pentafluorophenyl)acrylonitrile; (E)-2-[(4-methoxyphenyl)methanesulfonyl]-3-(2-benzoxazolon-5-yl)acrylonitrile; (E)-2-[(4-chlorophenyl)methanesulfonyl]-3-(3, 4-4-dihydroxyphenyl)acrylonitrile; (E)-2-[(4-methoxyphenyl)methanesulfonyl]-3-(3,4-dihydroxyphenyl)acrylonitrile; (E)-2-[(3,4-dimethoxyphenyl)methanesulfonyl]-3-(2,3,4,5,6-pentafluorophenyl)acrylonitrile; (E)-2-[(4-fluorophenyl)methanesulfonyl]-3-(2,3,4,5,6-pentafluorophenyl)acrylonitrile; (E)-2-[(3-nitro-4-methoxyphenyl)methanesulfonyl]-3-(2,3,4,5,6-pentafluorophenyl)-acrylonitrile; (E)-2-[(3-amino-4-methoxyphenyl)methanesulfonyl]-3-(2,4,6-trimethoxyphenyl)acrylonitrile; (E)-2-[(3-fluoro-4-methoxyphenyl)methanesulfonyl]-3-(2,4,6-trimethoxyphenyl)acrylonitrile; (E)-2-[(4-chlorophenyl)methanesulfonyl]-3-(2,5-dimethylphenyl)acrylonitrile; (E)-2-[(4-chlorophenyl)methanesulfonyl]-3-(2-fluoro-4-nitrophenyl)acrylonitrile; (E)-2-[(4-fluorophenyl)methanesulfonyl]-3-(4-acetoxyphenyl)acrylonitrile; (E)-2-[(phenyl)methanesulfonyl]-3-(2,5-dimethylphenyl)acrylonitrile; (E)-2-[(4-chlorophenyl)methanesulfonyl]-3-(3,4-dichlorophenyl)acrylonitrile; (E)-2-[(4-chlorophenyl)methanesulfonyl]-3-(4-ethoxy-3-methoxyphenyl)acrylonitrile; (E)-2-[(4-chlorophenyl)methanesulfonyl]-3-(3-indolyl)acrylonitrile; (E)-2-[(4-chlorophenyl)methanesulfonyl]-3-(5-chloro-3-indolyl)acrylonitrile; (E)-2-[(4-fluorophenyl)methanesulfonyl]-3-(3,4-dimethylphenyl)acrylonitrile; (E)-2-[(2,4-dichlorophenyl)methanesulfonyl]-3-(5-chloro-3-indolyl)acrylonitrile; (E)-2-[(2,4-dichlorophenyl)methanesulfonyl]-3-(3,4,5-trimethoxyphenyl)acrylonitrile; (E)-2-[(phenyl)methanesulfonyl]-3-(3,5-dimethoxyphenyl)acrylonitrile and salts thereof.

64. A compound according to claim 2, wherein D is —C(═O)NR⁸₂, G is CR¹₂, m is 0 or 1, and n is 2.

65. A compound according to claim 60, wherein D is —C(═O)NH₂.

66. A compound according to claim 60, wherein G is CH₂.

67. A compound according to claim 61, wherein G is CH₂.

68. A compound according to claim 2, wherein D is —NO₂, G is CH₂ m is 0 or 1, and n is 2.

69. A compound according to claim 2, wherein D is —C≡N, G is CR¹₂, m is 1, and n is 1.

70. A compound according to claim 2, wherein D is —C(═O)NR⁸₂, G is CR¹₂, m is 0 or 1, and n is 1.

71. A compound according to claim 2, wherein D is —NO₂; G is CH₂ m is 0 or 1, and n is 1.

72. A compound according to claim 2, wherein D is —C≡N, G is CR¹₂, m is 1, and n is 0.

73. A compound according to claim 2, wherein D is —C(═O)NR⁸₂, G is CR¹₂, m is 0 or 1, and n is 0.

74. A compound according to claim 2, wherein D is —NO₂, G is CH₂ m is 0 or 1, and n is 0.

75. A compound according to claim 2, wherein D is —C≡N, G is NR₁, m is 1, and n is 2.

76. A compound according to claim 75, or a salt thereof, wherein G is NH.

77. A compound according to claim 75, or a salt thereof, wherein Ar² is substituted or unsubstituted phenyl.

78. A compound according to claim 75, or a salt thereof, wherein G is NH.

79. A compound according to claim 77, or a salt thereof, provided that when Ar² is substituted phenyl, Ar² is substituted at the 4-position by other than hydroxy.

80. A compound according to claim 79, or a salt thereof, wherein G is NH.

81. A compound according to claim 29, or a salt thereof, wherein Ar² is substituted phenyl wherein the substituents of Ar² are independently selected from the group consisting of halogen, (C₁-C₆)alkoxy, —OAr³ and —O(C₁-C₃)alkylene-Ar³.

82. A compound according to claim 81, or a salt thereof, wherein G is NH.

83. A compound according to claim 81, or a salt thereof, wherein Ar² is selected from the group consisting of phenyl substituted with one, two or three (C₁-C₆)alkoxy groups and phenyl substituted with one, two, three, four, or five halogen atoms.

84. A compound according to claim 83, or a salt thereof, wherein G is NH.

85. A compound according to claim 75, or a salt thereof, wherein each occurrence of both R² and R³ is other than (C₁-C₃)perfluoroalkyl and R⁴ is other than (C₁-C₃)perfluoroalkyl.

86. A compound according to claim 85, or a salt thereof, wherein G is NH.

87. A compound according to claim 85, or a salt thereof, wherein Ar¹ is mono- or di-substituted phenyl wherein the substituents of Ar¹ are independently selected from the group consisting of (C₁-C₆)alkyl, halogen, —OR⁷, —OAr³, —O(C₁-C₃)alkylene-Ar³, —OSO₂(C₁-C₆)alkyl, —OSO₂Ar³ and —N(R⁶)-(M)ₙ-R⁵.

88. A compound according to claim 87, or a salt thereof, wherein G is NH.

89. A compound according to claim 88, or a salt thereof, wherein Ar¹ is: 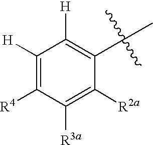
wherein:
R⁴ is selected from the group consisting of hydrogen, (C₁-C₆)alkyl, halogen and —OR⁷;
R^{3a} is selected from the group consisting of hydrogen, —OR⁷, —OAr³, —O(C₁-C₃)alkylene-Ar³, —OSO₂(C₁-C₆)alkyl, —OSO₂Ar³ and —N(R⁶)-(M)_{y}-R⁵;
R^{2a} is selected from the group consisting of hydrogen, (C₁-C₆)alkyl, halogen, —OR⁷, —OAr³, —O(C₁-C₃)alkylene-Ar³, —OSO₂(C₁-C₆)alkyl and —OSO₂Ar³; provided:
at least one of R^{2a}, R^{3a} and R⁴ is other than hydrogen; and
at least one of R^{2a} and R^{3a} is hydrogen.

90. A compound according to claim 89, or a salt thereof, wherein G is NH.

91. A compound according to claim 89, or a salt thereof, wherein
R⁴ is halogen or —OR⁷;
R^{3a} is selected from the group consisting of hydrogen, —OR⁷ and —N(R⁶)-(M)_{y}-R⁵; and
R^{2a} is hydrogen or halogen.

92. A compound according to claim 91 or a salt thereof, wherein G is NH.

93. A compound according to claim 91, or a salt thereof, wherein R^{2a} and R^{3a} are both hydrogen and R⁴ is halogen or —O(C₁-C₆)alkyl.

94. A compound according to claim 93, or a salt thereof, wherein G is NH.

95. A compound according to claim 81, or a salt thereof, wherein Ar¹ is mono- or di-substituted phenyl wherein the substituents of Ar¹ are independently selected from the group consisting of (C₁-C₆)alkyl, halogen, —OR⁷, —OAr³, —O(C₁-C₃)alkylene-Ar³, —OSO₂(C₁-C₆)alkyl, —OSO₂Ar³ and —N(R⁶)-(M)_{y}-R⁵.

96. A compound according to claim 95, or a salt thereof, wherein G is NH.

97. A compound according to claim 95, or a salt thereof, wherein Ar¹ is: 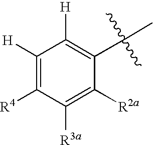
wherein:
R⁴ is selected from the group consisting of hydrogen, (C₁-C₆)alkyl, halogen and —OR⁷;
R^{3a} is selected from the group consisting of hydrogen, —OR⁷, —OAr³, —O(C₁-C₃)alkylene-Ar³, —OSO₂(C₁-C₆)alkyl, —OSO₂Ar¹ and —N(R⁶)-(M)_{y}-R⁵;
R^{2a} is selected from the group consisting of hydrogen, (C₁-C₆)alkyl, halogen, —OR⁷, —OAr³, —O(C₁-C₃)alkylene-Ar³, —OSO₂(C₁-C₆)alkyl and —OSO₂Ar³; provided:
at least one of R^{2a} and R^{3a} and R⁴ is other than hydrogen; and
at least one of R^{2a} and R^{3a} is hydrogen.

98. A compound according to claim 97, or a salt thereof, wherein G is NH.

99. A compound according to claim 97, or a salt thereof, wherein
R⁴ is halogen or —OR⁷;
R^{3a} is selected from the group consisting of hydrogen, —OR⁷ and —N(R⁶)-(M)_{y}-R⁵; and
R^{2a} is hydrogen or halogen.

100. A compound according to claim 99, or a salt thereof, wherein G is NH.

101. A compound according to claim 99, or a salt thereof, wherein Ar² is selected from the group consisting of phenyl substituted with one, two or three (C₁-C₆)alkoxy groups and phenyl substituted with one, two, three, four, or five halogen atoms.

102. A compound according to claim 101, or a salt thereof, wherein G is NH.

103. A compound according to claim 99, or a salt thereof, wherein R^{2a} and R^{3a} are both hydrogen and R⁴ is halogen or —O(C₁-C₆)alkyl.

104. A compound according to claim 103, or a salt thereof, wherein G is NH.

105. A compound according to claim 99, or a salt thereof, wherein R^{2a} is hydrogen, R^{3a} is —N(R⁶)-(M)_{y}-R⁵, and R⁴ is —O(C₁-C₆)alkyl.

106. A compound according to claim 105, or a salt thereof, wherein G is NH.

107. A compound according to claim 75, or a salt thereof, wherein Ar¹ is selected from the group consisting of 4-methoxyphenyl, 4-fluorophenyl, 4-chlorophenyl, 4-bromophenyl, 2-phenoxyphenyl, 2,4-dimethylphenyl, 4-methoxy-3-(4-methylsulfonyloxy)phenyl, 3,4-dimethoxyphenyl, 3-nitro-4-methoxyphenyl, 3-amino-4-methoxyphenyl, 3-fluoro-4-methoxyphenyl, and 2,4-dichlorophenyl.

108. A compound according to claim 107, or a salt thereof, wherein G is NH.

109. A compound according to claim 22, or a salt thereof, wherein Ar² is selected from the group consisting of 2,3,4,5,6-pentafluorophenyl; 2,3,4-trimethoxyphenyl; 2,3,5-trichlorophenyl; 2,4,5-trimethoxyphenyl; 2,4,6-trimethoxyphenyl; 2,4-difluorophenyl; 2,5-dimethoxyphenyl; 2,5-dimethylphenyl; 2,6-dimethoxyphenyl; 2-benzyloxyphenyl; 2-chlorophenyl; 2-fluoro-4-methoxyphenyl; 2-methoxyphenyl; 2-methoxyphenyl; 2-phenoxyphenyl; 3,4,5-trimethoxyphenyl; 3,4-dichlorophenyl; 3-chloro-4-fluorophenyl; 3-indolyl; 3-methylthiophen-2-yl; 3-methylthiophen-2-yl; 3-methylthiophen-2-yl; 3-nitro-4-hydroxyphenyl; 4-(N,N-dimethylamino)phenyl; 4-biphenyl-1-yl; 4-bromophenyl; 4-chlorophenyl; 4-ethoxy-3-methoxyphenyl; 4-fluorophenyl; 4-methoxyphenyl; 5-bromo-3-indolyl; and 5-methylthiophen-2-yl.

110. A compound according to claim 109, or a salt thereof, wherein G is NH.

111. A compound according to claim 109, or a salt thereof, wherein Ar² is selected from the group 2,4,6-trimethoxyphenyl and 2,3,4,5,6-pentafluorophenyl.

112. A compound according to claim 111, or a salt thereof, wherein G is NH.

113. A compound according to claim 75 selected from the group consisting of Other embodiments of the invention include the following compounds: (E)-2-[(4-methoxyphenyl)sulfamoyl]-3-(4-bromophenyl)acrylonitrile; (E)-2-[(4-methoxyphenyl)sulfamoyl]-3-(2,4,6-trimethoxyphenyl)acrylonitrile; (E)-2-[(4-methoxyphenyl)sulfamoyl]-3-(2,3,4,5,6-pentafluorophenyl)acrylonitrile; (E)-2-[(4-methoxyphenyl)sulfamoyl]-3-(2,6-dimethoxyphenyl)acrylonitrile; (E)-2-[(4-methoxy-3-(4-methylphenylsulfonyloxy)phenyl)sulfamoyl]-3-(2,3,4,5,6-pentafluorophenyl)acrylonitrile; (E)-2-[(4-chlorophenyl)sulfamoyl]-3-(4-methoxyphenyl)acrylonitrile; (E)-2-[(2-phenoxyphenyl)sulfamoyl]-3-(4-bromophenyl)acrylonitrile; (E)-2-[(2-phenoxyphenyl)sulfamoyl]-3-(3-indolyl)acrylonitrile; (E)-2-[(2-phenoxyphenyl)sulfamoyl]-3-(5-methylthiophen-2-yl)acrylonitrile; (E)-2-[(2-phenoxyphenyl)sulfamoyl]-3-(2-methoxyphenyl)acrylonitrile; (E)-2-[(4-methoxyphenyl)sulfamoyl]-3-(4-fluorophenyl)acrylonitrile; (E)-2-[(4-methoxyphenyl)sulfamoyl]-3-(2,5-dimethoxyphenyl)acrylonitrile; (E)-2-[(4-methoxyphenyl)sulfamoyl]-3-(2-methoxyphenyl)acrylonitrile; (E)-2-[(4-chlorophenyl)sulfamoyl]-3-(3-indolyl)acrylonitrile; (E)-2-[(2-phenoxyphenyl)sulfamoyl]-3-(4-chlorophenyl)acrylonitrile; (E)-2-[(2-phenoxyphenyl)sulfamoyl]-3-(2,4,6-trimethoxyphenyl)acrylonitrile; (E)-2-[(2-phenoxyphenyl)sulfamoyl]-3-(3-nitro-4-hydroxyphenyl)acrylonitrile; (E)-2-[(2-phenoxyphenyl)sulfamoyl]-3-(2-phenoxyphenyl)acrylonitrile; (E)-2-[(2-phenoxyphenyl)sulfamoyl]-3-(4-ethoxy-3-methoxyphenyl)acrylonitrile; (E)-2-[(4-bromophenyl)sulfamoyl]-3-(4-chlorophenyl)acrylonitrile; (E)-2-[(4-bromophenyl)sulfamoyl]-3-(2-benzyloxyphenyl)acrylonitrile; (E)-2-[(4-bromophenyl)sulfamoyl]-3-(3-indolyl)acrylonitrile; (E)-2-[(4-bromophenyl)sulfamoyl]-3-(2,4,6-trimethoxyphenyl)acrylonitrile; (E)-2-[(4-bromophenyl)sulfamoyl]-3-(4-(N,N-dimethylamino)phenyl)acrylonitrile; (E)-2-[(4-bromophenyl)sulfamoyl]-3-(4-bromophenyl)acrylonitrile; (E)-2-[(4-bromophenyl)sulfamoyl]-3-(2,3,4-trimethoxyphenyl)acrylonitrile; (E)-2-[(4-bromophenyl)sulfamoyl]-3-(4-ethoxy-3-methoxyphenyl)acrylonitrile; (E)-2-[(4-bromophenyl)sulfamoyl]-3-(4-biphenyl-1-yl)acrylonitrile; (E)-2-[(4-bromophenyl)sulfamoyl]-3-(2,3,4,5,6-pentafluorophenyl)acrylonitrile; (E)-2-[(4-bromophenyl)sulfamoyl]-3-(5-bromo-3-indolyl)acrylonitrile; (E)-2-[(4-bromophenyl)sulfamoyl]-3-(3-methylthiophen-2-yl)acrylonitrile; (E)-2-[(4-bromophenyl)sulfamoyl]-3-(2-methoxyphenyl)acrylonitrile; (E)-2-[(4-bromophenyl)sulfamoyl]-3-(2,4-difluorophenyl)acrylonitrile; (E)-2-[(4-bromophenyl)sulfamoyl]-3-(2-chlorophenyl)acrylonitrile; (E)-2-[(4-bromophenyl)sulfamoyl]-3-(2-hydroxyphenyl)acrylonitrile; (E)-2-[(4-bromophenyl)sulfamoyl]-3-(2,5-dimethylphenyl)acrylonitrile; (E)-2-[(4-fluorophenyl)sulfamoyl]-3-(2-benzyloxyphenyl)acrylonitrile; (E)-2-[(4-fluorophenyl)sulfamoyl]-3-(3-indolyl)acrylonitrile; (E)-2-[(4-fluorophenyl)sulfamoyl]-3-(2,4,6-trimethoxyphenyl)acrylonitrile; (E)-2-[(4-fluorophenyl)sulfamoyl]-3-(4-N,N-dimethylamino)phenyl)acrylonitrile; (E)-2-[(4-fluorophenyl)sulfamoyl]-3-(2,3,4,5,6-pentafluorophenyl)acrylonitrile; (E)-2-[(4-fluorophenyl)sulfamoyl]-3-(2-chlorophenyl)acrylonitrile; (E)-2-[(4-fluorophenyl)sulfamoyl]-3-(3-methylthiophen-2-yl)acrylonitrile; (E)-2-[(4-fluorophenyl)sulfamoyl]-3-(2,5-dimethylphenyl)acrylonitrile; (E)-2-[(4-fluorophenyl)sulfamoyl]-3-(4-biphenyl-1-yl)acrylonitrile; (E)-2-[(4-fluorophenyl)sulfamoyl]-3-(2,4-difluorophenyl)acrylonitrile; (E)-2-[(4-fluorophenyl)sulfamoyl]-3-(2-fluoro-4-methoxyphenyl)acrylonitrile; (E)-2-[(4-fluorophenyl)sulfamoyl]-3-(4-bromophenyl)acrylonitrile; (E)-2-[(4-fluorophenyl)sulfamoyl]-3-(2,3,5-trichlorophenyl)acrylonitrile; (E)-2-[(4-fluorophenyl)sulfamoyl]-3-(3-chloro-4-fluorophenyl)acrylonitrile; (E)-2-[(4-fluorophenyl)sulfamoyl]-3-(4-chlorophenyl)acrylonitrile; (E)-2-[(4-fluorophenyl)sulfamoyl]-3-(5-bromo-3-indolyl)acrylonitrile; (E)-2-[(2,4-dimethylphenyl)sulfamoyl]-3-(2-benzyloxyphenyl)acrylonitrile; (E)-2-[(2,4-dimethylphenyl)sulfamoyl]-3-(3-indolyl)acrylonitrile; (E)-2-[(2,4-dimethylphenyl)sulfamoyl]-3-(2,4,6-trimethoxyphenyl)acrylonitrile; (E)-2-[(2,4-dimethylphenyl)sulfamoyl]-3-(4-(N,N-dimethylamino)phenyl)acrylonitrile; (E)-2-[(2,4-dimethylphenyl)sulfamoyl]-3-(2-chlorophenyl)acrylonitrile; (E)-2-[(2,4-dimethylphenyl)sulfamoyl]-3-(3-methylthiophen-2-yl)acrylonitrile; (E)-2-[(2,4-dimethylphenyl)sulfamoyl]-3-(2,4,5-trimethoxyphenyl)acrylonitrile; (E)-2-[(2,4-dimethylphenyl)sulfamoyl]-3-(2,5-dimethylphenyl)acrylonitrile; (E)-2-[(2,4-dimethylphenyl)sulfamoyl]-3-(4-biphenyl-1-yl)acrylonitrile; (E)-2-[(2,4-dimethylphenyl)sulfamoyl]-3-(2-fluoro-4-methoxyphenyl)acrylonitrile; (E)-2-[(2,4-dimethylphenyl)sulfamoyl]-3-(4-bromophenyl)acrylonitrile; (E)-2-[(2,4-dimethylphenyl)sulfamoyl]-3-(2,3,5-trichlorophenyl)acrylonitrile; (E)-2-[(2,4-dimethylphenyl)sulfamoyl]-3-(5-methylthiophen-2-yl)acrylonitrile; (E)-2-[(2,4-dimethylphenyl)sulfamoyl]-3-(2-methoxyphenyl)acrylonitrile; (E)-2-[(2,4-dimethylphenyl)sulfamoyl]-3-(4-chlorophenyl)acrylonitrile; (E)-2-[(2,4-dimethylphenyl)sulfamoyl]-3-(3,4,5-trimethoxyphenyl)acrylonitrile; (E)-2-[(2,4-dimethylphenyl)sulfamoyl]-3-(5-bromo-3-indolyl)acrylonitrile; (E)-2-[(2,4-dimethylphenyl)sulfamoyl]-3-(3,4-dichlorophenyl)acrylonitrile; (E)-2-[(2,4-dimethylphenyl)sulfamoyl]-3-(3-ethoxy-4-hydroxyphenyl)acrylonitrile; (E)-2-[(4-methoxyphenyl)sulfamoyl]-3-(2-benzyloxyphenyl)acrylonitrile; (E)-2-[(4-methoxyphenyl)sulfamoyl]-3-(3-indolyl)acrylonitrile; (E)-2-[(4-methoxyphenyl)sulfamoyl]-3-(2,4,6-trimethoxyphenyl)acrylonitrile; (E)-2-[(4-methoxyphenyl)sulfamoyl]-3-(4-(N,N-dimethylamino)phenyl)acrylonitrile; (E)-2-[(4-methoxyphenyl)sulfamoyl]-3-(2,5-dimethylphenyl)acrylonitrile; (E)-2-[(4-methoxyphenyl)sulfamoyl]-3-(2,4,5-trimethoxyphenyl)acrylonitrile; (E)-2-[(4-methoxyphenyl)sulfamoyl]-3-(2,3,4-trimethoxyphenyl)acrylonitrile; (E)-2-[(4-methoxyphenyl)sulfamoyl]-3-(4-biphenyl-1-yl)acrylonitrile; (E)-2-[(4-methoxyphenyl)sulfamoyl]-3-(3,4-dichlorophenyl)acrylonitrile; (E)-2-[(4-methoxyphenyl)sulfamoyl]-3-(4-hydroxyphenyl)acrylonitrile; (E)-2-[(4-methoxyphenyl)sulfamoyl]-3-(4-chlorophenyl)acrylonitrile; (E)-2-[(4-methoxyphenyl)sulfamoyl]-3-(4-bromophenyl)acrylonitrile; (E)-2-[(4-methoxyphenyl)sulfamoyl]-3-(2-hydroxyphenyl)acrylonitrile; (E)-2-[(4-methoxyphenyl)sulfamoyl]-3-(2,3,5-trichlorophenyl)acrylonitrile and salts thereof; Preferred embodiments of the invention include the following compounds: (E)-2-[(4-methoxyphenyl)sulfamoyl]-3-(4-bromophenyl)acrylonitrile; (E)-2-[(4-methoxyphenyl)sulfamoyl]-3-(2,4,6-trimethoxyphenyl)acrylonitrile; (E)-2-[(4-methoxyphenyl)sulfamoyl]-3-(2,3,4,5,6-pentafluorophenyl)acrylonitrile; (E)-2-[(4-methoxy-3-(4-methylphenylsulfonyloxy)phenyl)sulfamoyl]-3-(2,3,4,5,6-pentafluorophenyl)acrylonitrile; (E)-2-[(2-phenoxyphenyl)sulfamoyl]-3-(2-phenoxyphenyl)acrylonitrile; (E)-2-[(2-phenoxyphenyl)sulfamoyl]-3-(4-ethoxy-3-methoxyphenyl)acrylonitrile; (E)-2-[(4-bromophenyl)sulfamoyl]-3-(2,4,6-trimethoxyphenyl)acrylonitrile; (E)-2-[(4-bromophenyl)sulfamoyl]-3-(4-biphenyl-1-yl)acrylonitrile; (E)-2-[(4-bromophenyl)sulfamoyl]-3-(2,3,4,5,6-pentafluorophenyl)acrylonitrile; (E)-2-[(4-fluorophenyl)sulfamoyl]-3-(2-benzyloxyphenyl)acrylonitrile; (E)-2-[(4-fluorophenyl)sulfamoyl]-3-(2,3,4,5,6-pentafluorophenyl)acrylonitrile; (E)-2-[(4-fluorophenyl)sulfamoyl]-3-(2-chlorophenyl)acrylonitrile; (E)-2-[(4-fluorophenyl)sulfamoyl]-3-(4-biphenyl-1-yl)acrylonitrile; (E)-2-[(4-fluorophenyl)sulfamoyl]-3-(2,3,5-trichlorophenyl)acrylonitrile; (E)-2-[(2,4-dimethylphenyl)sulfamoyl]-3-(2-benzyloxyphenyl)acrylonitrile; (E)-2-[(2,4-dimethylphenyl)sulfamoyl]-3-(2-chlorophenyl)acrylonitrile; (E)-2-[(2,4-dimethylphenyl)sulfamoyl]-3-(3-methylthiophen-2-yl)acrylonitrile; (E)-2-[(2,4-dimethylphenyl)sulfamoyl]-3-(4-bromophenyl)acrylonitrile; (E)-2-[(2,4-dimethylphenyl)sulfamoyl]-3-(2,3,5-trichlorophenyl)acrylonitrile; (E)-2-[(2,4-dimethylphenyl)sulfamoyl]-3-(5-bromo-3-indolyl)acrylonitrile; (E)-2-[(4-methoxyphenyl)sulfamoyl]-3-(2-benzyloxyphenyl)acrylonitrile; (E)-2-[(4-methoxyphenyl)sulfamoyl]-3-(2,4,6-trimethoxyphenyl)acrylonitrile; (E)-2-[(4-methoxyphenyl)sulfamoyl]-3-(2,3,5-trichlorophenyl)acrylonitrile; (E)-2-[(4-methoxyphenyl)sulfamoyl]-3-(2,5-dimethoxyphenyl)acrylonitrile; and salts thereof.

114. A compound according to claim 1, wherein D is —C(═O)NR⁸₂, G is NR¹, m is 1, and n is 2.

115. A compound according to claim 1, wherein D is —NO₂, G is NR¹, m is 1, and n is 2.

116. A process for the synthesis of a compound according to claim 1 comprising: condensing a compound of formula II: 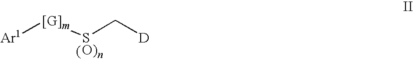
with an aromatic aldehyde of formula III: 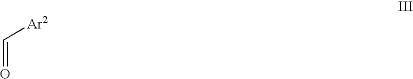
wherein Ar¹, Ar², G, D, m and n are as defined in claim 1.

117. A compound of formula IIA, or a salt thereof: 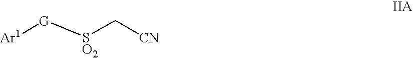
wherein G is CH₂ or NH, and Ar¹ is: 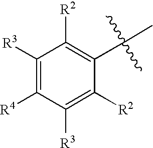
wherein:
each R² is independently selected from the group consisting of hydrogen, (C₁-C₆)alkyl, halogen, —OR⁷, —OAr³, —O(C₁-C₃)alkylene-Ar³, —C≡N, —NO₂, —C(═O)OR⁷, —C(═O)NR⁸₂, —C(═NR⁷)NR⁸₂, —(C₁-C₃)alkylene-C(═O)OR⁷, —O(C₁-C₃)alkylene-C(═O)OR⁷, —(C₁-C₆)alkylene-OR⁷, —P(═O)(OR⁷)₂, —OP(═O)(OR⁷)₂, —NHC(═O)(C₁-C₆)alkyl, —SO₂NR⁸₂, —OSO₂(C₁-C₆)alkyl, —OSO₂Ar³, —OC(═O)(C₁-C₃)alkyl, —O(C₂-C₆)alkylene-NR⁸₂, (C₁-C₃)perfluoroalkyl and —N(R⁶)-(M)_{y}-R⁵;
each R³ is independently selected from the group consisting of hydrogen, (C₁-C₆)alkyl, halogen, —OR⁷, —OAr³, —O(C₁-C₃)alkylene-Ar³, —C≡N, —NO₂, —C(═O)OR⁷, —C(═O)NR⁸₂, —C(═NR⁷)NR⁸₂, —(C₁-C₃)alkylene-C(═O)OR⁷, —O(C₁-C₃)alkylene-C(═O)OR⁷, —(C₁-C₆)alkylene-OR⁷, —P(═O)(OR⁷)₂, —OP(═O)(OR⁷)₂, —NHC(═O)(C₁-C₆)alkyl, —SO₂NR⁸², —OSO₂(C₁-C₆)alkyl, —OSO₂Ar³, —OC(═O)(C₁-C₃)alkyl, —O(C₂-C₆)alkylene-NR⁸₂, (C₁-C₃)perfluoroalkyl and —N(R⁶)-(M)_{y}-R⁵;
R⁴ is selected from the group consisting of hydrogen, (C₁-C₆)alkyl, halogen, —OR⁷, —OAr³, —O(C₁-C₃)alkylene-Ar³, —C≡N, —NO₂, —C(═O)OR⁷, —C(═O)NR⁸₂, C(═NR⁷)NR⁸, —(C₁-C₃)alkylene-C(═O)OR⁷, —O(C₁-C₃)alkylene-C(═O)OR⁷, —(C₁-C₆)alkylene-OR⁷, P(═O)(OR⁷)₂, —OP(═O)(OR⁷)₂, —NHC(═O)(C₁-C₆)alkyl, —SO₂NR⁸₂, —OSO₂(C₁-C₆)alkyl, —OSO₂Ar³, —OC(═O)(C₁-C₃)alkyl, —O(C₂-C₆)alkylene-NR⁸₂, (C₁-C₃)perfluoroalkyl and —N(R⁶)M_{y}(R⁵);
each Ar³ is phenyl optionally substituted with 1, 2, 3, 4, or 5 substituents independently selected from the group consisting of (C₁-C₃)alkyl, (C₁-C₃)alkoxy and halogen;
each M is a connecting group independently selected from the group consisting of —(C₁-C₆)alkylene-, —(CH₂)_{d}—V—(CH₂)ₑ—, —(CH₂)_{f}—W—(CH₂)_{g}— and -Z-;
each y is independently selected from the group consisting of 0 and 1;
each V is independently selected from the group consisting of arylene, heteroarylene, —C(═O)—, —C(═O)(C₁-C₆)perfluoroalkylene, —C(═O)—, —C(═S)—, —S(═O)—, —SO₂—, —C(═O)NR⁷—, —C(═S)NR⁷— and —SO₂NR⁷—;
each W is independently selected from the group consisting of —NR⁷—, —O— and —S—;
each d is independently selected from the group consisting of 0, 1 and 2;
each e is independently selected from the group consisting of 0, 1 and 2;
each f is independently selected from the group consisting of 1, 2 and 3;
each g is independently selected from the group consisting of 0, 1 and 2;
-Z- is 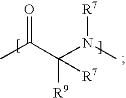
wherein the absolute configuration of -Z- is (S) or (R), or a mixture of (S) and (R);
each R⁵ is independently selected from the group consisting of hydrogen, unsubstituted aryl, substituted aryl, unsubstituted heterocyclic, substituted heterocyclic, —C(═O)OR⁷, —C(═O)NR⁸₂, —C(═NH)—NR⁸₂, —(C₁-C₆)perfluoroalkyl, —CF₂Cl, —P(═O)(OR⁷)₂, —CR⁷R¹⁰R¹¹ and a monovalent peptidyl group with a molecular weight of less than 1000, provided that when y is 0, R⁵ is not —C(═O)OH; and
each R⁶ is independently selected from the group consisting of —H, —(C₁-C₆)alkyl and aryl(C₁-C₃)alkyl; or
optionally, within any occurrence of —N(R⁶)M_{y}(R⁵) where y is 1, independently of any other occurrence of —N(R⁶)M_{y}(R⁵), R⁵ and R⁶ in combination represent a single bond and M is selected such that the resulting —N(R⁶)M_{y}(R⁵) moiety represents a 5, 6, or 7-membered ring heterocycle;
each R⁷ is independently selected from the group consisting of hydrogen and (C₁-C₆)alkyl;
each R⁸ is independently hydrogen or (C₁-C₆)alkyl; or, optionally, within any occurrence of NR⁸², independently of any other occurrence of NR⁸₂, two R⁸ groups in combination are —(CH₂)ₕ— or —(CH₂)ᵢX(CH₂)₂—;
wherein:
h is 4, 5, or 6;
i is 2 or 3;
X is O, S, NR⁷, or a single bond;
each R⁹ is independently selected from the group consisting of —H, —(C₁-C₆)alkyl, —(CH₂)₃—NH—C(NH₂)(═NH), —CH₂C(═O)NH₂, —CH₂C(═O)OH, —CH₂SH, —(CH₂)₂C(═O)—NH₂, —(CH₂)₂C(═O)OH, —CH₂-(2-imidazolyl), —(CH₂)₄—NH₂, —(CH₂)₂—S—CH₃, phenyl, CH₂-phenyl, —CH₂—OH, —CH(OH)—CH₃, —CH₂-(3-indolyl) and —CH₂-(4-hydroxyphenyl);
each R¹¹ is independently selected from the group consisting of —H, —(C₁-C₆)alkyl, —C(═O)OR⁷, —C(═O)NR⁸₂, —OR⁷, —SR⁷, —OC(═O)(CH₂)₂C(═O)OR⁷ guanidino, —NR², —NR⁷₃⁺, —N⁺(CH₂CH₂OR⁷)₃, phenyl, substituted phenyl, heterocyclyl and substituted heterocyclyl;
each R¹¹ is independently selected from the group consisting of R⁹, halogen, —NR⁸₂ and heterocycles containing two nitrogen atoms;
wherein the substituents for the substituted aryl and substituted heterocyclic groups comprising or included within R⁵ and R¹⁰ are independently selected from the group consisting of halogen, (C₁-C₆)alkyl, —OR⁷, —NO₂, —C≡N, —C(═O)OR⁷, —C(═O)NR⁸₂, —C(═NR⁷)NR⁸₂, —(C₁-C₃)alkylene-C(═O)OR⁷, —O(C₁-C₃)alkylene-C(═O)OR⁷, —(C₁-C₆)alkylene-OR⁷, —NR⁸₂, —P(═O)(OR⁷)₂, —OP(═O)(OR⁷)₂, —SO₂NR⁸₂, —NHC(═O)(C₁-C₆)alkyl, —OC(═O)(C₁-C₃)alkyl, —O(C₂-C₆)alkylene-NR⁸₂ and (C₁-C₃)perfluoroalkyl.

118. A pharmaceutical composition comprising a pharmaceutically acceptable carrier and at least one compound according to formula I, or a pharmaceutically acceptable salt thereof:
wherein: 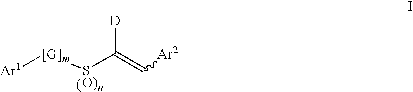
Ar¹ is: 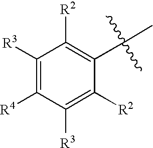
Ar² is selected from the group consisting of unsubstituted aryl, substituted aryl, unsubstituted heteroaryl and substituted heteroaryl; wherein, when Ar² is substituted aryl or substituted heteroaryl, said substituted aryl or substituted heteroaryl is aryl or heteroaryl substituted with one or more substituents independently selected from the group consisting of halogen, (C₁-C₆)alkyl, (C₂-C₆)alkenyl, (C₂-C₆)alkynyl, —(C₁-C₃)alkylene-Ar³, —OR⁷, —OAr³, —O(C₁-C₃)alkylene-Ar³, (C₁-C₃)fluoroalkoxy, —NO₂, —C≡N, —C(═O)(C₁-C₃)alkyl, —C(═O)OR⁷, —C(═O)NR⁸₂, —C(═NR⁷)NR⁸₂, —(C₁-C₃)alkylene-C(═O)OR⁷, —O(C₁-C₃)alkylene-C(═O)OR⁷, —(C₁-C₆)alkylene-OR⁷, NR⁸₂, —P(═O)(OR⁷)₂, —OP(═O)(OR⁷)₂, —S(C₁-C₆)alkyl, —S(O)(C₁-C₆)alkyl, —SO₂(C₁-C₆)alkyl, —SO₂NR⁸₂, —OSO₂(C₁-C₆)alkyl, —OSO₂Ar³, —NHC(═O)(C₁-C₆)alkyl, —OC(═O)(C₁-C₃)alkyl, —O(C₂-C₆)alkylene-NR⁸₂ and (C₁-C₃)perfluoroalkyl;
D is —C≡N, —C(═O)NR⁸₂, or NO₂;
G is CR¹₂ or NR¹;
R¹ is independently selected from the group consisting of hydrogen and (C₁-C₆)alkyl;
each R² is independently selected from the group consisting of hydrogen, (C₁-C₆)alkyl, halogen, —OR⁷, —OAr³, —O(C₁-C₃)alkylene-Ar³, —C≡N, —NO₂, —C(═O)OR⁷, —C(═O)NR⁸₂, —C(═NR⁷)NR⁸₂, —(C₁-C₃)alkylene-C(═O)OR⁷, —O(C₁-C₃)alkylene-C(═O)OR⁷, —(C₁-C₆)alkylene-OR⁷, —P(═O)(OR⁷)₂, —OP(═O)(OR⁷)₂, —NHC(═O)(C₁-C₆)alkyl, —SO₂NR⁸₂, —OSO₂(C₁-C₆)alkyl, —OSO₂Ar³, —OC(═O)(C₁-C₃)alkyl, —O(C₂-C₆)alkylene-NR⁸₂, (C₁-C₃)perfluoroalkyl and —N(R⁶)-(M)_{y}-R⁵;
each R³ is independently selected from the group consisting of hydrogen, (C₁-C₆)alkyl, halogen, —OR⁷, —OAr³, —O(C₁-C₃)alkylene-Ar³, —C≡N, —NO₂, —C(═O)OR⁷, —C(═O)NR⁸₂, —C(═NR⁷)NR⁸₂, —(C₁-C₃)alkylene-C(═O)OR⁷, —O(C₁-C₃)alkylene-C(═O)OR⁷, —(C₁-C₆)alkylene-OR⁷, —P(═O)(OR⁷)₂, —OP(═O)(OR⁷)₂, —NHC(═O)(C₁-C₆)alkyl, —SO₂NR⁸₂, —OSO₂(C₁-C₆)alkyl, —OSO₂Ar³, —OC(═O)(C₁-C₃)alkyl, —O(C₂-C₆)alkylene-NR⁸₂, (C₁-C₃)perfluoroalkyl and —N(R⁶)-(M)_{y}-R;
R⁴ is selected from the group consisting of hydrogen, (C₁-C₆)alkyl, halogen, —OR⁷, —OAr³, —O(C₁-C₃)alkylene-Ar³, —C≡N, —NO₂, —C(═O)OR⁷, —C(═O)NR⁸₂, —C(═NR⁷)NR⁸₂, —(C₁-C₃)alkylene-C(═O)OR⁷, —O(C₁-C₃)alkylene-C(═O)OR⁷, —(C₁-C₆)alkylene-OR⁷, —P(═O)(OR⁷)₂, —OP(═O)(OR⁷)₂, —NHC(═O)(C₁-C₆)alkyl, —SO₂NR⁸₂, —OSO₂(C₁-C₆)alkyl, —OSO₂Ar³, —OC(═O)(C₁-C₃)alkyl, —O(C₂-C₆)alkylene-NR⁸₂, (C₁-C₃)perfluoroalkyl and —N(R⁶)M_{y}(R⁵);
each Ar³ is phenyl optionally substituted with 1, 2, 3, 4, or 5 substituents independently selected from the group consisting of (C₁-C₃)alkyl, (C₁-C₃)alkoxy and halogen;
each M is a connecting group independently selected from the group consisting of —(C₁-C₆)alkylene-, —(CH₂)_{d}—V—(CH₂)ₑ—, —(CH₂)^{f}—W—(CH₂)_{g}— and -Z-;
each y is independently selected from the group consisting of 0 and 1;
each V is independently selected from the group consisting of arylene, heteroarylene, —C(═O)—, —C(═O)(C₁-C₆)perfluoroalkylene, —C(═O)—, —C(═S)—, —S(═O)—, —SO₂—, —C(═O)NR⁷—, —C(═S)NR⁷— and —SO₂NR⁷—;
each W is independently selected from the group consisting of —NR⁷—, —O— and —S—;
each d is independently selected from the group consisting of 0, 1 and 2;
each e is independently selected from the group consisting of 0, 1 and 2;
each f is independently selected from the group consisting of 1, 2 and 3;
each g is independently selected from the group consisting of 0, 1 and 2;
-Z- is 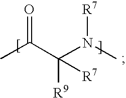
wherein the absolute configuration of -Z- is (S) or (R), or a mixture of (S) and (R);
each R⁵ is independently selected from the group consisting of hydrogen, unsubstituted aryl, substituted aryl, unsubstituted heterocyclic, substituted heterocyclic, —C(═O)OR⁷, —C(═O)NR⁸₂, —C(═NH)—NR⁸₂, —(C₁-C₆)perfluoroalkyl, —CF₂Cl, —P(═O)(OR⁷)₂, —CR⁷R¹⁰R¹¹ and a monovalent peptidyl group with a molecular weight of less than 1000, provided that when y is 0, R⁵ is not —C(═O)OH; and
each R⁶ is independently selected from the group consisting of —H, —(C₁-C₆)alkyl and aryl(C₁-C₃)alkyl; or
optionally, within any occurrence of —N(R⁶)M_{y}(R⁵) where y is 1, independently of any other occurrence of —N(R⁶)M_{y}(R⁵), R⁵ and R⁶ in combination represent a single bond and M is selected such that the resulting —N(R⁶)M_{y}(R⁵) moiety represents a 5, 6, or 7-membered ring heterocycle;
each R⁷ is independently selected from the group consisting of hydrogen and (C₁-C₆)alkyl; each R⁸ is independently hydrogen or (C₁-C₆)alkyl; or, optionally, within any occurrence of NR⁸₂, independently of any other occurrence of NR², two R groups in combination are —(CH₂)ₕ— or —(CH₂)ᵢX(CH₂)₂—;
wherein:
h is 4, 5, or 6;
i is 2 or 3;
X is O, S, NR⁷, or a single bond;
each R⁹ is independently selected from the group consisting of —H, —(C₁-C₆)alkyl, —(CH₂)₃—NH—C(NH₂)(═NH), —CH₂C(═O)NH₂, —CH₂C(═O)OH, —CH₂SH, —(CH₂)₂C(═O)—NH₂, —(CH₂)₂C(═O)OH, —CH₂-(2-imidazolyl), —(CH₂)₄—NH₂, —(CH₂)₂—S—CH₃, phenyl, CH₂-phenyl, —CH₂—OH, —CH(OH)—CH₃, —CH₂-(3-indolyl) and —CH₂-(4-hydroxyphenyl);
each R¹⁰ is independently selected from the group consisting of —H, —(C₁-C₆)alkyl, —C(═O)OR⁷, —C(═O)NR⁸₂, —OR⁷; —SR⁷, —OC(═O)(CH₂)₂C(═O)OR⁷, guanidino, —NR², —NR¹³₃⁺, —N⁺(CH₂CH₂OR⁷)₃, phenyl, substituted phenyl, heterocyclyl and substituted heterocyclyl;
each R¹¹ is independently selected from the group consisting of R⁹, halogen, —NR⁸₂ and heterocycles containing two nitrogen atoms;
wherein the substituents for the substituted aryl and substituted heterocyclic groups comprising or included within R⁵ and R¹⁰ are independently selected from the group consisting of halogen, (C₁-C₆)alkyl, —OR⁷, —NO₂, —C≡N, —C(═O)OR⁷, —C(═O)NR⁸₂, C(═NR⁷)NR⁸₂, —(C₁-C₃)alkylene-C(═O)OR⁷, —O(C₁-C₃)alkylene-C(═O)OR⁷, —(C₁-C₆)alkylene-OR⁷, —NR², —P(═O)(OR⁷)₂, —OP(═O)(OR⁷)₂, —SO₂NR⁸₂, —NHC(═O)(C₁-C₆)alkyl, —OC(═O)(C₁-C₃)alkyl, —O(C₂-C₆)alkylene-NR⁸₂ and (C₁-C₃)perfluoroalkyl;
m is 0 or 1, provided that if D is —C≡N, then m is 1;
n is 0, 1, or 2, provided that if G is NR¹ then n is 2; [symbol]
indicates a single bond, whereby the configuration of the S—C═C—Ar² double bond may be either E or Z;
with the provisos that:
(i) if Ar¹ is unsubstituted phenyl, D is CN, G is CH₂, m is 1, and n is 2, then Ar² is other than unsubstituted phenyl;
(ii) if Ar¹ is 4-chlorophenyl, D is CN, G is CH₂, m is 1, and n is 2, then Ar² is other than 3,5-di-(t-butyl)-4-hydroxyphenyl;
(iii) if Ar¹ is unsubstituted phenyl, D is CN, G is CH₂, m is 1, n is 2, and Ar² is substituted phenyl, then Ar² is substituted at the 4-position by other than hydroxyl; and
(iv) if Ar¹ is 3-trifluoromethoxyphenyl, D is CN, G is CH₂, m is 1, n is 2, and Ar² is substituted phenyl, then Ar² is substituted at the 4-position by other than hydroxyl and
(v) if Ar² is unsubstituted phenyl, D is C(═O)NH₂, m is 0, and n is 2, then R⁴ is other than NH₂, NHCHO or NHC(═O)alkyl.

119. A composition according to claim 118, wherein D is C(═O)NH₂, m is 0, and n is 2, and Ar¹ is unsubstituted phenyl.

120. A composition according to claim 118, wherein D is C(═O)NH₂, m is 0, and n is 2, and Ar¹ is other than unsubstituted phenyl.

121. A pharmaceutical composition comprising a compound according to claim 1, or a pharmaceutically acceptable salt thereof, and a pharmaceutically acceptable carrier.

122. A conjugate of formula I-L-Ab, wherein:
I is a compound according to claim 1, or a salt thereof;
Ab is an antibody; and
-L- is a single covalent bond or linking group covalently linking said compound to said antibody.

123. A conjugate according to claim 122 wherein the antibody is a monoclonal antibody or a monospecific polyclonal antibody.

124. A conjugate according to claim 123 wherein the antibody is a tumor specific antibody.

125. A method of treating an individual for a cellular proliferative disorder comprising administering to said individual an effective amount of at least one compound according to formula I, or a pharmaceutically acceptable salt thereof: 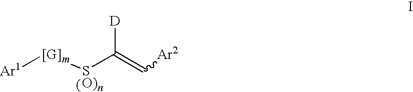
wherein:
Ar¹ is: 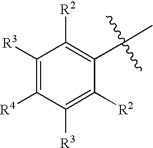
Ar² is selected from the group consisting of unsubstituted aryl, substituted aryl, unsubstituted heteroaryl and substituted heteroaryl; wherein, when Ar² is substituted aryl or substituted heteroaryl, said substituted aryl or substituted heteroaryl is aryl or heteroaryl substituted with one or more substituents independently selected from the group consisting of halogen, (C₁-C₆)alkyl, (C₂-C₆)alkenyl, (C₂-C₆)alkynyl, —(C₁-C₃)alkylene-Ar³, —OR⁷, —OAr³, —O(C₁-C₃)alkylene-Ar³; (C₁-C₃)fluoroalkoxy, —NO₂, —C≡N, —C(═O)(C₁-C₃)alkyl, —C(═O)OR⁷, —C(═O)NR⁸₂, —C(═NR⁷)NR⁸₂, —(C₁-C₃)alkylene-C(═O)OR⁷, —O(C₁-C₃)alkylene-C(═O)OR⁷, —(C₁-C₆)alkylene-OR⁷, —NR⁸₂, —P(═O)(OR⁷)₂, —OP(═O)(OR⁷)₂, —S(C₁-C₆)alkyl, —S(O)(C₁-C₆)alkyl, —SO₂(C₁-C₆)alkyl, —SO₂NR⁸₂, —OSO₂(C₁-C₆)alkyl, —OSO₂Ar³, —NHC(═O)(C₁-C₆)alkyl, —OC(═O)(C₁-C₃)alkyl, —O(C₂-C₆)alkylene-NR⁸₂ and (C₁-C₃)perfluoroalkyl;
D is —C≡N, —C(═O)NR⁸₂, or NO₂;
G is CR¹₂ or NR
R¹ is independently selected from the group consisting of hydrogen and (C₁-C₆)alkyl;
each R² is independently selected from the group consisting of hydrogen, (C₁-C₆)alkyl, halogen, —OR⁷, —OAr³, —O(C₁-C₃)alkylene-Ar³, —C≡N, —NO₂, —C(═O)OR⁷, —C(═O)NR⁸₂, —C(═NR⁷)NR⁸₂, —(C₁-C₃)alkylene-C(═O)OR⁷, —O(C₁-C₃)alkylene-C(═O)OR⁷, —(C₁-C₆)alkylene-OR⁷, —P(═O)(OR⁷)₂, —OP(═O)(OR⁷)₂, —NHC(═O)(C₁-C₆)alkyl, —SO₂NR², —OSO₂(C₁-C₆)alkyl, —OSO₂Ar³, —OC(═O)(C₁-C₃)alkyl, —O(C₂-C₆)alkylene-NR⁸₂, (C₁-C₃)perfluoroalkyl and —N(R⁶)-(M)_{y}-R⁵;
each R³ is independently selected from the group consisting of hydrogen, (C₁-C₆)alkyl, halogen, —OR⁷, —OAr³, —O(C₁-C₃)alkylene-Ar³, —C≡N, —NO₂, —C(═O)OR⁷, —C(═O)NR⁸₂, —C(═NR⁷)NR⁸₂, —(C₁-C₃)alkylene-C(═O)OR⁷, —O(C₁-C₃)alkylene-C(═O)OR⁷, —(C₁-C₆)alkylene-OR⁷, —P(═O)(OR⁷)₂, —OP(═O)(OR⁷)₂, —NHC(═O)(C₁-C₆)alkyl, —SO₂NR⁸₂, —OSO₂(C₁-C₆)alkyl, —OSO₂Ar³, —OC(═O)(C₁-C₃)alkyl, —O(C₂-C₆)alkylene-NR⁸₂, (C₁-C₃)perfluoroalkyl and —N(R⁶)-(M)_{y}-R⁵;
R⁴ is selected from the group consisting of hydrogen, (C₁-C₆)alkyl, halogen, —OR⁷, —OAr³, —O(C₁-C₃)alkylene-Ar³, —C≡N, —NO₂, —C(═O)OR⁷, —C(═O)NR⁸₂, —C(═NR⁷)NR⁸₂, —(C₁-C₃)alkylene-C(═O)OR⁷, O(C₁-C₃)alkylene-C(═O)R⁷, —(C₁-C₆)alkylene-OR⁷, —P(═O)(OR⁷)₂, —OP(═O)(OR⁷)₂, —NHC(═O)(C₁-C₆)alkyl, —SO₂NR⁸₂, —OSO₂(C₁-C₆)alkyl, —OSO₂Ar³, —OC(═O)(C₁-C₃)alkyl, —O(C₂-C₆)alkylene-NR⁸₂, (C₁-C₃)perfluoroalkyl and —N(R⁶)M_{y}(R⁵);
each Ar³ is phenyl optionally substituted with 1, 2, 3, 4, or 5 substituents independently selected from the group consisting of (C₁-C₃)alkyl, (C₁-C₃)alkoxy and halogen;
each M is a connecting group independently selected from the group consisting of —(C₁-C₆)alkylene-, —(CH₂)_{d}—V—(CH₂)ₑ—, —(CH₂)_{f}—W—(CH₂)_{g} and -Z-;
each y is independently selected from the group consisting of 0 and 1;
each V is independently selected from the group consisting of arylene, heteroarylene, —C(═O)—, —C(═O)(C₁-C₆)perfluoroalkylene, —C(═O)—, —C(═S)—, —S(═O)—, —SO₂—, —C(═O)NR⁷—, —C(═S)NR⁷— and —SO₂NR⁷—;
each W is independently selected from the group consisting of —NR⁷—, —O— and —S—;
each d is independently selected from the group consisting of 0, 1 and 2;
each e is independently selected from the group consisting of 0, 1 and 2;
each f is independently selected from the group consisting of 1, 2 and 3;
each g is independently selected from the group consisting of 0, 1 and 2;
-Z- is 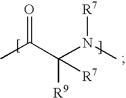
wherein the absolute configuration of -Z- is (S) or (R), or a mixture of (S) and (R);
each R⁵ is independently selected from the group consisting of hydrogen, unsubstituted aryl, substituted aryl, unsubstituted heterocyclic, substituted heterocyclic, —C(═O)OR⁷, —C(═O)NR⁸₂, —C(═NH)—NR⁸₂, —(C₁-C₆)perfluoroalkyl, —CF₂Cl, —P(═O)(OR⁷)₂, —CR⁷R¹⁰R¹¹ and a monovalent peptidyl group with a molecular weight of less than 1000, provided that when y is 0, R⁵ is not —C(═O)OH; and
each R⁶ is independently selected from the group consisting of —H, —(C₁-C₆)alkyl and aryl(C₁-C₃)alkyl; or
optionally, within any occurrence of —N(R⁶)M_{y}(R⁵) where y is 1, independently of any other occurrence of —N(R⁶)M_{y}(R⁵), R⁵ and R⁶ in combination represent a single bond and M is selected such that the resulting —N(R⁶)M_{y}(R⁵) moiety represents a 5, 6, or 7-membered ring heterocycle;
each R⁷ is independently selected from the group consisting of hydrogen and (C₁-C₆)alkyl;
each R⁸ is independently hydrogen or (C₁-C₆)alkyl; or, optionally, within any occurrence of NR⁸₂, independently of any other occurrence of NR⁸₂, two R⁸ groups in combination are —(CH₂)ₕ— or —(CH₂)ᵢX(CH₂)₂—;
wherein:
h is 4, 5, or 6;
i is 2 or 3;
X is O, S, NR⁷, or a single bond;
each R⁹ is independently selected from the group consisting of —H, —(C₁-C₆)alkyl, —(CH₂)₃—NH—C(NH₂)(═NH), —CH₂C(═O)NH₂, —CH₂C(═O)OH, —CH₂SH, —(CH₂)₂C(═O)—NH₂, —(CH₂)₂C(═O)OH, —CH₂-(2-imidazolyl), —(CH₂)₄—NH₂, —(CH₂)₂—S—CH₃, phenyl, CH₂-phenyl, —CH₂—OH, —CH(OH)—CH₃, —CH₂-(3-indolyl) and —CH₂-(4-hydroxyphenyl);
each R¹⁰ is independently selected from the group consisting of —H, —(C₁-C₆)alkyl, —C(═O)OR⁷, —C(═O)NR⁸₂, —OR⁷, —SR⁷, —OC(═O)(CH₂)₂C(═O)OR⁷, guanidino, —NR², —NR⁷₃⁺, —N⁺(CH₂CH₂OR⁷)₃, phenyl, substituted phenyl, heterocyclyl and substituted heterocyclyl;
each R¹¹ is independently selected from the group consisting of R⁹, halogen, —NR⁸₂ and heterocycles containing two nitrogen atoms;
wherein the substituents for the substituted aryl and substituted heterocyclic groups comprising or included within R⁵ and R¹⁰ are independently selected from the group consisting of halogen, (C₁-C₆)alkyl, —OR⁷, —NO₂, —C≡N, —C(═O)OR⁷, —C(NR⁷)NR⁸₂, —(C₁-C₃)alkylene-C(═O)OR⁷, —O(C₁-C₃)alkylene-C(═O)OR⁷, —(C₁-C₆)alkylene-OR⁷, —NR⁸₂, —P(═O)(OR⁷)₂, —OP(═O)(OR⁷)₂, —SO₂NR⁸₂, —NHC(═O)(C₁-C₆)alkyl, —OC(═O)(C₁-C₃)alkyl, —O(C₂-C₆)alkylene-NR⁸₂ and (C₁-C₃)perfluoroalkyl;
m is 0 or 1, provided that if D is —C≡N, then m is 1;
n is 0, 1, or 2, provided that if G is NR¹ then n is 2; [symbol]
indicates a single bond, whereby the configuration of the S—C═C—Ar² double bond may be either E or Z;
with the provisos that:
(i) if Ar¹ is unsubstituted phenyl, D is CN, G is CH₂, m is 1, and n is 2, then Ar² is other than unsubstituted phenyl;
(ii) if Ar¹ is 4-chlorophenyl, D is CN, G is CH₂, m is 1, and n is 2, then Ar² is other than 3,5-di-(t-butyl)-4-hydroxyphenyl;
(iii) if Ar¹ is unsubstituted phenyl, D is CN, G is CH₂, m is 1, n is 2, and Ar² is substituted phenyl, then Ar² is substituted at the 4-position by other than hydroxyl; and
(iv) if Ar¹ is 3-trifluoromethoxyphenyl, D is CN, G is CH₂, m is 1, n is 2, and Ar² is substituted phenyl, then Ar² is substituted at the 4-position by other than hydroxyl.

126. A method according to claim 125, wherein the cellular proliferative disorder is selected from the group consisting of hemangiomatosis in newborn, secondary progressive multiple sclerosis, atherosclerosis, chronic progressive myelodegenerative disease, neurofibromatosis, ganglioneuromatosis, keloid formation, Paget's disease of the bone, fibrocystic disease of the breast, uterine fibroids, Peyronie's disease, Dupuytren's disease, restenosis, benign proliferative breast disease, benign prostatic hyperplasia, X-linked lymphocellular proliferative disorder, post-transplantation lymphocellular proliferative disorder, macular degeneration, retinopathies, proliferative vitreoretinopathy and non-cancerous lymphocellular proliferative disorders.

127. A method according to claim 125, wherein the cellular proliferative disorder is cancer.

128. A method according to claim 127, wherein the cancer is selected from the group consisting of ovarian cancer; cervical cancer; breast cancer; prostate cancer; testicular cancer, lung cancer, renal cancer; colorectal cancer; skin cancer; brain cancer; leukemia, including acute myeloid leukemia, chronic myeloid leukemia, acute lymphoid leukemia, and chronic lymphoid leukemia.

129. A method of inducing apoptosis of tumor cells in an individual afflicted with cancer comprising administering to said individual an effective amount of a compound according of formula I, or a pharmaceutically acceptable salt thereof: 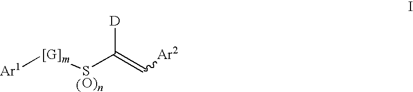
wherein:
Ar¹ is: 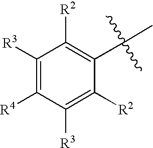
Ar¹ is selected from the group consisting of unsubstituted aryl, substituted aryl, unsubstituted heteroaryl and substituted heteroaryl; wherein, when Ar² is substituted aryl or substituted heteroaryl, said substituted aryl or substituted heteroaryl is aryl or heteroaryl substituted with one or more substituents independently selected from the group consisting of halogen, (C₁-C₆)alkyl, (C₂-C₆)alkenyl, (C₂-C₆)alkynyl, —(C₁-C₃)alkylene-Ar³, —OR⁷, —OAr³, —O(C₁-C₃)alkylene-Ar³, (C₁-C₃)fluoroalkoxy, —NO₂, —C≡N, —C(═O)(C₁-C₃)alkyl, —C(═O)OR⁷, —C(═O)NR⁸₂, —C(═NR⁷)NR⁸₂, —(C₁-C₃)alkylene-C(═O)OR⁷, —O(C₁-C₃)alkylene-C(═O)OR⁷, —(C₁-C₆)alkylene-OR⁷, —NR⁸₂, —P(═O)(OR⁷)₂, —OP(═O)(OR⁷)₂, —S(C₁-C₆)alkyl, —S(O)(C₁-C₆)alkyl, —SO₂(C₁-C₆)alkyl, —SO₂NR², —OSO₂(C₁-C₆)alkyl, —OSO₂Ar³, —NHC(═O)(C₁-C₆)alkyl, —OC(═O)(C₁-C₃)alkyl, —O(C₂-C₆)alkylene-NR⁸₂ and (C₁-C₃)perfluoroalkyl;
D is —C≡N, —C(═O)NR⁸₂, or NO₂;
G is CR⁸₂ or NR¹;
R¹ is independently selected from the group consisting of hydrogen and (C₁-C₆)alkyl;
each R² is independently selected from the group consisting of hydrogen, (C₁-C₆)alkyl, halogen, —OR⁷, —OAr³, —O(C₁-C₃)alkylene-Ar³, —C≡N, —NO₂, —C(═O)OR⁷, —C(═O)NR⁸₂, —C(═NR⁷)NR⁸₂, —(C₁-C₃)alkylene-C(═O)OR⁷, —O(C₁-C₃)alkylene-C(═O)OR⁷, —(C₁-C₆)alkylene-OR⁷, —P(═O)(OR⁷)₂, —OP(═O)(OR⁷)₂, —NHC(═O)(C₁-C₆)alkyl, —SO₂NR⁸₂, —OSO₂(C₁-C₆)alkyl, —OSO₂Ar³, —OC(═O)(C₁-C₃)alkyl, —O(C₂-C₆)alkylene-NR⁸₂, (C₁-C₃)perfluoroalkyl and —N(R⁶)-(M)_{y}-R⁵;
each R³ is independently selected from the group consisting of hydrogen, (C₁-C₆)alkyl, halogen, —OR⁷, —OAr³, —O(C₁-C₃)alkylene-Ar³, —C≡N, —NO₂, —C(═O)OR⁷, —C(═O)NR⁸₂, —C(═NR⁷)NR⁸₂, —(C₁-C₃)alkylene-C(═O)OR⁷, —O(C₁-C₃)alkylene-C(═O)OR⁷, —(C₁-C₆)alkylene-OR⁷, —P(═O)(OR⁷)₂, —OP(═O)(OR⁷)₂, —NHC(═O)(C₁-C₆)alkyl, —SO₂NR⁸₂, —OSO₂(C₁-C₆)alkyl, —OSO₂Ar³, —OC(═O)(C₁-C₃)alkyl, —O(C₂-C₆)alkylene-NR⁸₂, (C₁-C₃)perfluoroalkyl and —N(R⁶)-(M)_{y}-R⁵;
R⁴ is selected from the group consisting of hydrogen, (C₁-C₆)alkyl, halogen, —OR⁷, —OAr³, —O(C₁-C₃)alkylene, Ar³, —C≡N, —NO₂, —C(═O)OR⁷, —C(═O)NR⁸₂, —C(═NR⁷)NR⁸₂, —(C₁-C₃)alkylene-C(═O)OR⁷, —O(C₁-C₃)alkylene-C(═O)OR⁷, —(C₁-C₆)alkylene-OR⁷, —P(═O)(OR⁷)₂, —OP(═O)(OR⁷)₂, —NHC(═O)(C₁-C₆)alkyl, —SO₂NR⁸₂, —OSO₂(C₁-C₆)alkyl, —OSO₂Ar³, —OC(═O)(C₁-C₃)alkyl, —O(C₂-C₆)alkylene-NR⁸₂, (C₁-C₃)perfluoroalkyl and —N(R⁶)M (R⁵);
each Ar³ is phenyl optionally substituted with 1, 2, 3, 4, or 5 substituents independently selected from the group consisting of (C₁-C₃)alkyl, (C₁-C₃)alkoxy and halogen;
each M is a connecting group independently selected from the group consisting of —(C₁-C₆)alkylene-, —(CH₂)_{d}—V—(CH₂)ₑ—, —(CH₂)_{f}—W—(CH₂)_{g}— and -Z-;
each y is independently selected from the group consisting of 0 and 1;
each V is independently selected from the group consisting of arylene, heteroarylene, —C(═O)—, —C(═O)(C₁-C₆)perfluoroalkylene, —C(═O)—, —C(═S)—, —S(═O)—, —SO₂—, —C(═O)NR⁷—, —C(═S)NR⁷— and —SO₂NR⁷—;
each W is independently selected from the group consisting of —NR⁷—, —O— and —S—;
each d is independently selected from the group consisting of 0, 1 and 2;
each e is independently selected from the group consisting of 0, 1 and 2;
each f is independently selected from the group consisting of 1, 2 and 3;
each g is independently selected from the group consisting of 0, 1 and 2;
-Z- is 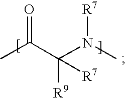
wherein the absolute configuration of -Z- is (S) or (R), or a mixture of (S) and (R);
each R⁵ is independently selected from the group consisting of hydrogen, unsubstituted aryl, substituted aryl, unsubstituted heterocyclic, substituted heterocyclic, —C(═O)OR⁷, —C(═O)NR⁸₂, —C(═NH)—NR⁸₂, —(C₁-C₆)perfluoroalkyl, —CF₂Cl, —P(═O)(OR⁷)₂, —CR⁷R¹⁰R¹¹ and a monovalent peptidyl group with a molecular weight of less than 1000, provided that when y is 0, R⁵ is not —C(═O)OH; and
each R⁶ is independently selected from the group consisting of —H, —(C₁-C₆)alkyl and aryl(C₁-C₃)alkyl; or
optionally, within any occurrence of —N(R⁶)M_{y}(R⁵) where y is 1, independently of any other occurrence of —N(R⁶)M_{y}(R⁵), R⁵ and R⁶ in combination represent a single bond and M is selected such that the resulting —N(R⁶)M_{y}(R⁵) moiety represents a 5, 6, or 7-membered ring heterocycle;
each R⁷ is independently selected from the group consisting of hydrogen and (C₁-C₆)alkyl;
each R⁸ is independently hydrogen or (C₁-C₆)alkyl; or, optionally, within any occurrence of NR⁸₂, independently of any other occurrence of NR⁸₂, two R⁸ groups in combination are —(CH₂)ₕ— or —(CH₂)ᵢX(CH₂)₂—;
wherein:
h is 4, 5, or 6;
i is 2 or 3;
X is O, S, NR⁷, or a single bond;
each R⁹ is independently selected from the group consisting of —H, —(C₁-C₆)alkyl, —(CH₂)₃—NH—C(NH₂)(═NH), —CH₂C(═O)NH₂, —CH₂C(═O)OH, —CH₂SH, —(CH₂)₂C(═O)—NH₂, —(CH₂)₂C(═O)OH, —CH₂-(2-imidazolyl), —(CH₂)₄—NH₂, —(CH₂)₂—S—CH₃, phenyl, CH₂-phenyl, —CH₂—OH, —CH(OH)—CH₃, —CH₂-(3-indolyl) and —CH₂-(4-hydroxyphenyl);
each R¹⁰ is independently selected from the group consisting of —H, —(C₁-C₆)alkyl, —C(═O)OR⁷, —C(═O)NR⁸₂, —OR⁷, —SR⁷, —OC(═O)(CH₂)₂C(═O)OR⁷, guanidino, —NR⁷₂, —NR⁷₃⁺, —N⁺(CH₂CH₂OR⁷)₃, phenyl, substituted phenyl, heterocyclyl and substituted heterocyclyl;
each R¹¹ is independently selected from the group consisting of R⁹, halogen, —NR² and heterocycles containing two nitrogen atoms;
wherein the substituents for the substituted aryl and substituted heterocyclic groups comprising or included within R⁵ and R¹⁰ are independently selected from the group consisting of halogen, (C₁-C₆)alkyl, —OR⁷, —NO₂, —C≡N, —C(═O)OR⁷, —C(═O)NR⁸₂, —C(═NR⁷)NR⁸₂, —(C₁-C₃)alkylene-C(═O)OR⁷, —O(C₁-C₃)alkylene-C(═O)OR⁷, —(C₁-C₆)alkylene-OR⁷, —NR⁸₂, —P(═O)(OR⁷)₂, —OP(═O)(OR⁷)₂, —SO₂NR⁸₂, —NHC(═O)(C₁-C₆)alkyl, —OC(═O)(C₁-C₃)alkyl, —O(C₂-C₆)alkylene-NR⁸₂ and (C₁-C₃)perfluoroalkyl;
m is 0 or 1, provided that if D is —C≡N, then m is 1;
n is 0, 1, or 2, provided that if G is NR¹ then n is 2; [symbol]
indicates a single bond, whereby the configuration of the S—C═C—Ar² double bond may be either E or Z;
with the provisos that:
(i) if Ar¹ is unsubstituted phenyl, D is CN, G is CH₂, m is 1, and n is 2, then Ar² is other than unsubstituted phenyl;
(ii) if Ar¹ is 4-chlorophenyl, D is CN, G is CH₂, m is 1, and n is 2, then Ar² is other than 3,5-di-(t-butyl)-4-hydroxyphenyl;
(iii) if Ar¹ is unsubstituted phenyl, D is CN, G is CH₂, m is 1, n is 2, and Ar² is substituted phenyl, then Ar² is substituted at the 4-position by other than hydroxyl; and
(iv) if Ar¹ is 3-trifluoromethoxyphenyl, D is CN, G is CH₂, m is 1, n is 2, and Ar² is substituted phenyl, then Ar² is substituted at the 4-position by other than hydroxyl.

130. A method according to claim 129, wherein the tumor cells are selected from the group consisting of ovarian, cervical, breast, prostate, testicular, lung, renal, colorectal, skin and brain tumor cells.

131. A method of treating an individual for a cellular proliferative disorder comprising administering to said individual an effective amount of at least one conjugate of according to claim 122.
